SEQUENCE LISTING (236 sequences)

SEQ ID NO: 1 - protein, 251 aa, Artificial Sequence
  synthetic construct; heavy chian of Garadacimab
EVQLLESGGGLVQPGGSLRLSCAASGFTFSKYIMQWVRQAPGKGLEWVSGIDIPTKGTVYADSVKGRFTISRDNSKNTLYLQMNSLRAEDTAVYYCARALPRSGYLISPHYYYYALDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFL

SEQ ID NO: 2 - protein, 215 aa, Artificial Sequence
  synthetic construct; light chain Garadacimab
QSVLTQPPSASGTPGQRVTISCSGSSSNIGRNYVYWYQQLPGTAPKLLIYSNNQRPSGVPDRFSGSKSGTSASLAISGLRSEDEADYYCAAWDASLRGVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 3 - protein, 119 aa, Artificial Sequence
  synthetic construct; heavy chain AB042/43
EVQLQQSGAELVRPGASVKLSCTASGFNIKDDYMHWVKQRPEQGLEWIGWIDPENGDTEYASKFQGKATITSDTSSNTAYLQLSSLTSEDTAVYYCTENYYGSSFFAYWGQGTLVTVSA

SEQ ID NO: 4 - protein, 112 aa, Artificial Sequence
  synthetic construct; light chain AB04 2/43
DIVMTQATPSVPVTPGESVSISCRSSKSLLHSNGITYLYWFLQRPGQSPQRLIYYMSNLASGVPDRFSGRGSGTDFTLRISRVEAEDVGVYYCMQSLEYPYTFGGGTKLEIK

SEQ ID NO: 5 - protein, 237 aa, Artificial Sequence
  synthetic construct; heavy chain AB054
  misc_feature (221)..(226): amino acids 221-226 can be present or absent
  misc_feature (227)..(237): amino acids 227-237 can be present or absent
QVQLQESGPGLVKPSQTLSLTCTVSGYSITSGYSWHWIRQHPGKGLEWIGYIQYSGNTNSNPSLKSRVTISRDTSKNQFSLKLSSVTAADTAVYYCARWGSFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFL

SEQ ID NO: 6 - protein, 213 aa, Artificial Sequence
  synthetic construct; light chain AB054
EIVLTQSPATLSLSPGERATLSCRASSSVNYLHWYQQKPGQAPRRLIYDTSKLATGIPARFSGSGSGTDYTLTISSLEPEDFAVYYCQQWSGNPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 248 aa, Artificial Sequence
  synthetic construct; heavy chain DX-4012
  misc (232)..(237): amino acids 232 to 237 can be present or absent
  misc (238)..(248): amino acids 238 to 248 can be present or absent
EVQLLESGGGLVQPGGSLRLSCAASGFTFSQYVMHWVRQAPGKGLEWVSSIWPSGGHTRYADSVKGRFTISRDNSKNTLYLQMNSLRAEDTAVYYCTRQRYRGPKYYYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFL

SEQ ID NO: 8 - protein, 123 aa, Artificial Sequence
  synthetic construct; light chain DX-4012
EVQLLESGGGLVQPGGSLRLSCAASGFTFSQYVMHWVRQAPGKGLEWVSSIWPSGGHTRYADSVKGRFTISRDNSKNTLYLQMNSLRAEDTAVYYCTRQRYRGPKYYYYMDVWGKGTTVTVSS

SEQ ID NO: 9 - protein, 713 aa, Artificial Sequence
  synthetic construct; scFv 620I-X0177-A01
EVQLLESGGGLVQPGGSLRLSCAASGFTFSHYIMMWVRQAPGKGLEWVSGIYSSGGITVYADSVKGRFTISRDNSKNTLYLQMNSLRAEDTAVYYCAYRRIGVPRRDEFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKSGGGSEVQLLESGGGLVQPGGSLRLSCAASGFTFSQYVMHWVRQAPGKCLEWVSSIWPSGGHTRYADSVKGRFTISRDNSKNTLYLQMNSLRAEDTAVYYCARQRYRGPKYYYYMDVWGQGTTVTVSSGGGGSGGGGSGGGGSGGGGSDIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPWTFGCGTKVEIKR

SEQ ID NO: 10 - protein, 125 aa, Artificial Sequence
  synthetic construct; heavy chain Ab26036
QVQLVQSGAEVKKPGASVTVSCKASGYTFTSYYLHWVRQAPGQGLEWMGIINPSGGSTSYAQKFQGRVTMTRDTSTSTVYMELSSLRSEDTAVYYCARGVTTVTTYYHYYNMDVWGQGTTVTVSS

SEQ ID NO: 11 - protein, 107 aa, Artificial Sequence
  synthetic construct; light chain Ab26036
DIQMTQSPSSLSASVGDRVTITCRASQGISNYLAWFQQKPGKAPKSLIYAASSLQSGVPSKFSGSGSGTDFTLTISSLQPEDFATYYCQKYNTYPLTFGGGTKVEIK

SEQ ID NO: 12 - protein, 121 aa, Artificial Sequence
  synthetic construct; heavy chain Ab26048
EVQLVESGGGLVQPRGSLRLSCVASGFTFSDYGMSWVRQAPGKGLEWVSVIGGAGHGTYYADSVKGRFTISRDNSRNTLYLQMNSLRDEDTAVYYCAKKYYWNYVGGMDVWGRGTTVTVSS

SEQ ID NO: 13 - protein, 107 aa, Artificial Sequence
  synthetic construct; light chian Ab26048
DIQMTQSPSSVSASVGDRVTITCRASQDINNWLAWYQQKPGKAPKLLISTASSLQSGVPSRFSGSGSGTDFTLTISSLQPEDFATYYCQQANSLPFTFGPGTKVDIK

SEQ ID NO: 14 - protein, 115 aa, Artificial Sequence
  synthetic construct; heavy chain Ab26049
EVQLVESGGDLVQPGGSLRLSCAASGFTFNTYAMHWVRQAPGKGLEYISAIDTKGGSTYYADSVKGRFTISRDNSKNAQYLQMDSLRVEDMAVYYCARGFGLDVWGQGTTVTVSS

SEQ ID NO: 15 - protein, 112 aa, Artificial Sequence
  synthetic construct; light chain Ab26049
DVVMTQSPLSLAVTLGQPASISCRSSQSLGYSDGNTYLNWFQQRPGQSPRRLIYKVSNRDSGVPDRFSGSGSGTDFTLKINRVETVDVGVYYCMQATHWPYTFGQGTKLEIK

SEQ ID NO: 16 - protein, 124 aa, Artificial Sequence
  synthetic construct; Ab26076
QVQLQQSGPGLVKPSQTLSLTCAISGDSVSSNSAAWNWIRQSPSRGLEWLGRTYYRSKWFNDYAVSVKSRIIINPDTTKNQFSLQVNSVTPEDTAVYYCARGEPARRGEYFHHWGQGTLVTVSS

SEQ ID NO: 17 - protein, 107 aa, Artificial Sequence
  synthetic construct; heavy chain Ab26076
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGKVEIK

SEQ ID NO: 18 - protein, 123 aa, Artificial Sequence
  synthetic construct; heavy chain 620I-X0173-A11
EVQLLESGGGLVQPGGSLRLSCAASGFTFSQYVMHWVRQAPGKCLEWVSSIWPSGGHTRYADSVKGRFTISRDNSKNTLYLQMNSLRAEDTAVYYCARQRYRGPKYYYYMDVWGQGTTVTVSS

SEQ ID NO: 19 - protein, 113 aa, Artificial Sequence
  synthetic construct; light chain 620I-X0173-A11
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPWTFGCGTKVEIKR

SEQ ID NO: 20 - protein, 123 aa, Artificial Sequence
  synthetic construct; heavy chain 620I-X0173-C07
EVQLLESGGGLVQPGGSLRLSCAASGFTFSWYVMHWVRQAPGKCLEWVSSIYPSGGKTSYADSVKGRFTISRDNSKNTLYLQMNSLRAEDTAVYYCARQRYRGPKYYYYMDVWGQGTTVTVSS

SEQ ID NO: 21 - protein, 112 aa, Artificial Sequence
  synthetic construct; light chain 620I-X0173-C07
DIVMTQSPLSLPVTPGEPASISCRSSQSLLHSNGYYLDWYLQKPGQSPQLLIYLGSNRASGVPDRFSGSGSGTDFTLKISRVEAEDVGVYYCMQALQTPWTFGCGTKVEIKR

SEQ ID NO: 22 - protein, 123 aa, Artificial Sequence
  synthetic construct; heavy chain 620I-X0173-E07
EVQLLESGGGLVQPGGSLRLSCAASGFTFSWYSMHWVRQAPGKCLEWVSVIYPSGGKTRYADSVKGRFTISRDNSKNTLYLQMNSLRAEDTAVYYCARQRYRGPKYYYYMDVWGQGTTVTVSS

SEQ ID NO: 23 - protein, 112 aa, Artificial Sequence; identical to SEQ ID NO: 21
  synthetic construct; light chain 620I-X0173-E07

SEQ ID NO: 24 - protein, 123 aa, Artificial Sequence
  synthetic construct; heavy chain 620I-X0173-G11
EVQLLESGGGLVQPGGSLRLSCAASGFTFSHYVMHWVRQAPGKCLEWVSSIYPSGGLTKYADSVKGRFTISRDNSKNTLYLQMNSLRAEDTAVYYCARQRYRGPKYYYYMDVWGQGTTVTVSS

SEQ ID NO: 25 - protein, 112 aa, Artificial Sequence; identical to SEQ ID NO: 21
  synthetic construct; light chain 620I-X0173-G11

SEQ ID NO: 26 - protein, 123 aa, Artificial Sequence
  synthetic construct; heavy chain 559C-M0071-F06
EVQLLESGGGLVQPGGSLRLSCAASGFTFSGYIMAWVRQAPGKGLEWVSYIYPSGGITVYADSVKGRFTISRDNSKNTLYLQMNSLRAEDTAVYYCTRQRYRGPKYYYYMDVWGKGTTVTVSS

SEQ ID NO: 27 - protein, 112 aa, Artificial Sequence
  synthetic construct; light chain 559C-M0071-F06
DIQMTQSPLSLPVTPGEPASISCRSSQSLLHSNGYYLDWYLQKPGQSPQLLIYLGSNRASGVPDRFSGSGSGTDFTLKISRVEAEDVGVYYCMQALQTPWTFGQGTKVEIKR

SEQ ID NO: 28 - protein, 112 aa, Artificial Sequence
  synthetic construct; light chain 559C-M0179-D04
DIQMTQSPLSLSVAPGEPASISCRSSQSLLHRNGHNYLDWYLQKPGQSPQLLIYLGSNRASGVPERFSGSGSGTDFTLRISRVEAEDVGVYYCMQALQARTFGQGTKVEIKR

SEQ ID NO: 29 - protein, 111 aa, Artificial Sequence
  synthetic construct; light chain 559C-M0181-C02
DIQMTQSPLSLPVTPGEPASISCRSSQSLLHSNGYYLDWYLQKPGQSPQLLIYLGSNRASGVPDRFSGSGSGTDFTLKISRVEAEDVGVYYCMQALQTRTFGQGTKVEIKR

SEQ ID NO: 30 - protein, 112 aa, Artificial Sequence
  synthetic construct; light chain 559C-M0180-G03
DIQMTQSPLSLPVTPGEPASISCRSSQSLLHSNGYYLDWYLQKPGQSPQIMIYLGSNRASGVPDRFSGSGSGTDFTLKISRVEAEDVGVYYCMQALQTPRTFGQGTKVEIKR

SEQ ID NO: 31 - protein, 123 aa, Artificial Sequence
  synthetic construct; heavy chian 559C-M0184-B04
EVQLLESGGGLVQPGGSLRLSCAASGFTFSFYSMHWVRQAPGKGLEWVSRIYPSGGVTKYADSVKGRFTISRDNSKNTLYLQMNSLRAEDTAVYYCTRQRYRGPKYYYYMDVWGKGTTVTVSS

SEQ ID NO: 32 - DNA, 753 bp, Artificial Sequence
  synthetic construct; heavy chain Garadacimab
gaggtgcagctgctggagagcggcggcggcctggtgcagcccggcggcagcctgaggctgagctgcgccgccagcggcttcaccttcagcaagtacatcatgcagtgggtgaggcaggcccccggcaagggcctggagtgggtgagcggcatcgacatccccaccaagggcaccgtgtacgccgacagcgtgaagggcaggttcaccatcagcagggacaacagcaagaacaccctgtacctgcagatgaacagcctgagggccgaggacaccgccgtgtactactgcgccagggccctgcccaggagcggctacctgatcagcccccactactactactacgccctggacgtgtggggccagggcaccaccgtgaccgtgagcagcgccagcaccaagggccccagcgtgttccccctggccccctgcagcaggagcaccagcgagagcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcaccaagacctacacctgcaacgtggaccacaagcccagcaacaccaaggtggacaagagggtggagagcaagtacggccccccctgccccccctgccccgcccccgagttcctgggcggccccagcgtgttcctg

SEQ ID NO: 33 - DNA, 645 bp, Artificial Sequence
  synthetic construct; light chain Garadacimab
cagagcgtgctgacccagccccccagcgccagcggcacccccggccagagggtgaccatcagctgcagcggcagcagcagcaacatcggcaggaactacgtgtactggtaccagcagctgcccggcaccgcccccaagctgctgatctacagcaacaaccagaggcccagcggcgtgcccgacaggttcagcggcagcaagagcggcaccagcgccagcctggccatcagcggcctgaggagcgaggacgaggccgactactactgcgccgcctgggacgccagcctgaggggcgtgttcggcggcggcaccaagctgaccgtgctgggccagcccaaggccgcccccagcgtgaccctgttcccccccagcagcgaggagctgcaggccaacaaggccaccctggtgtgcctgatcagcgacttctaccccggcgccgtgaccgtggcctggaaggccgacagcagccccgtgaaggccggcgtggagaccaccacccccagcaagcagagcaacaacaagtacgccgccagcagctacctgagcctgacccccgagcagtggaagagccacaggagctacagctgccaggtgacccacgagggcagcaccgtggagaagaccgtggcccccaccgagtgcagc

SEQ ID NO: 34 - DNA, 357 bp, Artificial Sequence
  synthetic construct; heavy chain AB042/43
gaggtgcagctgcagcagagcggcgccgagctggtgaggcccggcgccagcgtgaagctgagctgcaccgccagcggcttcaacatcaaggacgactacatgcactgggtgaagcagaggcccgagcagggcctggagtggatcggctggatcgaccccgagaacggcgacaccgagtacgccagcaagttccagggcaaggccaccatcaccagcgacaccagcagcaacaccgcctacctgcagctgagcagcctgaccagcgaggacaccgccgtgtactactgcaccgagaactactacggcagcagcttcttcgcctactggggccagggcaccctggtgaccgtgagcgcc

SEQ ID NO: 35 - DNA, 336 bp, Artificial Sequence
  synthetic construct; light chain AB042/43
gatattgtgatgactcaggctacaccctctgtacctgtcactcctggagagtcagtatccatctcctgcaggtctagtaagagtcttctgcatagtaatggcatcacttacttgtattggttcctgcagaggccaggccagtctcctcagcgcctgatatattatatgtccaaccttgcctcaggagtcccagacaggttcagtggcagagggtcaggaactgatttcacactgagaatcagtagagtggaggctgaggatgtgggtgtttattactgtatgcaaagtctagaatatccgtacacgttcggaggggggaccaagctggaaataaaa

SEQ ID NO: 36 - DNA, 1329 bp, Artificial Sequence
  synthetic construct; AB054
caggtgcagctccaggagagcggacccggtctggtgaagcccagccagaccctgagcctgacctgcaccgtgagcggctactcaatcacctctggctacagctggcactggatcaggcagcaccccggcaagggcctggagtggattggctatatccagtacagcggcaacaccaacagcaaccccagcctcaagagcagggtgaccatcagcagggatacaagcaagaaccagttcagcctgaagctgagcagcgtgaccgccgctgacaccgccgtgtactactgcgccaggtggggcagcttcgactactggggccagggcaccctggtgaccgtgtcttctgctagcaccaagggccccagcgtgtttcctctcgctccctgcagccggagcacatccgagagcaccgctgctctgggctgtctcgtgaaggactacttccctgaacccgtcaccgtcagctggaatagcggcgccctgacatccggcgtccacacattccccgctgtcctgcagagcagcggcctgtacagcctgagctccgtggtcaccgtgcctagcagcagcctgggaacaaagacctacacctgcaacgtggaccataagccctccaacaccaaggtggacaagcgggtggaatccaagtatggacccccctgtcctccttgccctgctcctgaatttctcggaggcccctccgtcttcctgtttccccccaagcccaaggacaccctgatgatctcccggacacccgaagtcacctgcgtcgtggtggatgtcagccaggaagatcccgaggtgcagttcaactggtacgtggacggagtggaggtgcataacgccaaaaccaagcccagggaagagcagttcaacagcacctatcgggtcgtgtccgtgctcaccgtcctgcatcaggattggctcaacggcaaggagtacaagtgcaaggtgtccaacaagggcctgccctcctccatcgagaagaccatctccaaggctaagggccaacctcgggagccccaagtgtataccctccctcccagccaggaggagatgaccaagaatcaagtgagcctgacctgcctcgtgaagggattttacccctccgacatcgctgtggaatgggaaagcaatggccaacctgagaacaactacaagaccacaccccccgtgctggactccgatggctccttcttcctgtacagcaggctgaccgtggacaaatcccggtggcaagagggaaacgtgttcagctgctccgtgatgcacgaggctctccacaaccactacacccagaagagcctctccctgagcctcggctagtaa

SEQ ID NO: 37 - DNA, 642 bp, Artificial Sequence
  synthetic construct; AB054
gagatcgtgctgacccagagcccagcaaccctgagcttgagccccggtgagagggccaccctgtcatgcagggccagcagcagcgtgaactacctgcactggtatcagcagaagcccggtcaagcccccaggaggctgatctacgacaccagcaagctggccaccggcatccccgccaggttttccggcagcgggtcaggcaccgactacaccctcaccataagcagcctggagcccgaggacttcgccgtgtactactgtcagcagtggagcggcaacccacctacctttggcggaggcactaaggtggagatcaagcggaccgtggccgcccccagcgtgttcatcttccctcccagcgacgagcagctgaagtctggcaccgccagcgtggtgtgcctgctgaacaacttctacccccgcgaggccaaggtgcagtggaaggtggacaacgccctgcagagcggcaacagccaggagagcgtgaccgagcaggactccaaggacagcacctacagcctgagcagcaccctgaccctgagcaaggccgactacgagaagcacaaggtgtacgcctgcgaggtgacccaccagggactgtctagccccgtgaccaagagcttcaaccggggcgagtgctaa

SEQ ID NO: 38 - DNA, 744 bp, Artificial Sequence
  synthetic construct; heavy chain DX-4012
gaggtgcagctgctggagagcggcggcggcctggtgcagcccggcggcagcctgaggctgagctgcgccgccagcggcttcaccttcagccagtacgtgatgcactgggtgaggcaggcccccggcaagggcctggagtgggtgagcagcatctggcccagcggcggccacaccaggtacgccgacagcgtgaagggcaggttcaccatcagcagggacaacagcaagaacaccctgtacctgcagatgaacagcctgagggccgaggacaccgccgtgtactactgcaccaggcagaggtacaggggccccaagtactactactacatggacgtgtggggcaagggcaccaccgtgaccgtgagcagcgccagcaccaagggccccagcgtgttccccctggcccccagcagcaagagcaccagcggcggcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagagggtggagcccaagagctgcgacaagacccacacctgccccccctgccccgcccccgagctgctgggcggccccagcgtgttcctg

SEQ ID NO: 39 - DNA, 369 bp, Artificial Sequence
  synthetic construct; light chain DX-4012
gaggtgcagctgctggagagcggcggcggcctggtgcagcccggcggcagcctgaggctgagctgcgccgccagcggcttcaccttcagccagtacgtgatgcactgggtgaggcaggcccccggcaagggcctggagtgggtgagcagcatctggcccagcggcggccacaccaggtacgccgacagcgtgaagggcaggttcaccatcagcagggacaacagcaagaacaccctgtacctgcagatgaacagcctgagggccgaggacaccgccgtgtactactgcaccaggcagaggtacaggggccccaagtactactactacatggacgtgtggggcaagggcaccaccgtgaccgtgagcagc

SEQ ID NO: 40 - DNA, 2139 bp, Artificial Sequence
  synthetic consturct; scFv 620I-X0177-A01
gaggtgcagctgctggagagcggcggcggcctggtgcagcccggcggcagcctgaggctgagctgcgccgccagcggcttcaccttcagccactacatcatgatgtgggtgaggcaggcccccggcaagggcctggagtgggtgagcggcatctacagcagcggcggcatcaccgtgtacgccgacagcgtgaagggcaggttcaccatcagcagggacaacagcaagaacaccctgtacctgcagatgaacagcctgagggccgaggacaccgccgtgtactactgcgcctacaggaggatcggcgtgcccaggagggacgagttcgacatctggggccagggcaccatggtgaccgtgagcagcgccagcaccaagggccccagcgtgttccccctggcccccagcagcaagagcaccagcggcggcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagagggtggagcccaagagctgcgacaagacccacacctgccccccctgccccgcccccgagctgctgggcggccccagcgtgttcctgttcccccccaagcccaaggacaccctgatgatcagcaggacccccgaggtgacctgcgtggtggtggacgtgagccacgaggaccccgaggtgaagttcaactggtacgtggacggcgtggaggtgcacaacgccaagaccaagcccagggaggagcagtacaacagcacctacagggtggtgagcgtgctgaccgtgctgcaccaggactggctgaacggcaaggagtacaagtgcaaggtgagcaacaaggccctgcccgcccccatcgagaagaccatcagcaaggccaagggccagcccagggagccccaggtgtacaccctgccccccagcagggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcaggtggcagcagggcaacgtgttcagctgcagcgtgatgcacgaggccctgcacaaccactacacccagaagagcctgagcctgagccccggcaagagcggcggcggcagcgaggtgcagctgctggagagcggcggcggcctggtgcagcccggcggcagcctgaggctgagctgcgccgccagcggcttcaccttcagccagtacgtgatgcactgggtgaggcaggcccccggcaagtgcctggagtgggtgagcagcatctggcccagcggcggccacaccaggtacgccgacagcgtgaagggcaggttcaccatcagcagggacaacagcaagaacaccctgtacctgcagatgaacagcctgagggccgaggacaccgccgtgtactactgcgccaggcagaggtacaggggccccaagtactactactacatggacgtgtggggccagggcaccaccgtgaccgtgagcagcggcggcggcggcagcggcggcggcggcagcggcggcggcggcagcggcggcggcggcagcgacatcgtgatgacccagagccccctgagcctgcccgtgacccccggcgagcccgccagcatcagctgcaggagcagccagagcctgctgcacagcaacggctacaactacctggactggtacctgcagaagcccggccagagcccccagctgctgatctacctgggcagcaacagggccagcggcgtgcccgacaggttcagcggcagcggcagcggcaccgacttcaccctgaagatcagcagggtggaggccgaggacgtgggcgtgtactactgcatgcaggccctgcagaccccctggaccttcggctgcggcaccaaggtggagatcaagagg

SEQ ID NO: 41 - DNA, 375 bp, Artificial Sequence
  synthetic construct; Ab26036
caggtgcagctggtgcagtctggggctgaggtgaagaagcctggggcctcagtgacggtttcctgcaaggcatctggatacaccttcaccagctactatttgcactgggtgcgacaggcccctggacaaggacttgagtggatgggaataatcaaccccagtggtggtagcacaagctacgcacagaagttccagggcagagtcaccatgaccagggacacgtccacgagcacagtctacatggagctgagcagcctgagatctgaggacacggccgtgtattactgtgcgagaggcgtgactacagtgactacttactaccactactacaatatggacgtctggggccaagggaccacggtcaccgtctcctca

SEQ ID NO: 42 - DNA, 321 bp, Artificial Sequence
  synthetic construct; light chain Ab26036
gacatccagatgacccagtctccatcctcactgtctgcatctgtaggagacagagtcaccatcacttgtcgggcgagtcagggcattagcaattatttagcctggtttcagcagaaaccagggaaagcccctaagtccctgatctatgctgcatccagtttgcaaagtggggtcccatcaaagttcagcggcagtggatctgggacagatttcactctcaccatcagcagcctgcagcctgaagattttgcaacttattactgccaaaagtataatacttaccctctcactttcggcggagggaccaaggtggagatcaaa

SEQ ID NO: 43 - DNA, 363 bp, Artificial Sequence
  synthetic construct; heavy chain Ab26048
gaggtgcagctggtggagtctgggggaggcttggtgcagcctcgggggtccctgagactctcctgtgtggcctctggattcacctttagcgactatggcatgagttgggtccgacaggctccagggaaggggctggagtgggtctcagttattggtggtgctggtcatggcacatattacgctgactccgtgaagggccggttcaccatctccagagacaattccaggaacacgttgtatctgcaaatgaacagcctgagagacgaggacacggccgtatattattgtgcgaaaaaatattactggaactacgtcggcggtatggacgtctggggccgagggaccacggtcaccgtctcctca

SEQ ID NO: 44 - DNA, 321 bp, Artificial Sequence
  synthetic construct; light chain Ab26048
gacatccagatgacccagtctccatcttccgtgtctgcatctgtaggagacagagtcaccatcacttgtcgggcgagtcaggatattaacaactggttagcctggtatcagcagaaaccagggaaagcccctaaactcctgatctctactgcatccagtttgcaaagtggggtcccatcaaggttcagcggcagtggatctgggacagacttcactctcaccatcagtagcctacagcctgaagattttgcaacttactattgtcaacaggctaacagtctcccattcactttcggccctgggaccaaagtggatatcaaa

SEQ ID NO: 45 - DNA, 345 bp, Artificial Sequence
  synthetic construct; heavy chain Ab26049
gaggtgcagctggtggagtctgggggagacttggtccagcctggggggtccctgagactctcctgtgcagcctctggattcaccttcaatacctatgctatgcactgggtccgccaggctccagggaagggactggaatatatttcagctattgataccaaagggggttccacatattatgcagactctgtgaagggcagattcaccatttccagagacaattccaagaacgcgcagtatcttcaaatggacagcctgagagttgaagacatggctgtttattattgtgcgagagggttcggtctggacgtctggggccaagggaccacggtcaccgtctcctca

SEQ ID NO: 46 - DNA, 336 bp, Artificial Sequence
  synthetic construct; ligtht chain Ab26049
gatgttgtgatgacccagtctccactctccctggccgtcacccttggacagccggcctccatctcctgcaggtctagtcaaagcctcggatacagtgatggaaacacctacttgaattggtttcaacagaggccaggccaatctccaaggcgcctaatttataaggtttctaaccgggactctggggtcccagacagattcagcggcagtgggtcaggcactgacttcacactgaaaatcaatagggtggagactgtggatgttggggtttactactgcatgcaagctacacactggccgtacacttttggccaggggaccaagctggagatcaaa

SEQ ID NO: 47 - DNA, 372 bp, Artificial Sequence
  synthetic construct; heavy chain Ab26076
caggtacagctgcagcagtcaggtccaggactggtgaagccctcgcagaccctctcactcacctgtgccatctccggggacagtgtctctagcaatagtgctgcttggaattggatcaggcagtccccctcgagaggccttgagtggctgggaaggacatactacaggtccaagtggtttaatgattatgcagtgtctgtgaaaagtcgaataatcatcaacccagacacaaccaagaaccagttctccctgcaggtgaactctgtgactcccgaagacacggctgtgtattactgtgcaagaggagagccagctcgtcggggtgaatacttccaccactggggccagggcaccctggtcaccgtctcctca

SEQ ID NO: 48 - DNA, 324 bp, Artificial Sequence
  synthetic construct; light chain Ab26076
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcaccttggacgttcggccaagggaccaaggtggaaatcaaa

SEQ ID NO: 49 - DNA, 369 bp, Artificial Sequence
  synthetic construct; heavy chain 620I-X0173-A11
gaggtgcagctgctggagagcggcggcggcctggtgcagcccggcggcagcctgaggctgagctgcgccgccagcggcttcaccttcagccagtacgtgatgcactgggtgaggcaggcccccggcaagtgcctggagtgggtgagcagcatctggcccagcggcggccacaccaggtacgccgacagcgtgaagggcaggttcaccatcagcagggacaacagcaagaacaccctgtacctgcagatgaacagcctgagggccgaggacaccgccgtgtactactgcgccaggcagaggtacaggggccccaagtactactactacatggacgtgtggggccagggcaccaccgtgaccgtgagcagc

SEQ ID NO: 50 - DNA, 339 bp, Artificial Sequence
  synthetic construct; light chain 620I-X0173-A11
gacatcgtgatgacccagagccccctgagcctgcccgtgacccccggcgagcccgccagcatcagctgcaggagcagccagagcctgctgcacagcaacggctacaactacctggactggtacctgcagaagcccggccagagcccccagctgctgatctacctgggcagcaacagggccagcggcgtgcccgacaggttcagcggcagcggcagcggcaccgacttcaccctgaagatcagcagggtggaggccgaggacgtgggcgtgtactactgcatgcaggccctgcagaccccctggaccttcggctgcggcaccaaggtggagatcaagagg

SEQ ID NO: 51 - DNA, 369 bp, Artificial Sequence
  synthetic construct; heavy chain 620I-X0173-C07
gaggtgcagctgctggagagcggcggcggcctggtgcagcccggcggcagcctgaggctgagctgcgccgccagcggcttcaccttcagctggtacgtgatgcactgggtgaggcaggcccccggcaagtgcctggagtgggtgagcagcatctaccccagcggcggcaagaccagctacgccgacagcgtgaagggcaggttcaccatcagcagggacaacagcaagaacaccctgtacctgcagatgaacagcctgagggccgaggacaccgccgtgtactactgcgccaggcagaggtacaggggccccaagtactactactacatggacgtgtggggccagggcaccaccgtgaccgtgagcagc

SEQ ID NO: 52 - DNA, 369 bp, Artificial Sequence
  synthetic construct; heavy chain 620I-X0173-E07
gaggtgcagctgctggagagcggcggcggcctggtgcagcccggcggcagcctgaggctgagctgcgccgccagcggcttcaccttcagctggtacagcatgcactgggtgaggcaggcccccggcaagtgcctggagtgggtgagcgtgatctaccccagcggcggcaagaccaggtacgccgacagcgtgaagggcaggttcaccatcagcagggacaacagcaagaacaccctgtacctgcagatgaacagcctgagggccgaggacaccgccgtgtactactgcgccaggcagaggtacaggggccccaagtactactactacatggacgtgtggggccagggcaccaccgtgaccgtgagcagc

SEQ ID NO: 53 - DNA, 369 bp, Artificial Sequence
  synthetic construct; heavy chain 620I-X0173-G11
gaggtgcagctgctggagagcggcggcggcctggtgcagcccggcggcagcctgaggctgagctgcgccgccagcggcttcaccttcagccactacgtgatgcactgggtgaggcaggcccccggcaagtgcctggagtgggtgagcagcatctaccccagcggcggcctgaccaagtacgccgacagcgtgaagggcaggttcaccatcagcagggacaacagcaagaacaccctgtacctgcagatgaacagcctgagggccgaggacaccgccgtgtactactgcgccaggcagaggtacaggggccccaagtactactactacatggacgtgtggggccagggcaccaccgtgaccgtgagcagc

SEQ ID NO: 54 - DNA, 369 bp, Artificial Sequence
  synthetic construct; heavy chian 559C-M0071-F06
gaggtgcagctgctggagagcggcggcggcctggtgcagcccggcggcagcctgaggctgagctgcgccgccagcggcttcaccttcagcggctacatcatggcctgggtgaggcaggcccccggcaagggcctggagtgggtgagctacatctaccccagcggcggcatcaccgtgtacgccgacagcgtgaagggcaggttcaccatcagcagggacaacagcaagaacaccctgtacctgcagatgaacagcctgagggccgaggacaccgccgtgtactactgcaccaggcagaggtacaggggccccaagtactactactacatggacgtgtggggcaagggcaccaccgtgaccgtgagcagc

SEQ ID NO: 55 - DNA, 336 bp, Artificial Sequence
  synthetic construct; light chain 559C-M0071-F06
gacatccagatgacccagagccccctgagcctgcccgtgacccccggcgagcccgccagcatcagctgcaggagcagccagagcctgctgcacagcaacggctactacctggactggtacctgcagaagcccggccagagcccccagctgctgatctacctgggcagcaacagggccagcggcgtgcccgacaggttcagcggcagcggcagcggcaccgacttcaccctgaagatcagcagggtggaggccgaggacgtgggcgtgtactactgcatgcaggccctgcagaccccctggaccttcggccagggcaccaaggtggagatcaagagg

SEQ ID NO: 56 - DNA, 336 bp, Artificial Sequence
  synthetic construct; light chain 559C-M0179-D04
gacatccagatgacccagagccccctgagcctgagcgtggcccccggcgagcccgccagcatcagctgcaggagcagccagagcctgctgcacaggaacggccacaactacctggactggtacctgcagaagcccggccagagcccccagctgctgatctacctgggcagcaacagggccagcggcgtgcccgagaggttcagcggcagcggcagcggcaccgacttcaccctgaggatcagcagggtggaggccgaggacgtgggcgtgtactactgcatgcaggccctgcaggccaggaccttcggccagggcaccaaggtggagatcaagagg

SEQ ID NO: 57 - DNA, 333 bp, Artificial Sequence
  synthetic construct; light chain 559C-M0181-C02
gacatccagatgacccagagccccctgagcctgcccgtgacccccggcgagcccgccagcatcagctgcaggagcagccagagcctgctgcacagcaacggctactacctggactggtacctgcagaagcccggccagagcccccagctgctgatctacctgggcagcaacagggccagcggcgtgcccgacaggttcagcggcagcggcagcggcaccgacttcaccctgaagatcagcagggtggaggccgaggacgtgggcgtgtactactgcatgcaggccctgcagaccaggaccttcggccagggcaccaaggtggagatcaagagg

SEQ ID NO: 58 - DNA, 336 bp, Artificial Sequence
  synthetic construct; light chain 559C-M0180-G03
gacatccagatgacccagagccccctgagcctgcccgtgacccccggcgagcccgccagcatcagctgcaggagcagccagagcctgctgcacagcaacggctactacctggactggtacctgcagaagcccggccagagcccccagatcatgatctacctgggcagcaacagggccagcggcgtgcccgacaggttcagcggcagcggcagcggcaccgacttcaccctgaagatcagcagggtggaggccgaggacgtgggcgtgtactactgcatgcaggccctgcagacccccaggaccttcggccagggcaccaaggtggagatcaagagg

SEQ ID NO: 59 - DNA, 369 bp, Artificial Sequence
  synthetic construct; heavy chain 559C-M0184-B04
gaggtgcagctgctggagagcggcggcggcctggtgcagcccggcggcagcctgaggctgagctgcgccgccagcggcttcaccttcagcttctacagcatgcactgggtgaggcaggcccccggcaagggcctggagtgggtgagcaggatctaccccagcggcggcgtgaccaagtacgccgacagcgtgaagggcaggttcaccatcagcagggacaacagcaagaacaccctgtacctgcagatgaacagcctgagggccgaggacaccgccgtgtactactgcaccaggcagaggtacaggggccccaagtactactactacatggacgtgtggggcaagggcaccaccgtgaccgtgagcagc

SEQ ID NO: 60 - protein, 205 aa, Artificial Sequence
  synthetic construct; IgG1
FPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 205 aa, Artificial Sequence
  synthetic construct; IgG2
FPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 62 - protein, 205 aa, Artificial Sequence
  synthetic construct; IgG4
FPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 63 - protein, 205 aa, Artificial Sequence; identical to SEQ ID NO: 62
  synthetic construct; Garadacimab

SEQ ID NO: 64 - protein, 204 aa, Artificial Sequence
  synthetic construct; AB054
FPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 65 - protein, 204 aa, Artificial Sequence
  synthetic construct; DX-4012
FPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 66 - protein, 205 aa, Artificial Sequence
  synthetic construct; DX-2930
FPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 67 - DNA, 1050 bp, Artificial Sequence
  synthetic construct; promoter
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtctagaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 68 - DNA, 1089 bp, Artificial Sequence
  synthetic construct; promoter
aggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagaggatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 69 - DNA, 1430 bp, Artificial Sequence
  synthetic construct; promoter
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtctagagcccttaagctagcaggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagaggatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 70 - DNA, 1318 bp, Artificial Sequence
  synthetic construct; promoter
aggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagaggatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtaaaacaggtaagtccgctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctggcctctactaaccatgttcatgttttctttttttttctacaggtcctgggtgacgaacag

SEQ ID NO: 71 - DNA, 1579 bp, Artificial Sequence
  synthetic construct; promoter
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggccactacgggtttaggctgcccatgtaaggaggcaaggcctggggacacccgagatgcctggttataattaacccagacatgtggctgccccccccccccccaacacctgctgcctctaaaaataaccctgtccctggtggatcccactacgggtttaggctgcccatgtaaggaggcaaggcctggggacacccgagatgcctggttataattaacccagacatgtggctgccccccccccccccaacacctgctgcctctaaaaataaccctgtccctggtggatcccactacgggtttaggctgcccatgtaaggaggcaaggcctggggacacccgagatgcctggttataattaacccagacatgtggctgccccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgtcgagatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 72 - DNA, 1080 bp, Artificial Sequence
  synthetic construct; promoter
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtctagaggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagcgtgagtatcgatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 73 - DNA, 568 bp, Artificial Sequence
  synthetic construct; promoter
gaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagcgatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 74 - DNA, 895 bp, Artificial Sequence
  synthetic construct; promoter
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtctagagaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagcgatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 75 - DNA, 1291 bp, Artificial Sequence
  synthetic construct; promoter
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggccactacgggtttaggctgcccatgtaaggaggcaaggcctggggacacccgagatgcctggttataattaacccagacatgtggctgccccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgtcgagatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 76 - DNA, 1147 bp, Artificial Sequence
  synthetic construct; promoter
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgtcgagatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 77 - DNA, 1490 bp, Artificial Sequence
  synthetic construct; promoter
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggcccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgaagctttggcccactacgggtttaggctgcccatgtaaggaggcaaggcctggggacacccgagatgcctggttataattaacccagacatgtggctgccccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgtcgagatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 78 - DNA, 723 bp, Artificial Sequence
  synthetic construct; promoter
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 79 - DNA, 100 bp, Artificial Sequence
  synthetic construct; enhancer
aggttaatttttaaaaagcagtcaaaagtccaagtggcccttggcagcatttactctctctgtttgctctggttaataatctcaggagcacaaacattcc

SEQ ID NO: 80 - DNA, 200 bp, Artificial Sequence
  syntehtic construct; enhancer
aggttaatttttaaaaagcagtcaaaagtccaagtggcccttggcagcatttactctctctgtttgctctggttaataatctcaggagcacaaacattccaggttaatttttaaaaagcagtcaaaagtccaagtggcccttggcagcatttactctctctgtttgctctggttaataatctcaggagcacaaacattcc

SEQ ID NO: 81 - DNA, 321 bp, Artificial Sequence
  synthetic construct; enhancer
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggg

SEQ ID NO: 82 - DNA, 554 bp, Artificial Sequence
  synthetic construct; enhancer
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtctagaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctg

SEQ ID NO: 83 - DNA, 393 bp, Artificial Sequence
  synthetic construct; promoter
gatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 84 - DNA, 393 bp, Artificial Sequence
  synthetic construct; promoter
gatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 85 - DNA, 144 bp, Artificial Sequence
  synthetic construct; promoter
ccactacgggtttaggctgcccatgtaaggaggcaaggcctggggacacccgagatgcctggttataattaacccagacatgtggctgccccccccccccccaacacctgctgcctctaaaaataaccctgtccctggtggatc

SEQ ID NO: 86 - DNA, 288 bp, Artificial Sequence
  synthetic construct; enhancer
ccactacgggtttaggctgcccatgtaaggaggcaaggcctggggacacccgagatgcctggttataattaacccagacatgtggctgccccccccccccccaacacctgctgcctctaaaaataaccctgtccctggtggatcccactacgggtttaggctgcccatgtaaggaggcaaggcctggggacacccgagatgcctggttataattaacccagacatgtggctgccccccccccccccaacacctgctgcctctaaaaataaccctgtccctggtggatc

SEQ ID NO: 87 - DNA, 432 bp, Artificial Sequence
  synthetic construct; enhancer
ccactacgggtttaggctgcccatgtaaggaggcaaggcctggggacacccgagatgcctggttataattaacccagacatgtggctgccccccccccccccaacacctgctgcctctaaaaataaccctgtccctggtggatcccactacgggtttaggctgcccatgtaaggaggcaaggcctggggacacccgagatgcctggttataattaacccagacatgtggctgccccccccccccccaacacctgctgcctctaaaaataaccctgtccctggtggatcccactacgggtttaggctgcccatgtaaggaggcaaggcctggggacacccgagatgcctggttataattaacccagacatgtggctgccccccccccccccaacacctgctgcctctaaaaataaccctgtccctggtggatc

SEQ ID NO: 88 - DNA, 199 bp, Artificial Sequence
  synthetic construct; enhancer
cccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgaagctttggc

SEQ ID NO: 89 - DNA, 1725 bp, Artificial Sequence
  synthetic construct; promoter
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaag

SEQ ID NO: 90 - DNA, 865 bp, Artificial Sequence
  synthetic construct; promoter
ccactacgggtttaggctgcccatgtaaggaggcaaggcctggggacacccgagatgcctggttataattaacccagacatgtggctgccccccccccccccaacacctgctgcctctaaaaataaccctgtccctggtggatcccactacgggtttaggctgcccatgtaaggaggcaaggcctggggacacccgagatgcctggttataattaacccagacatgtggctgccccccccccccccaacacctgctgcctctaaaaataaccctgtccctggtggatcccactacgggtttaggctgcccatgtaaggaggcaaggcctggggacacccgagatgcctggttataattaacccagacatgtggctgccccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgtcga

SEQ ID NO: 91 - DNA, 251 bp, Artificial Sequence
  synthetic construct; regulatory element
atggaggcggtactatgtagatgagaattcaggagcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcatact

SEQ ID NO: 92 - DNA, 212 bp, Artificial Sequence
  synthetic construct; regulatory element
gggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacag

SEQ ID NO: 93 - DNA, 460 bp, Artificial Sequence
  synthetic construct; promoter
gggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagat

SEQ ID NO: 94 - DNA, 133 bp, Artificial Sequence
  synthetic construct; chimeric intron
gtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 95 - DNA, 82 bp, Artificial Sequence
  synthetic construct; intron
gtgagtatctcagggatccagacatggggatatgggaggtgcctctgatcccagggctcactgtgggtctctctgttcacag

SEQ ID NO: 96 - DNA, 97 bp, Artificial Sequence
  synthetic construct; intron
gtaagtttagtctttttgtcttttatttcaggtcccggatccggtggtggtgcaaatcaaagaactgctcctcagtggatgttgcctttacttctag

SEQ ID NO: 97 - DNA, 55 bp, Artificial Sequence
  synthetic construct; poly A
ataaaggaaatttattttcattgcaatagtgtgttggaattttttgtgtctctca

SEQ ID NO: 98 - DNA, 127 bp, Artificial Sequence
  synthetic construct; polyA
gatctttttccctctgccaaaaattatggggacatcatgaagccccttgagcatctgacttctggctaataaaggaaatttattttcattgcaatagtgtgttggaattttttgtgtctctcactcg

SEQ ID NO: 99 - DNA, 130 bp, Artificial Sequence
  synthetic construct; 5′ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 100 - DNA, 106 bp, Artificial Sequence
  synthetic construct; 5′ITR
ctgcgcgctcgctcgctcactgaggccgcccgggcaaagcccgggcgtcgggcgacctttggtcgcccggcctcagtgagcgagcgagcgcgcagagagggagtgg

SEQ ID NO: 101 - DNA, 143 bp, Artificial Sequence
  synthetic construct; 3′ITR
gaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 102 - DNA, 110 bp, Artificial Sequence
  synthetic construct; 3′ITR
ttggccactccctctctgcgcgctcgctcgctcactgaggccgggcgaccaaaggtcgcccgacgcccgggctttgcccgggcggcctcagtgagcgagcgagcgcgcag

SEQ ID NO: 103 - protein, 20 aa, Artificial Sequence
  synthetic construct; signal peptide
MYRMQLLLLIALSLALVTNS

SEQ ID NO: 104 - DNA, 60 bp, Artificial Sequence
  synthetic construct; signal peptide
atgtataggatgcaactgctcctcctgattgctctgagcctggctcttgtgaccaactct

SEQ ID NO: 105 - protein, 18 aa, Artificial Sequence
  synthetic construct; signal peptide
MKWVTFISLLFLFSSAYS

SEQ ID NO: 106 - protein, 24 aa, Artificial Sequence
  synthetic construct; signal peptide
MPSSVSWGILLLAGLCCLVPVSLA

SEQ ID NO: 107 - protein, 18 aa, Artificial Sequence
  synthetic construct; signal peptide
MKAAVLTLAVLFLTGSQA

SEQ ID NO: 108 - protein, 18 aa, Artificial Sequence
  synthetic construct; signal peptide
MKLLAATVLLLTICSLEG

SEQ ID NO: 109 - protein, 27 aa, Artificial Sequence
  synthetic construct; signal peptide
MDPPRPALLALLALPALLLLLLAGARA

SEQ ID NO: 110 - protein, 28 aa, Artificial Sequence
  synthetic construct; signal peptide
MQRVNMIMAESPGLITICLLGYLLSAEC

SEQ ID NO: 111 - protein, 20 aa, Artificial Sequence
  synthetic construct; signal peptide
MGPLMVLFCLLFLYPGLADS

SEQ ID NO: 112 - protein, 18 aa, Artificial Sequence
  synthetic construct; signal peptide
MWLLVSVILISRISSVGG

SEQ ID NO: 113 - protein, 18 aa, Artificial Sequence
  synthetic construct; signal peptide
MLLLFSVILISWVSTVGG

SEQ ID NO: 114 - protein, 19 aa, Artificial Sequence
  synthetic construct; signal peptide
MFSMRIVCLVLSVVGTAWT

SEQ ID NO: 115 - protein, 30 aa, Artificial Sequence
  synthetic construct; signal peptide
MKRMVSWSFHKLKTMKHLLLLLLCVFLVKS

SEQ ID NO: 116 - protein, 26 aa, Artificial Sequence
  synthetic construct; signal peptide
MSWSLHPRNLILYFYALLFLSSTCVA

SEQ ID NO: 117 - protein, 18 aa, Artificial Sequence
  synthetic construct; signal peptide
MKSLVLLLCLAQLWGCHS

SEQ ID NO: 118 - protein, 23 aa, Artificial Sequence
  synthetic construct; signal peptide
MARVLGAPVALGLWSLCWSLAIA

SEQ ID NO: 119 - protein, 18 aa, Artificial Sequence
  synthetic construct; signal peptide
MKLITILFLCSRLLLSLT

SEQ ID NO: 120 - protein, 20 aa, Artificial Sequence
  synthetic construct; signal peptide
MSLFPSLPLLLLSMVAASYS

SEQ ID NO: 121 - protein, 19 aa, Artificial Sequence
  synthetic construct; signal peptide
MEHKEVVLLLLLFLKSGQG

SEQ ID NO: 122 - protein, 24 aa, Artificial Sequence
  synthetic construct; signal peptide
MAHVRGLQLPGCLALAALCSLVHS

SEQ ID NO: 123 - protein, 29 aa, Artificial Sequence
  synthetic construct; signal peptide
MISRMEKMTMMMKILIMFALGMNYWSCSG

SEQ ID NO: 124 - protein, 32 aa, Artificial Sequence
  synthetic construct; signal peptide
MYSNVIGTVTSGKRKVYLLSLLLIGFWDCVTC

SEQ ID NO: 125 - protein, 19 aa, Artificial Sequence
  synthetic construct; signal peptide
MRLAVGALLVCAVLGLCLA

SEQ ID NO: 126 - protein, 17 aa, Artificial Sequence
  synthetic construct; signal peptide
MRAWIFFLLCLAGRALA

SEQ ID NO: 127 - protein, 22 aa, Artificial Sequence
  synthetic construct; signal peptide
MFSFVDLRLLLLLAATALLTHG

SEQ ID NO: 128 - protein, 19 aa, Artificial Sequence
  synthetic construct; signal peptide
MKLVFLVLLFLGALGLCLA

SEQ ID NO: 129 - protein, 22 aa, Artificial Sequence
  synthetic construct; signal peptide
MGPTSGPSLLLLLLTHLPLALG

SEQ ID NO: 130 - protein, 18 aa, Artificial Sequence
  synthetic construct; signal peptide
MSLSAFTLFLALIGGTSG

SEQ ID NO: 131 - protein, 27 aa, Artificial Sequence
  synthetic construct; signal peptide
MAPHRPAPALLCALSLALCALSLPVRA

SEQ ID NO: 132 - protein, 22 aa, Artificial Sequence
  synthetic construct; signal peptide
MWATLPLLCAGAWLLGVPVCGA

SEQ ID NO: 133 - protein, 19 aa, Artificial Sequence
  synthetic construct; signal peptide
MQALVLLLCIGALLGHSSC

SEQ ID NO: 134 - protein, 23 aa, Artificial Sequence
  synthetic construct; signal peptide
MQMSPALTCLVLGLALVFGEGSA

SEQ ID NO: 135 - protein, 20 aa, Artificial Sequence
  synthetic construct; signal peptide
MQPSSLLPLALCLLAAPASA

SEQ ID NO: 136 - protein, 23 aa, Artificial Sequence
  synthetic construct; signal peptide
MAPFEPLASGILLLLWLIAPSRA

SEQ ID NO: 137 - protein, 31 aa, Artificial Sequence
  synthetic construct; signal peptide
MLRGPGPGLLLLAVQCLGTAVPSTGASKSKR

SEQ ID NO: 138 - protein, 15 aa, Artificial Sequence
  synthetic construct; signal peptide
MWCIVLFSLLAWVYA

SEQ ID NO: 139 - protein, 17 aa, Artificial Sequence
  synthetic construct; signal peptide
MNPTLILAAFCLGIASA

SEQ ID NO: 140 - protein, 17 aa, Artificial Sequence
  synthetic construct; signal peptide
MWQLWASLCCLLVLANA

SEQ ID NO: 141 - protein, 16 aa, Artificial Sequence
  synthetic construct; signal peptide
MLLILLSVALLAFSSA

SEQ ID NO: 142 - protein, 20 aa, Artificial Sequence
  synthetic construct; signal peptide
MWKRWLALALALVAVAWVRA

SEQ ID NO: 143 - protein, 4 aa, Artificial Sequence
  synthetic construct; linker
RKRR

SEQ ID NO: 144 - protein, 4 aa, Artificial Sequence
  synthetic construct; linker
RRRR

SEQ ID NO: 145 - protein, 4 aa, Artificial Sequence
  synthetic construct; linker
RRKR

SEQ ID NO: 146 - protein, 4 aa, Artificial Sequence
  synthetic construct; linker
RKKR

SEQ ID NO: 147 - protein, 18 aa, Artificial Sequence
  synthetic construct; linker
EGRGSLLTCGDVEENPGP

SEQ ID NO: 148 - protein, 21 aa, Artificial Sequence
  synthetic construct; linker
GSGEGRGSLLTCGDVEENPGP

SEQ ID NO: 149 - protein, 19 aa, Artificial Sequence
  synthetic construct; linker
ATNFSLLKQAGDVEENPGP

SEQ ID NO: 150 - protein, 22 aa, Artificial Sequence
  synthetic construct; linker
GSGATNFSLLKQAGDVEENPGP

SEQ ID NO: 151 - protein, 20 aa, Artificial Sequence
  synthetic construct; linker
QCTNYALLKLAGDVESNPGP

SEQ ID NO: 152 - protein, 23 aa, Artificial Sequence
  synthetic construct; linker
GSGQCTNYALLKLAGDVESNPGP

SEQ ID NO: 153 - protein, 24 aa, Artificial Sequence
  synthetic construct; linker
APVKQTLNFDLLKLAGDVESNPGP

SEQ ID NO: 154 - protein, 27 aa, Artificial Sequence
  synthetic construct; linker
GSGAPVKQTLNFDLLKLAGDVESNPGP

SEQ ID NO: 155 - protein, 22 aa, Artificial Sequence
  synthetic construct; linker
RKRREGRGSLLTCGDVEENPGP

SEQ ID NO: 156 - protein, 25 aa, Artificial Sequence
  synthetic construct; linker
RKRRGSGEGRGSLLTCGDVEENPGP

SEQ ID NO: 157 - protein, 28 aa, Artificial Sequence
  synthetic construct; linker
RKRRAPVKQTLNFDLLKLAGDVESNPGP

SEQ ID NO: 158 - protein, 31 aa, Artificial Sequence
  synthetic construct; linker
RKRRGSGAPVKQTLNFDLLKLAGDVESNPGP

SEQ ID NO: 159 - DNA, 75 bp, Artificial Sequence
  synthetic construct; linker
agaaagagaagaggctctggagaaggcagaggctccctgctgacatgtggggatgttgaagagaatcctgggcct

SEQ ID NO: 160 - DNA, 12 bp, Artificial Sequence
  synthetic construct; linker
agaaagagaaga

SEQ ID NO: 161 - DNA, 21 bp, Artificial Sequence
  synthetic construct; linker
agaaagagaagaggctctgga

SEQ ID NO: 162 - DNA, 9 bp, Artificial Sequence
  synthetic construct; linker
ggctctgga

SEQ ID NO: 163 - DNA, 54 bp, Artificial Sequence
  synthetic construct; linker
gaaggcagaggctccctgctgacatgtggggatgttgaagagaatcctgggcct

SEQ ID NO: 164 - protein, 22 aa, Artificial Sequence
  synthetic construct; linker
RKRREGRGSLLTCGDVEENPGP

SEQ ID NO: 165 - protein, 25 aa, Artificial Sequence
  synthetic construct; linker
RKRRGSGEGRGSLLTCGDVEENPGP

SEQ ID NO: 166 - protein, 28 aa, Artificial Sequence
  synthetic construct; linker
RKRRAPVKQTLNFDLLKLAGDVESNPGP

SEQ ID NO: 167 - protein, 31 aa, Artificial Sequence; identical to SEQ ID NO: 158
  synthetic construct; linker

SEQ ID NO: 168 - protein, 6 aa, Artificial Sequence
  synthetic construct; hinge region
CPPCPA

SEQ ID NO: 169 - protein, 5 aa, Artificial Sequence
  synthetic construct; hinge region
CPPCP

SEQ ID NO: 170 - protein, 5 aa, Artificial Sequence
  synthetic construct; hinge region
CPPCA

SEQ ID NO: 171 - protein, 22 aa, Artificial Sequence
  synthetic construct; hinge region
EPKSCDKTHTCPPCPAPELLGG

SEQ ID NO: 172 - protein, 22 aa, Artificial Sequence
  synthetic construct; hinge region
EPKSCDKTHLCPPCPAPELLGG

SEQ ID NO: 173 - protein, 10 aa, Artificial Sequence
  synthetic construct; hinge region
EPKSCDKTHL

SEQ ID NO: 174 - protein, 10 aa, Artificial Sequence
  synthetic construct; hinge region
EPKSCDKTHT

SEQ ID NO: 175 - protein, 16 aa, Artificial Sequence
  synthetic construct; hinge region
EPKSCDKTHTCPPCPA

SEQ ID NO: 176 - protein, 16 aa, Artificial Sequence
  synthetic construct; hinge region
EPKSCDKTHLCPPCPA

SEQ ID NO: 177 - protein, 27 aa, Artificial Sequence
  synthetic construct; hinge region
EPKSCDKTHTCPPCPAPELLGGPSVFL

SEQ ID NO: 178 - protein, 27 aa, Artificial Sequence
  synthetic construct; hinge region
EPKSCDKTHLCPPCPAPELLGGPSVFL

SEQ ID NO: 179 - protein, 22 aa, Artificial Sequence
  synthetic construct; hinge region
EPKSCDKTHTCPPCPAPEAAGG

SEQ ID NO: 180 - protein, 27 aa, Artificial Sequence
  synthetic construct; hinge region
EPKSCDKTHLCPPCPAPEAAGGPSVFL

SEQ ID NO: 181 - protein, 27 aa, Artificial Sequence
  synthetic construct; hinge region
EPKSCDKTHLCPPCPAPEAAGGPSVFL

SEQ ID NO: 182 - protein, 18 aa, Artificial Sequence
  synthetic construct; hinge region
ERKSCVECPPCPAPPVAG

SEQ ID NO: 183 - protein, 13 aa, Artificial Sequence
  synthetic construct; hinge region
ERKSCVECPPCPA

SEQ ID NO: 184 - protein, 19 aa, Artificial Sequence
  synthetic construct; hinge region
ESKYGPPCPPCPAPEFLGG

SEQ ID NO: 185 - protein, 13 aa, Artificial Sequence
  synthetic construct; hinge region
ESKYGPPCPPCPA

SEQ ID NO: 186 - protein, 13 aa, Artificial Sequence
  synthetic construct; hinge region
ESKYGPPCPSCPA

SEQ ID NO: 187 - protein, 24 aa, Artificial Sequence
  synthetic construct; hinge region
ESKYGPPCPSCPAPEFLGGPSVFL

SEQ ID NO: 188 - protein, 24 aa, Artificial Sequence
  synthetic construct; hinge region
ESKYGPPCPPCPAPEFLGGPSVFL

SEQ ID NO: 189 - protein, 18 aa, Artificial Sequence
  synthetic construct; hinge region
ERKCCVECPPCPAPPVAG

SEQ ID NO: 190 - protein, 13 aa, Artificial Sequence
  synthetic construct; hinge region
ERKCCVECPPCPA

SEQ ID NO: 191 - protein, 22 aa, Artificial Sequence
  synthetic construct; hinge region
EPKSCDKTHTCPPCPAPELAGA

SEQ ID NO: 192 - protein, 27 aa, Artificial Sequence
  synthetic construct; hinge region
EPKSCDKTHTCPPCPAPELAGAPSVFL

SEQ ID NO: 193 - protein, 27 aa, Artificial Sequence
  synthetic construct; hinge region
EPKSCDKTHLCPPCPAPELAGAPSVFL

SEQ ID NO: 194 - protein, 22 aa, Artificial Sequence
  synthetic construct; hinge region
EPKSCDKTHTCPPCPAPEFEGG

SEQ ID NO: 195 - protein, 27 aa, Artificial Sequence
  synthetic construct; hinge region
EPKSCDKTHTCPPCPAPEFEGGPSVFL

SEQ ID NO: 196 - protein, 27 aa, Artificial Sequence
  synthetic construct; hinge region
EPKSCDKTHLCPPCPAPEFEGGPSVFL

SEQ ID NO: 197 - protein, 735 aa, Artificial Sequence [sequence omitted]

SEQ ID NO: 198 - protein, 737 aa, Artificial Sequence
  synthetic construct; AAV7
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 199 - protein, 738 aa, Artificial Sequence
  synthetic construct; AAV8
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 200 - protein, 736 aa, Artificial Sequence
  synthetic construct; AAV9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 201 - protein, 738 aa, Artificial Sequence
  synthetic construct; rh10
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 202 - protein, 738 aa, Artificial Sequence
  synthetic construct; rh46
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPIGEPPAAPSSVGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFSFSYTFEDVPFHSSYAHSQSLDRLMNPLIDQYLYYLSRTQSTGGTAGTQQLLFSQAGPSNMSAQARNWLPGPCYRQQRVSTTLSQNNNSNFAWTGATKYHLNGRDSLVNPGVAMATNKDDEDRFFPSSGILMFGKQGAGKDNVDYSNVMLTSEEEIKATNPVATEQYGVVADNLQQQNTAPIVGAVNSQGALPGMVWQNRDVYLQGPIWAKIPHTDGNFHPSPLMGGFGLKHPPPQILIKNTPVPADPPTAFNQAKLNSFITQYSTGQVSVEIEWELQKENSKRWNPEIQYTSNYYKSTNVDFAVNTEGVYSEPRPIGTRYLTRNL

SEQ ID NO: 203 - protein, 738 aa, Artificial Sequence
  synthetic construct; rh73
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSSVGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFSFSYTFEDVPFHSSYAHSQSLDRLMNPLIDQYLYYLSRTQSTGGTAGTQQLLFSQAGPSNMSAQARNWLPGPCYRQQRVSTTLSQNNNSNFAWTGATKYHLNGRDSLVNPGVAMATNKDDEDRFFPSSGILMFGKQGAGKDNVDYSNVMLTSEEEIKTTNPVATEQYGVVADNLQRQNTAPIVGAVNSQGALPGMVWQNRDVYLQGPIWAKIPHTDGNFHPSPLMGGFGLKHPPPQILIKNTPVPADPPTAFNQAKLNSFITQYSTGQVSVEIEWELQKENSKRWNPEIQYTSNYYKSTNVDFAVNTEGVYSEPRPIGTRYLTRNL

SEQ ID NO: 204 - protein, 736 aa, Artificial Sequence
  synthetic construct; LK03
MAADGYLPDWLEDNLSEGIREWWALQPGAPKPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRPL

SEQ ID NO: 205 - protein, 736 aa, Artificial Sequence
  synthetic construct; AAVS3
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 206 - protein, 7 aa, Artificial Sequence
  synthetic construct; Y-sulfation site
EDTAVYY

SEQ ID NO: 207 - protein, 7 aa, Artificial Sequence
  synthetic construct; Y-sulfation site
EDEADYY

SEQ ID NO: 208 - protein, 7 aa, Artificial Sequence
  synthetic construct; Y-sulfation site
EDVGVYY

SEQ ID NO: 209 - protein, 6 aa, Artificial Sequence
  synthetic construct; Y-sulfation site
DTAVYY

SEQ ID NO: 210 - protein, 7 aa, Artificial Sequence
  synthetic construct; Y-sulfation site
EDFAVYY

SEQ ID NO: 211 - protein, 7 aa, Artificial Sequence
  synthetic construct; Y-sulfation site
EDFATYY

SEQ ID NO: 212 - protein, 7 aa, Artificial Sequence
  synthetic construct; Y-sulfation site
EDMAVYY

SEQ ID NO: 213 - protein, 6 aa, Artificial Sequence
  synthetic construct; Y-sulfation site
DVGVYY

SEQ ID NO: 214 - protein, 247 aa, Artificial Sequence
  synthetic construct; Lanadelumab Heavy
  misc (227)..(230): amino acids 227 to 230 are present or absent
  misc (231)..(236): amino acids 231 to 236 are present or absent
  misc (237)..(247): amino acids 237 to 247 are present or absent
EVQLLESGGGLVQPGGSLRLSCAASGFTFSHYIMMWVRQAPGKGLEWVSGIYSSGGITVYADSVKGRFTISRDNSKNTLYLQMNSLRAEDTAVYYCAYRRIGVPRRDEFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFL

SEQ ID NO: 215 - protein, 213 aa, Artificial Sequence
  synthetic construct; Lanadelumab Light
DIQMTQSPSTLSASVGDRVTITCRASQSISSWLAWYQQKPGKAPKLLIYKASTLESGVPSRFSGSGSGTEFTLTISSLQPDDFATYYCQQYNTYWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 216 - DNA, 741 bp, Artificial Sequence
  synthetic construct; Lanadelumab Heavy
  misc (679)..(690): amino acids 679 to 690 are present or absent
  misc (691)..(708): amino acids 691 to 708 are present or absent
  misc (709)..(741): amino acids 709 to 741 are present or absent
gaggtgcagctgctggagagcggcggcggcctggtgcagcccggcggcagcctgaggctgagctgcgccgccagcggcttcaccttcagccactacatcatgatgtgggtgaggcaggcccccggcaagggcctggagtgggtgagcggcatctacagcagcggcggcatcaccgtgtacgccgacagcgtgaagggcaggttcaccatcagcagggacaacagcaagaacaccctgtacctgcagatgaacagcctgagggccgaggacaccgccgtgtactactgcgcctacaggaggatcggcgtgcccaggagggacgagttcgacatctggggccagggcaccatggtgaccgtgagcagcgccagcaccaagggccccagcgtgttccccctggcccccagcagcaagagcaccagcggcggcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagagggtggagcccaagagctgcgacaagacccacacctgccccccctgccccgcccccgagctgctgggcggccccagcgtgttcctg

SEQ ID NO: 217 - DNA, 639 bp, Artificial Sequence
  synthetic construct; Lanadelumab Light
gacatccagatgacccagagccccagcaccctgagcgccagcgtgggcgacagggtgaccatcacctgcagggccagccagagcatcagcagctggctggcctggtaccagcagaagcccggcaaggcccccaagctgctgatctacaaggccagcaccctggagagcggcgtgcccagcaggttcagcggcagcggcagcggcaccgagttcaccctgaccatcagcagcctgcagcccgacgacttcgccacctactactgccagcagtacaacacctactggaccttcggccagggcaccaaggtggagatcaagaggaccgtggccgcccccagcgtgttcatcttcccccccagcgacgagcagctgaagagcggcaccgccagcgtggtgtgcctgctgaacaacttctaccccagggaggccaaggtgcagtggaaggtggacaacgccctgcagagcggcaacagccaggagagcgtgaccgagcaggacagcaaggacagcacctacagcctgagcagcaccctgaccctgagcaaggccgactacgagaagcacaaggtgtacgcctgcgaggtgacccaccagggcctgagcagccccgtgaccaagagcttcaacaggggcgagtgc

SEQ ID NO: 218 - DNA, 2205 bp, Artificial Sequence
  synthetic construct; antibody fragment
atgtaccggatgcagctgctgctgctcattgccctgtctctggccctggtcaccaatagcgaagtccagctgctggaatctggcggcggacttgttcaacctggcggctctctgagactgagctgtgccgcttccggcttcaccttcagccactatatcatgatgtgggtccgacaggcccctggcaaaggccttgaatgggtgtccggcatctatagcagcggcggcatcacagtgtacgccgactctgtgaagggcagattcaccatcagccgggacaacagcaagaacaccctgtacctgcagatgaacagcctgagagccgaggacaccgccgtgtactactgcgcctatagaagaatcggcgtgcccagacgggacgagttcgatatttggggccagggcaccatggtcaccgtgtctagcgcctctacaaagggccctagcgtgttccctctggctcctagcagcaagagcacaagcggaggaacagccgctctgggctgtctggtcaaggactactttcccgagcctgtgaccgtgtcctggaatagcggagcactgacaagcggcgtgcacacctttccagctgtgctgcaaagcagcggcctgtactctctgagcagcgtggtcacagtgccaagctctagcctgggcacccagacctacatctgcaatgtgaaccacaagcctagcaacaccaaggtggacaagagagtggaacccaagagctgcgacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagccgggaagagatgaccaagaaccaggtgtccctgacctgcctcgtgaagggcttctacccttccgatatcgccgtggaatgggagagcaatggccagccagagaacaactacaagacaacccctcctgtgctggacagcgacggctcattcttcctgtacagcaagctgacagtggacaagtccagatggcagcagggcaacgtgttcagctgcagcgtgatgcacgaggccctgcacaaccactacacccagaagagtctgagcctgtctccaggcctgcggaagagaagggctcctgtgaagcagaccctgaacttcgacctgctgaaactggccggcgacgtggaaagcaaccccggacctatgtatagaatgcagctcctcctgctgatcgcactgagcctggctctcgtgaccaacagcgacatccagatgacacagagccccagcacactgtctgccagcgtgggagacagagtgaccatcacctgtagagccagccagtccatctcctcttggctggcctggtatcagcaaaagcctggcaaggcccctaagctgctgatctacaaggcctccacactggaaagcggggtgccctccagattttctggcagcggatctggcaccgagttcaccctgaccatcagtagcctgcagcctgacgacttcgccacctactactgccagcagtacaatacctactggaccttcggccagggaacaaaggtggaaatcaagcggactgtggccgctccaagcgtgttcatctttccacctagcgacgagcagctgaagtccggcacagcctctgttgtgtgcctgctgaacaacttctaccccagagaagccaaggtgcagtggaaagtggacaatgccctgcagagcggcaactcccaagagagcgtgacagagcaggactccaaggattccacctacagcctgtctagcaccctgacactgagcaaggccgactacgagaagcacaaagtgtatgcctgcgaagtgacccaccagggccttagctctccagtgaccaagagcttcaaccggggcgagtgttgataa

SEQ ID NO: 219 - DNA, 2193 bp, Artificial Sequence
  synthetic construct; antibody fragment
atgtacagaatgcagctgctgctgctcattgccctgtctctggccctggtcaccaattctgaggtccagctgcttgagagtggtggtggactggttcagcctggtggcagcctgagactgtcttgtgctgcctctggcttcaccttcagccactatatcatgatgtgggtcagacaggcccctggcaaaggcctggaatgggtgtcaggcatctacagcagtggtggcatcacagtgtatgctgactctgtgaagggcagattcaccatcagcagagacaacagcaagaacaccctgtacctgcagatgaactccctgagagctgaggacacagcagtgtactactgtgcctatagaagaattggggtgcccagaagggatgagtttgacatctggggccagggcaccatggttacagtgtcctctgccagcacaaagggcccctctgtttttccactggctcccagcagcaagagcaccagtggtggaacagctgccctgggctgtctggtcaaggattacttccctgagcctgtgactgtgtcctggaactctggggctctgacttctggggtgcacacctttccagctgtgctgcagtcctctggcctgtactctctgtcctctgtggtcacagtgcctagctctagcctgggcacacagacctacatctgcaatgtgaaccacaagcctagcaacaccaaggtggacaagagagtggaacccaagagctgtgacaagacccacacctgtcctccatgtcctgctccagaactgcttggaggcccttctgtgttcctgtttcctccaaagcctaaggacaccctgatgatcagcagaacccctgaagtgacctgtgtggtggttgatgtgtcccatgaggacccagaagtgaagttcaattggtatgtggatggggttgaagtgcacaatgccaagaccaagcctagagaggaacagtacaacagcacctacagagtggtttctgtgctgacagtgctgcaccaggactggctgaatggcaaagagtacaagtgcaaggtgtccaacaaggccctgcctgctcctattgagaaaaccatctccaaggccaagggccagccaagagaaccccaggtttacaccctgccacctagcagagaagagatgaccaagaaccaggtgtccctgacctgcctggttaagggcttctacccctctgacattgctgtggaatgggagagcaatggccagcctgaaaacaactacaagacaacccctcctgtgctggactctgatggctcattcttcctgtacagcaagctgactgtggacaagtccagatggcagcagggaaatgtgttcagctgctctgtgatgcatgaggccctgcacaaccactacacccagaaaagtctgtctctgtcccctggcagaaagagaagaggctctggagaaggcagaggctccctgctgacatgtggggatgttgaagagaatcctgggcctatgtataggatgcaactgctcctcctgattgctctgagcctggctcttgtgaccaactctgacatccagatgacacagagcccctccacactgtctgcatctgtgggagacagagtgaccatcacctgtagagccagccagtctatctctagctggctggcctggtatcagcaaaagcctggcaaggcccctaagctgctgatctacaaggccagcacacttgagtcaggggtgccctccagattttctggctctggatctggcacagagttcaccctgaccatcagctccctgcagccagatgactttgccacctactactgccagcagtacaatacctactggacctttggccagggaacaaaggtggaaatcaagagaacagtggctgccccatctgtgttcatcttcccaccatctgatgaacagctgaagtctggcactgcctctgttgtgtgcctgctgaacaacttttaccccagagaagccaaggtgcagtggaaagtggataatgccctgcagtctggcaatagccaagaatctgtgacagagcaggactccaaggattccacctacagcctgagcagcaccctgacactgagcaaggctgactatgagaagcacaaagtgtatgcctgtgaagtgacacaccagggactgagcagcccagtgaccaagagcttcaacaggggagagtgctgataa

SEQ ID NO: 220 - DNA, 2211 bp, Artificial Sequence
  synthetic construct; antibody fragment
atgtacagaatgcagctgctgctgctcattgccctgtctctggccctggtcaccaattctgaggtccagctgcttgagagtggtggtggactggttcagcctggtggcagcctgagactgtcttgtgctgcctctggcttcaccttcagccactatatcatgatgtgggtcagacaggcccctggcaaaggcctggaatgggtgtcaggcatctacagcagtggtggcatcacagtgtatgctgactctgtgaagggcagattcaccatcagcagagacaacagcaagaacaccctgtacctgcagatgaactccctgagagctgaggacacagcagtgtactactgtgcctatagaagaattggggtgcccagaagggatgagtttgacatctggggccagggcaccatggttacagtgtcctctgccagcacaaagggcccctctgtttttccactggctcccagcagcaagagcaccagtggtggaacagctgccctgggctgtctggtcaaggattacttccctgagcctgtgactgtgtcctggaactctggggctctgacctctggggtgcacacctttccagctgtgctgcagtcctctggcctgtactctctgtcctctgtggtcacagtgcctagctctagcctgggcacacagacctacatctgcaatgtgaaccacaagcctagcaacaccaaggtggacaagagagtggaacccaagagctgtgacaagacccacacctgtcctccatgtcctgctccagaactgcttggaggcccttctgtgttcctgtttcctccaaagcctaaggacaccctgatgatcagcagaacccctgaagtgacctgtgtggtggttgatgtgtcccatgaggacccagaagtgaagttcaattggtatgtggatggggttgaagtgcacaatgccaagaccaagcctagagaggaacagtacaacagcacctacagagtggtttctgtgctgacagtgctgcaccaggactggctgaatggcaaagagtacaagtgcaaggtgtccaacaaggccctgcctgctcctattgagaaaaccatctccaaggccaagggccagccaagagaaccccaggtttacaccctgccacctagcagagaagagatgaccaagaaccaggtgtccctgacctgcctggttaagggcttctacccctctgacattgctgtggaatgggagagcaatggccagcctgaaaacaactacaagacaacccctcctgtgctggactctgatggctcattcttcctgtacagcaagctgactgtggacaagtccagatggcagcagggaaatgtgttcagctgctctgtgatgcatgaggccctgcacaaccactacacccagaaaagtctgtctctgtcccctggcagaaagagaagaggatcaggggccccagtgaagcagaccctgaactttgatctgctgaagctggctggggatgttgagagcaaccctggacctatgtataggatgcaactgctcctcctgattgctctgagcctggctcttgtgaccaactctgacatccagatgacacagagcccctccacactgtctgcatctgtgggagacagagtgaccatcacctgtagagccagccagtctatctcttcctggctggcctggtatcagcaaaagcctggcaaggcccctaagctgctgatctacaaggccagcacacttgagtcaggggtgccctccagattttctggctctggatctggcacagagttcaccctgaccatcagctccctgcagccagatgactttgccacctactactgccagcagtacaatacctactggacctttggccagggaacaaaggtggaaatcaagagaacagtggctgccccatctgtgttcatcttcccaccatctgatgaacagctgaagtctggcactgcctctgttgtgtgcctgctgaacaacttttaccctagagaagccaaggtgcagtggaaagtggataatgccctgcagtctggcaatagccaagaatctgtgacagagcaggactccaaggattccacctacagcctgagcagcaccctgacactgagcaaggctgactatgagaagcacaaagtgtatgcctgtgaagtgacacaccagggactgagcagcccagtgaccaagagcttcaacaggggagagtgctgataa

SEQ ID NO: 221 - DNA, 4196 bp, Artificial Sequence
  synthetic construct; antibody fragment
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgctagcgggcactttgcactggaacttacaacacccgagcaaggacgcgactctccaccatgtaccggatgcagctgctgctgctcattgccctgtctctggccctggtcaccaatagcgaagtccagctgctggaatctggcggcggacttgttcaacctggcggctctctgagactgagctgtgccgcttccggcttcaccttcagccactatatcatgatgtgggtccgacaggcccctggcaaaggccttgaatgggtgtccggcatctatagcagcggcggcatcacagtgtacgccgactctgtgaagggcagattcaccatcagccgggacaacagcaagaacaccctgtacctgcagatgaacagcctgagagccgaggacaccgccgtgtactactgcgcctatagaagaatcggcgtgcccagacgggacgagttcgatatttggggccagggcaccatggtcaccgtgtctagcgcctctacaaagggccctagcgtgttccctctggctcctagcagcaagagcacaagcggaggaacagccgctctgggctgtctggtcaaggactactttcccgagcctgtgaccgtgtcctggaatagcggagcactgacaagcggcgtgcacacctttccagctgtgctgcaaagcagcggcctgtactctctgagcagcgtggtcacagtgccaagctctagcctgggcacccagacctacatctgcaatgtgaaccacaagcctagcaacaccaaggtggacaagagagtggaacccaagagctgcgacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagccgggaagagatgaccaagaaccaggtgtccctgacctgcctcgtgaagggcttctacccttccgatatcgccgtggaatgggagagcaatggccagccagagaacaactacaagacaacccctcctgtgctggacagcgacggctcattcttcctgtacagcaagctgacagtggacaagtccagatggcagcagggcaacgtgttcagctgcagcgtgatgcacgaggccctgcacaaccactacacccagaagagtctgagcctgtctccaggcctgcggaagagaagggctcctgtgaagcagaccctgaacttcgacctgctgaaactggccggcgacgtggaaagcaaccccggacctatgtatagaatgcagctcctcctgctgatcgcactgagcctggctctcgtgaccaacagcgacatccagatgacacagagccccagcacactgtctgccagcgtgggagacagagtgaccatcacctgtagagccagccagtccatctcctcttggctggcctggtatcagcaaaagcctggcaaggcccctaagctgctgatctacaaggcctccacactggaaagcggggtgccctccagattttctggcagcggatctggcaccgagttcaccctgaccatcagtagcctgcagcctgacgacttcgccacctactactgccagcagtacaatacctactggaccttcggccagggaacaaaggtggaaatcaagcggactgtggccgctccaagcgtgttcatctttccacctagcgacgagcagctgaagtccggcacagcctctgttgtgtgcctgctgaacaacttctaccccagagaagccaaggtgcagtggaaagtggacaatgccctgcagagcggcaactcccaagagagcgtgacagagcaggactccaaggattccacctacagcctgtctagcaccctgacactgagcaaggccgactacgagaagcacaaagtgtatgcctgcgaagtgacccaccagggccttagctctccagtgaccaagagcttcaaccggggcgagtgttgataaagcggccgcggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 222 - DNA, 4106 bp, Artificial Sequence
  synthetic construct; antibody fragment
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccgccaccatgtacagaatgcagctgctgctgctcattgccctgtctctggccctggtcaccaattctgaggtccagctgcttgagagtggtggtggactggttcagcctggtggcagcctgagactgtcttgtgctgcctctggcttcaccttcagccactatatcatgatgtgggtcagacaggcccctggcaaaggcctggaatgggtgtcaggcatctacagcagtggtggcatcacagtgtatgctgactctgtgaagggcagattcaccatcagcagagacaacagcaagaacaccctgtacctgcagatgaactccctgagagctgaggacacagcagtgtactactgtgcctatagaagaattggggtgcccagaagggatgagtttgacatctggggccagggcaccatggttacagtgtcctctgccagcacaaagggcccctctgtttttccactggctcccagcagcaagagcaccagtggtggaacagctgccctgggctgtctggtcaaggattacttccctgagcctgtgactgtgtcctggaactctggggctctgacctctggggtgcacacctttccagctgtgctgcagtcctctggcctgtactctctgtcctctgtggtcacagtgcctagctctagcctgggcacacagacctacatctgcaatgtgaaccacaagcctagcaacaccaaggtggacaagagagtggaacccaagagctgtgacaagacccacacctgtcctccatgtcctgctccagaactgcttggaggcccttctgtgttcctgtttcctccaaagcctaaggacaccctgatgatcagcagaacccctgaagtgacctgtgtggtggttgatgtgtcccatgaggacccagaagtgaagttcaattggtatgtggatggggttgaagtgcacaatgccaagaccaagcctagagaggaacagtacaacagcacctacagagtggtttctgtgctgacagtgctgcaccaggactggctgaatggcaaagagtacaagtgcaaggtgtccaacaaggccctgcctgctcctattgagaaaaccatctccaaggccaagggccagccaagagaaccccaggtttacaccctgccacctagcagagaagagatgaccaagaaccaggtgtccctgacctgcctggttaagggcttctacccctctgacattgctgtggaatgggagagcaatggccagcctgaaaacaactacaagacaacccctcctgtgctggactctgatggctcattcttcctgtacagcaagctgactgtggacaagtccagatggcagcagggaaatgtgttcagctgctctgtgatgcatgaggccctgcacaaccactacacccagaaaagtctgtctctgtcccctggcagaaagagaagaggatcaggggccccagtgaagcagaccctgaactttgatctgctgaagctggctggggatgttgagagcaaccctggacctatgtataggatgcaactgctcctcctgattgctctgagcctggctcttgtgaccaactctgacatccagatgacacagagcccctccacactgtctgcatctgtgggagacagagtgaccatcacctgtagagccagccagtctatctcttcctggctggcctggtatcagcaaaagcctggcaaggcccctaagctgctgatctacaaggccagcacacttgagtcaggggtgccctccagattttctggctctggatctggcacagagttcaccctgaccatcagctccctgcagccagatgactttgccacctactactgccagcagtacaatacctactggacctttggccagggaacaaaggtggaaatcaagagaacagtggctgccccatctgtgttcatcttcccaccatctgatgaacagctgaagtctggcactgcctctgttgtgtgcctgctgaacaacttttaccctagagaagccaaggtgcagtggaaagtggataatgccctgcagtctggcaatagccaagaatctgtgacagagcaggactccaaggattccacctacagcctgagcagcaccctgacactgagcaaggctgactatgagaagcacaaagtgtatgcctgtgaagtgacacaccagggactgagcagcccagtgaccaagagcttcaacaggggagagtgctgataactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctca

SEQ ID NO: 223 - protein, 4092 aa, Artificial Sequence
  synthetic construct; antibody fragment
GACATTGATTATTGACTAGTTATTAATAGTAATCAATTACGGGGTCATTAGTTCATAGCCCATATATGGAGTTCCGCGTTACATAACTTACGGTAAATGGCCCGCCTGGCTGACCGCCCAACGACCCCCGCCCATTGACGTCAATAATGACGTATGTTCCCATAGTAACGCCAATAGGGACTTTCCATTGACGTCAATGGGTGGAGTATTTACGGTAAACTGCCCACTTGGCAGTACATCAAGTGTATCATATGCCAAGTACGCCCCCTATTGACGTCAATGACGGTAAATGGCCCGCCTGGCATTATGCCCAGTACATGACCTTATGGGACTTTCCTACTTGGCAGTACATCTACGTATTAGTCATCGCTATTACCATGGTCGAGGTGAGCCCCACGTTCTGCTTCACTCTCCCCATCTCCCCCCCCTCCCCACCCCCAATTTTGTATTTATTTATTTTTTAATTATTTTGTGCAGCGATGGGGGCGGGGGGGGGGGGGGGGCGCGCGCCAGGCGGGGCGGGGCGGGGCGAGGGGCGGGGCGGGGCGAGGCGGAGAGGTGCGGCGGCAGCCAATCAGAGCGGCGCGCTCCGAAAGTTTCCTTTTATGGCGAGGCGGCGGCGGCGGCGGCCCTATAAAAAGCGAAGCGCGCGGCGGGCGGGAGTCGCTGCGCGCTGCCTTCGCCCCGTGCCCCGCTCCGCCGCCGCCTCGCGCCGCCCGCCCCGGCTCTGACTGACCGCGTTACTCCCACAGGTGAGCGGGCGGGACGGCCCTTCTCCTCCGGGCTGTAATTAGCGCTTGGTTTAATGACGGCTTGTTTCTTTTCTGTGGCTGCGTGAAAGCCTTGAGGGGCTCCGGGAGGGCCCTTTGTGCGGGGGGAGCGGCTCGGGGGGTGCGTGCGTGTGTGTGTGCGTGGGGAGCGCCGCGTGCGGCTCCGCGCTGCCCGGCGGCTGTGAGCGCTGCGGGCGCGGCGCGGGGCTTTGTGCGCTCCGCAGTGTGCGCGAGGGGAGCGCGGCCGGGGGCGGTGCCCCGCGGTGCGGGGGGGGCTGCGAGGGGAACAAAGGCTGCGTGCGGGGTGTGTGCGTGGGGGGGTGAGCAGGGGGTGTGGGCGCGTCGGTCGGGCTGCAACCCCCCCTGCACCCCCCTCCCCGAGTTGCTGAGCACGGCCCGGCTTCGGGTGCGGGGCTCCGTACGGGGCGTGGCGCGGGGCTCGCCGTGCCGGGCGGGGGGTGGCGGCAGGTGGGGGTGCCGGGCGGGGCGGGGCCGCCTCGGGCCGGGGAGGGCTCGGGGGAGGGGCGCGGCGGCCCCCGGAGCGCCGGCGGCTGTCGAGGCGCGGCGAGCCGCAGCCATTGCCTTTTATGGTAATCGTGCGAGAGGGCGCAGGGACTTCCTTTGTCCCAAATCTGTGCGGAGCCGAAATCTGGGAGGCGCCGCCGCACCCCCTCTAGCGGGCGCGGGGCGAAGCGGTGCGGCGCCGGCAGGAAGGAAATGGGCGGGGAGGGCCTTCGTGCGTCGCCGCGCCGCCGTCCCCTTCTCCCTCTCCAGCCTCGGGGCTGTCCGCGGGGGGACGGCTGCCTTCGGGGGGGACGGGGCAGGGCGGGGTTCGGCTTCTGGCGTGTGACCGGCGGCTCTAGAGCCTCTGCTAACCATGTTCATGCCTTCTTCTTTTTCCTACAGCTCCTGGGCAACGTGCTGGTTATTGTGCTGTCTCATCATTTTGGCAAAGAATTCGCCGCCACCATGTACAGAATGCAGCTGCTGCTGCTCATTGCCCTGTCTCTGGCCCTGGTCACCAATTCTGAGGTCCAGCTGCTTGAGAGTGGTGGTGGACTGGTTCAGCCTGGTGGCAGCCTGAGACTGTCTTGTGCTGCCTCTGGCTTCACCTTCAGCCACTATATCATGATGTGGGTCAGACAGGCCCCTGGCAAAGGCCTGGAATGGGTGTCAGGCATCTACAGCAGTGGTGGCATCACAGTGTATGCTGACTCTGTGAAGGGCAGATTCACCATCAGCAGAGACAACAGCAAGAACACCCTGTACCTGCAGATGAACTCCCTGAGAGCTGAGGACACAGCAGTGTACTACTGTGCCTATAGAAGAATTGGGGTGCCCAGAAGGGATGAGTTTGACATCTGGGGCCAGGGCACCATGGTTACAGTGTCCTCTGCCAGCACAAAGGGCCCCTCTGTTTTTCCACTGGCTCCCAGCAGCAAGAGCACCAGTGGTGGAACAGCTGCCCTGGGCTGTCTGGTCAAGGATTACTTCCCTGAGCCTGTGACTGTGTCCTGGAACTCTGGGGCTCTGACTTCTGGGGTGCACACCTTTCCAGCTGTGCTGCAGTCCTCTGGCCTGTACTCTCTGTCCTCTGTGGTCACAGTGCCTAGCTCTAGCCTGGGCACACAGACCTACATCTGCAATGTGAACCACAAGCCTAGCAACACCAAGGTGGACAAGAGAGTGGAACCCAAGAGCTGTGACAAGACCCACACCTGTCCTCCATGTCCTGCTCCAGAACTGCTTGGAGGCCCTTCTGTGTTCCTGTTTCCTCCAAAGCCTAAGGACACCCTGATGATCAGCAGAACCCCTGAAGTGACCTGTGTGGTGGTTGATGTGTCCCATGAGGACCCAGAAGTGAAGTTCAATTGGTATGTGGATGGGGTTGAAGTGCACAATGCCAAGACCAAGCCTAGAGAGGAACAGTACAACAGCACCTACAGAGTGGTTTCTGTGCTGACAGTGCTGCACCAGGACTGGCTGAATGGCAAAGAGTACAAGTGCAAGGTGTCCAACAAGGCCCTGCCTGCTCCTATTGAGAAAACCATCTCCAAGGCCAAGGGCCAGCCAAGAGAACCCCAGGTTTACACCCTGCCACCTAGCAGAGAAGAGATGACCAAGAACCAGGTGTCCCTGACCTGCCTGGTTAAGGGCTTCTACCCCTCTGACATTGCTGTGGAATGGGAGAGCAATGGCCAGCCTGAAAACAACTACAAGACAACCCCTCCTGTGCTGGACTCTGATGGCTCATTCTTCCTGTACAGCAAGCTGACTGTGGACAAGTCCAGATGGCAGCAGGGAAATGTGTTCAGCTGCTCTGTGATGCATGAGGCCCTGCACAACCACTACACCCAGAAAAGTCTGTCTCTGTCCCCTGGCAGAAAGAGAAGAGGCTCTGGAGAAGGCAGAGGCTCCCTGCTGACATGTGGGGATGTTGAAGAGAATCCTGGGCCTATGTATAGGATGCAACTGCTCCTCCTGATTGCTCTGAGCCTGGCTCTTGTGACCAACTCTGACATCCAGATGACACAGAGCCCCTCCACACTGTCTGCATCTGTGGGAGACAGAGTGACCATCACCTGTAGAGCCAGCCAGTCTATCTCTAGCTGGCTGGCCTGGTATCAGCAAAAGCCTGGCAAGGCCCCTAAGCTGCTGATCTACAAGGCCAGCACACTTGAGTCAGGGGTGCCCTCCAGATTTTCTGGCTCTGGATCTGGCACAGAGTTCACCCTGACCATCAGCTCCCTGCAGCCAGATGACTTTGCCACCTACTACTGCCAGCAGTACAATACCTACTGGACCTTTGGCCAGGGAACAAAGGTGGAAATCAAGAGAACAGTGGCTGCCCCATCTGTGTTCATCTTCCCACCATCTGATGAACAGCTGAAGTCTGGCACTGCCTCTGTTGTGTGCCTGCTGAACAACTTTTACCCCAGAGAAGCCAAGGTGCAGTGGAAAGTGGATAATGCCCTGCAGTCTGGCAATAGCCAAGAATCTGTGACAGAGCAGGACTCCAAGGATTCCACCTACAGCCTGAGCAGCACCCTGACACTGAGCAAGGCTGACTATGAGAAGCACAAAGTGTATGCCTGTGAAGTGACACACCAGGGACTGAGCAGCCCAGTGACCAAGAGCTTCAACAGGGGAGAGTGCTGATAACTCGAGGACGGGGTGAACTACGCCTGAGGATCCGATCTTTTTCCCTCTGCCAAAAATTATGGGGACATCATGAAGCCCCTTGAGCATCTGACTTCTGGCTAATAAAGGAAATTTATTTTCATTGCAATAGTGTGTTGGAATTTTTTGTGTCTCTCACTCG

SEQ ID NO: 224 - DNA, 3397 bp, Artificial Sequence
  synthetic construct; antibody fragment
aggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagagacagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgagaattcacgcgtgccgccaccatgtacagaatgcagctgctgctgctcattgccctgtctctggccctggtcaccaattctgaggtccagctgcttgagagtggtggtggactggttcagcctggtggcagcctgagactgtcttgtgctgcctctggcttcaccttcagccactatatcatgatgtgggtcagacaggcccctggcaaaggcctggaatgggtgtcaggcatctacagcagtggtggcatcacagtgtatgctgactctgtgaagggcagattcaccatcagcagagacaacagcaagaacaccctgtacctgcagatgaactccctgagagctgaggacacagcagtgtactactgtgcctatagaagaattggggtgcccagaagggatgagtttgacatctggggccagggcaccatggttacagtgtcctctgccagcacaaagggcccctctgtttttccactggctcccagcagcaagagcaccagtggtggaacagctgccctgggctgtctggtcaaggattacttccctgagcctgtgactgtgtcctggaactctggggctctgacttctggggtgcacacctttccagctgtgctgcagtcctctggcctgtactctctgtcctctgtggtcacagtgcctagctctagcctgggcacacagacctacatctgcaatgtgaaccacaagcctagcaacaccaaggtggacaagagagtggaacccaagagctgtgacaagacccacacctgtcctccatgtcctgctccagaactgcttggaggcccttctgtgttcctgtttcctccaaagcctaaggacaccctgatgatcagcagaacccctgaagtgacctgtgtggtggttgatgtgtcccatgaggacccagaagtgaagttcaattggtatgtggatggggttgaagtgcacaatgccaagaccaagcctagagaggaacagtacaacagcacctacagagtggtttctgtgctgacagtgctgcaccaggactggctgaatggcaaagagtacaagtgcaaggtgtccaacaaggccctgcctgctcctattgagaaaaccatctccaaggccaagggccagccaagagaaccccaggtttacaccctgccacctagcagagaagagatgaccaagaaccaggtgtccctgacctgcctggttaagggcttctacccctctgacattgctgtggaatgggagagcaatggccagcctgaaaacaactacaagacaacccctcctgtgctggactctgatggctcattcttcctgtacagcaagctgactgtggacaagtccagatggcagcagggaaatgtgttcagctgctctgtgatgcatgaggccctgcacaaccactacacccagaaaagtctgtctctgtcccctggcagaaagagaagaggctctggagaaggcagaggctccctgctgacatgtggggatgttgaagagaatcctgggcctatgtataggatgcaactgctcctcctgattgctctgagcctggctcttgtgaccaactctgacatccagatgacacagagcccctccacactgtctgcatctgtgggagacagagtgaccatcacctgtagagccagccagtctatctctagctggctggcctggtatcagcaaaagcctggcaaggcccctaagctgctgatctacaaggccagcacacttgagtcaggggtgccctccagattttctggctctggatctggcacagagttcaccctgaccatcagctccctgcagccagatgactttgccacctactactgccagcagtacaatacctactggacctttggccagggaacaaaggtggaaatcaagagaacagtggctgccccatctgtgttcatcttcccaccatctgatgaacagctgaagtctggcactgcctctgttgtgtgcctgctgaacaacttttaccccagagaagccaaggtgcagtggaaagtggataatgccctgcagtctggcaatagccaagaatctgtgacagagcaggactccaaggattccacctacagcctgagcagcaccctgacactgagcaaggctgactatgagaagcacaaagtgtatgcctgtgaagtgacacaccagggactgagcagcccagtgaccaagagcttcaacaggggagagtgctgataagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctca

SEQ ID NO: 225 - DNA, 3226 bp, Artificial Sequence
  synthetic construct; antibody fragment
ggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtcaggtaagtatcaaggttacaagacaggtttaaggagaccaatagaaactgggcttgtcgagacagagaagactcttgcgtttctgataggcacctattggtcttactgacatccactttgcctttctctccacaggttcgaattcgccgccaccatgtacagaatgcagctgctgctgctcattgccctgtctctggccctggtcaccaattctgaggtccagctgcttgagagtggtggtggactggttcagcctggtggcagcctgagactgtcttgtgctgcctctggcttcaccttcagccactatatcatgatgtgggtcagacaggcccctggcaaaggcctggaatgggtgtcaggcatctacagcagtggtggcatcacagtgtatgctgactctgtgaagggcagattcaccatcagcagagacaacagcaagaacaccctgtacctgcagatgaactccctgagagctgaggacacagcagtgtactactgtgcctatagaagaattggggtgcccagaagggatgagtttgacatctggggccagggcaccatggttacagtgtcctctgccagcacaaagggcccctctgtttttccactggctcccagcagcaagagcaccagtggtggaacagctgccctgggctgtctggtcaaggattacttccctgagcctgtgactgtgtcctggaactctggggctctgacttctggggtgcacacctttccagctgtgctgcagtcctctggcctgtactctctgtcctctgtggtcacagtgcctagctctagcctgggcacacagacctacatctgcaatgtgaaccacaagcctagcaacaccaaggtggacaagagagtggaacccaagagctgtgacaagacccacacctgtcctccatgtcctgctccagaactgcttggaggcccttctgtgttcctgtttcctccaaagcctaaggacaccctgatgatcagcagaacccctgaagtgacctgtgtggtggttgatgtgtcccatgaggacccagaagtgaagttcaattggtatgtggatggggttgaagtgcacaatgccaagaccaagcctagagaggaacagtacaacagcacctacagagtggtttctgtgctgacagtgctgcaccaggactggctgaatggcaaagagtacaagtgcaaggtgtccaacaaggccctgcctgctcctattgagaaaaccatctccaaggccaagggccagccaagagaaccccaggtttacaccctgccacctagcagagaagagatgaccaagaaccaggtgtccctgacctgcctggttaagggcttctacccctctgacattgctgtggaatgggagagcaatggccagcctgaaaacaactacaagacaacccctcctgtgctggactctgatggctcattcttcctgtacagcaagctgactgtggacaagtccagatggcagcagggaaatgtgttcagctgctctgtgatgcatgaggccctgcacaaccactacacccagaaaagtctgtctctgtcccctggcagaaagagaagaggctctggagaaggcagaggctccctgctgacatgtggggatgttgaagagaatcctgggcctatgtataggatgcaactgctcctcctgattgctctgagcctggctcttgtgaccaactctgacatccagatgacacagagcccctccacactgtctgcatctgtgggagacagagtgaccatcacctgtagagccagccagtctatctctagctggctggcctggtatcagcaaaagcctggcaaggcccctaagctgctgatctacaaggccagcacacttgagtcaggggtgccctccagattttctggctctggatctggcacagagttcaccctgaccatcagctccctgcagccagatgactttgccacctactactgccagcagtacaatacctactggacctttggccagggaacaaaggtggaaatcaagagaacagtggctgccccatctgtgttcatcttcccaccatctgatgaacagctgaagtctggcactgcctctgttgtgtgcctgctgaacaacttttaccccagagaagccaaggtgcagtggaaagtggataatgccctgcagtctggcaatagccaagaatctgtgacagagcaggactccaaggattccacctacagcctgagcagcaccctgacactgagcaaggctgactatgagaagcacaaagtgtatgcctgtgaagtgacacaccagggactgagcagcccagtgaccaagagcttcaacaggggagagtgctgataactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctca

SEQ ID NO: 226 - DNA, 3472 bp, Artificial Sequence
  synthetic construct; antibody fragment
aggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgtctagaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtcaggtaagtatcaaggttacaagacaggtttaaggagaccaatagaaactgggcttgtcgagacagagaagactcttgcgtttctgataggcacctattggtcttactgacatccactttgcctttctctccacaggttgaattcgccgccaccatgtacagaatgcagctgctgctgctcattgccctgtctctggccctggtcaccaattctgaggtccagctgcttgagagtggtggtggactggttcagcctggtggcagcctgagactgtcttgtgctgcctctggcttcaccttcagccactatatcatgatgtgggtcagacaggcccctggcaaaggcctggaatgggtgtcaggcatctacagcagtggtggcatcacagtgtatgctgactctgtgaagggcagattcaccatcagcagagacaacagcaagaacaccctgtacctgcagatgaactccctgagagctgaggacacagcagtgtactactgtgcctatagaagaattggggtgcccagaagggatgagtttgacatctggggccagggcaccatggttacagtgtcctctgccagcacaaagggcccctctgtttttccactggctcccagcagcaagagcaccagtggtggaacagctgccctgggctgtctggtcaaggattacttccctgagcctgtgactgtgtcctggaactctggggctctgacttctggggtgcacacctttccagctgtgctgcagtcctctggcctgtactctctgtcctctgtggtcacagtgcctagctctagcctgggcacacagacctacatctgcaatgtgaaccacaagcctagcaacaccaaggtggacaagagagtggaacccaagagctgtgacaagacccacacctgtcctccatgtcctgctccagaactgcttggaggcccttctgtgttcctgtttcctccaaagcctaaggacaccctgatgatcagcagaacccctgaagtgacctgtgtggtggttgatgtgtcccatgaggacccagaagtgaagttcaattggtatgtggatggggttgaagtgcacaatgccaagaccaagcctagagaggaacagtacaacagcacctacagagtggtttctgtgctgacagtgctgcaccaggactggctgaatggcaaagagtacaagtgcaaggtgtccaacaaggccctgcctgctcctattgagaaaaccatctccaaggccaagggccagccaagagaaccccaggtttacaccctgccacctagcagagaagagatgaccaagaaccaggtgtccctgacctgcctggttaagggcttctacccctctgacattgctgtggaatgggagagcaatggccagcctgaaaacaactacaagacaacccctcctgtgctggactctgatggctcattcttcctgtacagcaagctgactgtggacaagtccagatggcagcagggaaatgtgttcagctgctctgtgatgcatgaggccctgcacaaccactacacccagaaaagtctgtctctgtcccctggcagaaagagaagaggctctggagaaggcagaggctccctgctgacatgtggggatgttgaagagaatcctgggcctatgtataggatgcaactgctcctcctgattgctctgagcctggctcttgtgaccaactctgacatccagatgacacagagcccctccacactgtctgcatctgtgggagacagagtgaccatcacctgtagagccagccagtctatctctagctggctggcctggtatcagcaaaagcctggcaaggcccctaagctgctgatctacaaggccagcacacttgagtcaggggtgccctccagattttctggctctggatctggcacagagttcaccctgaccatcagctccctgcagccagatgactttgccacctactactgccagcagtacaatacctactggacctttggccagggaacaaaggtggaaatcaagagaacagtggctgccccatctgtgttcatcttcccaccatctgatgaacagctgaagtctggcactgcctctgttgtgtgcctgctgaacaacttttaccccagagaagccaaggtgcagtggaaagtggataatgccctgcagtctggcaatagccaagaatctgtgacagagcaggactccaaggattccacctacagcctgagcagcaccctgacactgagcaaggctgactatgagaagcacaaagtgtatgcctgtgaagtgacacaccagggactgagcagcccagtgaccaagagcttcaacaggggagagtgctgataactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctca

SEQ ID NO: 227 - DNA, 3553 bp, Artificial Sequence
  synthetic construct; antibody fragment
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtctagaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtcaggtaagtatcaaggttacaagacaggtttaaggagaccaatagaaactgggcttgtcgagacagagaagactcttgcgtttctgataggcacctattggtcttactgacatccactttgcctttctctccacaggttgaattcgccgccaccatgtacagaatgcagctgctgctgctcattgccctgtctctggccctggtcaccaattctgaggtccagctgcttgagagtggtggtggactggttcagcctggtggcagcctgagactgtcttgtgctgcctctggcttcaccttcagccactatatcatgatgtgggtcagacaggcccctggcaaaggcctggaatgggtgtcaggcatctacagcagtggtggcatcacagtgtatgctgactctgtgaagggcagattcaccatcagcagagacaacagcaagaacaccctgtacctgcagatgaactccctgagagctgaggacacagcagtgtactactgtgcctatagaagaattggggtgcccagaagggatgagtttgacatctggggccagggcaccatggttacagtgtcctctgccagcacaaagggcccctctgtttttccactggctcccagcagcaagagcaccagtggtggaacagctgccctgggctgtctggtcaaggattacttccctgagcctgtgactgtgtcctggaactctggggctctgacttctggggtgcacacctttccagctgtgctgcagtcctctggcctgtactctctgtcctctgtggtcacagtgcctagctctagcctgggcacacagacctacatctgcaatgtgaaccacaagcctagcaacaccaaggtggacaagagagtggaacccaagagctgtgacaagacccacacctgtcctccatgtcctgctccagaactgcttggaggcccttctgtgttcctgtttcctccaaagcctaaggacaccctgatgatcagcagaacccctgaagtgacctgtgtggtggttgatgtgtcccatgaggacccagaagtgaagttcaattggtatgtggatggggttgaagtgcacaatgccaagaccaagcctagagaggaacagtacaacagcacctacagagtggtttctgtgctgacagtgctgcaccaggactggctgaatggcaaagagtacaagtgcaaggtgtccaacaaggccctgcctgctcctattgagaaaaccatctccaaggccaagggccagccaagagaaccccaggtttacaccctgccacctagcagagaagagatgaccaagaaccaggtgtccctgacctgcctggttaagggcttctacccctctgacattgctgtggaatgggagagcaatggccagcctgaaaacaactacaagacaacccctcctgtgctggactctgatggctcattcttcctgtacagcaagctgactgtggacaagtccagatggcagcagggaaatgtgttcagctgctctgtgatgcatgaggccctgcacaaccactacacccagaaaagtctgtctctgtcccctggcagaaagagaagaggctctggagaaggcagaggctccctgctgacatgtggggatgttgaagagaatcctgggcctatgtataggatgcaactgctcctcctgattgctctgagcctggctcttgtgaccaactctgacatccagatgacacagagcccctccacactgtctgcatctgtgggagacagagtgaccatcacctgtagagccagccagtctatctctagctggctggcctggtatcagcaaaagcctggcaaggcccctaagctgctgatctacaaggccagcacacttgagtcaggggtgccctccagattttctggctctggatctggcacagagttcaccctgaccatcagctccctgcagccagatgactttgccacctactactgccagcagtacaatacctactggacctttggccagggaacaaaggtggaaatcaagagaacagtggctgccccatctgtgttcatcttcccaccatctgatgaacagctgaagtctggcactgcctctgttgtgtgcctgctgaacaacttttaccccagagaagccaaggtgcagtggaaagtggataatgccctgcagtctggcaatagccaagaatctgtgacagagcaggactccaaggattccacctacagcctgagcagcaccctgacactgagcaaggctgactatgagaagcacaaagtgtatgcctgtgaagtgacacaccagggactgagcagcccagtgaccaagagcttcaacaggggagagtgctgataactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctca

SEQ ID NO: 228 - DNA, 3592 bp, Artificial Sequence
  synthetic construct; antibody fragment
aggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagaggatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtcaggtaagtatcaaggttacaagacaggtttaaggagaccaatagaaactgggcttgtcgagacagagaagactcttgcgtttctgataggcacctattggtcttactgacatccactttgcctttctctccacaggttgaattcgccgccaccatgtacagaatgcagctgctgctgctcattgccctgtctctggccctggtcaccaattctgaggtccagctgcttgagagtggtggtggactggttcagcctggtggcagcctgagactgtcttgtgctgcctctggcttcaccttcagccactatatcatgatgtgggtcagacaggcccctggcaaaggcctggaatgggtgtcaggcatctacagcagtggtggcatcacagtgtatgctgactctgtgaagggcagattcaccatcagcagagacaacagcaagaacaccctgtacctgcagatgaactccctgagagctgaggacacagcagtgtactactgtgcctatagaagaattggggtgcccagaagggatgagtttgacatctggggccagggcaccatggttacagtgtcctctgccagcacaaagggcccctctgtttttccactggctcccagcagcaagagcaccagtggtggaacagctgccctgggctgtctggtcaaggattacttccctgagcctgtgactgtgtcctggaactctggggctctgacttctggggtgcacacctttccagctgtgctgcagtcctctggcctgtactctctgtcctctgtggtcacagtgcctagctctagcctgggcacacagacctacatctgcaatgtgaaccacaagcctagcaacaccaaggtggacaagagagtggaacccaagagctgtgacaagacccacacctgtcctccatgtcctgctccagaactgcttggaggcccttctgtgttcctgtttcctccaaagcctaaggacaccctgatgatcagcagaacccctgaagtgacctgtgtggtggttgatgtgtcccatgaggacccagaagtgaagttcaattggtatgtggatggggttgaagtgcacaatgccaagaccaagcctagagaggaacagtacaacagcacctacagagtggtttctgtgctgacagtgctgcaccaggactggctgaatggcaaagagtacaagtgcaaggtgtccaacaaggccctgcctgctcctattgagaaaaccatctccaaggccaagggccagccaagagaaccccaggtttacaccctgccacctagcagagaagagatgaccaagaaccaggtgtccctgacctgcctggttaagggcttctacccctctgacattgctgtggaatgggagagcaatggccagcctgaaaacaactacaagacaacccctcctgtgctggactctgatggctcattcttcctgtacagcaagctgactgtggacaagtccagatggcagcagggaaatgtgttcagctgctctgtgatgcatgaggccctgcacaaccactacacccagaaaagtctgtctctgtcccctggcagaaagagaagaggctctggagaaggcagaggctccctgctgacatgtggggatgttgaagagaatcctgggcctatgtataggatgcaactgctcctcctgattgctctgagcctggctcttgtgaccaactctgacatccagatgacacagagcccctccacactgtctgcatctgtgggagacagagtgaccatcacctgtagagccagccagtctatctctagctggctggcctggtatcagcaaaagcctggcaaggcccctaagctgctgatctacaaggccagcacacttgagtcaggggtgccctccagattttctggctctggatctggcacagagttcaccctgaccatcagctccctgcagccagatgactttgccacctactactgccagcagtacaatacctactggacctttggccagggaacaaaggtggaaatcaagagaacagtggctgccccatctgtgttcatcttcccaccatctgatgaacagctgaagtctggcactgcctctgttgtgtgcctgctgaacaacttttaccccagagaagccaaggtgcagtggaaagtggataatgccctgcagtctggcaatagccaagaatctgtgacagagcaggactccaaggattccacctacagcctgagcagcaccctgacactgagcaaggctgactatgagaagcacaaagtgtatgcctgtgaagtgacacaccagggactgagcagcccagtgaccaagagcttcaacaggggagagtgctgataactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctca

SEQ ID NO: 229 - DNA, 4083 bp, Artificial Sequence
  synthetic construct; antibody fragment
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggccactacgggtttaggctgcccatgtaaggaggcaaggcctggggacacccgagatgcctggttataattaacccagacatgtggctgccccccccccccccaacacctgctgcctctaaaaataaccctgtccctggtggatcccactacgggtttaggctgcccatgtaaggaggcaaggcctggggacacccgagatgcctggttataattaacccagacatgtggctgccccccccccccccaacacctgctgcctctaaaaataaccctgtccctggtggatcccactacgggtttaggctgcccatgtaaggaggcaaggcctggggacacccgagatgcctggttataattaacccagacatgtggctgccccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgtcgagatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtcaggtaagtatcaaggttacaagacaggtttaaggagaccaatagaaactgggcttgtcgagacagagaagactcttgcgtttctgataggcacctattggtcttactgacatccactttgcctttctctccacaggttcgaattcgccgccaccatgtacagaatgcagctgctgctgctcattgccctgtctctggccctggtcaccaattctgaggtccagctgcttgagagtggtggtggactggttcagcctggtggcagcctgagactgtcttgtgctgcctctggcttcaccttcagccactatatcatgatgtgggtcagacaggcccctggcaaaggcctggaatgggtgtcaggcatctacagcagtggtggcatcacagtgtatgctgactctgtgaagggcagattcaccatcagcagagacaacagcaagaacaccctgtacctgcagatgaactccctgagagctgaggacacagcagtgtactactgtgcctatagaagaattggggtgcccagaagggatgagtttgacatctggggccagggcaccatggttacagtgtcctctgccagcacaaagggcccctctgtttttccactggctcccagcagcaagagcaccagtggtggaacagctgccctgggctgtctggtcaaggattacttccctgagcctgtgactgtgtcctggaactctggggctctgacttctggggtgcacacctttccagctgtgctgcagtcctctggcctgtactctctgtcctctgtggtcacagtgcctagctctagcctgggcacacagacctacatctgcaatgtgaaccacaagcctagcaacaccaaggtggacaagagagtggaacccaagagctgtgacaagacccacacctgtcctccatgtcctgctccagaactgcttggaggcccttctgtgttcctgtttcctccaaagcctaaggacaccctgatgatcagcagaacccctgaagtgacctgtgtggtggttgatgtgtcccatgaggacccagaagtgaagttcaattggtatgtggatggggttgaagtgcacaatgccaagaccaagcctagagaggaacagtacaacagcacctacagagtggtttctgtgctgacagtgctgcaccaggactggctgaatggcaaagagtacaagtgcaaggtgtccaacaaggccctgcctgctcctattgagaaaaccatctccaaggccaagggccagccaagagaaccccaggtttacaccctgccacctagcagagaagagatgaccaagaaccaggtgtccctgacctgcctggttaagggcttctacccctctgacattgctgtggaatgggagagcaatggccagcctgaaaacaactacaagacaacccctcctgtgctggactctgatggctcattcttcctgtacagcaagctgactgtggacaagtccagatggcagcagggaaatgtgttcagctgctctgtgatgcatgaggccctgcacaaccactacacccagaaaagtctgtctctgtcccctggcagaaagagaagaggctctggagaaggcagaggctccctgctgacatgtggggatgttgaagagaatcctgggcctatgtataggatgcaactgctcctcctgattgctctgagcctggctcttgtgaccaactctgacatccagatgacacagagcccctccacactgtctgcatctgtgggagacagagtgaccatcacctgtagagccagccagtctatctctagctggctggcctggtatcagcaaaagcctggcaaggcccctaagctgctgatctacaaggccagcacacttgagtcaggggtgccctccagattttctggctctggatctggcacagagttcaccctgaccatcagctccctgcagccagatgactttgccacctactactgccagcagtacaatacctactggacctttggccagggaacaaaggtggaaatcaagagaacagtggctgccccatctgtgttcatcttcccaccatctgatgaacagctgaagtctggcactgcctctgttgtgtgcctgctgaacaacttttaccccagagaagccaaggtgcagtggaaagtggataatgccctgcagtctggcaatagccaagaatctgtgacagagcaggactccaaggattccacctacagcctgagcagcaccctgacactgagcaaggctgactatgagaagcacaaagtgtatgcctgtgaagtgacacaccagggactgagcagcccagtgaccaagagcttcaacaggggagagtgctgataactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctca

SEQ ID NO: 230 - protein, 4 aa, Artificial Sequence
  synthetic construct; linker
  misc (2)..(2): Xaa is any amino acid
RXKR

SEQ ID NO: 231 - protein, 4 aa, Artificial Sequence
  synthetic construct; linker
  misc (2)..(2): Xaa is any amino acid
RXRR

SEQ ID NO: 232 - protein, 738 aa, Artificial Sequence
  synthetic construct; rh64R1
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPIGEPPAAPSSVGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFSFSYTFEDVPFHSSYAHSQSLDRLMNPLIDQYLYYLSRTQSTGGTAGTQQLLFSQAGPSNMSAQARNWLPGPCYRQQRVSTTLSQNNNSNFAWTGATKYHLNGRDSLVNPGVAMATNKDDEDRFFPSSGILMFGKQGAGKDNVDYSNVMLTSEEEIKTTNPVATEQYGVVADNLQQQNTAPIVGAVNSQGALPGMVWQNRDVYLQGPIWAKIPHTDGNFHPSPLMGGFGLKHPPPQILIKNTPVPADPPTAFNQAKLNSFITQYSTGQVSVEIVWELQKENSKRWNPEIQYTSNYYKSTNVDFAVNTEGVYSEPRPIGTRYLTRNL

SEQ ID NO: 233 - DNA, 19 bp, Artificial Sequence
  synthetic construct; regulatory sequence
ggtacaatgactcctttcg

SEQ ID NO: 234 - DNA, 19 bp, Artificial Sequence
  synthetic construct; regulatory sequence
ggtacagtgactcctttcg

SEQ ID NO: 235 - DNA, 10 bp, Artificial Sequence
  synthetic construct; regulatory sequence
tcattctacc

SEQ ID NO: 236 - DNA, 969 bp, Artificial Sequence
  synthetic construct; promoter
aggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgtctagaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

(19) United States
(12) Patent Application Publication
(10) Pub. No.: US 2023/0390418 A1
(43) Pub. Date: Dec. 7, 2023

(54) VECTORIZED FACTOR XII ANTIBODIES AND ADMINISTRATION THEREOF

(71) Applicant: REGENXBIO INC., Rockville, MD (US)

(72) Inventors: Joseph Bruder, Gaithersburg, MD (US); Ye Liu, Clarksville, MD (US)

(21) Appl. No.: 18/034,334

(22) PCT Filed: Oct. 29, 2021
(86) PCT No.: PCT/US21/57319

§ 371 Date: Apr. 27, 2023

Related U.S. Application Data
(60) Provisional application No. 63/107,391, filed on Oct. 29, 2020.

(51) Int. Cl. A61K 48/00; C12N 15/86; C07K 16/36; A61P 7/10

(52) U.S. Cl. CPC A61K 48/0058; C12N 15/86; A61K 48/0041; A61K 48/0066; C07K 16/36; C07K 2317/20; C12N 2750/14143; C12N 2750/14151; C12N 2750/14171; C12N 2750/14122; C12N 2830/15; A61P 7/10

(57) ABSTRACT

Compositions and methods are described for the delivery of a fully human post-translationally modified therapeutic monoclonal antibody that binds to factor XII to a human subject diagnosed with a disease or condition indicated for treatment with an anti-factor XII antibody. Such diseases include hereditary angioedema, as well as thrombosis and hypercoagulation.

### 1. INTRODUCTION

Compositions and methods are described for the delivery of a fully human post-translationally modified (HuPTM) therapeutic monoclonal antibody (“mAb”) that binds to factor XII and/or Factor XIIa or the HuPTM antigen-binding fragment of a therapeutic mAb that binds to factor XII and/or Factor XIIa—e.g., a fully human-glycosylated (HuGly) Fab of the therapeutic mAb—to a human subject diagnosed with a disease or condition indicated for treatment with the therapeutic mAb. Such diseases include hereditary angioedema, as well as thrombosis and hypercoagulation.

### 2. BACKGROUND OF THE INVENTION

Therapeutic mAbs have been shown to be effective in treating a number of diseases and conditions. However, because these agents are effective for only a short period of time, repeated injections for long durations are often required, thereby creating considerable treatment burden for patients. Garadacimab is a therapeutic antibody that binds to the factor XII and may be used for treatment of hereditary angioedema as well as thrombosis and hypercoagulation. Currently, garadacimab, as being investigated for the treatment of hereditary angioedema, is dosed by subcutaneous injection once monthly at doses of 75 mg, 200 mg or 600 mg. There is a need for more effective treatments that reduce the treatment burden on patients suffering from hereditary angioedema, thrombosis, or hypercoagulation.

### 3. SUMMARY OF THE INVENTION

Therapeutic antibodies delivered by gene therapy have several advantages over injected or infused therapeutic antibodies that dissipate over time resulting in peak and trough levels. Sustained expression of the transgene product antibody, as opposed to injecting an antibody repeatedly, allows for a more consistent level of antibody to be present at the site of action, and is less risky and more convenient for patients, since fewer injections need to be made. Furthermore, antibodies expressed from transgenes are post-translationally modified in a different manner than those that are directly injected because of the different microenvironment present during and after translation. Without being bound by any particular theory, this results in antibodies that have different diffusion, bioactivity, distribution, affinity, pharmacokinetic, and immunogenicity characteristics, such that the antibodies delivered to the site of action are “biobetters” in comparison with directly injected antibodies. Accordingly, provided herein are compositions and methods for anti-factor XII gene therapy, particularly recombinant AAV gene therapy, designed to target the liver and/or muscle and generate a depot of transgenes for expression of anti-factor XII antibodies, particularly garadacimab, AB042/AB043, AB054, DX-4012, Ab26036, Ab26048, Ab260489, Ab26076, 559C-M0071-F06, 559C-M0179-D04, 559C-M0181-C02, 559C-M0180-G03, 559C-M0184-B04, 620I-X0173-A11, 620I-X0173-C07, 620I-X0173-E07, or 620I-X0173-G11, or an antigen binding fragment thereof, that result in a therapeutic or prophylactic serum levels of the antibody within 20 days, 30 days, 40 days, days, 60 days, or 90 days of administration of the rAAV composition.

Compositions and methods are described for the systemic delivery of an anti-factor XII HuPTM mAb or an anti-factor XII HuPTM antigen-binding fragment of a therapeutic mAb (for example, a fully human-glycosylated Fab (HuGlyFab) of a therapeutic mAb, to a patient (human subject) diagnosed with hereditary angioedema or other condition indicated for treatment with the therapeutic anti-factor XII mAb. Such antigen-binding fragments of therapeutic mAbs include a Fab, F(ab′)₂, or scFv (single-chain variable fragment) (collectively referred to herein as “antigen-binding fragment”). “HuPTM Fab” as used herein may include other antigen binding fragments of a mAb. In an alternative embodiment, full-length mAbs can be used. In yet another embodiment, the therapeutic anti-factor XII mAb may be part of a bispecific antibody which also binds pKal. Delivery may be advantageously accomplished via gene therapy—e.g., by administering a viral vector or other DNA expression construct encoding a therapeutic anti-factor XII mAb or its antigen-binding fragment (or a hyperglycosylated derivative of either) diagnosed with a condition indicated for treatment with the therapeutic anti-factor XII mAb—to create a permanent depot in liver, or in alternative embodiments, muscle, of the patient that continuously supplies the HuPTM mAb or antigen-binding fragment of the therapeutic mAb, e.g., a human-glycosylated transgene product, or peptide to the circulation of the subject where the mAb or antigen-binding fragment thereof or peptide exerts its therapeutic or prophylactic effect.

Provided are gene therapy vectors, particularly rAAV gene therapy vectors, which when administered to a human subject result in expression of an anti-factor XII antibody to achieve a therapeutic serum concentration, for example, 20, 30, 40, 50, 60 or 90 days after administration. In certain embodiments, the antibody binds to its target, for example, in an antibody binding assay (e.g. enzyme-linked immunosorbent assay (ELISA) binding assay or surface plasmon resonance (SPR)-based real-time kinetics assay), preferably in the picomolar or nanomolar range, and/or exhibits biological activity in an appropriate assay. Dosages include 1E11 to 1E14 vector genomes per kilogram body weight (vg/kg) administered, particularly, parenterally, including intravenously. Dosages result in sufficient copy number of viral genomes incorporated into liver and/or muscle cells, for example, from at least 10, 20, 50, 60 or 80 vg/dg liver and/or muscle tissue and up to 100, 150, 200, 500 or 100 vg/dg liver and/or muscle tissue by 30, 60, 90 or 100 days or one year after administration. The dosage achieves the therapeutic or prophylactic serum levels of the anti-Factor XII antibody while minimizing or avoiding adverse effects such as transaminitis and/or the presence of anti-drug antibodies.

The recombinant vector used for delivering the transgene includes non-replicating recombinant adeno-associated virus vectors (“rAAV”). In embodiments, the AAV type has a tropism for liver and/or muscle cells, or example AAV8 subtype of AAV. However, other viral vectors may be used, including but not limited to lentiviral vectors; vaccinia viral vectors, or non-viral expression vectors referred to as “naked DNA” constructs. Expression of the transgene can be controlled by constitutive or tissue-specific expression control elements, particularly elements that are liver and/or muscle specific control elements, for example one or more elements of Table 1.

In certain embodiments, the HuPTM mAb or HuPTM antigen-binding fragment encoded by the transgene can include, but is not limited to, a full-length or an antigen-binding fragment of a therapeutic antibody that binds to factor XII, particularly garadacimab, AB042/AB043, AB054, DX-4012, Ab26036, Ab26048, Ab260489, Ab26076, 559C-M0071-F06, 559C-M0179-D04, 559C-M0181-C02, 559C-M0180-G03, 559C-M0184-B04, 620I-X0173-A11, 620I-X0173-C07, 620I-X0173-E07, or 620I-X0173-G11, see, for example FIGS. 2A-2I.

Gene therapy constructs for the therapeutic antibodies are designed such that both the heavy and light chains are expressed. The coding sequences for the heavy and light chains can be engineered in a single construct in which the heavy and light chains are separated by a cleavable linker or IRES so that separate heavy and light chain polypeptides are expressed. In particular embodiments, the linker is a Furin T2A linker (SEQ ID NOS: 155 or 156). In certain embodiments, the coding sequences encode for a Fab or F(ab′)₂ or an scFv. In certain embodiments the full length heavy and light chains of the antibody are expressed. In other embodiments, the constructs express an scFv in which the heavy and light chain variable domains are connected via a flexible, non-cleavable linker. In certain embodiments, the construct expresses, from the N-terminus, NH₂-V_{L}-linker-V_{H}—COOH or NH₂-V_{H}-linker-V_{L}—COOH.

In addition, antibodies expressed from transgenes in vivo are not likely to contain degradation products associated with antibodies produced by recombinant technologies, such as protein aggregation and protein oxidation. Aggregation is an issue associated with protein production and storage due to high protein concentration, surface interaction with manufacturing equipment and containers, and purification with certain buffer systems. These conditions, which promote aggregation, do not exist in transgene expression in gene therapy. Oxidation, such as methionine, tryptophan, and histidine oxidation, is also associated with protein production and storage, and is caused by stressed cell culture conditions, metal and air contact, and impurities in buffers and excipients. The proteins expressed from transgenes in vivo may also oxidize in a stressed condition. However, humans, and many other organisms, are equipped with an antioxidation defense system, which not only reduces the oxidation stress, but sometimes also repairs and/or reverses the oxidation. Thus, proteins produced in vivo are not likely to be in an oxidized form. Both aggregation and oxidation could affect the potency, pharmacokinetics (clearance), and immunogenicity.

The production of HuPTM mAb or HuPTM Fab in liver and/or muscle cells of the human subject should result in a “biobetter” molecule for the treatment of disease accomplished via gene therapy—e.g., by administering a viral vector or other DNA expression construct encoding a full-length HuPTM mAb or HuPTM Fab of a therapeutic mAb to a patient (human subject) diagnosed with a disease indication for that mAb, to create a permanent depot in the subject that continuously supplies the human-glycosylated, sulfated transgene product produced by the subject's transduced cells. The cDNA construct for the HuPTMmAb or HuPTM Fab should include a signal peptide that ensures proper co- and post-translational processing (glycosylation and protein sulfation) by the transduced human cells.

As an alternative, or an additional treatment to gene therapy, the full-length HuPTM mAb or HuPTM Fab can be produced in human cell lines by recombinant DNA technology, and the glycoprotein can be administered to patients.

Combination therapies involving systemic delivery of the full-length HuPTM anti-Factor XII mAb or HuPTM anti-Factor XII Fab to the patient accompanied by administration of other available treatments are encompassed by the methods provided herein. The additional treatments may be administered before, concurrently or subsequent to the gene therapy treatment. Such additional treatments can include but are not limited to co-therapy with the therapeutic mAb.

Also provided are methods of manufacturing the viral vectors, particularly the AAV based viral vectors. In specific embodiments, provided are methods of producing recombinant AAVs comprising culturing a host cell containing an artificial genome comprising a cis expression cassette flanked by AAV ITRs, wherein the cis expression cassette comprises a transgene encoding a therapeutic antibody operably linked to expression control elements that will control expression of the transgene in human cells; a trans expression cassette lacking AAV ITRs, wherein the trans expression cassette encodes an AAV rep and capsid protein operably linked to expression control elements that drive expression of the AAV rep and capsid proteins in the host cell in culture and supply the rep and cap proteins in trans; sufficient adenovirus helper functions to permit replication and packaging of the artificial genome by the AAV capsid proteins; and recovering recombinant AAV encapsidating the artificial genome from the cell culture.

The inventors found that intravenous administration of an AAV8-based vector comprising an optimized expression cassette containing a liver-specific promoter and a codon optimized and CpG depleted transgene with a modified furin-T2A processing signal results in dose-dependent and sustained serum antibody concentrations in non-human primates. Accordingly, provided are compositions comprising rAAV vectors which comprise an optimized expression cassette containing a liver-specific promoter and a codon optimized and CpG depleted transgene with a modified furin-T2A processing signal that express a transgene, for example, heavy and light chains of an anti-Factor XII therapeutic antibody. Methods of administration and manufacture are also provided.

### 3.1 Illustrative Embodiments

1. A pharmaceutical composition for treating hereditary angioedema, thrombosis, or hypercoagulation in a human subject in need thereof, comprising an adeno-associated virus (AAV) vector having:
   (a) a viral capsid that has a tropism for liver and/or muscle cells; and
   (b) an artificial genome comprising an expression cassette flanked by AAV inverted terminal repeats (ITRs), wherein the expression cassette comprises a transgene encoding a heavy chain and a light chain of a substantially full-length or full-length anti-factor XII/XIIa mAb, operably linked to one or more regulatory sequences that promote expression of the transgene in human liver and/or muscle cells;
wherein said AAV vector is formulated for systemic administration to said human subject.
2. The pharmaceutical composition of paragraph 1, wherein the viral capsid is at least 95% identical to the amino acid sequence of AAV3B, AAV5, AAV7 (SEQ ID NO:198), AAV8 (SEQ ID NO:199), AAV9 (SEQ ID NO:200), AAVrh10 (SEQ ID NO:201), AAVrh46 (SEQ ID NO:202), AAVrh73 (SEQ ID NO:203), AAVS3 (SEQ ID NO:205), AAV-LK03 (SEQ ID NO:204), AAVrh8, AAV64R1, or AAVhu37.
3. The pharmaceutical composition of paragraphs 1 or 2 wherein the AAV capsid is AAV8 or AAVS3.
4. The pharmaceutical composition of paragraphs 1 to 3, wherein the regulatory sequence includes a regulatory sequence from Table 1.
5. The pharmaceutical composition of paragraph 4, wherein the regulator sequence is an ApoE.hAAT (SEQ ID NO:78) regulatory sequence, a LSPX1 promoter (SEQ ID NO:66), a LSPX2 promoter (SEQ ID NO:67), a LTP1 promoter (SEQ ID NO:68), a LTP2 (SEQ ID NO:69) promoter, a LTP3 (SEQ ID NO:70) promoter, or an LMTP6 promoter (SEQ ID NO:71).
6. The pharmaceutical composition of any of paragraphs 1 to 5, wherein the transgene comprises a Furin/2A linker between the nucleotide sequences coding for the heavy and light chains of said mAb.
7. The pharmaceutical composition of paragraph 6, wherein said Furin 2A linker is a Furin/T2A linker having the amino acid sequence RKRR(GSG)EGRGSLLTCGDVEENPGP (SEQ ID NOS:155 or 156).
8. The pharmaceutical composition of any of paragraphs 1 to 7, wherein the transgene encodes a signal sequence at the N-terminus of the heavy chain and the light chain of said antigen-binding fragment that directs secretion and post translational modification in said human liver and/or muscle cells.
9. The pharmaceutical composition of paragraph 8, wherein said signal sequence is MYRMQLLLLIALSLALVTNS (SEQ ID NO:103) or a signal sequence from Table 2.
10. The pharmaceutical composition of any of paragraphs 1 to 9, wherein transgene has the structure: signal sequence-Heavy chain-Furin site-2A site-signal sequence-Light chain-PolyA.
11. The pharmaceutical composition of any of paragraphs 1 to 10, wherein the anti-Factor XII antibody is garadacimab, AB042/AB043, AB054, DX-4012, Ab26036, Ab26048, Ab260489, Ab26076, 559C-M0071-F06, 559C-M0179-D04, 559C-M0181-C02, 559C-M0180-G03, 559C-M0184-B04, 620I-X0173-A11, 620I-X0173-C07, 620I-X0173-E07, or 620I-X0173-G11 or an antigen binding fragment thereof
12. The pharmaceutical composition of any of paragraphs 1 to 11, wherein the full-length mAb or the antigen-binding fragment comprises a heavy chain with an amino acid sequence of SEQ ID NO: 1 and optionally an Fc polypeptide with an amino acid sequence of SEQ ID NO: 42 and a light chain with an amino acid sequence of SEQ ID NO: 2; a heavy chain with an amino acid sequence of SEQ ID NO: 3 and a light chain with an amino acid sequence of SEQ ID NO: 4; a heavy chain with an amino acid sequence of SEQ ID NO: 5 and optionally an Fc polypeptide with an amino acid sequence of SEQ ID NO:64 and a light chain with an amino acid sequence of SEQ ID NO: 6; a heavy chain with an amino acid sequence of SEQ ID NO: 7 and optionally an Fc polypeptide with an amino acid sequence of SEQ ID NO:65 and a light chain with an amino acid sequence of SEQ ID NO: 8; a heavy chain with an amino acid sequence of SEQ ID NO: 10 and a light chain with an amino acid sequence of SEQ ID NO: 11; a heavy chain with an amino acid sequence of SEQ ID NO: 12 and a light chain with an amino acid sequence of SEQ ID NO: 13; a heavy chain with an amino acid sequence of SEQ ID NO: 14 and a light chain with an amino acid sequence of SEQ ID NO: 15; a heavy chain with an amino acid sequence of SEQ ID NO: 16 and a light chain with an amino acid sequence of SEQ ID NO: 17; a heavy chain with an amino acid sequence of SEQ ID NOS: 18, 20, 22, or 24 and a light chain with an amino acid sequence of SEQ ID NO: 19; a heavy chain with an amino acid sequence of SEQ ID NO: 26 and a light chain with an amino acid sequence of SEQ ID NO: 27; a heavy chain with an amino acid sequence of SEQ ID NO: 26 and a light chain with an amino acid sequence of SEQ ID NO: 27, 28, 29, or 30; or a heavy chain with an amino acid sequence of SEQ ID NO: 31 and a light chain with an amino acid sequence of SEQ ID NO: 27.
13. A composition comprising an adeno-associated virus (AAV) vector having:
   a. a viral AAV capsid, that is optionally at least 95% identical to the amino acid sequence of AAV3B, AAV5, AAV7 (SEQ ID NO: 198), AAV8 (SEQ ID NO: 199), AAV9 (SEQ ID NO: 200), AAVrh10 (SEQ ID NO: 201), AAVrh46 (SEQ ID NO: 202), AAVrh73 (SEQ ID NO: 203), AAVS3 (SEQ ID NO: 205), AAV-LK03 (SEQ ID NO: 204), AAVrh8, AAV64R1, or AAVhu37; and
   b. an artificial genome comprising an expression cassette flanked by AAV inverted terminal repeats (ITRs), wherein the expression cassette comprises a transgene encoding a heavy and a light chain of a substantially full-length or full-length anti-factor XII mAb, operably linked to one or more regulatory sequences that promote expression of the transgene in human liver and/or muscle cells;
   c. wherein the transgene encodes a signal sequence at the N-terminus of the heavy chain and the light chain of said mAb that directs secretion and post translational modification of said mAb in liver and/or muscle cells.
14. The composition of paragraph 13, wherein the anti-factor XII antibody is garadacimab, AB042/AB043, AB054, DX-4012, Ab26036, Ab26048, Ab260489, Ab26076, 559C-M0071-F06, 559C-M0179-D04, 559C-M0181-C02, 559C-M0180-G03, 559C-M0184-B04, 620I-X0173-A11, 620I-X0173-C07, 620I-X0173-E07, or 620I-X0173-G11 or an antigen binding fragment thereof
15. The composition of paragraphs 13 or 14, wherein the full-length mAb or the antigen-binding fragment comprises a heavy chain with an amino acid sequence of SEQ ID NO: 1 and optionally an Fc polypeptide with an amino acid sequence of SEQ ID NO:63 and a light chain with an amino acid sequence of SEQ ID NO: 2; a heavy chain with an amino acid sequence of SEQ ID NO: 3 and a light chain with an amino acid sequence of SEQ ID NO: 4; a heavy chain with an amino acid sequence of SEQ ID NO: 5 and optionally an Fc polypeptide with an amino acid sequence of SEQ ID NO:64 and a light chain with an amino acid sequence of SEQ ID NO: 6; a heavy chain with an amino acid sequence of SEQ ID NO: 7 and optionally an Fc polypeptide with an amino acid sequence of SEQ ID NO:65 and a light chain with an amino acid sequence of SEQ ID NO: 8; a heavy chain with an amino acid sequence of SEQ ID NO: 10 and a light chain with an amino acid sequence of SEQ ID NO: 11; a heavy chain with an amino acid sequence of SEQ ID NO: 12 and a light chain with an amino acid sequence of SEQ ID NO: 13; a heavy chain with an amino acid sequence of SEQ ID NO: 14 and a light chain with an amino acid sequence of SEQ ID NO: 15; a heavy chain with an amino acid sequence of SEQ ID NO: 16 and a light chain with an amino acid sequence of SEQ ID NO: 17; a heavy chain with an amino acid sequence of SEQ ID NOS: 18, 20, 22, or 24 and a light chain with an amino acid sequence of SEQ ID NO: 19; a heavy chain with an amino acid sequence of SEQ ID NO: 26 and a light chain with an amino acid sequence of SEQ ID NO: 27; a heavy chain with an amino acid sequence of SEQ ID NO: 26 and a light chain with an amino acid sequence of SEQ ID NO: 27, 28, 29, or 30; or a heavy chain with an amino acid sequence of SEQ ID NO: 31 and a light chain with an amino acid sequence of SEQ ID NO: 27.
16. The composition of any of paragraphs 13 to 15, wherein the transgene comprises a Furin/2A linker between the nucleotide sequences coding for the heavy and light chains of said mAb.
17. The composition of paragraph 16, wherein the nucleic acid encoding a Furin 2A linker is incorporated into the expression cassette in between the nucleotide sequences encoding the heavy and light chain sequences, resulting in a construct with the structure: Signal sequence-Heavy chain-Furin site-2A site-Signal sequence-Light chain-PolyA.
18. The composition of paragraphs 13 to 17, wherein said Furin 2A linker is a Furin/T2A linker having the amino acid sequence RKRR(GSG)EGRGSLLTCGDVEENPGP (SEQ ID NOS:155 or 156).
19. The composition of any of paragraphs 13 to 18, wherein said signal sequence is MYRMQLLLLIALSLALVTNS (SEQ ID NO:103) or a signal sequence from Tables 2 or 3.

### Method of Treatment

20. A method of treating hereditary angioedema, thrombosis, or hypercoagulation in a human subject in need thereof, comprising intravenously administering to the subject a therapeutically effective amount of a composition comprising a recombinant AAV comprising a transgene encoding garadacimab, AB042/AB043, AB054, DX-4012, Ab26036, Ab26048, Ab260489, Ab26076, 559C-M0071-F06, 559C-M0179-D04, 559C-M0181-C02, 559C-M0180-G03, 559C-M0184-B04, 620I-X0173-A11, 620I-X0173-C07, 620I-X0173-E07, or 620I-X0173-G11, operably linked to one or more regulatory sequences that control expression of the transgene in liver and/or muscle cells, in an amount sufficient to result in expression from the transgene and secretion of garadacimab, AB042/AB043, AB054, DX-4012, Ab26036, Ab26048, Ab260489, Ab26076, 559C-M0071-F06, 559C-M0179-D04, 559C-M0181-C02, 559C-M0180-G03, 559C-M0184-B04, 620I-X0173-A11, 620I-X0173-C07, 620I-X0173-E07, or 620I-X0173-G11 into the bloodstream of the human subject.
21. The method of paragraph 20, wherein the full-length mAb or the antigen-binding fragment comprises a heavy chain with an amino acid sequence of SEQ ID NO: 1 and optionally an Fc polypeptide with an amino acid sequence of SEQ ID NO:63 and a light chain with an amino acid sequence of SEQ ID NO: 2; a heavy chain with an amino acid sequence of SEQ ID NO: 3 and a light chain with an amino acid sequence of SEQ ID NO: 4; a heavy chain with an amino acid sequence of SEQ ID NO: 5 and optionally an Fc polypeptide with an amino acid sequence of SEQ ID NO:64 and a light chain with an amino acid sequence of SEQ ID NO: 6; a heavy chain with an amino acid sequence of SEQ ID NO: 7 and optionally an Fc polypeptide with an amino acid sequence of SEQ ID NO:65 and a light chain with an amino acid sequence of SEQ ID NO: 8; a heavy chain with an amino acid sequence of SEQ ID NO: 10 and a light chain with an amino acid sequence of SEQ ID NO: 11; a heavy chain with an amino acid sequence of SEQ ID NO: 12 and a light chain with an amino acid sequence of SEQ ID NO: 13; a heavy chain with an amino acid sequence of SEQ ID NO: 14 and a light chain with an amino acid sequence of SEQ ID NO: 15; a heavy chain with an amino acid sequence of SEQ ID NO: 16 and a light chain with an amino acid sequence of SEQ ID NO: 17; a heavy chain with an amino acid sequence of SEQ ID NOS: 18, 20, 22, or 24 and a light chain with an amino acid sequence of SEQ ID NO: 19; a heavy chain with an amino acid sequence of SEQ ID NO: 26 and a light chain with an amino acid sequence of SEQ ID NO: 27; a heavy chain with an amino acid sequence of SEQ ID NO: 26 and a light chain with an amino acid sequence of SEQ ID NO: 27, 28, 29, or 30; or a heavy chain with an amino acid sequence of SEQ ID NO: 31 and a light chain with an amino acid sequence of SEQ ID NO: 27.
22. The method of paragraphs 20 or 21 wherein the recombinant AAV has a viral capsid which is at least 95% identical to the amino acid sequence of AAV3B, AAV5, AAV7 (SEQ ID NO: 198), AAV8 (SEQ ID NO: 199), AAV9 (SEQ ID NO: 200), AAVrh10 (SEQ ID NO: 201), AAVrh46 (SEQ ID NO: 202), AAVrh73 (SEQ ID NO: 203), AAVS3 (SEQ ID NO: 205), AAV-LK03 (SEQ ID NO: 204), AAVrh8, AAV64R1, or AAVhu37.
23. The method of any of paragraphs 20 to 22, wherein the AAV capsid is AAV8 or AAVS3.
24. The method of any of paragraphs 20 to 23, wherein the regulatory sequence includes a regulatory sequence from Table 1.
25. The method of paragraph 24, wherein the regulator sequence is an ApoE.hAAT (SEQ ID NO: 58) regulatory sequence, a LSPX1 promoter (SEQ ID NO: 46), a LSPX2 promoter (SEQ ID NO: 67), a LTP1 promoter (SEQ ID NO: 68), a LTP2 (SEQ ID NO: 69) promoter, a LTP3 (SEQ ID NO: 70) promoter or an LMTP6 (SEQ ID NO: 71) promoter.
26. The method of any of paragraphs 20 to 25, wherein the transgene comprises a Furin/2A linker between the nucleotide sequences coding for the heavy and light chains of said mAb.
27. The method of paragraph 26, wherein said Furin 2A linker is a Furin/T2A linker having the amino acid sequence RKRR(GSG)EGRGSLLTCGDVEENPGP (SEQ ID NOS:155 or 156).
28. The method of any of paragraphs 20 to 27, wherein the transgene encodes a signal sequence at the N-terminus of the heavy chain and the light chain of said antigen-binding fragment that directs secretion and post translational modification in said human liver and/or muscle cells.
29. The method of paragraph 28, wherein said signal sequence is MYRMQLLLLIALSLALVTNS (SEQ ID NO:103) or a signal sequence from Tables 2 or 3.
30. The method of any of paragraphs 20 to 29, wherein transgene has the structure: Signal sequence-Heavy chain-Furin site-2A site-Signal sequence-Light chain-PolyA.
31. The method of any of paragraphs 20 to 30, wherein the mAb is a hyperglycosylated mutant or wherein the Fc polypeptide of the mAb is glycosylated or aglycosylated.
32. The method of paragraphs 20 to 31 wherein the mAb contains an alpha 2,6-sialylated glycan.
33. The method of any of paragraphs 20 to 32 wherein the mAb is glycosylated but does not contain detectable NeuGc and/or α-Gal.
34. The method of any of paragraphs 20 to 32 wherein the mAb contains a tyrosine sulfation.
35. The method of any of paragraphs 20 to 34 in which production of said HuPTM form of said mAb or antigen-binding fragment thereof is confirmed by transducing human liver and/or muscle cells in culture with said recombinant nucleotide expression vector and expressing said mAb or antigen-binding fragment thereof
36. The method of any of paragraphs 20 to 35, wherein the therapeutically effective amount is determined to be sufficient to reduce HAE attack frequency, reduce progression of angioedema, reduction in coagulation frequency, reduction in thrombosis formation.

### Method of Manufacture

37. A method of producing recombinant AAVs comprising:
   (a) culturing a host cell containing:
      (i) an artificial genome comprising a cis expression cassette flanked by AAV ITRs, wherein the cis expression cassette comprises comprising a transgene encoding a substantially full-length or full-length anti-factor XII mAb, operably linked to one or more regulatory sequences that promote expression of the transgene in human liver and/or muscle cells;
      (ii) a trans expression cassette lacking AAV ITRs, wherein the trans expression cassette encodes an AAV rep and an AAV capsid protein operably linked to expression control elements that drive expression of the AAV rep and the AAV capsid protein in the host cell in culture and supply the AAV rep and the AAV capsid protein in trans, wherein the capsid has liver and/or muscle tropism;
      (iii) sufficient adenovirus helper functions to permit replication and packaging of the artificial genome by the AAV capsid protein; and
   (b) recovering recombinant AAV encapsidating the artificial genome from the cell culture.
38. The method of paragraph 37, wherein the transgene encodes a substantially full-length or full-length mAb or antigen binding fragment that comprises the heavy and light chain variable domains of garadacimab, AB042/AB043, AB054, DX-4012, Ab26036, Ab26048, Ab260489, Ab26076, 559C-M0071-F06, 559C-M0179-D04, 559C-M0181-C02, 559C-M0180-G03, 559C-M0184-B04, 620I-X0173-A11, 620I-X0173-C07, 620I-X0173-E07, or 620I-X0173-G11.
39. A host cell containing:
   a. an artificial genome comprising a cis expression cassette flanked by AAV ITRs, wherein the cis expression cassette comprises comprising a transgene encoding a substantially full-length or full-length anti-Factor XII mAb, operably linked to one or more regulatory sequences that promote expression of the transgene in human liver and/or muscle cells;
   b. a trans expression cassette lacking AAV ITRs, wherein the trans expression cassette encodes an AAV rep and an AAV capsid protein operably linked to expression control elements that drive expression of the AAV rep and the AAV capsid protein in the host cell in culture and supply the AAV rep and the AAV capsid protein in trans, wherein the capsid has liver and/or muscle tropism;
   c. sufficient adenovirus helper functions to permit replication and packaging of the artificial genome by the AAV capsid protein.
40. The host cell of paragraph 39, wherein the transgene encodes a substantially full-length or full-length mAb or antigen binding fragment that comprises the heavy and light chain variable domains of garadacimab, AB042/AB043, AB054, DX-4012, Ab26036, Ab26048, Ab260489, Ab26076, 559C-M0071-F06, 559C-M0179-D04, 559C-M0181-C02, 559C-M0180-G03, 559C-M0184-B04, 620I-X0173-A11, 620I-X0173-C07, 620I-X0173-E07, or 620I-X0173-G11.
41. The host cell of paragraphs 39 to 40, wherein the AAV capsid protein is an AAV3B, AAV5, AAV7 (SEQ ID NO: 198), AAV8 (SEQ ID NO: 199), AAV9 (SEQ ID NO: 200), AAVrh10 (SEQ ID NO:201), AAVrh46 (SEQ ID NO: 202), AAVrh73 (SEQ ID NO: 203), AAVS3 (SEQ ID NO: 205), AAV-LK03 (SEQ ID NO: 204), AAVrh8, AAV64R1, or AAVhu37 capsid protein.

### 4. BRIEF DESCRIPTION OF THE DRAWINGS

FIGS. 1A and 1B. A) A schematic of an rAAV vector genome construct containing an expression cassette encoding the heavy and light chains of a therapeutic mAb separated by a Furin-2A linker, operably linked to a liver-specific enhancer and/or promoter, controlled by expression elements, flanked by the AAV ITRs. B) A schematic of an rAAV vector genome construct containing an expression cassette encoding the heavy and light chains of a therapeutic mAb separated by a Furin-T2A linker, controlled by expression elements, flanked by the AAV ITRs. The transgene can comprise nucleotide sequences encoding the heavy and light chains of the Fab portion or the full-length heavy (CH1 plus hinge) and light chains with Fc regions.

FIGS. 2A-2J. The amino acid sequence of a transgene construct for the variable or Fab region of anti-factor XII antibodies, garadacimab (A), AB042/AB043 (B), AB054 (C), DX-4012 (D), Ab26036 (E), Ab26048 (F), Ab260489 (G), Ab26076 (H), 620I-X0173-A01 (I and J, as part of bi-specific HC+scFv antibody). Glycosylation sites are boldface. Glutamine glycosylation sites; asparaginal (N) glycosylation sites, non-consensus asparaginal (N) glycosylation sites; and tyrosine-sites (italics) are as indicated in the legend. Complementarity-determining regions (CDR) are underscored. The hinge region is highlighted in grey.

FIG. 3. Clustal Multiple Sequence Alignment of various capsids with liver and/or muscle tropism. Amino acid substitutions (shown in bold in the bottom rows) can be made to AAV8 capsids by “recruiting” amino acid residues from the corresponding position of other aligned AAV capsids. Sequence shown in gray=hypervariable regions. The amino acid sequences of the AAV capsids are assigned SEQ ID NOs as follows: AAV2 is SEQ ID NO: 197; AAV7 is SEQ ID NO: 198; AAV8 is SEQ ID NO: 199; AAV9 is SEQ ID NO: 200; AAVrh10 is SEQ ID NO: 201; AAVrh46 (SEQ ID NO: 202), AAVrh73 (SEQ ID NO: 203), AAVS3 (SEQ ID NO: 205), and AAV-LK03 (SEQ ID NO: 204).

FIG. 4. Clustal Multiple Sequence Alignment of constant heavy chain regions (CH2 and CH3) of IgG1 (SEQ ID NO: 60), IgG2 (SEQ ID NO: 61), and IgG4 (SEQ ID NO: 62). The hinge region, from residue 219 to residue 230 of the heavy chain, is shown in italics. The numbering of the amino acids is in EU-format.

FIG. 5. The indicated AAV9 and AAV8 vectors (n=5 per group) were administered to NGS mice via either intravenous (IV) or intramuscular (IM) routes. IV administrations were into the tail vein and IM administrations were bilateral into the gastrocnemius muscles. Mice treated with vehicle were included as controls. Seven weeks post administration mice were sacrificed, and serum human antibody levels were determined by ELISA.

FIG. 6. A time course of antibody expression (lanadelumab serum levels) in NGS mice post-AAV9 administration (n=5 per group) is shown. AAV9 vectors (2e11 gc) were injected either IV or IM and serum antibody levels were determined by ELISA at day 7 (D7), day 21 (D21), day 35 (D35), and day 49 (D49).

FIGS. 7A and 7B. A Serum expression levels (μg/ml) of lanadelumab upon intravenous injection of C/57BL6 mice with 2.5×10¹² vg/kg of AAV8 vectors encoding a lanadelumab regulated by different liver-specific, liver-tandem and liver-muscle regulatory elements (see Table 1). CAG (SEQ ID NO: 89) and TBG (SEQ ID NO: 93) promoters were used as controls. Data from the blood draw at 1, 3, 5 and 7 weeks post injection are shown. LSPX1, liver-specific promoter 1 (SEQ ID NO: 66); LSXP2, liver-specific promoter 2 (SEQ ID NO: 67); LTP1, liver-specific tandem promoter 1 (SEQ ID NO: 68); LMTP6, liver and muscle dual-specific tandem promoter 6 (SEQ ID NO: 71). Protein expression levels were quantified by ELISA from biweekly serum collections. N=5 mice per vector. Numbers on x-axis represent the weeks post vector administration. Data represent mean+SEM. B. Quantification of viral genomes in liver. C57B1/6 mice were administrated intravenously with AAV8 vectors driven by different liver-specific promoters at equivalent doses (2.5×10¹² vg/kg). N=5 mice per group. Vector DNA was analyzed by ddPCR in mouse liver samples collected at 49 days post vector administration. Data represent mean+SEM.

FIGS. 8A and 8B. A. Route of administration and dose selection in Wistar rats. AAV8 vectors encoding vectorized lanadelumab driven by CAG promoters were injected intramuscularly at 1×10¹³ vg/kg (body weight) or intravenously at 1×10¹³ vg/kg and 1×10¹⁴ vg/kg into SD rats. Protein expression was quantified by ELISA from serum collected every three to seven days. N=3 rats per vector. Data represent mean+SEM. * indicates p<0.05, ** indicates p<0.01 with Welch's t test. B. AAV8 vectors encoding vectorized lanadelumab driven by CAG (SEQ ID NO: 89) or ApoE.hAAT (SEQ ID NO: 78) promoters were injected intravenously at 5×10¹³ vg/kg into Wistar and SD rats. Protein expression was quantified by ELISA from weekly serum collection. N=3 rats per vector. Data represent mean+SEM. P value: *, p<0.05; **, p<0.01. Serum antibody concentrations (mean and SEM) in animals of each group at each time point are presented in the table.

FIGS. 9A-9D. A. Serum anti-kallikrein (pKal) (lanadelumab) antibody concentration following AAV8 delivery. Animals received bilateral injections of 5×10¹⁰ vg/kg into the GA muscle. Serum was collected biweekly and vectorized antibody concentration was quantified with ELISA. B. Vector genome quantification from relevant tissues with digital droplet PCR (ddPCR). C. Comparison of vector gene expression from liver. Data represent relative fold gene expression as quantified by the ΔΔCT method. D. Comparison of AAV transgene expression from tissues using digital droplet PCR (ddPCR). Anti-pKal antibody mRNA copies were normalized to GAPDH mRNA copies across tissues. Data are represented as mean±SEM. Statistical significance was determined using a one-way ANOVA followed by Tukey's HSD post-test. *P<0.05, **P<0.01.

FIGS. 10A-10L. Quantification of mouse paw volumes and paw swellings in carrageenan-induced paw edema mice treated with test articles. Bar charts show the paw volumes (A, C, E, G, I, and K) measured at 2 (A), 4 (C), 6 (E), 8 (G), 24 (I) and 48 (K) hours after carrageenan injection in C57BL/6 mice. Paw swelling difference (B, D, F, H, J, and L) was evaluated by calculating the difference of paw volumes measured at each time point and the baseline. N=10 mice per group. Data analysis was done with One-way ANOVA with Dunnett's post-hoc test for multiple comparisons. Data represent mean+S.DEM. P values: *, p<0.05; **, p<0.01; ***, p<0.001; ****, p<0.0001.

### 5. DETAILED DESCRIPTION OF THE INVENTION

Compositions and methods are described for the systemic delivery of a fully human post-translationally modified (HuPTM) therapeutic monoclonal antibody (mAb) or a HuPTM antigen-binding fragment of a therapeutic anti-factor XII mAb (for example, a fully human-glycosylated Fab (HuGlyFab) of a therapeutic mAb) to a patient (human subject) diagnosed with a hereditary angioedema, thrombosis, or hypercoagulation, or other indication indicated for treatment with the therapeutic mAb. Delivery may be advantageously accomplished via gene therapy—e.g., by administering a viral vector or other DNA expression construct encoding a therapeutic mAb or its antigen-binding fragment (or a hyperglycosylated derivative of either) to a patient (human subject) diagnosed with a condition indicated for treatment with the therapeutic mAb—to create a permanent depot in a tissue or organ of the patient, particularly liver and/or muscle that continuously supplies the HuPTM mAb or antigen-binding fragment of the therapeutic mAb, e.g., a human-glycosylated transgene product, into the circulation of the subject to where the mAb or antigen-binding fragment there of exerts its therapeutic effect.

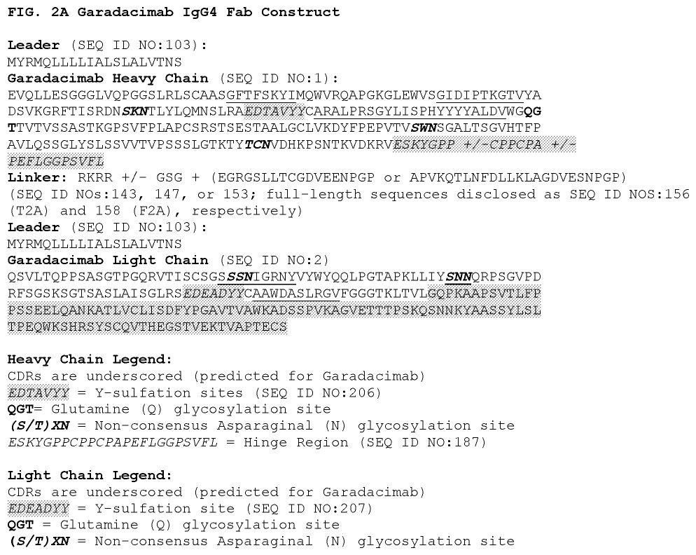

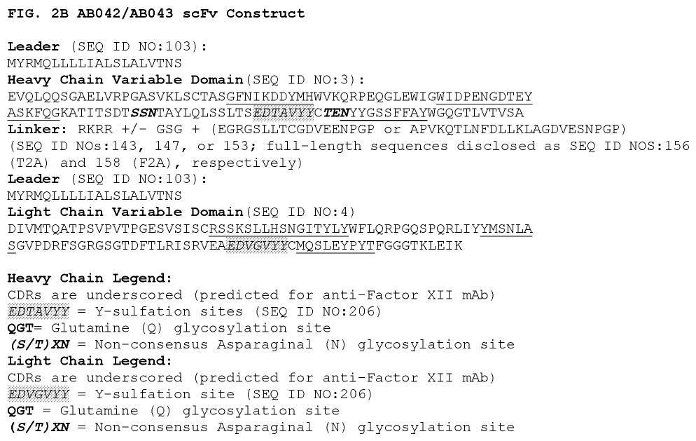

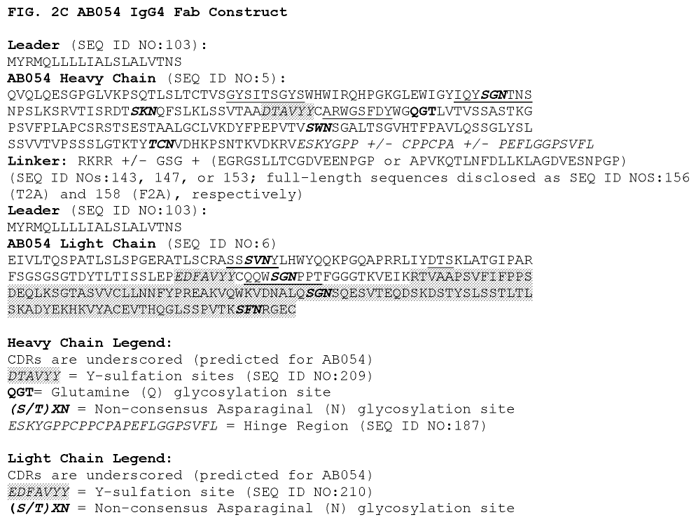

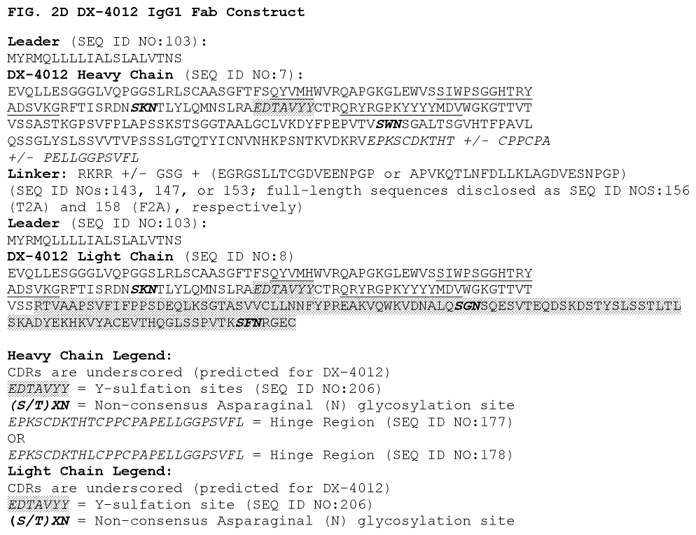

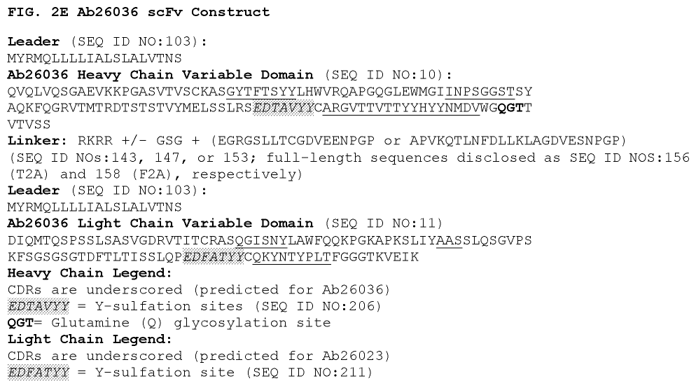

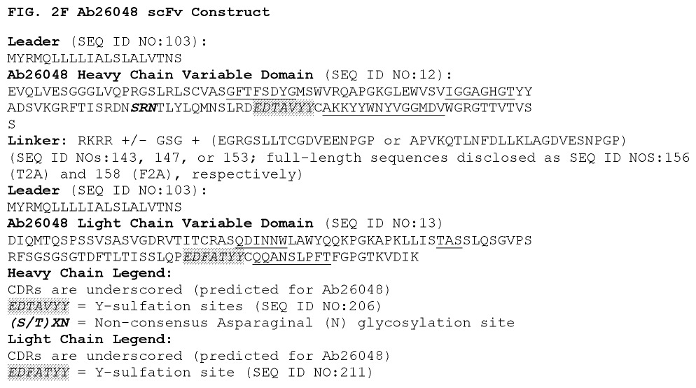

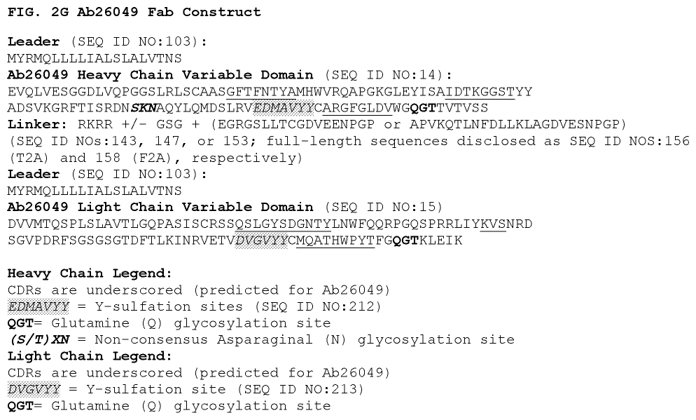

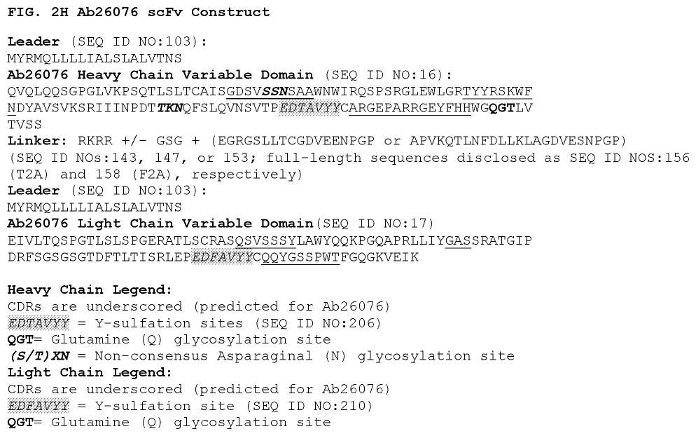

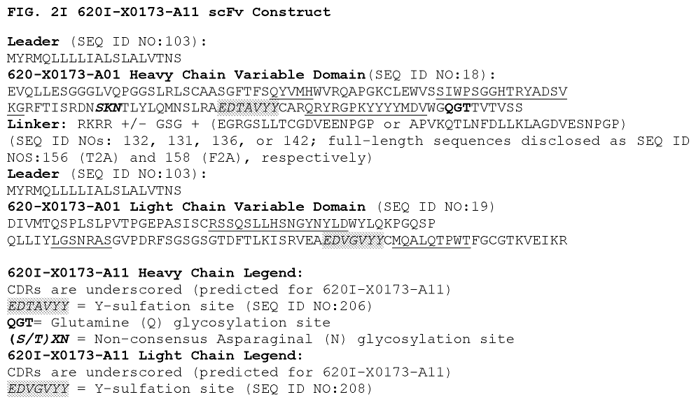

In certain embodiments, the HuPTM mAb or HuPTM antigen-binding fragment, encoded by a transgene, can be, but it not limited to, a full-length or an antigen-binding fragment of a HuPTM mAb or HuPTM that binds factor XII, particularly garadacimab, AB042/AB043, AB054, DX-4012, Ab26036, Ab26048, Ab260489, Ab26076, 559C-M0071-F06, 559C-M0179-D04, 559C-M0181-C02, 559C-M0180-G03, 559C-M0184-B04, 620I-X0173-A11, 620I-X0173-C07, 620I-X0173-E07, or 620I-X0173-G11 (see FIGS. 2A-2H for the variable heavy and variable light chain sequences of garadacimab, AB042/AB043, AB054, DX-4012, Ab26036, Ab26048, Ab260489, Ab26076, 620I-X0173-A01).

The compositions and methods provided herein systemically deliver a transgene encoding anti-factor XII antibodies, particularly, garadacimab, AB042/AB043, AB054, DX-4012, Ab26036, Ab26048, Ab260489, Ab26076, 559C-M0071-F06, 559C-M0179-D04, 559C-M0181-C02, 559C-M0180-G03, 559C-M0184-B04, 620I-X0173-A11, 620I-X0173-C07, 620I-X0173-E07, or 620I-X0173-G11, from a depot of viral genomes, for example, in the subject's liver (or muscle). In some embodiments, the viral genomes comprising the transgenes encoding anti-factor XII antibodies is at a serum level that is therapeutically or prophylactically effective to treat or ameliorate the symptoms of hereditary angioedema or other indication that may be treated with an anti-factor XII antibody. Identified herein are viral vectors for delivery of transgenes encoding the therapeutic anti-factor XII antibodies to cells in the human subject, including, in embodiments, liver cells, and regulatory elements operably linked to the nucleotide sequence encoding the heavy and light chains of the anti-factor XII antibody that promote the expression of the antibody in the cells, in embodiments, in the liver cells. Such regulatory elements, including liver specific regulatory elements, are provided in Table 1 herein.

The HuPTM mAb or HuPTM antigen-binding fragment encoded by a transgene can include, but is not limited to, a full-length or an antigen-binding fragment of a therapeutic antibody that binds to factor XII, including but not limited to, AB042/AB043, AB054, DX-4012, Ab26036, Ab26048, Ab260489, Ab26076, 559C-M0071-F06, 559C-M0179-D04, 559C-M0181-C02, 559C-M0180-G03, 559C-M0184-B04, 620I-X0173-A11, 620I-X0173-C07, 620I-X0173-E07, or 620I-X0173-G11. The amino acid sequences of the heavy chain variable domain and light chain variable domain of the foregoing are provided in Table 7, infra. Heavy chain variable domain having amino acid sequences of SEQ ID NOS: 1, 3, 5, 7, 10, 12, 14, 16, or 18, 20, 22, 24, 26, and 28 and light chain variable domain having amino acid sequences of SEQ ID NOS: 2, 4, 6, 8, 11, 13, 15, 17, 19, 27, 29, 30. The HuPTM mAb or HuPTM antigen-binding fragment encoded by the transgene can include, but is not limited to, a full-length or an antigen-binding fragment of a therapeutic antibody or antigen-binding fragments engineered to contain additional glycosylation sites on the Fab domain (e.g., see Courtois et al., 2016, mAbs 8: 99-112 which is incorporated by reference herein in its entirety for its description of derivatives of antibodies that are hyperglycosylated on the Fab domain of the full-length antibody).

A recombinant vector used for delivering a transgene includes non-replicating recombinant adeno-associated virus vectors (“rAAV”). rAAVs are particularly attractive vectors for a number of reasons—they can be modified to preferentially target a specific organ of choice; and there are hundreds of capsid serotypes to choose from to obtain the desired tissue specificity, and/or to avoid neutralization by pre-existing patient antibodies to some AAVs. The AAV types for use herein preferentially target the liver and/or muscle, i.e., have a tropism for liver and/or muscle cells. Such rAAVs include but are not limited to AAV based vectors comprising capsid components from one or more of AAV2, AAV3B, AAV-LK03, AAVS3, AAV7, AAV8, AAV9, AAV10, AAV11, AAVrh10, AAVrh46 or AAVrh73. In certain embodiments, AAV based vectors provided herein comprise capsids from one or more of AAV8, AAVrh46, AAVrh73, or AAVS3, or AAV-LK03 serotypes.

However, other viral vectors may be used, including but not limited to lentiviral vectors and vaccinia viral vectors. Non-viral expression vectors referred to as “naked DNA” constructs can also be used. Expression of a transgene carried by a recombinant vector can be controlled by constitutive or tissue-specific expression control elements.

Gene therapy constructs are designed such that both the heavy and light chains are expressed. In certain embodiments, the full length heavy and light chains of the antibody are expressed. In certain embodiments, the coding sequences encode for a Fab or F(ab′)₂ or an scFv. The heavy and light chains should be expressed at about equal amounts, in other words, the heavy and light chains are expressed at approximately a 1:1 ratio of heavy chains to light chains. The coding sequences for the heavy and light chains can be engineered in a single construct in which the heavy and light chains are separated by a cleavable linker or IRES so that separate heavy and light chain polypeptides are expressed. In specific embodiments, the linker separating the heavy and light chains is a Furin-2A linker, for example a Furin-F2A linker RKRR(GSG)APVKQTLNFDLLKLAGDVESNPGP (SEQ ID NOS:166 or 167) or a Furin-T2A linker RKRR(GSG)EGRGSLLTCGDVEENPGP (SEQ ID NOS:164 or 165). In certain embodiments, the construct expresses, from the N-terminus to C-terminus, NH2-VL-linker-VH-COOH or NH2-VH-linker-VL-COOH. In other embodiments, the construct expresses, from the N-terminus to C-terminus, NH2-signal or localization sequence-VL-linker-VH-COOH or NH2-signal or localization sequence-VH-linker-VL-COOH. In other embodiments, the constructs express an scFv in which the heavy and light chain variable domains are connected via a flexible, non-cleavable linker.

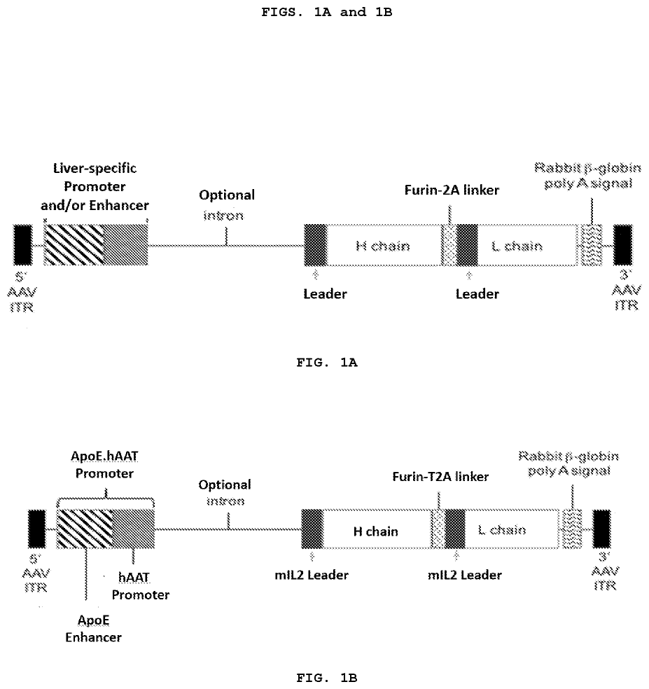

In certain embodiments, nucleic acids (e.g., polynucleotides) and nucleic acid sequences disclosed herein may be codon-optimized, for example, via any codon-optimization technique known to one of skill in the art (see, e.g., review by Quax et al., 2015, Mol Cell 59:149-161) and may also be optimized to reduce CpG dimers. Each heavy and light chain requires a signal sequence to ensure proper post-translation processing and secretion (unless expressed as an scFv, in which only the N-terminal chain requires a signal sequence sequence). Useful signal sequences for the expression of the heavy and light chains of the therapeutic antibodies in human cells are disclosed herein. An exemplary recombinant expression construct is shown in FIGS. 1A and B.

The production of HuPTM mAb or HuPTM Fab (including an HuPTM scFv) should result in a “biobetter” molecule for the treatment of disease accomplished via gene therapy—e.g., by administering a viral vector or other DNA expression construct encoding a full-length HuPTM mAb or HuPTM Fab or other antigen binding fragment, such as an scFv, of a therapeutic mAb to a patient (human subject) diagnosed with a disease indication for that mAb, to create a permanent depot in the subject that continuously supplies the human-glycosylated, sulfated transgene product produced by the subject's transduced cells. The cDNA construct for the HuPTM mAb or HuPTM Fab or HuPTM scFv should include a signal peptide that ensures proper co- and post-translational processing (glycosylation and protein sulfation) by the transduced human cells.

Pharmaceutical compositions suitable for administration to human subjects comprise a suspension of the recombinant vector in a formulation buffer comprising a physiologically compatible aqueous buffer, a surfactant and optional excipients. Such formulation buffer can comprise one or more of a polysaccharide, a surfactant, polymer, or oil.

As an alternative, or an additional treatment to gene therapy, the full-length HuPTM mAb or HuPTM Fab or other antigen binding fragment thereof can be produced in human cell lines by recombinant DNA technology, and the glycoprotein can be administered to patients. Human cell lines that can be used for such recombinant glycoprotein production include but are not limited to human embryonic kidney 293 cells (HEK293), fibrosarcoma HT-1080, HKB-11, CAP, HuH-7, and retinal cell lines, PER.C6, or RPE to name a few (e.g., see Dumont et al., 2015, Crit. Rev. Biotechnol. 36(6):1110-1122, which is incorporated by reference in its entirety for a review of the human cell lines that could be used for the recombinant production of the HuPTM mAb, HuPTM Fab or HuPTM scFv product, e.g., HuPTM Fab glycoprotein). To ensure complete glycosylation, especially sialylation, and tyrosine-sulfation, the cell line used for production can be enhanced by engineering the host cells to co-express α-2,6-sialyltransferase (or both α-2,3- and α-2,6-sialyltransferases) and/or TPST-1 and TPST-2 enzymes responsible for tyrosine-O-sulfation in human cells.

It is not essential that every molecule produced either in the gene therapy or protein therapy approach be fully glycosylated and sulfated. Rather, the population of glycoproteins produced should have sufficient glycosylation (including 2,6-sialylation) and sulfation to demonstrate efficacy. The goal of gene therapy treatment of the invention is to slow or arrest the progression of disease.

Combination therapies involving delivery of the full-length HuPTM mAb or HuPTM Fab or antigen binding fragment thereof to the patient accompanied by administration of other available treatments are encompassed by the methods of the invention. The additional treatments may be administered before, concurrently or subsequent to the gene therapy treatment. Such additional treatments can include but are not limited to co-therapy with the therapeutic mAb.

Also provided are methods of manufacturing the viral vectors, particularly the AAV based viral vectors. In specific embodiments, provided are methods of producing recombinant AAVs comprising culturing a host cell containing an artificial genome comprising a cis expression cassette flanked by AAV ITRs, wherein the cis expression cassette comprises a transgene encoding a therapeutic antibody operably linked to expression control elements that will control expression of the transgene in human cells; a trans expression cassette lacking AAV ITRs, wherein the trans expression cassette encodes an AAV rep and capsid protein operably linked to expression control elements that drive expression of the AAV rep and capsid proteins in the host cell in culture and supply the rep and cap proteins in trans; sufficient adenovirus helper functions to permit replication and packaging of the artificial genome by the AAV capsid proteins; and recovering recombinant AAV encapsidating the artificial genome from the cell culture.

5.1 Constructs

Viral vectors or other DNA expression constructs encoding an anti-Factor XII HuPTM mAb or antigen-binding fragment thereof, particularly a HuGlyFab, or a hyperglycosylated derivative of a HuPTM mAb antigen-binding fragment, are provided herein. The viral vectors and other DNA expression constructs provided herein include any suitable method for delivery of a transgene to a target cell. The means of delivery of a transgene include viral vectors, liposomes, other lipid-containing complexes, other macromolecular complexes, synthetic modified mRNA, unmodified mRNA, small molecules, non-biologically active molecules (e.g., gold particles), polymerized molecules (e.g., dendrimers), naked DNA, plasmids, phages, transposons, cosmids, or episomes. In some embodiments, the vector is a targeted vector, e.g., a vector targeting liver and/or muscle cells or a vector that has a tropism for liver and/or muscle cells.

In some aspects, the disclosure provides for a nucleic acid for use, wherein the nucleic acid comprises a nucleotide sequence that encodes a HuPTM mAb or HuGlyFab or other antigen-binding fragment thereof, as a transgene described herein, operatively linked to an ubiquitous promoter, a liver-specific and/or muscle-specific promoter, or an inducible promoter, wherein the promoter is selected for expression in tissue targeted for expression of the transgene. Promoters may, for example, be a CB7/CAG promoter (SEQ ID NO:89) and associated upstream regulatory sequences, cytomegalovirus (CMV) promoter, EF-1 alpha promoter (SEQ ID NO:92), mU1a (SEQ ID NO:91), UB6 promoter, chicken beta-actin (CBA) promoter, and liver-specific promoters, such as TBG (Thyroxine-binding Globulin) promoter (SEQ ID NO:93), APOA2 promoter, SERPINA1 (hAAT) promoter, ApoE.hAAT (SEQ ID NO:78), or muscle-specific promoters, such as a human desmin promoter, CK8 promoter (SEQ ID NO:90) or Pitx3 promoter, inducible promoters, such as a hypoxia-inducible promoter or a rapamycin-inducible promoter, or a combination thereof. In preferred embodiments, the promoter is a liver-specific promoter or a liver- and muscle-specific (dual) promoter. In preferred embodiments, the promoter is the liver-specific ApoE.hAAT (SEQ ID NO:78) promoter.

In some aspects herein, transgene expression is controlled by engineered nucleic acid regulatory elements that have more than one regulatory element (promoter or enhancer), including regulatory elements that are arranged in tandem (two or three copies) that promote liver-specific expression, or both liver-specific expression and muscle-specific expression. These regulatory elements include for the liver-specific expression, LSPX1 (SEQ ID NO:66), LSPX2 (SEQ ID NO:67), LTP1 (SEQ ID NO:68), LTP2 (SEQ ID NO:69), or LTP3 (SEQ ID NO:70), and for the liver and muscle expression, LMTP6 (SEQ ID NO:71), LMTP13 (SEQ ID NO:72), LMTP14 (SEQ ID NO:73), LMTP15 (SEQ ID NO:74), LMTP18 (SEQ ID NO:75), LMTP19 (SEQ ID NO:76), or LMTP20 (SEQ ID NO:77), the sequences of which are provided in Table 1.

In certain embodiments, provided herein are recombinant vectors that comprise one or more nucleic acids (e.g., polynucleotides). The nucleic acids may comprise DNA, RNA, or a combination of DNA and RNA. In certain embodiments, the DNA comprises one or more of the sequences selected from the group consisting of promoter sequences, the sequence of the gene of interest (the transgene, e.g., the nucleotide sequences encoding the heavy and light chains of the HuPTMmAb or HuGlyFab or other antigen-binding fragment), untranslated regions, and termination sequences. In certain embodiments, viral vectors provided herein comprise a promoter operably linked to the gene of interest.

In certain embodiments, nucleic acids (e.g., polynucleotides) and nucleic acid sequences disclosed herein may be codon-optimized, for example, via any codon-optimization technique known to one of skill in the art (see, e.g., review by Quax et al., 2015, Mol Cell 59:149-161).

In a specific embodiment, the constructs described herein comprise the following components: (1) AAV2 inverted terminal repeats that flank the expression cassette; (2) one or more control elements, b) optionally, a chicken β-actin or other intron and c) a rabbit β-globin poly A signal; and (3) nucleic acid sequences coding for the heavy and light chains of a mAb or Fab, separated by a self-cleaving furin (F)/(F/T)2A linker (SEQ ID NOS:164, 165, 166 or 167), ensuring expression of equal amounts of the heavy and the light chain polypeptides. An exemplary construct is shown in FIG. 1A.

In a specific embodiment, the constructs described herein comprise the following components: (1) AAV2 inverted terminal repeats that flank the expression cassette; (2) ApoE.hAAT promoter, b) optionally, a chicken β-actin or other intron and c) a rabbit β-globin polyA signal; and (3) nucleic acid sequences coding for a full-length antibody comprising the heavy and light chain sequences using sequences that encode the Fab portion of the heavy chain, including the hinge region sequence, plus the Fc polypeptide of the heavy chain for the appropriate isotype and the light chain, wherein heavy and light chain nucleotide sequences are separated by a self-cleaving furin (F)/(F/T)2A linker (SEQ ID NOS:164, 165, 166 or 167), ensuring expression of equal amounts of the heavy and the light chain polypeptides. An exemplary construct is shown in FIG. 1B.

5.1.1 mRNA Vectors

In certain embodiments, as an alternative to DNA vectors, the vectors provided herein are modified mRNA encoding for the gene of interest (e.g., the transgene, for example, HuPTMmAb or HuGlyFab or other antigen binding fragment thereof). The synthesis of modified and unmodified mRNA for delivery of a transgene to retinal pigment epithelial cells is taught, for example, in Hansson et al., J. Biol. Chem., 2015, 290(9):5661-5672, which is incorporated by reference herein in its entirety. In certain embodiments, provided herein is a modified mRNA encoding for a HuPTMmAb, HuPTM Fab, or HuPTM scFv.

5.1.2 Viral Vectors

Viral vectors include adenovirus, adeno-associated virus (AAV, e.g., AAV8, AAV9, AAVrh10, AAVS3), lentivirus, helper-dependent adenovirus, herpes simplex virus, poxvirus, hemagglutinin virus of Japan (HVJ), alphavirus, vaccinia virus, and retrovirus vectors. Retroviral vectors include murine leukemia virus (MLV) and human immunodeficiency virus (HIV)-based vectors. Alphavirus vectors include semliki forest virus (SFV) and sindbis virus (SIN). In certain embodiments, the viral vectors provided herein are recombinant viral vectors. In certain embodiments, the viral vectors provided herein are altered such that they are replication-deficient in humans. In certain embodiments, the viral vectors are hybrid vectors, e.g., an AAV vector placed into a “helpless” adenoviral vector. In certain embodiments, provided herein are viral vectors comprising a viral capsid from a first virus and viral envelope proteins from a second virus. In specific embodiments, the second virus is vesicular stomatitus virus (VSV). In more specific embodiments, the envelope protein is VSV-G protein.

In certain embodiments, the viral vectors provided herein are HIV based viral vectors. In certain embodiments, HIV-based vectors provided herein comprise at least two polynucleotides, wherein the gag and pol genes are from an HIV genome and the env gene is from another virus.

In certain embodiments, the viral vectors provided herein are herpes simplex virus-based viral vectors. In certain embodiments, herpes simplex virus-based vectors provided herein are modified such that they do not comprise one or more immediately early (IE) genes, rendering them non-cytotoxic.

In certain embodiments, the viral vectors provided herein are MLV based viral vectors. In certain embodiments, MLV-based vectors provided herein comprise up to 8 kb of heterologous DNA in place of the viral genes.

In certain embodiments, the viral vectors provided herein are lentivirus-based viral vectors. In certain embodiments, lentiviral vectors provided herein are derived from human lentiviruses. In certain embodiments, lentiviral vectors provided herein are derived from non-human lentiviruses. In certain embodiments, lentiviral vectors provided herein are packaged into a lentiviral capsid. In certain embodiments, lentiviral vectors provided herein comprise one or more of the following elements: long terminal repeats, a primer binding site, a polypurine tract, att sites, and an encapsidation site.

In certain embodiments, the viral vectors provided herein are alphavirus-based viral vectors. In certain embodiments, alphavirus vectors provided herein are recombinant, replication-defective alphaviruses. In certain embodiments, alphavirus replicons in the alphavirus vectors provided herein are targeted to specific cell types by displaying a functional heterologous ligand on their virion surface.

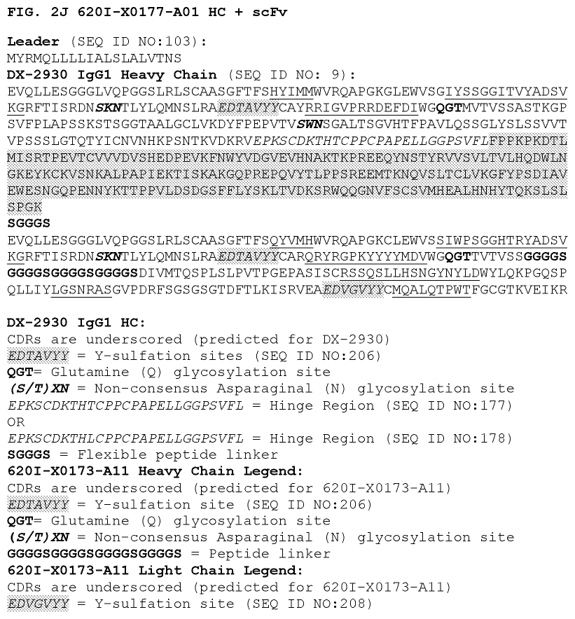

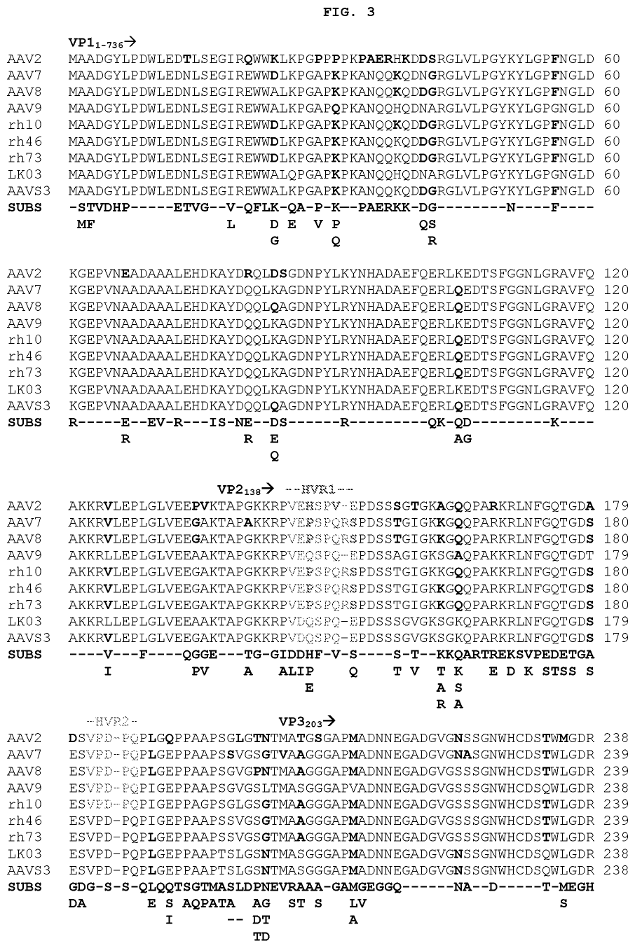

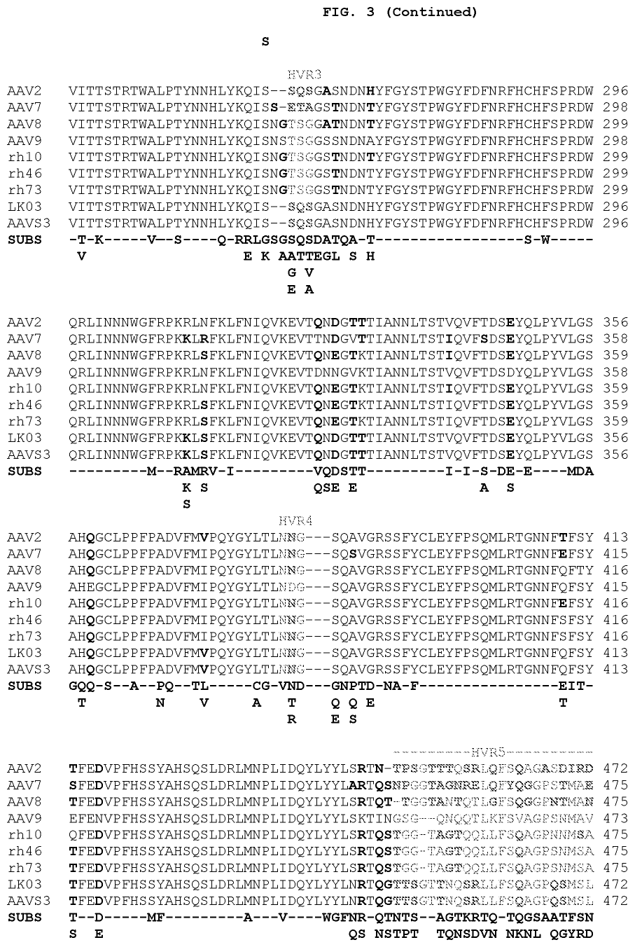

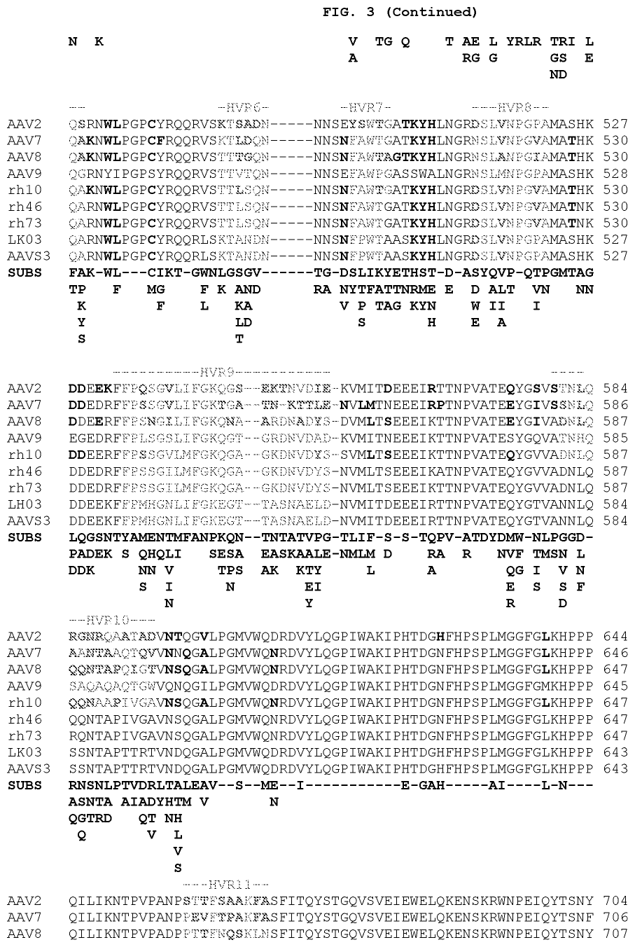

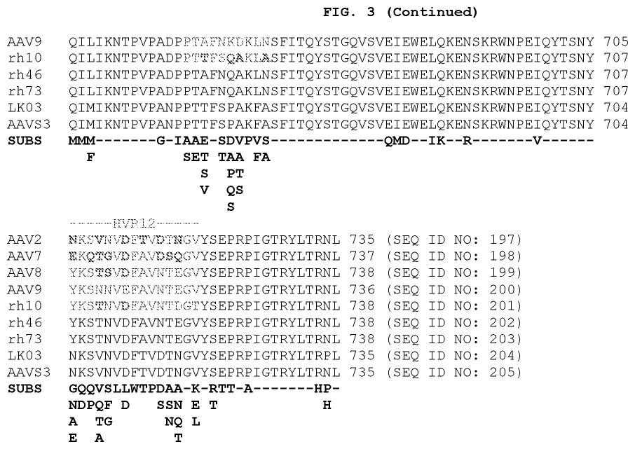

In certain embodiments, the viral vectors provided herein are AAV based viral vectors. In certain embodiments, the AAV-based vectors provided herein do not encode the AAV rep gene (required for replication) and/or the AAV cap gene (required for synthesis of the capsid proteins) (the rep and cap proteins may be provided by the packaging cells in trans). Multiple AAV serotypes have been identified. Throughout the specification, AAV “serotype” refers to an AAV having an immunologically distinct capsid, a naturally-occurring capsid, or an engineered capsid. In certain embodiments, AAV-based vectors provided herein comprise components from one or more serotypes of AAV. In preferred embodiments, AAV-based vectors provided herein comprise components from one or more serotypes of AAV with tropism to liver and/or muscle. In certain embodiments, AAV based vectors provided herein comprise capsid components from one or more of AAV3B, AAV5, AAV7 (SEQ ID NO:198), AAV8 (SEQ ID NO:199), AAV9 (SEQ ID NO:200), AAVrh10 (SEQ ID NO:201), AAVrh46 (SEQ ID NO:202), AAVrh73 (SEQ ID NO:203), AAVS3 (SEQ ID NO:205), AAV-LK03 (SEQ ID NO:204), AAVrh8, AAV64R1, or AAVhu37. In certain embodiments, AAV based vectors provided herein are or comprise components from one or more of AAV8, AAVS3, AAV-LK03, AAVrh46, AAVrh73, or AAVrh10 serotypes. Provided are viral vectors in which the capsid protein is a variant of the AAV8 capsid protein (SEQ ID NO:199), AAVS3 capsid protein (SEQ ID NO:205), or AAV-LK03 capsid protein (SEQ ID NO:204), and the capsid protein is e.g., at least 95%, 96%, 97%, 98%, 99% or 99.9% identical to the amino acid sequence of the AAV8 capsid protein (SEQ ID NO:199), AAVS3 capsid protein (SEQ ID NO:205), or AAV-LK03 capsid protein (SEQ ID NO:204), while retaining the biological function of the native capsid. In certain embodiments, the encoded AAV capsid has the sequence of SEQ ID NO:199 with 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acid substitutions and retaining the biological function of the AAV8, AAVS3, or AAV-LK03 capsid. FIG. 3 provides a comparative alignment of the amino acid sequences of the capsid proteins of different AAV serotypes with potential amino acids that may be substituted at certain positions in the aligned sequences based upon the comparison in the row labeled SUBS. Accordingly, in specific embodiments, the AAV vector comprises an AAV8, AAVS3, or AAV-LK03, capsid variant that has 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acid substitutions that are not present at that position in the native AAV capsid sequence as identified in the SUBS row of FIG. 3. Amino acid sequence for AAV8, AAVS3, or AAV-LK03 capsids are provided in FIG. 3.

The amino acid sequence of hu37 capsid can be found in SEQ ID NO:88 of international application PCT WO 2005/033321 and the amino acid sequence for the rh8 capsid is SEQ ID NO:97 of international application PCT WO 03/042397. The amino acid sequence for the rh64R1 sequence is found in WO2006/110689 (a R697W substitution of the Rh.64 sequence, which is SEQ ID NO: 43 of WO 2006/110689). The rh64R1 sequence is:

(SEQ ID NO: 232)
MAADGYLPDWLEDNLSEGIREWWDLKPGAPKPKANQQKQDD
GRGLVLPGYKYLGPFNGLDKGEPVNAADAAALEHDKAYDQ
QLKAGDNPYLRYNHADAEFQERLQEDTSFGGNLGRAVFQA
KKRVLEPLGLVEEGAKTAPGKKRPVEPSPQRSPDSSTGIG
KKGQQPARKRLNFGQTGDSESVPDPQPIGEPPAAPSSVGS
GTMAAGGGAPMADNNEGADGVGSSSGNWHCDSTWLGDRVI
TTSTRTWALPTYNNHLYKQISNGTSGGSTNDNTYFGYSTP
WGYFDFNRFHCHFSPRDWQRLINNNWGFRPKRLSFKLFNI
QVKEVTQNEGTKTIANNLTSTIQVFTDSEYQLPYVLGSAH
QGCLPPFPADVFMIPQYGYLTLNNGSQAVGRSSFYCLEYF
PSQMLRTGNNFSFSYTFEDVPFHSSYAHSQSLDRLMNPLI
DQYLYYLSRTQSTGGTAGTQQLLFSQAGPSNMSAQARNWL
PGPCYRQQRVSTTLSQNNNSNFAWTGATKYHLNGRDSLVN
PGVAMATNKDDEDRFFPSSGILMFGKQGAGKDNVDYSNVM
LTSEEEIKTTNPVATEQYGVVADNLQQQNTAPIVGAVNSQ
GALPGMVWQNRDVYLQGPIWAKIPHTDGNFHPSPLMGGFG
LKHPPPQILIKNTPVPADPPTAFNQAKLNSFITQYSTGQV
SVEIVWELQKENSKRWNPEIQYTSNYYKSTNVDFAVNTEG
VYSEPRPIGTRYLTRNL.

In some embodiments, AAV-based vectors comprise components from one or more serotypes of AAV. In some embodiments, AAV based vectors provided herein comprise capsid components from one or more of AAV1, AAV2, AAV3, AAV4, AAV5, AAV6, AAV7, AAV8, AAV9, AAV10, AAV11, AAV12, AAV13, AAV14, AAV15, AAV16, AAVS3, AAV.rh8, AAV.rh10, AAV.rh20, AAV.rh39, AAV.rh46, AAV.rh73, AAV.Rh74, AAV.RHM4-1, AAV.hu37, AAV.Anc80, AAV.Anc80L65, AAV. 7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16 or other rAAV particles, or combinations of two or more thereof. In some embodiments, AAV based vectors provided herein comprise components from one or more of AAV1, AAV2, AAV3, AAV4, AAV5, AAV6, AAV7, AAV8, AAV9, AAV10, AAV11, AAV12, AAV13, AAV14, AAV15, AAV16, AAVS3, AAV.rh8, AAV.rh10, AAV.rh20, AAV.rh39, AAV.rh46, AAV.rh73, AAV.Rh74, AAV.RHM4-1, AAV.hu37, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16 or other rAAV particles, or combinations of two or more thereof serotypes. In some embodiments, rAAV particles comprise a capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to e.g., VP1, VP2 and/or VP3 sequence of an AAV capsid serotype selected from AAV1, AAV2, AAV3, AAV4, AAV5, AAV6, AAV7, AAV8, AAV9, AAV10, AAV11, AAV12, AAV13, AAV14, AAV15, AAV16, AAVS3, AAV.rh8, AAV.rh10, AAV.rh20, AAV.rh39, AAV.rh46, AAV.rh73, AAV.Rh74, AAV.RHM4-1, AAV.hu37, AAV.Anc80, rAAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16, or a derivative, modification, or pseudotype thereof.

In particular embodiments, the recombinant AAV for use in compositions and methods herein is AAVS3 (including variants thereof) (see e.g., US Patent Application No. 20200079821, which is incorporated herein by reference in its entirety). In particular embodiments, rAAV particles comprise the capsids of AAV-LK03 or AAV3B, as described in Puzzo et al., 2017, Sci. Transl. Med. 29(9): 418, which is incorporated by reference in its entirety. In particular embodiments, the AAV for use in compositions and methods herein is any AAV disclosed in U.S. Pat. No. 10,301,648, such as AAV.rh46 or AAV.rh73. In some embodiments, the recombinant AAV for use in compositions and methods herein is Anc80 or Anc80L65 (see, e.g., Zinn et al., 2015, Cell Rep. 12(6): 1056-1068, which is incorporated by reference in its entirety). In particular embodiments, the AAV for use in compositions and methods herein is any AAV disclosed in U.S. Pat. No. 9,585,971, such as AAV-PHP.B. In particular embodiments, the AAV for use in compositions and methods herein is an AAV2/Rec2 or AAV2/Rec3 vector, which has hybrid capsid sequences derived from AAV8 and serotypes cy5, rh20 or rh39 (see, e.g., Issa et al., 2013, PLoS One 8(4): e60361, which is incorporated by reference herein for these vectors). In particular embodiments, the AAV for use in compositions and methods herein is an AAV disclosed in any of the following, each of which is incorporated herein by reference in its entirety: U.S. Pat. Nos. 7,282,199; 7,906,111; 8,524,446; 8,999,678; 8,628,966; 8,927,514; 8,734,809; 9,284,357; 9,409,953; 9,169,299; 9,193,956; 9,458,517; 9,587,282; US 2015/0374803; US 2015/0126588; US 2017/0067908; US 2013/0224836; US 2016/0215024; US 2017/0051257; PCT/US2015/034799; and PCT/EP2015/053335. In some embodiments, rAAV particles have a capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to the VP1, VP2 and/or VP3 sequence of an AAV capsid disclosed in any of the following patents and patent applications, each of which is incorporated herein by reference in its entirety: U.S. Pat. Nos. 7,282,199; 7,906,111; 8,524,446; 8,999,678; 8,628,966; 8,927,514; 8,734,809; 9,284,357; 9,409,953; 9,169,299; 9,193,956; 9,458,517; and 9,587,282; US patent application publication nos. 2015/0374803; 2015/0126588; 2017/0067908; 2013/0224836; 2016/0215024; 2017/0051257; and International Patent Application Nos. PCT/US2015/034799; PCT/EP2015/053335.

In some embodiments, rAAV particles comprise any AAV capsid disclosed in U.S. Pat. No. 9,840,719 and WO 2015/013313, such as AAV.Rh74 and RHM4-1, each of which is incorporated herein by reference in its entirety. In some embodiments, rAAV particles comprise any AAV capsid disclosed in WO 2014/172669, such as AAV rh.74, which is incorporated herein by reference in its entirety. In some embodiments, rAAV particles comprise the capsid of AAV2/5, as described in Georgiadis et al., 2016, Gene Therapy 23: 857-862 and Georgiadis et al., 2018, Gene Therapy 25: 450, each of which is incorporated by reference in its entirety. In some embodiments, rAAV particles comprise any AAV capsid disclosed in WO 2017/070491, such as AAV2tYF, which is incorporated herein by reference in its entirety. In some embodiments, rAAV particles comprise any AAV capsid disclosed in U.S. Pat. Nos. 8,628,966; 8,927,514; 9,923,120 and WO 2016/049230, such as HSC1, HSC2, HSC3, HSC4, HSC5, HSC6, HSC7, HSC8, HSC9, HSC10, HSC11, HSC12, HSC13, HSC14, HSC15, or HSC16, each of which is incorporated by reference in its entirety.

In some embodiments, rAAV particles have a capsid protein disclosed in Intl. Appl. Publ. No. WO 2003/052051 (see, e.g., SEQ ID NO: 2 of '051 publication), WO 2005/033321 (see, e.g., SEQ ID NOs: 123 and 88 of '321 publication), WO 03/042397 (see, e.g., SEQ ID NOs: 2, 81, 85, and 97 of '397 publication), WO 2006/068888 (see, e.g., SEQ ID NOs: 1 and 3-6 of '888 publication), WO 2006/110689, (see, e.g., SEQ ID NOs: 5-38 of '689 publication) WO2009/104964 (see, e.g., SEQ ID NOs: 1-5, 7, 9, 20, 22, 24 and 31 of '964 publication), WO 2010/127097 (see, e.g., SEQ ID NOs: 5-38 of '097 publication), and WO 2015/191508 (see, e.g., SEQ ID NOs: 80-294 of '508 publication), and U.S. Appl. Publ. No. 20150023924 (see, e.g., SEQ ID NOs: 1, 5-10 of '924 publication), the contents of each of which is herein incorporated by reference in its entirety. In some embodiments, rAAV particles have a capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to the VP1, VP2 and/or VP3 sequence of an AAV capsid disclosed in Intl. Appl. Publ. No. WO 2003/052051 (see, e.g., SEQ ID NO: 2 of '051 publication), WO 2005/033321 (see, e.g., SEQ ID NOs: 123 and 88 of '321 publication), WO 03/042397 (see, e.g., SEQ ID NOs: 2, 81, 85, and 97 of '397 publication), WO 2006/068888 (see, e.g., SEQ ID NOs: 1 and 3-6 of '888 publication), WO 2006/110689 (see, e.g., SEQ ID NOs: 5-38 of '689 publication) WO2009/104964 (see, e.g., SEQ ID NOs: 1-5, 7, 9, 20, 22, 24 and 31 of 964 publication), WO 2010/127097 (see, e.g., SEQ ID NOs: 5-38 of '097 publication), and WO 2015/191508 (see, e.g., SEQ ID NOs: 80-294 of '508 publication), and U.S. Appl. Publ. No. 20150023924 (see, e.g., SEQ ID NOs: 1, 5-10 of '924 publication).

In additional embodiments, rAAV particles comprise a pseudotyped AAV capsid. In some embodiments, the pseudotyped AAV capsids are rAAV2/8 or rAAV2/9 pseudotyped AAV capsids. Methods for producing and using pseudotyped rAAV particles are known in the art (see, e.g., Duan et al., J. Virol., 75:7662-7671 (2001); Halbert et al., J. Virol., 74:1524-1532 (2000); Zolotukhin et al., Methods 28:158-167 (2002); and Auricchio et al., Hum. Molec. Genet. 10:3075-3081, (2001).

AAV8-based, AAV9-based, and AAVrh10-based viral vectors are used in certain of the methods described herein. Nucleotide sequences of AAV based viral vectors and methods of making recombinant AAV and AAV capsids are taught, for example, in U.S. Pat. No. 7,282,199 B2, U.S. Pat. No. 7,790,449 B2, U.S. Pat. No. 8,318,480 B2, U.S. Pat. No. 8,962,332 B2 and International Patent Application No. PCT/EP2014/076466, each of which is incorporated herein by reference in its entirety. In one aspect, provided herein are AAV (e.g., AAV8, AAV9 or AAVrh10)-based viral vectors encoding a transgene (e.g., an HuPTM Fab). The amino acid sequences of AAV capsids, including AAV8, AAV9 and AAVrh10 are provided in FIG. 21.

In certain embodiments, a single-stranded AAV (ssAAV) may be used supra. In certain embodiments, a self-complementary vector, e.g., scAAV, may be used (see, e.g., Wu, 2007, Human Gene Therapy, 18(2):171-82, McCarty et al, 2001, Gene Therapy, Vol 8, Number 16, Pages 1248-1254; and U.S. Pat. Nos. 6,596,535; 7,125,717; and 7,456,683, each of which is incorporated herein by reference in its entirety).

In certain embodiments, the viral vectors used in the methods described herein are adenovirus based viral vectors. A recombinant adenovirus vector may be used to transfer in the transgene encoding the HuPTMmAb or HuGlyFab or antigen-binding fragment. The recombinant adenovirus can be a first-generation vector, with an E1 deletion, with or without an E3 deletion, and with the expression cassette inserted into either deleted region. The recombinant adenovirus can be a second-generation vector, which contains full or partial deletions of the E2 and E4 regions. A helper-dependent adenovirus retains only the adenovirus inverted terminal repeats and the packaging signal (phi). The transgene is inserted between the packaging signal and the 3′ITR, with or without stuffer sequences to keep the genome close to wild-type size of approximately 36 kb. An exemplary protocol for production of adenoviral vectors may be found in Alba et al., 2005, “Gutless adenovirus: last generation adenovirus for gene therapy,” Gene Therapy 12:S18-S27, which is incorporated by reference herein in its entirety.

In certain embodiments, the viral vectors used in the methods described herein are lentivirus based viral vectors. A recombinant lentivirus vector may be used to transfer in the transgene encoding the HuPTM mAb antigen binding fragment. Four plasmids are used to make the construct: Gag/pol sequence containing plasmid, Rev sequence containing plasmids, Envelope protein containing plasmid (e.g., VSV-G), and Cis plasmid with the packaging elements and the anti-VEGF antigen-binding fragment gene.

For lentiviral vector production, the four plasmids are co-transfected into cells (e.g., HEK293 based cells), whereby polyethylenimine or calcium phosphate can be used as transfection agents, among others. The lentivirus is then harvested in the supernatant (lentiviruses need to bud from the cells to be active, so no cell harvest needs/should be done). The supernatant is filtered (0.45 μm) and then magnesium chloride and benzonase added. Further downstream processes can vary widely, with using TFF and column chromatography being the most GMP compatible ones. Others use ultracentrifugation with/without column chromatography. Exemplary protocols for production of lentiviral vectors may be found in Lesch et al., 2011, “Production and purification of lentiviral vector generated in 293T suspension cells with baculoviral vectors,” Gene Therapy 18:531-538, and Ausubel et al., 2012, “Production of CGMP-Grade Lentiviral Vectors,” Bioprocess Int. 10(2):32-43, both of which are incorporated by reference herein in their entireties.

In a specific embodiment, a vector for use in the methods described herein is one that encodes a HuPTM mAb, such that, upon introduction of the vector into a relevant cell, a glycosylated and/or tyrosine sulfated variant of the HuPTM mAb is expressed by the cell.

5.1.3 Promoters and Modifiers of Gene Expression

In certain embodiments, the vectors provided herein comprise components that modulate gene delivery or gene expression (e.g., “expression control elements”). In certain embodiments, the vectors provided herein comprise components that modulate gene expression. In certain embodiments, the vectors provided herein comprise components that influence binding or targeting to cells. In certain embodiments, the vectors provided herein comprise components that influence the localization of the polynucleotide (e.g., the transgene) within the cell after uptake. In certain embodiments, the vectors provided herein comprise components that can be used as detectable or selectable markers, e.g., to detect or select for cells that have taken up the polynucleotide.

In certain embodiments, the viral vectors provided herein comprise one or more promoters that control expression of the transgene. These promoters (and other regulatory elements that control transcription, such as enhancers) may be constitutive (promote ubiquitous expression) or may specifically or selectively express in the liver (including promoting expression in the liver only or expressing in the liver at least at 1 to 100 fold greater levels than in a non-liver tissue). In certain embodiments, the promoter is a constitutive promoter.

In certain embodiments, the promoter is a CB7 (also referred to as a CAG promoter) (see Dinculescu et al., 2005, Hum Gene Ther 16: 649-663, incorporated by reference herein in its entirety). In some embodiments, the CAG or CB7 promoter (SEQ ID NO: 69) includes other expression control elements that enhance expression of the transgene driven by the vector. In certain embodiments, the other expression control elements include chicken β-actin intron and/or rabbit (3-globin polyA signal. In certain embodiments, the promoter comprises a TATA box. In certain embodiments, the promoter comprises one or more elements. In certain embodiments, the one or more promoter elements may be inverted or moved relative to one another. In certain embodiments, the elements of the promoter are positioned to function cooperatively. In certain embodiments, the elements of the promoter are positioned to function independently. In certain embodiments, the viral vectors provided herein comprise one or more promoters selected from the group consisting of the human CMV immediate early gene promoter, the SV40 early promoter, the Rous sarcoma virus (RS) long terminal repeat, and rat insulin promoter. In certain embodiments, the vectors provided herein comprise one or more long terminal repeat (LTR) promoters selected from the group consisting of AAV, MLV, MMTV, SV40, RSV, HIV-1, and HIV-2 LTRs.

In certain embodiments, the vectors provided herein comprise one or more tissue specific promoters (e.g., a liver-specific promoter or a dual liver-muscle specific promoter). In particular embodiments, the viral vectors provided herein comprises a liver cell specific promoter, such as, a TBG (Thyroxine-binding Globulin) promoter (SEQ ID NO: 73), an APOA2 promoter, a SERPINA1 (hAAT) promoter, or an ApoE.hAAT promoter (SEQ ID NO: 58). In certain embodiments, the viral vector provided herein comprises a muscle specific promoter, such as a human desmin promoter (Jonuschies et al., 2014, Curr. Gene Ther. 14:276-288), a CK8 promoter (SEQ ID NO: 70; Himeda et al., 2011 Muscle Gene Therapy: Methods and Protocols, Methods in Molecular Biology, Dongsheng Duan (ed.), 709:3-19), or a Pitx3 promoter (Coulon et al., 2007, JBC 282:33192). In other embodiments, the viral vector comprises a VMD2 promoter.

Provided are nucleic acid regulatory elements that are chimeric with respect to arrangements of elements in tandem in the expression cassette. Regulatory elements, in general, have multiple functions as recognition sites for transcription initiation or regulation, coordination with cell-specific machinery to drive expression upon signaling, and to enhance expression of the downstream gene.

Also provided are arrangements of combinations of nucleic acid regulatory elements that promote transgene expression in liver tissue, or liver and muscle (skeletal and/or cardiac) tissue. In particular, certain elements are arranged with two or more copies of the individual enhancer and promoter elements arranged in tandem and operably linked to a transgene to promote expression, particularly tissue specific expression. Exemplary nucleotide sequences of the individual promoter and enhancer elements are provided in Table 1. Also provided in Table 1 are exemplary composite nucleic acid regulatory elements comprising the individual tandem promoter and enhancer elements. In certain embodiments the downstream promoter is an hAAT promoter (in certain embodiments the hAAT promoter is an hAAT(ΔATG) promoter) and the other promoter is another hAAT promoter or is a TBG promoter).

These combinations of promoter and enhancer sequences provided herein improve transgene expression while maintaining tissue specificity. Transgene expression from tandem promoters (i.e. two promoter sequences driving expression of the same transgene) is improved by depleting the 3′ promoter sequence of potential ‘ATG’ initiation sites. This approach was employed to improve transgene expression from tandem tissue-specific promoter cassettes (such as those targeting the liver) as well as promoter cassettes to achieve dual expression in two separate tissue populations (such as liver and skeletal muscle, and in certain embodiments cardiac muscle, and liver and bone). Ultimately, these designs aim to improve the therapeutic efficacy of gene transfer by providing more robust levels of transgene expression, improved stability/persistence, and induction of immune tolerance to the transgene product. In certain aspects the hAAT promoter with the start codon deleted (ΔATG) is used in an expression cassette provided herein.

Accordingly, with respect to liver and muscle specific expression, provided are nucleic acid regulatory elements that comprise or consist of promoters and/or other nucleic acid elements, such as enhancers, that promote liver expression, such as ApoE enhancers, Mic/BiKE elements or hAAT promoters. These may be present as single copies or with two or more copies in tandem. The nucleic acid regulatory element may also comprise, in addition to the one or more elements that promote liver specific expression, one or more elements that promote muscle specific expression (including skeletal and/or cardiac muscle), for example, one or more copies, for example two copies, of the MckE element, which may be arranged as two or more copies in tandem or an MckE and MhcE elements arranged in tandem. In certain embodiments, a promoter element is deleted for the initiation codon to prevent translation initiation at that site, and preferably, the element with the modified start codon is the promoter that is the element at the 3′ end or the downstream end of the nucleic acid regulatory element, for example, closest within the nucleic acid sequence of the expression cassette to the transgene. In certain embodiments, the composite nucleic acid regulatory element comprises an hAAT promoter, in embodiments an hAAT which is start-codon modified (ΔATG) as the downstream promoter, and a second promoter in tandem with the hAAT promoter, which is an hAAT promoter, a CK8 promoter, an Spc5.12 promoter or an minSpc5.12 promoter. Nucleotide sequences are provided in Table 1.

In certain embodiments, the nucleotide sequence encoding the anti-Factor XII antibody heavy and light chains is operably linked to a composite nucleic acid regulatory element comprising a) two copies of Mic/BiKE arranged in tandem or two copies of ApoE arranged in tandem or two copies of Mic/BiKE arranged in tandem with one copy of ApoE, b) one promoter or, in tandem promoter embodiments, two promoters arranged in tandem comprising at least one copy of hAAT which is start-codon modified (ΔATG) (where in certain embodiments the hAAT promoter is the downstream or 3′ promoter). In some embodiments, the composite nucleic acid regulatory element comprises LSPX1, LSPX2, LTP1, LTP2, or LTP3 of Table 1.

Also provided are recombinant expression cassettes in which the nucleotide sequence encoding the heavy and light chains of the anti-Factor XII antibody is operably linked to a nucleic acid regulatory element comprising a) one copy of ApoE, two or three copies of MckE arranged in tandem, one copy of each MckE, MhcE, and ApoE arrange in tandem, or two or three copies of MckE arranged in tandem with one copy of ApoE, b) two copies of a promoter arranged in tandem comprising at least one copy of hAAT which is start-codon modified (ΔATG). In certain embodiments, the second and upstream promoter is a CK8 promoter, an Spc5.12 promoter or a minSpc5.12 promoter. In some embodiments, the composite nucleic acid regulatory element comprises LMTP6, LMTP13, LMTP14, LMTP15, LMTP18, LMTP19, or LMTP20 of Table 1.

In certain embodiments, the anti-Factor XII therapeutic antibody coding sequence is operably linked to composite nucleic acid regulatory elements for enhancing gene expression in the liver LSPX1 (SEQ ID NO:66), LSPX2 (SEQ ID NO:67), LTP1 (SEQ ID NO:68), LTP2 (SEQ ID NO:69), or LTP3 (SEQ ID NO:70), liver and muscle expression, LMTP6 (SEQ ID NO:71), LMTP13 (SEQ ID NO:72), LMTP14 (SEQ ID NO:73), LMTP15 (SEQ ID NO:74), LMTP18 (SEQ ID NO:75), LMTP19 (SEQ ID NO:76), or LMTP20 (SEQ ID NO:77), the sequences of which are provided in Table 1 below. Also included are composite regulatory elements that enhance gene expression in the liver, and in certain embodiments, also muscle or bone, which have 99%, 95%, 90%, 85% or 80% sequence identity with one of nucleic acid LSPX1 (SEQ ID NO:66), LSPX2 (SEQ ID NO:67), LTP1 (SEQ ID NO:68), LTP2 (SEQ ID NO:69), or LTP3 (SEQ ID NO:70), liver and muscle expression, LMTP6 (SEQ ID NO:71), LMTP13 (SEQ ID NO:72), LMTP14 (SEQ ID NO:73), LMTP15 (SEQ ID NO:74), LMTP18 (SEQ ID NO:75), LMTP19 (SEQ ID NO:76), or LMTP20 (SEQ ID NO:77).

The tandem and composite promoters described herein result in preferred transcription start sites within the promoter region. Thus, in certain embodiments, the constructs described herein have a tandem or composite nucleic acid regulatory sequence that comprises an hAAT promoter (particularly a modified start codon hAAT promoter) and has a transcription start site of TCTCC (corresponding to nt 1541-1545 of LMTP6 (SEQ ID NO:71), which overlaps with the active TTS found in hAAT (nt 355-359 of SEQ ID NO: 83) or GGTACAATGACTCCTTTCG (SEQ ID NO:233), which corresponds to nucleotides 139-157 of SEQ ID NO: 83, or GGTACAGTGACTCCTTTCG (SEQ ID NO:234), which corresponds to nucleotides 139-157 of SEQ ID NO: 84. In other embodiments, the constructs described herein have a tandem or composite regulatory sequence that comprises a CK8 promoter and has a transcription start site at TCATTCTACC (SEQ ID NO:235), which corresponds to nucleotides 377-386 of SEQ ID NO: 90, particularly starting at the nucleotide corresponding to nucleotide 377 of SEQ ID NO: 90 or corresponding to nucleotide 1133 of SEQ ID NO: 71.

In certain embodiments, the promoter is an inducible promoter. In certain embodiments the promoter is a hypoxia-inducible promoter. In certain embodiments, the promoter comprises a hypoxia-inducible factor (HIF) binding site. In certain embodiments, the promoter comprises a HIF-1α binding site. In certain embodiments, the promoter comprises a HIF-2a binding site. In certain embodiments, the HIF binding site comprises an RCGTG motif. For details regarding the location and sequence of HIF binding sites, see, e.g., Schödel, et al., Blood, 2011, 117(23):e207-e217, which is incorporated by reference herein in its entirety. In certain embodiments, the promoter comprises a binding site for a hypoxia induced transcription factor other than a HIF transcription factor. In certain embodiments, the viral vectors provided herein comprise one or more IRES sites that is preferentially translated in hypoxia. For teachings regarding hypoxia-inducible gene expression and the factors involved therein, see, e.g., Kenneth and Rocha, Biochem J., 2008, 414:19-29, which is incorporated by reference herein in its entirety. In specific embodiments, the hypoxia-inducible promoter is the human N-WASP promoter, see, e.g., Salvi, 2017, Biochemistry and Biophysics Reports 9:13-21 (incorporated by reference for the teaching of the N-WASP promoter) or is the hypoxia-induced promoter of human Epo, see, e.g., Tsuchiya et al., 1993, J. Biochem. 113:395-400 (incorporated by reference for the disclosure of the Epo hypoxia-inducible promoter). In other embodiments, the promoter is a drug inducible promoter, for example, a promoter that is induced by administration of rapamycin or analogs thereof. See, e.g., the disclosure of rapamycin inducible promoters in PCT publications WO94/18317, WO 96/20951, WO 96/41865, WO 99/10508, WO 99/10510, WO 99/36553, and WO 99/41258, and U.S. Pat. No. 7,067,526, which are hereby incorporated by reference in their entireties for the disclosure of drug inducible promoters.

Provided herein are constructs containing certain ubiquitous and tissue-specific promoters. Such promoters include synthetic and tandem promoters. Examples and nucleotide sequences of promoters are provided in Table 1 below. Table 1 also includes the nucleotide sequences of other regulatory elements useful for the expression cassettes provided herein

TABLE 1
Promoter and Other Regulatory Element Sequences

| Name/ | |
|---|---|
| SEQ ID NO. | Sequence |
| LSPX1 | aggttaatttttaaaaagcagtcaaaagtccaagtggcccttggcagcatttactctctctgt |
| SEQ ID NO: 66 | ttgctctggttaataatctcaggagcacaaacattccagatccaggttaatttttaaaaagca |
| | gtcaaaagtccaagtggcccttggcagcatttactctctctgtttgctctggttaataatctc |
| | aggagcacaaacattccagatccggcgcgccagggctggaagctacctttgtctagaaggctc |
| | agaggcacacaggagtttctgggctcaccctgcccccttccaacccctcagttcccatcctcc |
| | agcagctgtttgtgtgctgcctctgaagtccacactgaacaaacttcagcctactcatgtccc |
| | taaaatgggcaaacattgcaagcagcaaacagcaaacacacagccctccctgcctgctgacct |
| | tggagctggggcagaggtcagagacctctctgggcccatgccacctccaacatccactcgacc |
| | ccttggaatttcggtggagaggagcagaggttgtcctggcgtggtttaggtagtgtgagaggg |
| | gtacccggggatcttgctaccagtggaacagccactaaggattctgcagtgagagcagagggc |
| | cagctaagtggtactctcccagagactgtctgactcacgccaccccctccaccttggacacag |
| | gacgctgtggtttctgagccaggtacaatgactcctttcggtaagtgcagtggaagctgtaca |
| | ctgcccaggcaaagcgtccgggcagcgtaggcgggcgactcagatcccagccagtggacttag |
| | cccctgtttgctcctccgataactggggtgaccttggttaatattcaccagcagcctcccccg |
| | ttgcccctctggatccactgcttaaatacggacgaggacagggccctgtctcctcagcttcag |
| | gcaccaccactgacctgggacagt |
| LSXP2 | aggctcagaggcacacaggagtttctgggctcaccctgcccccttccaacccctcagttccca |
| SEQ ID NO: 67 | tcctccagcagctgtttgtgtgctgcctctgaagtccacactgaacaaacttcagcctactca |
| | tgtccctaaaatgggcaaacattgcaagcagcaaacagcaaacacacagccctccctgcctgc |
| | tgaccttggagctggggcagaggtcagagacctctctgggcccatgccacctccaacatccac |
| | tcgaccccttggaatttcggtggagaggagcagaggttgtcctggcgtggtttaggtagtgtg |
| | agagggtctagaaggctcagaggcacacaggagtttctgggctcaccctgcccccttccaacc |
| | cctcagttcccatcctccagcagctgtttgtgtgctgcctctgaagtccacactgaacaaact |
| | tcagcctactcatgtccctaaaatgggcaaacattgcaagcagcaaacagcaaacacacagcc |
| | ctccctgcctgctgaccttggagctggggcagaggtcagagacctctctgggcccatgccacc |
| | tccaacatccactcgaccccttggaatttcggtggagaggagcagaggttgtcctggcgtggt |
| | ttaggtagtgtgagaggggtacccggggatottgctaccagtggaacagccactaaggattct |
| | gcagtgagagcagagggccagctaagtggtactctcccagagactgtctgactcacgccaccc |
| | cctccaccttggacacaggacgctgtggtttctgagccaggtacaatgactcctttcggtaag |
| | tgcagtggaagctgtacactgcccaggcaaagcgtccgggcagcgtaggcgggcgactcagat |
| | cccagccagtggacttagcccctgtttgctcctccgataactggggtgaccttggttaatatt |
| | caccagcagcctcccccgttgcccctctggatccactgcttaaatacggacgaggacagggcc |
| | ctgtctcctcagcttcaggcaccaccactgacctgggacagt |
| LTP1 | aggttaatttttaaaaagcagtcaaaagtccaagtggcccttggcagcatttactctctctgt |
| SEQ ID NO: 68 | ttgctctggttaataatctcaggagcacaaacattccagatccaggttaatttttaaaaagca |
| | gtcaaaagtccaagtggcccttggcagcatttactctctctgtttgctctggttaataatctc |
| | aggagcacaaacattccagatccggcgcgccagggctggaagctacctttgacatcatttcct |
| | ctgcgaatgcatgtataatttctacagaacctattagaaaggatcacccagcctctgcttttg |
| | tacaactttcccttaaaaaactgccaattccactgctgtttggcccaatagtgagaacttttt |
| | cctgctgcctcttggtgcttttgcctatggcccctattctgcctgctgaagacactcttgcca |
| | gcatggacttaaacccctccagctctgacaatcctctttctcttttgttttacatgaagggtc |
| | tggcagccaaagcaatcactcaaagttcaaaccttatcattttttgctttgttcctcttggcc |
| | ttggttttgtacatcagctttgaaaataccatcccagggttaatgctggggttaatttataac |
| | taagagtgctctagttttgcaatacaggacatgctataaaaatggaaagatgttgctttctga |
| | gaggatcttgctaccagtggaacagccactaaggattctgcagtgagagcagagggccagcta |
| | agtggtactctcccagagactgtctgactcacgccaccccctccaccttggacacaggacgct |
| | gtggtttctgagccaggtacagtgactcctttcggtaagtgcagtggaagctgtacactgccc |
| | aggcaaagcgtccgggcagcgtaggcgggcgactcagatcccagccagtggacttagcccctg |
| | tttgctcctccgataactggggtgaccttggttaatattcaccagcagcctcccccgttgccc |
| | ctctggatccactgcttaaatacggacgaggacagggccctgtctcctcagcttcaggcacca |
| | ccactgacctgggacagt |
| LTP2 | aggctcagaggcacacaggagtttctgggctcaccctgcccccttccaacccctcagttccca |
| SEQ ID NO: 69 | tcctccagcagctgtttgtgtgctgcctctgaagtccacactgaacaaacttcagcctactca |
| | tgtccctaaaatgggcaaacattgcaagcagcaaacagcaaacacacagccctccctgcctgc |
| | tgaccttggagctggggcagaggtcagagacctctctgggcccatgccacctccaacatccac |
| | tcgaccccttggaatttcggtggagaggagcagaggttgtcctggcgtggtttaggtagtgtg |
| | agagggtctagagcccttaagctagcaggttaatttttaaaaagcagtcaaaagtccaagtgg |
| | cccttggcagcatttactctctctgtttgctctggttaataatctcaggagcacaaacattcc |
| | agatccaggttaatttttaaaaagcagtcaaaagtccaagtggcccttggcagcatttactct |
| | ctctgtttgctctggttaataatctcaggagcacaaacattccagatccggcgcgccagggct |
| | ggaagctacctttgacatcatttcctctgcgaatgcatgtataatttctacagaacctattag |
| | aaaggatcacccagcctctgcttttgtacaactttcccttaaaaaactgccaattccactgct |
| | gtttggcccaatagtgagaactttttcctgctgcctcttggtgcttttgcctatggcccctat |
| | tctgcctgctgaagacactcttgccagcatggacttaaacccctccagctctgacaatcctct |
| | ttctcttttgttttacatgaagggtctggcagccaaagcaatcactcaaagttcaaaccttat |
| | cattttttgctttgttcctcttggccttggttttgtacatcagctttgaaaataccatcccag |
| | ggttaatgctggggttaatttataactaagagtgctctagttttgcaatacaggacatgctat |
| | aaaaatggaaagatgttgctttctgagaggatcttgctaccagtggaacagccactaaggatt |
| | ctgcagtgagagcagagggccagctaagtggtactctcccagagactgtctgactcacgccac |
| | cccctccaccttggacacaggacgctgtggtttctgagccaggtacagtgactcctttcggta |
| | agtgcagtggaagctgtacactgcccaggcaaagcgtccgggcagcgtaggcgggcgactcag |
| | atcccagccagtggacttagcccctgtttgctcctccgataactggggtgaccttggttaata |
| | ttcaccagcagcctcccccgttgcccctctggatccactgcttaaatacggacgaggacaggg |
| | ccctgtctcctcagcttcaggcaccaccactgacctgggacagt |
| LTP3 | aggttaatttttaaaaagcagtcaaaagtccaagtggcccttggcagcatttactctctctgt |
| SEQ ID NO: 70 | ttgctctggttaataatctcaggagcacaaacattccagatccaggttaatttttaaaaagca |
| | gtcaaaagtccaagtggcccttggcagcatttactctctctgtttgctctggttaataatctc |
| | aggagcacaaacattccagatccggcgcgccagggctggaagctacctttgacatcatttcct |
| | ctgcgaatgcatgtataatttctacagaacctattagaaaggatcacccagcctctgcttttg |
| | tacaactttcccttaaaaaactgccaattccactgctgtttggcccaatagtgagaacttttt |
| | cctgctgcctcttggtgcttttgcctatggcccctattctgcctgctgaagacactcttgcca |
| | gcatggacttaaacccctccagctctgacaatcctctttctcttttgttttacatgaagggtc |
| | tggcagccaaagcaatcactcaaagttcaaaccttatcattttttgctttgttcctcttggcc |
| | ttggttttgtacatcagctttgaaaataccatcccagggttaatgctggggttaatttataac |
| | taagagtgctctagttttgcaatacaggacatgctataaaaatggaaagatgttgctttctga |
| | gaggatcttgctaccagtggaacagccactaaggattctgcagtgagagcagagggccagcta |
| | agtggtactctcccagagactgtctgactcacgccaccccctccaccttggacacaggacgct |
| | gtggtttctgagccaggtacagtgactcctttcggtaagtgcagtggaagctgtacactgccc |
| | aggcaaagcgtccgggcagcgtaggcgggcgactcagatcccagccagtggacttagcccctg |
| | tttgctcctccgataactggggtgaccttggttaatattcaccagcagcctcccccgttgccc |
| | ctctggatccactgcttaaatacggacgaggacagggccctgtctcctcagcttcaggcacca |
| | ccactgacctgggacagtaaaacaggtaagtccgctgtttgtgtgctgcctctgaagtccaca |
| | ctgaacaaacttcagcctactcatgtccctaaaatgggcaaacattgcaagcagcaaacagca |
| | aacacacagccctccctgcctgctgaccttggagctggggcagaggtcagagacctctctggc |
| | ctctactaaccatgttcatgttttctttttttttctacaggtcctgggtgacgaacag |
| LMTP6 | aggctcagaggcacacaggagtttctgggctcaccctgcccccttccaacccctcagttccca |
| SEQ ID NO: 71 | tcctccagcagctgtttgtgtgctgcctctgaagtccacactgaacaaacttcagcctactca |
| | tgtccctaaaatgggcaaacattgcaagcagcaaacagcaaacacacagccctccctgcctgc |
| | tgaccttggagctggggcagaggtcagagacctctctgggcccatgccacctccaacatccac |
| | tcgaccccttggaatttcggtggagaggagcagaggttgtcctggcgtggtttaggtagtgtg |
| | agagggccactacgggtttaggctgcccatgtaaggaggcaaggcctggggacacccgagatg |
| | cctggttataattaacccagacatgtggctgccccccccccccccaacacctgctgcctctaa |
| | aaataaccctgtccctggtggatcccactacgggtttaggctgcccatgtaaggaggcaaggc |
| | ctggggacacccgagatgcctggttataattaacccagacatgtggctgcccccccccccccc |
| | aacacctgctgcctctaaaaataaccctgtccctggtggatcccactacgggtttaggctgcc |
| | catgtaaggaggcaaggcctggggacacccgagatgcctggttataattaacccagacatgtg |
| | gctgccccccccccccccaacacctgctgcctctaaaaataaccctgtccctggtggatcccc |
| | tgcatgcgaagatcttcgaacaaggctgtgggggactgagggcaggctgtaacaggcttgggg |
| | gccagggcttatacgtgcctgggactcccaaagtattactgttccatgttcccggcgaagggc |
| | cagctgtcccccgccagctagactcagcacttagtttaggaaccagtgagcaagtcagccctt |
| | ggggcagcccatacaaggccatggggctgggcaagctgcacgcctgggtccggggtgggcacg |
| | gtgcccgggcaacgagctgaaagctcatctgctctcaggggcccctccctggggacagcccct |
| | cctggctagtcacaccctgtaggctcctctatataacccaggggcacaggggctgccctcatt |
| | ctaccaccacctccacagcacagacagacactcaggagccagccagcgtcgagatcttgctac |
| | cagtggaacagccactaaggattctgcagtgagagcagagggccagctaagtggtactctccc |
| | agagactgtctgactcacgccaccccctccaccttggacacaggacgctgtggtttctgagcc |
| | aggtacagtgactcctttcggtaagtgcagtggaagctgtacactgcccaggcaaagcgtccg |
| | ggcagcgtaggcgggcgactcagatcccagccagtggacttagcccctgtttgctcctccgat |
| | aactggggtgaccttggttaatattcaccagcagcctcccccgttgcccctctggatccactg |
| | cttaaatacggacgaggacagggccctgtctcctcagcttcaggcaccaccactgacctggga |
| | cagt |
| LMTP13 | aggctcagaggcacacaggagtttctgggctcaccctgcccccttccaacccctcagttccca |
| SEQ ID NO: 72 | tcctccagcagctgtttgtgtgctgcctctgaagtccacactgaacaaacttcagcctactca |
| | tgtccctaaaatgggcaaacattgcaagcagcaaacagcaaacacacagccctccctgcctgc |
| | tgaccttggagctggggcagaggtcagagacctctctgggcccatgccacctccaacatccac |
| | tcgaccccttggaatttcggtggagaggagcagaggttgtcctggcgtggtttaggtagtgtg |
| | agaggggtacccggggatcttgctaccagtctagaggccgtccgccctcggcaccatcctcac |
| | gacacccaaatatggcgacgggtgaggaatggtggggagttatttttagagcggtgaggaagg |
| | tgggcaggcagcaggtgttggcgctctaaaaataactcccgggagttatttttagagcggagg |
| | aatggtggacacccaaatatggcgacggttcctcacccgtcgccatatttgggtgtccgccct |
| | cggccggggccgcattcctgggggccgggcggtgctcccgcccgcctcgataaaaggctccgg |
| | ggccggcggcggcccacgagctacccggaggagcgggaggcgccaagcgtgagtatcgatctt |
| | gctaccagtggaacagccactaaggattctgcagtgagagcagagggccagctaagtggtact |
| | ctcccagagactgtctgactcacgccaccccctccaccttggacacaggacgctgtggtttct |
| | gagccaggtacagtgactcctttcggtaagtgcagtggaagctgtacactgcccaggcaaagc |
| | gtccgggcagcgtaggcgggcgactcagatcccagccagtggacttagcccctgtttgctcct |
| | ccgataactggggtgaccttggttaatattcaccagcagcctcccccgttgcccctctggatc |
| | cactgcttaaatacggacgaggacagggccctgtctcctcagcttcaggcaccaccactgacc |
| | tgggacagt |
| LMTP14 | gaatggtggacacccaaatatggcgacggttcctcacccgtcgccatatttgggtgtccgccc |
| SEQ ID NO: 73 | tcggccggggccgcattcctgggggccgggcggtgctcccgcccgcctcgataaaaggctccg |
| | gggccggcggcggcccacgagctacccggaggagcgggaggcgccaagcgatcttgctaccag |
| | tggaacagccactaaggattctgcagtgagagcagagggccagctaagtggtactctcccaga |
| | gactgtctgactcacgccaccccctccaccttggacacaggacgctgtggtttctgagccagg |
| | tacagtgactcctttcggtaagtgcagtggaagctgtacactgcccaggcaaagcgtccgggc |
| | agcgtaggcgggcgactcagatcccagccagtggacttagcccctgtttgctcctccgataac |
| | tggggtgaccttggttaatattcaccagcagcctcccccgttgcccctctggatccactgctt |
| | aaatacggacgaggacagggccctgtctcctcagcttcaggcaccaccactgacctgggacag |
| | t |
| LMTP15 | aggctcagaggcacacaggagtttctgggctcaccctgcccccttccaacccctcagttccca |
| SEQ ID NO: 74 | tcctccagcagctgtttgtgtgctgcctctgaagtccacactgaacaaacttcagcctactca |
| | tgtccctaaaatgggcaaacattgcaagcagcaaacagcaaacacacagccctccctgcctgc |
| | tgaccttggagctggggcagaggtcagagacctctctgggcccatgccacctccaacatccac |
| | tcgaccccttggaatttcggtggagaggagcagaggttgtcctggcgtggtttaggtagtgtg |
| | agagggtctagagaatggtggacacccaaatatggcgacggttcctcacccgtcgccatattt |
| | gggtgtccgccctcggccggggccgcattcctgggggccgggcggtgctcccgcccgcctcga |
| | taaaaggctccggggccggcggcggcccacgagctacccggaggagcgggaggcgccaagcga |
| | tcttgctaccagtggaacagccactaaggattctgcagtgagagcagagggccagctaagtgg |
| | tactctcccagagactgtctgactcacgccaccccctccaccttggacacaggacgctgtggt |
| | ttctgagccaggtacagtgactcctttcggtaagtgcagtggaagctgtacactgcccaggca |
| | aagcgtccgggcagcgtaggcgggcgactcagatcccagccagtggacttagcccctgtttgc |
| | tcctccgataactggggtgaccttggttaatattcaccagcagcctcccccgttgcccctctg |
| | gatccactgcttaaatacggacgaggacagggccctgtctcctcagcttcaggcaccaccact |
| | gacctgggacagt |
| LMTP18 | aggctcagaggcacacaggagtttctgggctcaccctgcccccttccaacccctcagttccca |
| SEQ ID NO: 75 | tcctccagcagctgtttgtgtgctgcctctgaagtccacactgaacaaacttcagcctactca |
| | tgtccctaaaatgggcaaacattgcaagcagcaaacagcaaacacacagccctccctgcctgc |
| | tgaccttggagctggggcagaggtcagagacctctctgggcccatgccacctccaacatccac |
| | tcgaccccttggaatttcggtggagaggagcagaggttgtcctggcgtggtttaggtagtgtg |
| | agagggccactacgggtttaggctgcccatgtaaggaggcaaggcctggggacacccgagatg |
| | cctggttataattaacccagacatgtggctgccccccccccccccaacacctgctgcctctaa |
| | aaataaccctgtccctggtggatcccctgcatgcgaagatcttcgaacaaggctgtgggggac |
| | tgagggcaggctgtaacaggcttgggggccagggcttatacgtgcctgggactcccaaagtat |
| | tactgttccatgttcccggcgaagggccagctgtcccccgccagctagactcagcacttagtt |
| | taggaaccagtgagcaagtcagcccttggggcagcccatacaaggccatggggctgggcaagc |
| | tgcacgcctgggtccggggtgggcacggtgcccgggcaacgagctgaaagctcatctgctctc |
| | aggggcccctccctggggacagcccctcctggctagtcacaccctgtaggctcctctatataa |
| | cccaggggcacaggggctgccctcattctaccaccacctccacagcacagacagacactcagg |
| | agccagccagcgtcgagatcttgctaccagtggaacagccactaaggattctgcagtgagagc |
| | agagggccagctaagtggtactctcccagagactgtctgactcacgccaccccctccaccttg |
| | gacacaggacgctgtggtttctgagccaggtacagtgactcctttcggtaagtgcagtggaag |
| | ctgtacactgcccaggcaaagcgtccgggcagcgtaggcgggcgactcagatcccagccagtg |
| | gacttagcccctgtttgctcctccgataactggggtgaccttggttaatattcaccagcagcc |
| | tcccccgttgcccctctggatccactgcttaaatacggacgaggacagggccctgtctcctca |
| | gcttcaggcaccaccactgacctgggacagt |
| LMTP19 | aggctcagaggcacacaggagtttctgggctcaccctgcccccttccaacccctcagttccca |
| SEQ ID NO: 76 | tcctccagcagctgtttgtgtgctgcctctgaagtccacactgaacaaacttcagcctactca |
| | tgtccctaaaatgggcaaacattgcaagcagcaaacagcaaacacacagccctccctgcctgc |
| | tgaccttggagctggggcagaggtcagagacctctctgggcccatgccacctccaacatccac |
| | tcgaccccttggaatttcggtggagaggagcagaggttgtcctggcgtggtttaggtagtgtg |
| | agagggccctgcatgcgaagatcttcgaacaaggctgtgggggactgagggcaggctgtaaca |
| | ggcttgggggccagggcttatacgtgcctgggactcccaaagtattactgttccatgttcccg |
| | gcgaagggccagctgtcccccgccagctagactcagcacttagtttaggaaccagtgagcaag |
| | tcagcccttggggcagcccatacaaggccatggggctgggcaagctgcacgcctgggtccggg |
| | gtgggcacggtgcccgggcaacgagctgaaagctcatctgctctcaggggcccctccctgggg |
| | acagcccctcctggctagtcacaccctgtaggctcctctatataacccaggggcacaggggct |
| | gccctcattctaccaccacctccacagcacagacagacactcaggagccagccagcgtcgaga |
| | tcttgctaccagtggaacagccactaaggattctgcagtgagagcagagggccagctaagtgg |
| | tactctcccagagactgtctgactcacgccaccccctccaccttggacacaggacgctgtggt |
| | ttctgagccaggtacagtgactcctttcggtaagtgcagtggaagctgtacactgcccaggca |
| | aagcgtccgggcagcgtaggcgggcgactcagatcccagccagtggacttagcccctgtttgc |
| | tcctccgataactggggtgaccttggttaatattcaccagcagcctcccccgttgcccctctg |
| | gatccactgcttaaatacggacgaggacagggccctgtctcctcagcttcaggcaccaccact |
| | gacctgggacagt |
| LMTP20 | aggctcagaggcacacaggagtttctgggctcaccctgcccccttccaacccctcagttccca |
| SEQ ID NO: 77 | tcctccagcagctgtttgtgtgctgcctctgaagtccacactgaacaaacttcagcctactca |
| | tgtccctaaaatgggcaaacattgcaagcagcaaacagcaaacacacagccctccctgcctgc |
| | tgaccttggagctggggcagaggtcagagacctctctgggcccatgccacctccaacatccac |
| | tcgaccccttggaatttcggtggagaggagcagaggttgtcctggcgtggtttaggtagtgtg |
| | agagggcccttcagattaaaaataactgaggtaagggcctgggtaggggaggtggtgtgagac |
| | gctcctgtctctcctctatctgcccatcggccctttggggaggaggaatgtgcccaaggacta |
| | aaaaaaggccatggagccagaggggcgagggcaacagacctttcatgggcaaaccttggggcc |
| | ctgctgaagctttggcccactacgggtttaggctgcccatgtaaggaggcaaggcctggggac |
| | acccgagatgcctggttataattaacccagacatgtggctgccccccccccccccaacacctg |
| | ctgcctctaaaaataaccctgtccctggtggatcccctgcatgcgaagatcttcgaacaaggc |
| | tgtgggggactgagggcaggctgtaacaggcttgggggccagggcttatacgtgcctgggact |
| | cccaaagtattactgttccatgttcccggcgaagggccagctgtcccccgccagctagactca |
| | gcacttagtttaggaaccagtgagcaagtcagcccttggggcagcccatacaaggccatgggg |
| | ctgggcaagctgcacgcctgggtccggggtgggcacggtgcccgggcaacgagctgaaagctc |
| | atctgctctcaggggcccctccctggggacagcccctcctggctagtcacaccctgtaggctc |
| | ctctatataacccaggggcacaggggctgccctcattctaccaccacctccacagcacagaca |
| | gacactcaggagccagccagcgtcgagatcttgctaccagtggaacagccactaaggattctg |
| | cagtgagagcagagggccagctaagtggtactctcccagagactgtctgactcacgccacccc |
| | ctccaccttggacacaggacgctgtggtttctgagccaggtacagtgactcctttcggtaagt |
| | gcagtggaagctgtacactgcccaggcaaagcgtccgggcagcgtaggcgggcgactcagatc |
| | ccagccagtggacttagcccctgtttgctcctccgataactggggtgaccttggttaatattc |
| | accagcagcctcccccgttgcccctctggatccactgcttaaatacggacgaggacagggccc |
| | tgtctcctcagcttcaggcaccaccactgacctgggacagt |
| ApoE.hAAT | aggctcagaggcacacaggagtttctgggctcaccctgcccccttccaacccctcagttccca |
| SEQ ID NO: 78 | tcctccagcagctgtttgtgtgctgcctctgaagtccacactgaacaaacttcagcctactca |
| | tgtccctaaaatgggcaaacattgcaagcagcaaacagcaaacacacagccctccctgcctgc |
| | tgaccttggagctggggcagaggtcagagacctctctgggcccatgccacctccaacatccac |
| | tcgaccccttggaatttcggtggagaggagcagaggttgtcctggcgtggtttaggtagtgtg |
| | agaggggtacccggggatottgctaccagtggaacagccactaaggattctgcagtgagagca |
| | gagggccagctaagtggtactctcccagagactgtctgactcacgccaccccctccaccttgg |
| | acacaggacgctgtggtttctgagccaggtacaatgactcctttcggtaagtgcagtggaagc |
| | tgtacactgcccaggcaaagcgtccgggcagcgtaggcgggcgactcagatcccagccagtgg |
| | acttagcccctgtttgctcctccgataactggggtgaccttggttaatattcaccagcagcct |
| | cccccgttgcccctctggatccactgcttaaatacggacgaggacagggccctgtctcctcag |
| | cttcaggcaccaccactgacctgggacagt |
| Alpha-Mic/Bik | aggttaatttttaaaaagcagtcaaaagtccaagtggcccttggcagcatttactctctctgt |
| Enhancer | ttgctctggttaataatctcaggagcacaaacattcc |
| (Mic/BikE) | |
| SEQ ID NO: 79 | |
| Tandem (2) | aggttaatttttaaaaagcagtcaaaagtccaagtggcccttggcagcatttactctctctgt |
| alpha-Mic/Bik | ttgctctggttaataatctcaggagcacaaacattccaggttaatttttaaaaagcagtcaaa |
| Enhancers | agtccaagtggcccttggcagcatttactctctctgtttgctctggttaataatctcaggagc |
| (2 Mic/BikE) | acaaacattcc |
| SEQ ID NO: 80 | |
| | aggctcagaggcacacaggagtttctgggctcaccctgcccccttccaacccctcagttccca |
| ApoE Hepatic | tcctccagcagctgtttgtgtgctgcctctgaagtccacactgaacaaacttcagcctactca |
| Control Region | tgtccctaaaatgggcaaacattgcaagcagcaaacagcaaacacacagccctccctgcctgc |
| containing ApoE | tgaccttggagctggggcagaggtcagagacctctctgggcccatgccacctccaacatccac |
| Enhancer | tcgaccccttggaatttcggtggagaggagcagaggttgtcctggcgtggtttaggtagtgtg |
| SEQ ID NO: 81 | agaggg |
| Tandem (2) | aggctcagaggcacacaggagtttctgggctcaccctgcccccttccaacccctcagttccca |
| ApoE Enhancers | tcctccagcagctgtttgtgtgctgcctctgaagtccacactgaacaaacttcagcctactca |
| SEQ ID NO: 82 | tgtccctaaaatgggcaaacattgcaagcagcaaacagcaaacacacagccctccctgcctgc |
| | tgaccttggagctggggcagaggtcagagacctctctgggcccatgccacctccaacatccac |
| | tcgaccccttggaatttcggtggagaggagcagaggttgtcctggcgtggtttaggtagtgtg |
| | agagggtctagaaggctcagaggcacacaggagtttctgggctcaccctgcccccttccaacc |
| | cctcagttcccatcctccagcagctgtttgtgtgctgcctctgaagtccacactgaacaaact |
| | tcagcctactcatgtccctaaaatgggcaaacattgcaagcagcaaacagcaaacacacagcc |
| | ctccctgcctgctgaccttggagctggggcagaggtcagagacctctctg |
| hAAT Promoter | gatcttgctaccagtggaacagccactaaggattctgcagtgagagcagagggccagctaagt |
| SEQ ID NO: 83 | ggtactctcccagagactgtctgactcacgccaccccctccaccttggacacaggacgctgtg |
| | gtttctgagccaggtaca [symbol] actcctttcggtaagtgcagtggaagctgtacactgcccagg |
| | caaagcgtccgggcagcgtaggcgggcgactcagatcccagccagtggacttagcccctgttt |
| | gctcctccgataactggggtgaccttggttaatattcaccagcagcctcccccgttgcccctc |
| | tggatccactgcttaaatacggacgaggacagggccctgtctcctcagcttcaggcaccacca |
| | ctgacctgggacagt |
| hAAT(ΔATG) | gatcttgctaccagtggaacagccactaaggattctgcagtgagagcagagggccagctaagt |
| Promoter | ggtactctcccagagactgtctgactcacgccaccccctccaccttggacacaggacgctgtg |
| SEQ ID NO: 84 | gtttctgagccaggtaca [symbol] actcctttcggtaagtgcagtggaagctgtacactgcccagg |
| | caaagcgtccgggcagcgtaggcgggcgactcagatcccagccagtggacttagcccctgttt |
| | gctcctccgataactggggtgaccttggttaatattcaccagcagcctcccccgttgcccctc |
| | tggatccactgcttaaatacggacgaggacagggccctgtctcctcagcttcaggcaccacca |
| | ctgacctgggacagt |
| Mck Enhancer | ccactacgggtttaggctgcccatgtaaggaggcaaggcctggggacacccgagatgcctggt |
| (MckE) | tataattaacccagacatgtggctgccccccccccccccaacacctgctgcctctaaaaataa |
| SEQ ID NO: 85 | ccctgtccctggtggatc |
| Tandem (2) Mck | ccactacgggtttaggctgcccatgtaaggaggcaaggcctggggacacccgagatgcctggt |
| Enhancers | tataattaacccagacatgtggctgccccccccccccccaacacctgctgcctctaaaaataa |
| (2 MckE) | ccctgtccctggtggatcccactacgggtttaggctgcccatgtaaggaggcaaggcctgggg |
| SEQ ID NO: 86 | acacccgagatgcctggttataattaacccagacatgtggctgccccccccccccccaacacc |
| | tgctgcctctaaaaataaccctgtccctggtggatc |
| Tandem Mck (3) | ccactacgggtttaggctgcccatgtaaggaggcaaggcctggggacacccgagatgcctggt |
| Enhancers | tataattaacccagacatgtggctgccccccccccccccaacacctgctgcctctaaaaataa |
| (3 MckE) | ccctgtccctggtggatcccactacgggtttaggctgcccatgtaaggaggcaaggcctgggg |
| SEQ ID NO: 87 | acacccgagatgcctggttataattaacccagacatgtggctgccccccccccccccaacacc |
| | tgctgcctctaaaaataaccctgtccctggtggatcccactacgggtttaggctgcccatgta |
| | aggaggcaaggcctggggacacccgagatgcctggttataattaacccagacatgtggctgcc |
| | ccccccccccccaacacctgctgcctctaaaaataaccctgtccctggtggatc |
| Myosin heavy | cccttcagattaaaaataactgaggtaagggcctgggtaggggaggtggtgtgagacgctcct |
| chain enhancer | gtctctcctctatctgcccatcggccctttggggaggaggaatgtgcccaaggactaaaaaaa |
| (MhcE) | ggccatggagccagaggggcgagggcaacagacctttcatgggcaaaccttggggccctgctg |
| SEQ ID NO: 88 | aagctttggc |
| CAG/CB7 | gacattgattattgactagttattaatagtaatcaattacggggtcattagttcatagcccat |
| SEQ ID NO: 89 | atatggagttccgcgttacataacttacggtaaatggcccgcctggctgaccgcccaacgacc |
| | cccgcccattgacgtcaataatgacgtatgttcccatagtaacgccaatagggactttccatt |
| | gacgtcaatgggtggagtatttacggtaaactgcccacttggcagtacatcaagtgtatcata |
| | tgccaagtacgccccctattgacgtcaatgacggtaaatggcccgcctggcattatgcccagt |
| | acatgaccttatgggactttcctacttggcagtacatctacgtattagtcatcgctattacca |
| | tggtcgaggtgagccccacgttctgcttcactctccccatctcccccccctccccacccccaa |
| | ttttgtatttatttattttttaattattttgtgcagcgatgggggcggggggggggggggggc |
| | gcgcgccaggcggggcggggcggggcgaggggcggggcggggcgaggcggagaggtgcggcgg |
| | cagccaatcagagcggcgcgctccgaaagtttccttttatggcgaggcggcggcggcggcggc |
| | cctataaaaagcgaagcgcgcggcgggcgggagtcgctgcgcgctgccttcgccccgtgcccc |
| | gctccgccgccgcctcgcgccgcccgccccggctctgactgaccgcgttactcccacaggtga |
| | gcgggcgggacggcccttctcctccgggctgtaattagcgcttggtttaatgacggcttgttt |
| | cttttctgtggctgcgtgaaagccttgaggggctccgggagggccctttgtgcggggggagcg |
| | gctcggggggtgcgtgcgtgtgtgtgtgcgtggggagcgccgcgtgcggctccgcgctgcccg |
| | gcggctgtgagcgctgcgggcgcggcgcggggctttgtgcgctccgcagtgtgcgcgagggga |
| | gcgcggccgggggcggtgccccgcggtgcggggggggctgcgaggggaacaaaggctgcgtgc |
| | ggggtgtgtgcgtgggggggtgagcagggggtgtgggcgcgtcggtcgggctgcaaccccccc |
| | tgcacccccctccccgagttgctgagcacggcccggcttcgggtgcggggctccgtacggggc |
| | gtggcgcggggctcgccgtgccgggcggggggtggcggcaggtgggggtgccgggcggggcgg |
| | ggccgcctcgggccggggagggctcgggggaggggcgcggcggcccccggagcgccggcggct |
| | gtcgaggcgcggcgagccgcagccattgccttttatggtaatcgtgcgagagggcgcagggac |
| | ttcctttgtcccaaatctgtgcggagccgaaatctgggaggcgccgccgcaccccctctagcg |
| | ggcgcggggcgaagcggtgcggcgccggcaggaaggaaatgggcggggagggccttcgtgcgt |
| | cgccgcgccgccgtccccttctccctctccagcctcggggctgtccgcggggggacggctgcc |
| | ttcgggggggacggggcagggcggggttcggcttctggcgtgtgaccggcggctctagagcct |
| | ctgctaaccatgttcatgccttcttctttttcctacagctcctgggcaacgtgctggttattg |
| | tgctgtctcatcattttggcaaag |
| CK8 | ccactacgggtttaggctgcccatgtaaggaggcaaggcctggggacacccgagatgcctggt |
| SEQ ID NO: 90 | tataattaacccagacatgtggctgccccccccccccccaacacctgctgcctctaaaaataa |
| | ccctgtccctggtggatcccactacgggtttaggctgcccatgtaaggaggcaaggcctgggg |
| | acacccgagatgcctggttataattaacccagacatgtggctgccccccccccccccaacacc |
| | tgctgcctctaaaaataaccctgtccctggtggatcccactacgggtttaggctgcccatgta |
| | aggaggcaaggcctggggacacccgagatgcctggttataattaacccagacatgtggctgcc |
| | ccccccccccccaacacctgctgcctctaaaaataaccctgtccctggtggatcccctgcatg |
| | cgaagatcttcgaacaaggctgtgggggactgagggcaggctgtaacaggcttgggggccagg |
| | gcttatacgtgcctgggactcccaaagtattactgttccatgttcccggcgaagggccagctg |
| | tcccccgccagctagactcagcacttagtttaggaaccagtgagcaagtcagcccttggggca |
| | gcccatacaaggccatggggctgggcaagctgcacgcctgggtccggggtgggcacggtgccc |
| | gggcaacgagctgaaagctcatctgctctcaggggcccctccctggggacagcccctcctggc |
| | tagtcacaccctgtaggctcctctatataacccaggggcacaggggctgccctcattctacca |
| | ccacctccacagcacagacagacactcaggagccagccagcgtcga |
| mU1a | atggaggcggtactatgtagatgagaattcaggagcaaactgggaaaagcaactgcttccaaa |
| SEQ ID NO: 91 | tatttgtgatttttacagtgtagttttggaaaaactcttagcctaccaattcttctaagtgtt |
| | ttaaaatgtgggagccagtacacatgaagttatagagtgttttaatgaggcttaaatatttac |
| | cgtaactatgaaatgctacgcatatcatgctgttcaggctccgtggccacgcaactcatact |
| EF-1α | gggcagagcgcacatcgcccacagtccccgagaagttggggggaggggtcggcaattgaacgg |
| SEQ ID NO: 92 | gtgcctagagaaggtggcgcggggtaaactgggaaagtgatgtcgtgtactggctccgccttt |
| | ttcccgagggtgggggagaaccgtatataagtgcagtagtcgccgtgaacgttctttttcgca |
| | acgggtttgccgccagaacacag |
| TBG | gggctggaagctacctttgacatcatttcctctgcgaatgcatgtataatttctacagaacct |
| SEQ ID NO: 93 | attagaaaggatcacccagcctctgcttttgtacaactttcccttaaaaaactgccaattcca |
| | ctgctgtttggcccaatagtgagaactttttcctgctgcctcttggtgcttttgcctatggcc |
| | cctattctgcctgctgaagacactcttgccagcatggacttaaacccctccagctctgacaat |
| | cctctttctcttttgttttacatgaagggtctggcagccaaagcaatcactcaaagttcaaac |
| | cttatcattttttgctttgttcctcttggccttggttttgtacatcagctttgaaaataccat |
| | cccagggttaatgctggggttaatttataactaagagtgctctagttttgcaatacaggacat |
| | gctataaaaat |
| | ggaaagat |
| Chimeric Intron | gtaagtatcaaggttacaagacaggtttaaggagaccaatagaaactgggcttgtcgagacag |
| SEQ ID NO: 94 | agaagactcttgcgtttctgataggcacctattggtcttactgacatccactttgcctttctc |
| | tccacag |
| VH4 Intron | gtgagtatctcagggatccagacatggggatatgggaggtgcctctgatcccagggctcactg |
| SEQ ID NO: 95 | tgggtctctctgttcacag |
| SV40 Intron | gtaagtttagtctttttgtcttttatttcaggtcccggatccggtggtggtgcaaatcaaaga |
| SEQ ID NO: 96 | actgctcctcagtggatgttgcctttacttctag |
| β-globin PolyA | ataaaggaaatttattttcattgcaatagtgtgttggaattttttgtgtctctca |
| signal | |
| SEQ ID NO: 97 | |
| Rabbit β-globin | gatctttttccctctgccaaaaattatggggacatcatgaagccccttgagcatctgacttct |
| polyA | ggctaataaaggaaatttattttcattgcaatagtgtgttggaattttttgtgtctctcactc |
| SEQ ID NO: 98 | g |
| 5′ITR | ctgcgcgctcgctcgctcactgaggccgcccgggcaaagcccgggcgtcgggcgacctttggt |
| SEQ ID NO: 99 | cgcccggcctcagtgagcgagcgagcgcgcagagagggagtggccaactccatcactaggggt |
| | tcct |
| 5′-ITR | ctgcgcgctcgctcgctcactgaggccgcccgggcaaagcccgggcgtcgggcgacctttggt |
| (Deleted D- | cgcccggcctcagtgagcgagcgagcgcgcagagagggagtgg |
| sequence for self- | |
| complimentary | |
| AAV) | |
| SEQ ID NO: 100 | |
| 3′-ITR AAV | gaacccctagtgatggagttggccactccctctctgcgcgctcgctcgctcactgaggccgcc |
| SEQ ID NO: 101 | cgggcaaagcccgggcgtcgggcgacctttggtcgcccggcctcagtgagcgagcgagcgcgc |
| | agagagggagtggccaa |
| 3′-ITR | ttggccactccctctctgcgcgctcgctcgctcactgaggccgggcgaccaaaggtcgcccga |
| (Deleted D- | cgcccgggctttgcccgggcggcctcagtgagcgagcgagcgcgcag |
| sequence for self- | |
| complimentary | |
| AAV) | |
| SEQ ID NO: 102 | |

In certain embodiments, the viral vectors provided herein comprise one or more regulatory elements other than a promoter. In certain embodiments, the viral vectors provided herein comprise an enhancer. In certain embodiments, the viral vectors provided herein comprise a repressor. In certain embodiments, the viral vectors provided herein comprise an intron (e.g. VH4 intron (SEQ ID NO:95) SV40 Intron (SEQ ID NO:96) or a chimeric intron (β-globin/Ig Intron) (SEQ ID NO:94).

In certain embodiments, the viral vectors provided herein comprise a polyadenylation sequence downstream of the coding region of the transgene. Any polyA site that signals termination of transcription and directs the synthesis of a polyA tail is suitable for use in AAV vectors of the present disclosure. Exemplary polyA signals are derived from, but not limited to, the following: the SV40 late gene, the rabbit β-globin gene (SEQ ID NO:97), the bovine growth hormone (BPH) gene, the human growth hormone (hGH) gene, the synthetic polyA (SPA) site, and the bovine growth hormone (bGH) gene. See, e.g., Powell and Rivera-Soto, 2015, Discov. Med., 19(102):49-57.

5.1.4 Signal Peptides

In certain embodiments, the vectors provided herein comprise components that modulate protein delivery. In certain embodiments, the viral vectors provided herein comprise one or more signal peptides. Signal peptides (also referred to as “signal sequences”) may also be referred to herein as “leader sequences” or “leader peptides”. In certain embodiments, the signal peptides allow for the transgene product to achieve the proper packaging (e.g., glycosylation) in the cell. In certain embodiments, the signal peptides allow for the transgene product to achieve the proper localization in the cell. In certain embodiments, the signal peptides allow for the transgene product to achieve secretion from the cell.

There are two general approaches to select a signal sequence for protein production in a gene therapy context or in cell culture. One approach is to use a signal peptide from proteins homologous to the protein being expressed. For example, a human antibody signal peptide may be used to express IgGs in CHO or other cells. Another approach is to identify signal peptides optimized for the particular host cells used for expression. Signal peptides may be interchanged between different proteins or even between proteins of different organisms, but usually the signal sequences of the most abundant secreted proteins of that cell type are used for protein expression. For example, the signal peptide of human albumin, the most abundant protein in plasma, was found to substantially increase protein production yield in CHO cells. However, certain signal peptides may retain function and exert activity after being cleaved from the expressed protein as “post-targeting functions”. Thus, in specific embodiments, the signal peptide is selected from signal peptides of the most abundant proteins secreted by the cells used for expression to avoid the post-targeting functions. In a certain embodiment, the signal sequence is fused to both the heavy and light chain sequences. An exemplary sequence is MYRMQLLLLIALSLALVTNS (SEQ ID NO:103) which can be encoded by a nucleotide sequence of SEQ ID NO:104 (see Table 2, FIGS. 2A-2I). Alternatively, signal sequences that are appropriate for expression, and may cause selective expression or directed expression of the HuPTM mAb or Fab or scFv in muscle, or liver are provided in Tables 2 and 3, respectively, below.

TABLE 2
Signal peptides for expression in liver cells.

| | SEQ ID | |
|---|---|---|
| Signal Peptide Origin | NO: | Sequence |
| Mutant interleukin 2 | 103 | MYRMQLLLLIALSLALVINS |
| signal peptide | | |
| Mutant interleukin 2 | 104 | atgtataggatgcaactgctcctcctgattg |
| signal peptide coding | | ctctgagcctggctcttgtgaccaactct |
| sequence | | |
| Human Serum albumin | 105 | MKWVTFISLLFLFSSAYS |
| Human α-1 Antitrypsin | 106 | MPSSVSWGILLLAGLCCLVPVSLA |
| (SERPINA1) | | |
| Human Apolipoprotein | 107 | MKAAVLTLAVLFLTGSQA |
| A-1 | | |
| Human Apolipoprotein | 108 | MKLLAATVLLLTICSLEG |
| A-2 | | |
| Human Apolipoprotein | 109 | MDPPRPALLALLALPALLLLLLAGARA |
| B-100 | | |
| Human Coagulation | 110 | MQRVNMIMAESPGLITICLLGYLLSAEC |
| Factor IX | | |
| Human Complement | 111 | MGPLMVLFCLLFLYPGLADS |
| C2 | | |
| Human Complement | 112 | MWLLVSVILISRISSVGG |
| Factor H-related | | |
| Protein 2 (CFHR2) | | |
| Human Complement | 113 | MLLLFSVILISWVSTVGG |
| Factor H-related | | |
| Protein 5 (CFHR5) | | |
| Human Fibrinogen α- | 114 | MFSMRIVCLVLSVVGTAWT |
| chain (FGA) | | |
| Human Fibrinogen β- | 115 | MKRMVSWSFHKLKTMKHLLLLLLCVFLVKS |
| chain (FGB) | | |
| Human Fibrinogen γ- | 116 | MSWSLHPRNLILYFY ALLFLSSTCVA |
| chain (FGG) | | |
| Human α-2-HS- | 117 | MKSLVLLLCLAQLWGCHS |
| Glycoprotein (AHSG) | | |
| Human Hemopexin | 118 | MARVLGAPVALGLWSLCWSLAIA |
| (HPX) | | |
| Human Kininogen-1 | 119 | MKLITILFLCSRLLLSLT |
| Human Mannose- | 120 | MSLFPSLPLLLLSMVAASYS |
| binding protein C | | |
| (MBL2) | | |
| Human Plasminogen | 121 | MEHKEVVLLLLLFLKSGQG |
| (PLMN) | | |
| Human Prothrombin | 122 | MAHVRGLQLPGCLALAALCSLVHS |
| (Coagulation Factor II) | | |
| Human Secreted | 123 | MISRMEKMTMMMKILIMFALGMNYWSCSG |
| Phosphoprotein 24 | | |
| Human Anti-thrombin- | 124 | MYSNVIGTVTSGKRKVYLLSLLLIGFWDCVTC |
| III (SERPINC1) | | |
| Human Serotransferrin | 125 | MRLAVGALLVCAVLGLCLA |
| (TF) | | |

TABLE 3
Signal peptides for expression in muscle cells.

| | SEQ | |
|---|---|---|
| Signal Peptide | ID | |
| Origin | NO: | Sequence |
| Human SPARC | 126 | MRAWIFFLLCLAGRALA |
| Human Collagen | 127 | MFSFVDLRLLLLLAATAL |
| alpha-1 (I) | | LTHG |
| chain | | |
| Human | 128 | MKLVFLVLLFLGALGLCLA |
| Lactotransferrin | | |
| Human Complement | 129 | MGPTSGPSLLLLLLTHLP |
| C3 | | LALG |
| Human Lumican | 130 | MSLSAFTLFLALIGGTSG |
| Human Gelsolin | 131 | MAPHRPAPALLCALSL |
| isoform 1 | | ALCALSLPVRA |
| Human Pro- | 132 | MWATLPLLCAGAWLLGVPV |
| cathepsin H | | CGA |
| Human SERPINF1 | 133 | MQALVLLLCIGALLGHSSC |
| Human SERPINE1 | 134 | MQMSPALTCLVLGLALVFG |
| | | EGSA |
| Human Cathepsin D | 135 | MQPSSLLPLALCLLAAPASA |
| Human TIMP1 | 136 | MAPFEPLASGILLLLWLIA |
| | | PSRA |
| Human Fibronectin | 137 | MLRGPGPGLLLLAVQCLGT |
| | | AVPSTGASKSKR |
| Human Complement C1s | 138 | MWCIVLFSLLAWVYA |
| subcomponent | | |
| Human Cathepsin L1 | 139 | MNPTLILAAFCLGIASA |
| Human Cathepsin B | 140 | MWQLWASLCCLLVLANA |
| Human Salivary acidic | 141 | MLLILLSVALLAFSSA |
| proline-rich | | |
| phosphoprotein ½ | | |
| Human Follistatin- | 142 | MWKRWLALALALVAVAWVRA |
| related | | |
| protein 1 | | |

5.1.5 Polycistronic Messages—IRES and 2A Linkers and scFv Constructs

Internal ribosome entry sites. A single construct can be engineered to encode both the heavy and light chains separated by a cleavable linker or IRES so that separate heavy and light chain polypeptides are expressed by the transduced cells. In certain embodiments, the viral vectors provided herein provide polycistronic (e.g., bicistronic) messages. For example, the viral construct can encode the heavy and light chains separated by an internal ribosome entry site (IRES) elements (for examples of the use of IRES elements to create bicistronic vectors see, e.g., Gurtu et al., 1996, Biochem. Biophys. Res. Comm. 229(1):295-8, which is herein incorporated by reference in its entirety). IRES elements bypass the ribosome scanning model and begin translation at internal sites. The use of IRES in AAV is described, for example, in Furling et al., 2001, Gene Ther 8(11): 854-73, which is herein incorporated by reference in its entirety. In certain embodiments, the bicistronic message is contained within a viral vector with a restraint on the size of the polynucleotide(s) therein. In certain embodiments, the bicistronic message is contained within an AAV virus-based vector (e.g., an AAV8-based, AAV9-based or AAVrh10-based vector).

Furin-2A linkers. In other embodiments, the viral vectors provided herein encode the heavy and light chains separated by a cleavable linker such as the self-cleaving 2A and 2A-like peptides, with or without upstream furin cleavage sites, e.g. Furin/2A linkers, such as furin/F2A (F/F2A) or furin/T2A (F/T2A) linkers (Fang et al., 2005, Nature Biotechnology 23: 584-590, Fang, 2007, Mol Ther 15: 1153-9, and Chang, J. et al, MAbs 2015, 7(2):403-412, each of which is incorporated by reference herein in its entirety). For example, a furin/2A linker may be incorporated into an expression cassette to separate the heavy and light chain coding sequences, resulting in a construct with the structure:

Signal sequence-Heavy chain-Furin site-2A site-Signal Sequence-Light chain-PolyA.

A 2A site or 2A-like site, such as an F2A site comprising the amino acid sequence RKRR(GSG)APVKQTLNFDLLKLAGDVESNPGP (SEQ ID NOS:166 or 167) or a T2A site comprising the amino acid sequence RKRR(GSG)EGRGSLLTCGDVEENPGP (SEQ ID NOS:164 or 165), is self-processing, resulting in “cleavage” between the final G and P amino acid residues. Several linkers, with or without an upstream flexible Gly-Ser-Gly (GSG) linker sequence, that could be used include but are not limited to:

T2A:
(SEQ ID NOS: 147 or 148)
(GSG)EGRGSLLTCGDVEENPGP;
P2A:
(SEQ ID NOS: 149 or 150)
(GSG)ATNFSLLKQAGDVEENPGP;
E2A:
(SEQ ID NOS: 151 or 152)
(GSG)QCTNYALLKLAGDVESNPGP;
F2A:
(SEQ ID NOS: 153 or 154)
(GSG)APVKQTLNFDLLKLAGDVESNPGP

(see also, e.g., Szymczak, et al., 2004, Nature Biotechnol 22(5):589-594, and Donnelly, et al., 2001, J Gen Virol, 82:1013-1025, each of which is incorporated herein by reference). Exemplary amino acid and nucleotide sequences encoding different parts of the flexible linker are described in Table 4.

TABLE 4
Linker Sequences

| | SEQ | |
|---|---|---|
| | ID | |
| ID | NO: | Sequence |
| GSG linker | | GSG |
| Furin linker | 143 | RKRR |
| Furin linker | 144 | RRRR |
| Furin linker | 145 | RRKR |
| Furin linker | 146 | RKKR |
| T2A | 147 | EGRGSLLTCGDVEEN |
| | | PGP |
| T2A | 148 | GSGEGRGSLLTCGDV |
| | | EENPGP |
| P2A | 149 | ATNFSLLKQAGDVEE |
| | | NPGP |
| P2A | 150 | GSGATNFSLLKQAGD |
| | | VEENPGP |
| E2A | 151 | QCTNYALLKLAGDVE |
| | | SNPGP |
| E2A | 152 | GSGQCTNYALLKLAG |
| | | DVESNPGP |
| F2A | 153 | APVKQTLNFDLLKLA |
| | | GDVESNPGP |
| F2A | 154 | GSGAPVKQTLNFDLL |
| | | KLAGDVESNPGP |
| Furin-T2A | 155 | RKRREGRGSLLTCGD |
| | | VEENPGP |
| Furin-GSG-T2A | 156 | RKRRGSGEGRGSLLT |
| | | CGDVEENPGP |
| Furin-F2A | 157 | RKRRAPVKQTLNFDL |
| | | LKLAGDVESNPGP |
| Furin-GSG-F2A | 158 | RKRRGSGAPVKQTLN |
| | | FDLLKLAGDVESNPGP |
| Furin-GSG-T2A | 159 | agaaagagaagaggct |
| | | ctggagaaggcagaggc |
| | | tccctgctgacatgtgg |
| | | ggatgttgaagagaatc |
| | | ctgggcct |
| Furin | 160 | agaaagagaaga |
| Furin-GSG linker | 161 | agaaagagaagaggctc |
| | | tgga |
| GSG linker | 162 | ggctctgga |
| T2A | 163 | gaaggcagaggctccctg |
| | | ctgacatgtggggatgtt |
| | | gaagagaatcctgggcct |

In certain embodiments an additional proteolytic cleavage site, e.g. a furin cleavage site, is incorporated into the expression construct adjacent to the self-processing cleavage site (e.g. 2A or 2A like sequence), thereby providing a means to remove additional amino acids that remain following cleavage by the self processing cleavage sequence. Without being bound to any one theory, a peptide bond is skipped when the ribosome encounters the 2A sequence in the open reading frame, resulting in the termination of translation, or continued translation of the downstream sequence (the light chain). This self-processing sequence results in a string of additional amino acids at the end of the C-terminus of the heavy chain. However, such additional amino acids can then be cleaved by host cell Furin at the furin cleavage site(s), e.g. located immediately prior to the 2A site and after the heavy chain sequence, and further cleaved by carboxypeptidases. The resultant heavy chain may have one, two, three, or more additional amino acids included at the C-terminus, or it may not have such additional amino acids, depending on the sequence of the Furin linker used and the carboxypeptidase that cleaves the linker in vivo (See, e.g., Fang et al., 17 Apr. 2005, Nature Biotechnol. Advance Online Publication; Fang et al., 2007, Molecular Therapy 15(6):1153-1159; Luke, 2012, Innovations in Biotechnology, Ch. 8, 161-186). Furin linkers that may be used comprise a series of four basic amino acids, for example, RKRR (SEQ ID NO:143), RRRR (SEQ ID NO:144), RRKR (SEQ ID NO:145), or RKKR (SEQ ID NO:146). Once this linker is cleaved by a carboxypeptidase, additional amino acids may remain, such that an additional zero, one, two, three or four amino acids may remain on the C-terminus of the heavy chain, for example, R, RR, RK, RKR, RRR, RRK, RKK, RKRR (SEQ ID NO:143), RRRR (SEQ ID NO:144), RRKR (SEQ ID NO:145), or RKKR (SEQ ID NO:146). In certain embodiments, once the linker is cleaved by a carboxypeptidase, no additional amino acids remain. In certain embodiments, 0.5% to 1%, 1% to 2%, 5%, 10%, 15%, or 20% of the antibody, e.g., antigen-binding fragment, population produced by the constructs for use in the methods described herein has one, two, three, or four amino acids remaining on the C-terminus of the heavy chain after cleavage. In certain embodiments, the furin linker has the sequence R-X-K/R-R (SEQ ID NO:230 or 231), such that the additional amino acids on the C-terminus of the heavy chain are R, RX, RXK, RXR, RXKR (SEQ ID NO:230), or RXRR (SEQ ID NO:231), where X is any amino acid, for example, alanine (A). In certain embodiments, no additional amino acids may remain on the C-terminus of the heavy chain.

Flexible peptide linker. In some embodiments, a single construct can be engineered to encode both the heavy and light chains (e.g. the heavy and light chain variable domains) separated by a flexible peptide linker such as those encoding a scFv. A flexible peptide linker can be composed of flexible residues like glycine and serine so that the adjacent heavy chain and light chain domains are free to move relative to one another. The construct may be arranged such that the heavy chain variable domain is at the N-terminus of the scFv, followed by the linker and then the light chain variable domain. Alternatively, the construct may be arranged such that the light chain variable domain is at the N-terminus of the scFv, followed by the linker and then the heavy chain variable domain. That is, the components may be arranged as NH₂-V_{L}-linker-V_{H}-COOH or NH₂-V_{H}-linker-V_{L}-COOH.

In certain embodiments, an expression cassette described herein is contained within a viral vector with a restraint on the size of the polynucleotide(s) therein. In certain embodiments, the expression cassette is contained within an AAV virus-based vector. Due to the size restraints of certain vectors, the vector may or may not accommodate the coding sequences for the full heavy and light chains of the therapeutic antibody but may accommodate the coding sequences of the heavy and light chains of antigen binding fragments, such as the heavy and light chains of a Fab or F(ab′)₂ fragment or an scFv. In particular, the AAV vectors described herein may accommodate a transgene of approximately 4.7 kilobases. Substitution of smaller expression elements would permit the expression of larger protein products, such as full-length therapeutic antibodies.

5.1.6 Untranslated Regions

In certain embodiments, the viral vectors provided herein comprise one or more untranslated regions (UTRs), e.g., 3′ and/or 5′ UTRs. In certain embodiments, the UTRs are optimized for the desired level of protein expression. In certain embodiments, the UTRs are optimized for the mRNA half-life of the transgene. In certain embodiments, the UTRs are optimized for the stability of the mRNA of the transgene. In certain embodiments, the UTRs are optimized for the secondary structure of the mRNA of the transgene.

5.1.7 Inverted Terminal Repeats

In certain embodiments, the viral vectors provided herein comprise one or more inverted terminal repeat (ITR) sequences. ITR sequences may be used for packaging the recombinant gene expression cassette into the virion of the viral vector. In certain embodiments, the ITR is from an AAV, e.g., AAV8 or AAV2 (see, e.g., Yan et al., 2005, J. Virol., 79(1):364-379; U.S. Pat. No. 7,282,199 B2, U.S. Pat. No. 7,790,449 B2, U.S. Pat. No. 8,318,480 B2, U.S. Pat. No. 8,962,332 B2 and International Patent Application No. PCT/EP2014/076466, each of which is incorporated herein by reference in its entirety). In preferred embodiments, nucleotide sequences encoding the ITRs may, for example, comprise the nucleotide sequences of SEQ ID NOS:99 (5′-ITR) or 101 (3′-ITR). In certain embodiments, the modified ITRs used to produce self-complementary vector, e.g., scAAV, may be used (see, e.g., Wu, 2007, Human Gene Therapy, 18(2):171-82, McCarty et al, 2001, Gene Therapy, Vol 8, Number 16, Pages 1248-1254; and U.S. Pat. Nos. 6,596,535; 7,125,717; and 7,456,683, each of which is incorporated herein by reference in its entirety). In preferred embodiments, nucleotide sequences encoding the modified ITRs may, for example, comprise the nucleotide sequences of SEQ ID NOS:100 (5′-ITR) or 102 (3′-ITR).

5.1.8 Transgenes

The transgenes encode a HuPTM mAb, either as a full-length antibody or an antigen binding fragment thereof, e.g. a Fab fragment (an HuGlyFab) or a F(ab′)₂, nanobody, or an scFv based upon a therapeutic antibody disclosed herein. In specific embodiments, the HuPTM mAb or antigen binding fragment, particularly the HuGlyFab, are engineered to contain additional glycosylation sites on the Fab domain (e.g., see Courtois et al., 2016, mAbs 8: 99-112 which is incorporated by reference herein in its entirety for it description of sites of hyperglycosylation on a Fab domain). In addition, for the HuPTM mAb comprising an Fc domain, the Fc domain may be engineered to alter the glycosylation site at N297 to prevent glycosylation at that site (for example, a substitution at N297 for another amino acid and/or a substitution at T297 for a residue that is not a T or S to knock out the glycosylation site). Such Fc domains are “aglycosylated”.

### 5.1.8.1 Constructs for Expression of Full Length HuPTM mAb

In certain embodiments, the transgenes encode a full length heavy chain (including the heavy chain variable domain, the heavy chain constant domain 1 (CH1), the hinge and Fc domain) and a full length light chain (light chain variable domain and light chain constant domain) that upon expression associate to form antigen-binding antibodies with Fc domains. The recombinant AAV constructs express the intact (i.e., full length) or substantially intact HuPTM mAb in a cell, cell culture, or in a subject. (“Substantially intact” refers to mAb having a sequence that is at least 95% identical to the full-length mAb sequence.) The nucleotide sequences encoding the heavy and light chains may be codon optimized for expression in human cells and have reduced incidence of CpG dimers in the sequence to promote expression in human cells. The transgenes may encode any full-length antibody. In preferred embodiments, the transgenes encode a full-length form of any of the therapeutic antibodies disclosed herein, for example, the Fab fragment or scFv of which depicted in FIG. 2A-I herein and including, in certain embodiments, the associated Fc domain provided in Table 6.

The full length mAb encoded by the transgene described herein preferably have the Fc domain of the full-length therapeutic antibody or is an Fc domain of the same type of immunoglobulin as the therapeutic antibody to be expressed. In certain embodiments, the Fc region is an IgG Fc region, but in other embodiments, the Fc region may be an IgA, IgD, IgE, or IgM. The Fc domain is preferably of the same isotype as the therapeutic antibody to be expressed, for example, if the therapeutic antibody is an IgG1 isotype, then the antibody expressed by the transgene comprises an IgG1 Fc domain. The antibody expressed from the transgene may have an IgG1, IgG2, IgG3 or IgG4 Fc domain.

The Fc region of the intact mAb has one or more effector functions that vary with the antibody isotype. The effector functions can be the same as that of the wild-type or the therapeutic antibody or can be modified therefrom to add, enhance, modify, or inhibit one or more effector functions using the Fc modifications disclosed in Section 5.1.9, infra. In certain embodiments, the HuPTM mAb transgene encodes a mAb comprising an Fc polypeptide comprising an amino acid sequence that is at least 85%, 86%, 87%, 88%, 89%, 90%, 91%, 92%, 93%, 94%, 95%, 96%, 97%, 98% or 99% identical to the sequence set forth in the Fc domain polypeptides of the therapeutic antibodies described herein as set forth in Table 6 or an exemplary Fc domain of an IgG1, IgG2 or IgG4 isotype as set forth in Table 6. In some embodiments, the HuPTM mAb comprises a Fc polypeptide of a sequence that is a variant of the Fc polypeptide sequence in Table 6 in that the sequence has been modified with one or more of the techniques described in Section 5.1.9, infra, to alter the Fc polypeptide's effector function.

In specific embodiments, provided are recombinant AAV constructs such as the constructs shown in FIGS. 1A and 1B, for gene therapy administration to a human subject in order to express an intact or substantially intact HuPTM mAb in the subject. Gene therapy constructs are designed such that both the heavy and light chains are expressed in tandem from the vector including the Fc domain polypeptide of the heavy chain. In certain embodiments, the transgene encodes a transgene with heavy and light chain Fab fragment polypeptides or scFv as shown in Table 7, yet have a heavy chain that further comprises an Fc domain polypeptide C terminal to the hinge region of the heavy chain (or, in the case of an scFv, each heavy and light chain has the CH1 domain as well) (including an IgG1, IgG2 or IgG4 Fc domain or the antibody specific Fc domains as in Table 6). In specific embodiments, the transgene is a nucleotide sequence that encodes the following: Signal sequence-heavy chain Fab portion (including hinge region)-heavy chain Fc polypeptide-Furin-2A linker-signal sequence-light chain Fab portion.

In specific embodiments for expressing an intact or substantially intact mAb in muscle or liver cell types, the constructs described herein comprise the following components: (1) AAV2 inverted terminal repeats that flank the expression cassette; (2) Control elements, which include a) an inducible promoter, preferably a hypoxia-inducible promoter, b) a chicken β-actin intron and c) a rabbit β-globin poly A signal; and (3) nucleic acid sequences coding for the heavy chain Fab of an anti-Factor XII mAb; an Fc polypeptide associated with the therapeutic antibody (Table 6) or of the same isotype as the native form of the therapeutic antibody, such as an IgG isotype amino acid sequence from Table 6; and the light chain of an anti-Factor XII antibody, wherein the heavy chain (Fab and Fc region) and the light chain are separated by a self-cleaving furin (F)/F2A or T2A or flexible linker, ensuring expression of equal amounts of the heavy and the light chain polypeptides. Exemplary constructs are provided in FIGS. 1A and 1B.

In specific embodiments, provided are AAV vectors comprising a viral capsid that is at least 95% identical to the amino acid sequence of an AAV8 capsid (SEQ ID NO:124); and an artificial genome comprising an expression cassette flanked by AAV inverted terminal repeats (ITRs), wherein the expression cassette comprises a transgene encoding an intact or substantially intact anti-Factor XII mAb; operably linked to one or more regulatory sequences that control expression of the transgene in human liver or muscle cells.

The rAAV vectors that encode and express the full-length therapeutic antibodies may be administered to treat or prevent or ameliorate symptoms of a disease or condition amenable to treatment, prevention or amelioration of symptoms with the therapeutic antibodies. Also provided are methods of expressing HuPTM mAbs in human cells using the rAAV vectors and constructs encoding them.

### 5.1.8.2 Constructs for Expression of Antigen Binding Fragments

In some embodiments, the transgenes express antigen binding fragments, e.g. a Fab fragment (an HuGlyFab) or a F(ab′)₂, nanobody, or an scFv based upon a therapeutic antibody disclosed herein. FIGS. 2A-2I and section 5.4. provide the amino acid sequence of the heavy and light chains of the Fab fragments (or scFvs) of the therapeutic antibodies (see also Table 7, which provides the amino acid sequences of the Fab heavy and light chains of the therapeutic antibodies).

The transgene may encode a Fab fragment using nucleotide sequences encoding the amino acid sequences provided in Table 7, but not including the portion of the hinge region on the heavy chain that forms interchain di-sulfide bonds (e.g., the portion containing the sequence CPPCPA (SEQ ID NO:168)). Heavy chain Fab domain sequences that do not contain a CPPCP (SEQ ID NO:169) sequence of the hinge region at the C-terminus will not form intrachain disulfide bonds and, thus, will form Fab fragments with the corresponding light chain Fab domain sequences, whereas those heavy chain Fab domain sequences with a portion of the hinge region at the C-terminus containing the sequence CPPCP (SEQ ID NO:169) will form intrachain disulfide bonds and, thus, will form Fab₂ fragments. For example, in some embodiments, the transgene may encode a scFv comprising a light chain variable domain and a heavy chain variable domain connected by a flexible linker in between (where the heavy chain variable domain may be either at the N-terminal end or the C-terminal end of the scFv), and optionally, may further comprise a Fc polypeptide (e.g., IgG1, IgG2, IgG3, or IgG4) on the C-terminal end of the heavy chain. Alternatively, in other embodiments, the transgene may encode F(ab′)₂ fragments comprising a nucleotide sequence that encodes the light chain and the heavy chain sequence that includes at least the sequence CPPCA (SEQ ID NO:170) of the hinge region, as depicted in FIGS. 2A, 2C, and 2D which depict various regions of the hinge region that may be included at the C-terminus of the heavy chain sequence. Pre-existing anti-hinge antibodies (AHA) may cause immunogenicity and reduce efficacy. Thus, in certain embodiments, for the IgG1 isotype, C-terminal ends with D221 or ends with a mutation T225L or with L242 can reduce binding to AHA. (See, e.g., Brerski, 2008, J Immunol 181: 3183-92 and Kim, 2016, 8: 1536-1547). For IgG2, the risk of AHA is lower since the hinge region of IgG2 is not as susceptible to enzymatic cleavage required to generate endogenous AHA. (See, e.g., Brerski, 2011, MAbs 3: 558-567).

TABLE 5
Hinge Regions

| SEQ ID NO: | Sequence |
|---|---|
| 169 | CPPCP |
| 168 | CPPCPA |
| 170 | CPPCA |
| 171 | EPKSCDKTHTCPPCPAPELLGG |
| 172 | EPKSCDKTHLCPPCPAPELLGG |
| 173 | EPKSCDKTHL |
| 174 | EPKSCDKTHT |
| 175 | EPKSCDKTHTCPPCPA |
| 176 | EPKSCDKTHLCPPCPA |
| 177 | EPKSCDKTHTCPPCPAPELLGGPSVFL |
| 178 | EPKSCDKTHLCPPCPAPELLGGPSVFL |
| 179 | EPKSCDKTHTCPPCPAPEAAGG |
| 180 | EPKSCDKTHTCPPCPAPEAAGGPSVFL |
| 181 | EPKSCDKTHLCPPCPAPEAAGGPSVFL |
| 182 | ERKSCVECPPCPAPPVAG |
| 183 | ERKSCVECPPCPA |
| 184 | ESKYGPPCPPCPAPEFLGG |
| 185 | ESKYGPPCPPCPA |
| 186 | ESKYGPPCPSCPA |
| 187 | ESKYGPPCPSCPAPEFLGGPSVFL |
| 188 | ESKYGPPCPPCPAPEFLGGPSVFL |
| 189 | ERKCCVECPPCPAPPVAG |
| 190 | ERKCCVECPPCPA |
| 191 | EPKSCDKTHTCPPCPAPELAGA |
| 192 | EPKSCDKTHTCPPCPAPELAGAPSVFL |
| 193 | EPKSCDKTHLCPPCPAPELAGAPSVFL |
| 194 | EPKSCDKTHTCPPCPAPEFEGG |
| 195 | EPKSCDKTHTCPPCPAPEFEGGPSVFL |
| 196 | EPKSCDKTHLCPPCPAPEFEGGPSVFL |

In certain embodiments, the viral vectors provided herein comprise the following elements in the following order: a) a constitutive or inducible (e.g., hypoxia-inducible or rifamycin-inducible) promoter sequence or a tissue specific promoter/regulatory region, for example, one of the regulatory regions provided in Table 1, and b) a sequence encoding the transgene (e.g., a HuGlyFab). In certain embodiments, the sequence encoding the transgene comprises multiple ORFs separated by IRES elements. In certain embodiments, the ORFs encode the heavy and light chain domains of the HuGlyFab. In certain embodiments, the sequence encoding the transgene comprises multiple subunits in one ORF separated by F/F2A sequences or F/T2A sequences. In certain embodiments, the sequence comprising the transgene encodes the heavy and light chain domains of the HuGlyFab separated by an F/F2A sequence or a F/T2A sequence. In certain embodiments, the sequence comprising the transgene encodes the heavy and light chain variable domains of the HuGlyFab separated by a flexible peptide linker (as an scFv). In certain embodiments, the viral vectors provided herein comprise the following elements in the following order: a) a constitutive or tissue specific promoter sequence or a tissue specific promoter, such as one of the promoters or regulatory regions in Table 1, and b) a sequence encoding the transgene (e.g., a HuGlyFab), wherein the transgene comprises a nucleotide sequence encoding a signal peptide, a light chain and a heavy chain Fab portion separated by an IRES element. In certain embodiments, the viral vectors provided herein comprise the following elements in the following order: a) a constitutive or a tissue specific promoter sequence or regulatory element listed in Table 1, and b) a sequence encoding the transgene comprising a signal peptide, a light chain and a heavy chain sequence separated by a cleavable F/F2A sequence (SEQ ID NOS:166 or 167) or a F/T2A sequence (SEQ ID NOS:164 or 165) or a flexible peptide linker.

In certain embodiments, the viral vectors provided herein comprise the following elements in the following order: a) a first ITR sequence, b) a first linker sequence, c) a constitutive or tissue specific promoter sequence or a tissue specific promoter or regulatory region, d) a second linker sequence, e) an intron sequence, f) a third linker sequence, g) a first UTR sequence, h) a sequence encoding the transgene (e.g., a HuGlyFab), i) a second UTR sequence, j) a fourth linker sequence, k) a poly A sequence, l) a fifth linker sequence, and m) a second ITR sequence.

In certain embodiments, the viral vectors provided herein comprise the following elements in the following order: a) a first ITR sequence, b) a first linker sequence, c) a constitutive or tissue specific promoter sequence or a tissue specific regulatory region, d) a second linker sequence, e) an intron sequence, f) a third linker sequence, g) a first UTR sequence, h) a sequence encoding the transgene (e.g., HuGlyFab), i) a second UTR sequence, j) a fourth linker sequence, k) a poly A sequence, l) a fifth linker sequence, and m) a second ITR sequence, wherein the transgene comprises a signal, and wherein the transgene encodes a light chain and a heavy chain sequence separated by a cleavable F/2A sequence.

5.1.9. Fc Region Modifications

In certain embodiments, the transgenes encode full length or substantially full length heavy and light chains that associate to form a full length or intact antibody. (“Substantially intact” or “substantially full length” refers to a mAb having a heavy chain sequence that is at least 95% identical to the full-length heavy chain mAb amino acid sequence and a light chain sequence that is at least 95% identical to the full-length light chain mAb amino acid sequence). Accordingly, the transgenes comprise nucleotide sequences that encode, for example, the light and heavy chains of the Fab fragments including the hinge region of the heavy chain and C-terminal of the heavy chain of the Fab fragment, an Fc domain peptide. Table 6 provides the amino acid sequence of the Fc polypeptides for the anti-Factor XII antibodies. Alternatively, an IgG1, IgG2, or IgG4 Fc domain, the sequences of which are provided in Table 6 may be utilized.

The term “Fc region” refers to a dimer of two “Fc polypeptides” (or “Fc domains”), each “Fc polypeptide” comprising the heavy chain constant region of an antibody excluding the first constant region immunoglobulin domain. In some embodiments, an “Fc region” includes two Fc polypeptides linked by one or more disulfide bonds, chemical linkers, or peptide linkers. “Fc polypeptide” refers to at least the last two constant region immunoglobulin domains of IgA, IgD, and IgG, or the last three constant region immunoglobulin domains of IgE and IgM and may also include part or all of the flexible hinge N-terminal to these domains. For IgG, e.g., “Fc polypeptide” comprises immunoglobulin domains Cgamma2 (Cy2, often referred to as CH2 domain) and Cgamma3 (Cy3, also referred to as CH3 domain) and may include the lower part of the hinge domain between Cgamma1 (Cγ1, also referred to as CH1 domain) and CH2 domain. Although the boundaries of the Fc polypeptide may vary, the human IgG heavy chain Fc polypeptide is usually defined to comprise residues starting at T223 or C226 or P230, to its carboxyl-terminus, wherein the numbering is according to the EU index as in Kabat et al. (1991, NIH Publication 91-3242, National Technical Information Services, Springfield, Va.). For IgA, e.g., Fc polypeptide comprises immunoglobulin domains Calpha2 (Cα2) and Calpha3 (Cα3) and may include the lower part of the hinge between Calpha1 (Cα1) and Cα2.

In certain embodiments, the Fc polypeptide is that of the therapeutic antibody or is the Fc polypeptide corresponding to the isotype of the therapeutic antibody). In still other embodiments, the Fc polypeptide is an IgG Fc polypeptide. The Fc polypeptide may be from the IgG1, IgG2, or IgG4 isotype (see Table 6) or may be an IgG3 Fc domain, depending, for example, upon the desired effector activity of the therapeutic antibody. In some embodiments, the engineered heavy chain constant region (CH), which includes the Fc domain, is chimeric. As such, a chimeric CH region combines CH domains derived from more than one immunoglobulin isotype and/or subtype. For example, the chimeric (or hybrid) CH region comprises part or all of an Fc region from IgG, IgA and/or IgM. In other examples, the chimeric CH region comprises part or all a CH2 domain derived from a human IgG1, human IgG2, or human IgG4 molecule, combined with part or all of a CH3 domain derived from a human IgG1, human IgG2, or human IgG4 molecule. In other embodiments, the chimeric CH region contains a chimeric hinge region.

TABLE 6
Table of Fc Domain Amino Acid Sequences

| | Chain/ | |
|---|---|---|
| | SEQ ID | |
| mAb | NO. | Sequence |
| IgG1 | SEQ ID | FPPKPKDTLMISRTPEVTCVVVDVSHEDPEVKENWYVDGVEVHNAKTKPREE |
| | NO: 60 | QYNSTYRVVSVLTVLHQDWLNGKEYKCKVSNKALPAPIEKTISKAKGQPREP |
| | | QVYTLPPSRDELTKNQVSLTCLVKGFYPSDIAVEWESNGQPENNYKTTPPVL |
| | | DSDGSFFLYSKLTVDKSRWQQGNVFSCSVMHEALHNHYTQKSLSLSPGK |
| IgG2 | SEQ ID | FPPKPKDTLMISRTPEVTCVVVDVSHEDPEVQFNWYVDGVEVHNAKTKPREE |
| | NO: 61 | QFNSTFRVVSVLTVVHQDWLNGKEYKCKVSNKGLPAPIEKTISKTKGQPREP |
| | | QVYTLPPSREEMTKNQVSLTCLVKGFYPSDISVEWESNGQPENNYKTTPPML |
| | | DSDGSFFLYSKLTVDKSRWQQGNVFSCSVMHEALHNHYTQKSLSLSPGK |
| IgG4 | SEQ ID | FPPKPKDTLMISRTPEVTCVVVDVSQEDPEVQFNWYVDGVEVHNAKTKPREE |
| | NO: 62 | QFNSTYRVVSVLTVLHQDWLNGKEYKCKVSNKGLPSSIEK |
| | | TISKAKGQPREPQVYTLPPSQEEMTKNQVSLTCLVKGFYPSDIAVEWESNGQ |
| | | PENNYKTTPPVLDSDGSFFLYSRLTVDKSRWQEGNVFSCSVMHEALHNHYTQ |
| | | KSLSLSLGK |
| Garadacimab | SEQ ID | FPPKPKDTLMISRTPEVTCVVVDVSQEDPEVQFNWYVDGVEVHNAKTKPREE |
| | NO: 63 | QFNSTYRVVSVLTVLHQDWLNGKEYKCKVSNKGLPSSIEKTISKAKGQPREP |
| | | QVYTLPPSQEEMTKNQVSLTCLVKGFYPSDIAVEWESNGQPENNYKTTPPVL |
| | | DSDGSFFLYSRLTVDKSRWQEGNVFSCSVMHEALHNHYTQKSLSLSLGK |
| AB054 | SEQ ID | FPPKPKDTLMISRTPEVTCVVVDVSQEDPEVQFNWYVDGVEVHNAKTKPREE |
| | NO: 64 | QFNSTYRVVSVLTVLHQDWLNGKEYKCKVSNKGLPSSIEKTISKAKGQPREP |
| | | QVYTLPPSQEEMTKNQVSLTCLVKGFYPSDIAVEWESNGQPENNYKTTPPVL |
| | | DSDGSFFLYSRLTVDKSRWQEGNVFSCSVMHEALHNHYTQKSLSLSLG |
| DX-4012 | SEQ ID | FPPKPKDTLMISRTPEVTCVVVDVSHEDPEVKENWYVDGVEVHNAKTKPREE |
| | NO: 65 | QYNSTYRVVSVLTVLHQDWLNGKEYKCKVSNKALPAPIEKTISKAKGQPREP |
| | | QVYTLPPSREEMTKNQVSLTCLVKGFYPSDIAVEWESNGQPENNYKTTPPVL |
| | | DSDGSFFLYSKLTVDKSRWQQGNVFSCSVMHEALHNHYTQKSLSLSPG |
| DX-2930 | SEQ ID | FPPKPKDTLMISRTPEVTCVVVDVSHEDPEVKENWYVDGVEVHNAKTKPREE |
| | NO: 66 | QYNSTYRVVSVLTVLHQDWLNGKEYKCKVSNKALPAPIEKTISKAKGQPREP |
| | | QVYTLPPSREEMTKNQVSLTCLVKGFYPSDIAVEWESNGQPENNYKTTPPVL |
| | | DSDGSFFLYSKLTVDKSRWQQGNVFSCSVMHEALHNHYTQKSLSLSPGK |

In some embodiments, the recombinant vectors encode therapeutic antibodies comprising an engineered (mutant) Fc regions, e.g. engineered Fc regions of an IgG constant region. Modifications to an antibody constant region, Fc region or Fc fragment of an IgG antibody may alter one or more effector functions such as Fc receptor binding or neonatal Fc receptor (FcRn) binding and thus half-life, CDC activity, ADCC activity, and/or ADPC activity, compared to a corresponding antibody having a wild-type IgG constant region, or an IgG heavy chain constant region without the recited modification(s). Accordingly, in some embodiments, the antibody may be engineered to provide an antibody constant region, Fc region or Fc fragment of an IgG antibody that exhibits altered binding (as compared to a reference or wild-type constant region without the recited modification(s)) to one or more Fc receptors (e.g., FcγRI, FcγRIIA, FcγRIIB, FcγRIIIA, FcγRIIIB, FcγRIV, or FcRn receptor). In some embodiments, the antibody an antibody constant region, Fc region or Fc fragment of an IgG antibody that exhibits a one or more altered effector functions such as CDC, ADCC, or ADCP activity, compared to a corresponding antibody having a wild-type IgG constant region, or an IgG constant without the recited modification(s).

“Effector function” refers to a biochemical event that results from the interaction of an antibody Fc region with an Fc receptor or ligand. Effector functions include FcγR-mediated effector functions such as ADCC and ADCP and complement-mediated effector functions such as CDC.

An “effector cell” refers to a cell of the immune system that expresses one or more Fc receptors and mediates one or more effector functions. Effector cells include but are not limited to monocytes, macrophages, neutrophils, dendritic cells, eosinophils, mast cells, platelets, B cells, large granular lymphocytes, Langerhans' cells, natural killer (NK) cells, and T cells, and may be from any organism including but not limited to humans, mice, rats, rabbits, and monkeys.

“ADCC” or “antibody dependent cell-mediated cytotoxicity” refers to the cell-mediated reaction wherein nonspecific cytotoxic effector (immune) cells that express FcγRs recognize bound antibody on a target cell and subsequently cause lysis of the target cell.

“ADCP” or “antibody dependent cell-mediated phagocytosis” refers to the cell-mediated reaction wherein nonspecific cytotoxic effector (immune) cells that express FcγRs recognize bound antibody on a target cell and subsequently cause phagocytosis of the target cell.

“CDC” or “complement-dependent cytotoxicity” refers to the reaction wherein one or more complement protein components recognize bound antibody on a target cell and subsequently cause lysis of the target cell.

In some embodiments, the modifications of the Fc domain include, but are not limited to, the following modifications and combinations thereof, with reference to EU numbering of an IgG constant region (see FIG. 4): 233, 234, 235, 236, 237, 238, 239, 248, 249, 250, 252, 254, 255, 256, 258, 265, 267, 268, 269, 270, 272, 276, 278, 280, 283, 285, 286, 289, 290, 292, 293, 294, 295, 296, 297, 298, 301, 303, 305, 307, 308, 309, 311, 312, 315, 318, 320, 322, 324, 326, 327, 328, 329, 330, 331, 332, 333, 334, 335, 337, 338, 339, 340, 342, 344, 356, 358, 359, 360, 361, 362, 373, 375, 376, 378, 380, 382, 383, 384, 386, 388, 389, 398, 414, 416, 419, 428, 430, 433, 434, 435, 437, 438, and 439.

In certain embodiments, the Fc region comprises an amino acid addition, deletion, or substitution of one or more of amino acid residues 251-256, 285-290, 308-314, 385-389, and 428-436 of the IgG. In some embodiments, 251-256, 285-290, 308-314, 385-389, and 428-436 (EU numbering of Kabat; see FIG. 4 is substituted with histidine, arginine, lysine, aspartic acid, glutamic acid, serine, threonine, asparagine, or glutamine. In some embodiments, a non-histidine residue is substituted with a histidine residue. In some embodiments, a histidine residue is substituted with a non-histidine residue.

Enhancement of FcRn binding by an antibody having an engineered Fc leads to preferential binding of the affinity-enhanced antibody to FcRn as compared to antibody having wild-type Fc, and thus leads to a net enhanced recycling of the FcRn-affinity-enhanced antibody, which results in further increased antibody half-life. An enhanced recycling approach allows highly effective targeting and clearance of antigens, including e.g. “high titer” circulating antigens, such as C5, cytokines, or bacterial or viral antigens.

Provided in certain embodiments are modified constant region, Fc region or Fc fragment of an IgG antibody with enhanced binding to FcRn in serum as compared to a wild-type Fc region (without engineered modifications). In some instances, antibodies, e.g. IgG antibodies, are engineered to bind to FcRn at a neutral pH, e.g., at or above pH 7.4, to enhance pH-dependence of binding to FcRn as compared to a wild-type Fc region (without engineered modifications). In some instances, antibodies, e.g. IgG antibodies, are engineered to exhibit enhanced binding (e.g. increased affinity or K_{D}) to FcRn in endosomes (e.g., at an acidic pH, e.g., at or below pH 6.0) relative to a wild-type IgG and/or reference antibody binding to FcRn at an acidic pH, as well as in comparison to binding to FcRn in serum (e.g., at a neutral pH, e.g., at or above pH 7.4). Provided are antibodies with an engineered antibody constant region, Fc region or Fc fragment of an IgG antibody that exhibits an improved serum or resident tissue half-life, compared to a corresponding antibody having a wild-type IgG constant region, or an IgG constant without the recited modification(s);

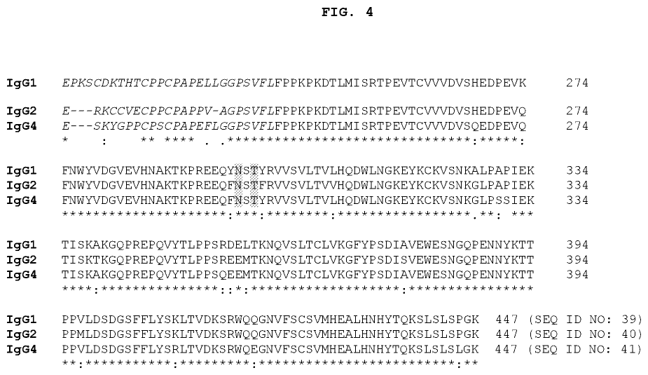

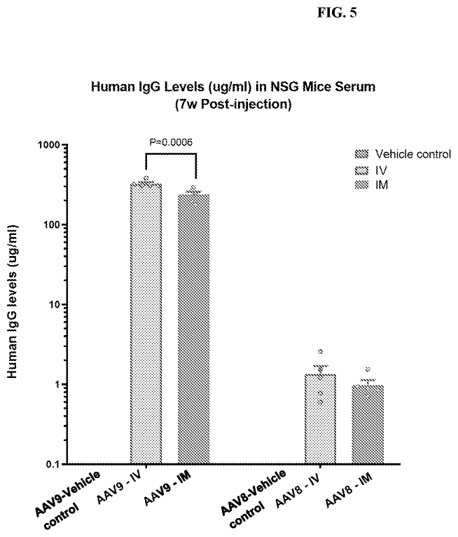

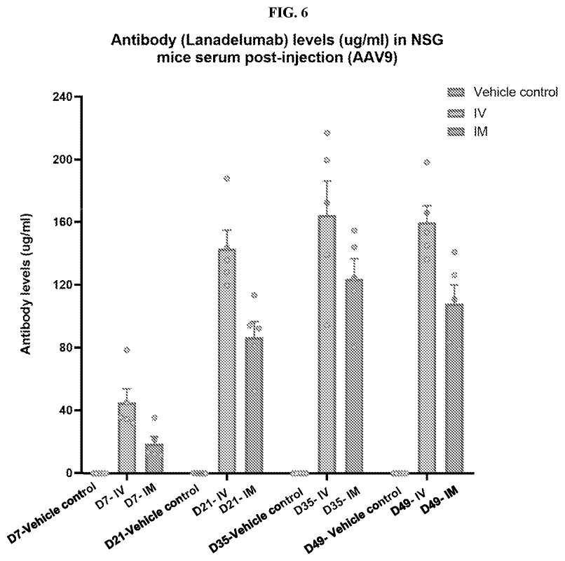

Non-limiting examples of such Fc modifications include, e.g., a modification at position 250 (e.g., E or Q); 250 and 428 (e.g., L or F); 252 (e.g., LN/Y/W or T), 254 (e.g., S or T), and 256 (e.g., S/R/Q/E/D or T); or a modification at position 428 and/or 433 (e.g., H/L/R/S/P/Q or K) and/or 434 (e.g., H/F or Y); or a modification at position 250 and/or 428; or a modification at position 307 or 308 (e.g., 308F, V308F), and 434. In one embodiment, the modification comprises a 428L (e.g., M428L) and 434S (e.g., N434S) modification; a 428L, 2591 (e.g., V2591), and 308F (e.g., V308F) modification; a 433K (e.g., H433K) and a 434 (e.g., 434Y) modification; a 252, 254, and 256 (e.g., 252Y, 254T, and 256E) modification; a 250Q and 428L modification (e.g., T250Q and M428L); and a 307 and/or 308 modification (e.g., 308F or 308P) (EU numbering; see FIG. 6).

In some embodiments, the Fc region can be a mutant form such as hIgG1 Fc including M252 mutations, e.g. M252Y and S254T and T256E (“YTE mutation”) exhibit enhanced affinity for human FcRn (Dall'Acqua, et al., 2002, J Immunol 169:5171-5180) and subsequent crystal structure of this mutant antibody bound to hFcRn resulting in the creation of two salt bridges (Oganesyan, et al. 2014, JBC 289(11): 7812-7824). Antibodies having the YTE mutation have been administered to monkeys and humans, and have significantly improved pharmacokinetic properties (Haraya, et al., 2019, Drug Metabolism and Pharmacokinetics, 34(1):25-41).

In some embodiments, modifications to one or more amino acid residues in the Fc region may reduce half-life in systemic circulation (serum), however result in improved retainment in tissues (e.g. in the eye) by disabling FcRn binding (e.g. H435A, EU numbering of Kabat) (Ding et al., 2017, MAbs 9:269-284; and Kim, 1999, Eur J Immunol 29:2819).

In some embodiments, the Fc domain may be engineered to activate all, some, or none of the normal Fc effector functions, without affecting the Fc polypeptide's (e.g. antibody's) desired pharmacokinetic properties. Fc polypeptides having altered effector function may be desirable as they may reduce unwanted side effects, such as activation of effector cells, by the therapeutic protein.

Methods to alter or even ablate effector function may include mutation(s) or modification(s) to the hinge region amino acid residues of an antibody. For example, IgG Fc domain mutants comprising 234A, 237A, and 238S substitutions, according to the EU numbering system, exhibit decreased complement dependent lysis and/or cell mediated destruction. Deletions and/or substitutions in the lower hinge, e.g. where positions 233-236 within a hinge domain (EU numbering) are deleted or modified to glycine, have been shown in the art to significantly reduce ADCC and CDC activity.

In specific embodiments, the Fc domain is an aglycosylated Fc domain that has a substitution at residue 297 or 299 to alter the glycosylation site at 297 such that the Fc domain is not glycosylated. Such aglycosylated Fc domains may have reduced ADCC or other effector activity.

Non-limiting examples of proteins comprising mutant and/or chimeric CH regions having altered effector functions, and methods of engineering and testing mutant antibodies, are described in the art, e.g. K. L. Amour, et al., Eur. J. Immunol. 1999, 29:2613-2624; Lazar et al., Proc. Natl. Acad. Sci. USA 2006, 103:4005; US Patent Application Publication No. 20070135620A1 published Jun. 14, 2007; US Patent Application Publication No. 20080154025 A1, published Jun. 26, 2008; US Patent Application Publication No. 20100234572 A1, published Sep. 16, 2010; US Patent Application Publication No. 20120225058 A1, published Sep. 6, 2012; US Patent Application Publication No. 20150337053 A1, published Nov. 26, 2015; International Publication No. WO20/16161010A2 published Oct. 6, 2016; U.S. Pat. No. 9,359,437, issued Jun. 7, 2016; and U.S. Pat. No. 10,053,517, issued Aug. 21, 2018, all of which are herein incorporated by reference.

The C-terminal lysines (−K) conserved in the heavy chain genes of all human IgG subclasses are generally absent from antibodies circulating in serum—the C-terminal lysines are cleaved off in circulation, resulting in a heterogeneous population of circulating IgGs. (van den Bremer et al., 2015, mAbs 7:672-680). In the vectored constructs for full length mAbs, the DNA encoding the C-terminal lysine (−K) or glycine-lysine (−GK) of the Fc terminus can be deleted to produce a more homogeneous antibody product in situ. (See, Hu et al., 2017 Biotechnol. Prog. 33: 786-794 which is incorporated by reference herein in its entirety).

5.1.10 Manufacture and Testing of Vectors

The viral vectors provided herein may be manufactured using host cells. The viral vectors provided herein may be manufactured using mammalian host cells, for example, A549, WEHI, BHK, MDCK, COS1, COS7, BSC 1, BSC 40, BMT 10, VERO, W138, HeLa, 293, Saos, C2C12, L, HT1080, HepG2, primary fibroblast, hepatocyte, and myoblast cells. The viral vectors provided herein may be manufactured using host cells from human, monkey, mouse, rat, rabbit, or hamster.

The host cells are stably transformed with the sequences encoding the transgene and associated elements (e.g., the vector genome), and the means of producing viruses in the host cells, for example, the replication and capsid genes (e.g., the rep and cap genes of AAV). For a method of producing recombinant AAV vectors with AAV8 capsids, see Section IV of the Detailed Description of U.S. Pat. No. 7,282,199 B2, which is incorporated herein by reference in its entirety. Genome copy titers of said vectors may be determined, for example, by TAQMAN® analysis. Virions may be recovered, for example, by CsCl₂ sedimentation.

Alternatively, baculovirus expression systems in insect cells may be used to produce AAV vectors. For a review, see Aponte-Ubillus et al., 2018, Appl. Microbiol. Biotechnol. 102:1045-1054 which is incorporated by reference herein in its entirety for manufacturing techniques.

In vitro assays, e.g., cell culture assays, can be used to measure transgene expression from a vector described herein, thus indicating, e.g., potency of the vector. In addition, in vitro neutralization assays can be used to measure the activity of the transgene expressed from a vector described herein. For example, Vero-E6 cells, a cell line derived from the kidney of an African green monkey, or HeLa cells engineered to stably express the ACE2 receptor (HeLa-ACE2), can be used to assess neutralization activity of transgenes expressed from a vector described herein. In addition, other characteristics of the expressed product can be determined, for example determination of the glycosylation and tyrosine sulfation patterns associated with the HuGlyFab. Glycosylation patterns and methods of determining the same are discussed in Section 5.3, while tyrosine sulfation patterns and methods of determining the same are discussed in Section 5.3. In addition, benefits resulting from glycosylation/sulfation of the cell-expressed HuGlyFab can be determined using assays known in the art, e.g., the methods described in Section 5.3.

Vector genome concentration (GC) or vector genome copies can be evaluated using digital PCR (dPCR) or ddPCR™ (BioRad Technologies, Hercules, CA, USA). In one example, ocular tissue samples, such as aqueous and/or vitreous humor samples, are obtained at several timepoints. In another example, several mice are sacrificed at various timepoints post injection. Ocular tissue samples are subjected to total DNA extraction and dPCR assay for vector copy numbers. Copies of vector genome (transgene) per gram of tissue may be measured in a single biopsy sample, or measured in various tissue sections at sequential timepoints will reveal spread of AAV throughout the eye. Total DNA from collected ocular fluid or tissue is extracted with the DNeasy Blood & Tissue Kit and the DNA concentration measured using a Nanodrop spectrophotometer. To determine the vector copy numbers in each tissue sample, digital PCR is performed with Naica Crystal Digital PCR system (Stilla technologies). Two color multiplexing system is applied to simultaneously measure the transgene AAV and an endogenous control gene. In brief, the transgene probe can be labelled with FAM (6-carboxyfluorescein) dye while the endogenous control probe can be labelled with VIC fluorescent dye. The copy number of delivered vector in a specific tissue section per diploid cell is calculated as: (vector copy number)/(endogenous control)×2. Vector copy in specific cell types or tissues, such as liver, over time may indicate sustained expression of the transgene by the tissue.

### 5.1.11 Compositions

Pharmaceutical compositions suitable for administration to human subjects comprise a suspension of the recombinant vector in a formulation buffer comprising a physiologically compatible aqueous buffer, a surfactant and optional excipients. Such formulation buffer can comprise one or more of a polysaccharide, a surfactant, polymer, or oil. In some embodiments, the pharmaceutical composition comprises rAAV combined with a pharmaceutically acceptable carrier for administration to a subject. In one embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's complete and incomplete adjuvant), excipient, or vehicle with which the agent is administered. Such pharmaceutical carriers can be sterile liquids, such as water and oils, including those of petroleum, animal, vegetable, or synthetic origin, including, e.g., peanut oil, soybean oil, mineral oil, sesame oil and the like. Water is a common carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Additional examples of pharmaceutically acceptable carriers, excipients, and stabilizers include, but are not limited to, buffers such as phosphate, citrate, and other organic acids; antioxidants including ascorbic acid; low molecular weight polypeptides; proteins, such as serum albumin and gelatin; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN′, polyethylene glycol (PEG), and PLURONICS' as known in the art. The pharmaceutical composition of the present invention can also include a lubricant, a wetting agent, a sweetener, a flavoring agent, an emulsifier, a suspending agent, and a preservative, in addition to the above ingredients. These compositions can take the form of solutions, suspensions, emulsion, tablets, pills, capsules, powders, sustained-release formulations and the like.

### 5.2 Methods of Treating Hereditary Angioedema and Thrombotic Diseases

In another aspect, methods for treating hereditary angioedema or other indication that can be treated with an anti-Factor XII antibody in a subject in need thereof comprising the administration of recombinant AAV particles comprising an expression cassette encoding anti-Factor XII antibodies, antibody-binding fragments, or variants thereof, are provided herein. A subject in need thereof includes a subject suffering from hereditary angioedema, or a subject pre-disposed thereto, e.g., a subject at risk of developing or having a recurrence of the hereditary angioedema, hypercoagulation or thrombosis or other indication that may be treated with an anti-Factor XII antibody. Subjects to whom such gene therapy is administered can be those responsive to anti-Factor XII therapy. In particular embodiments, the methods encompass treating patients who have been diagnosed with hereditary angioedema, hypercoagulation, or thrombosis, and, in certain embodiments, identified as responsive to treatment with an anti-Factor XII antibody or considered a good candidate for therapy with an anti-Factor XII antibody. In specific embodiments, the patients have previously been treated with an anti-Factor XII antibody. To determine responsiveness, the anti-Factor XII antibody or antigen-binding fragment transgene product (e.g., produced in human cell culture, bioreactors, etc.) may be administered directly to the subject.

In specific embodiments, provided are methods of treating hereditary angioedema, hypercoagulation, or thrombosis or other indication amenable to treatment with an anti-Factor XII antibody in a human subject in need thereof comprising: administering to the liver or muscle of said subject a therapeutically effective amount of a recombinant nucleotide expression vector comprising a transgene encoding a substantially full-length or full-length anti-Factor XII mAb having an Fc region, or an antigen-binding fragment thereof, operably linked to one or more regulatory sequences that control expression of the transgene in human liver and/or muscle cells, so that a depot is formed that releases a HuPTM form of mAb or antigen-binding fragment thereof. Recombinant vectors and pharmaceutical compositions for treating diseases or disorders in a subject in need thereof are described in Section 5.1. Such vectors should have a tropism for human liver and/or muscle cells and can include non-replicating rAAV, particularly those bearing an AAV3B, AAVrh8, AAVru37, AAV64R, AAV8, AAAV9, AAVS3, AAV-LK03, AAVrh46, or AAVrh73 capsid. The recombinant vectors can be administered in any manner such that the recombinant vector enters liver and or muscle cells, e.g., by introducing the recombinant vector into circulation. Such vectors should further comprise one or more regulatory sequences that control expression of the transgene in human liver cells and/or human liver and muscle cells include, but are not limited to, an ApoE.hAAT (SEQ ID NO:78) regulatory sequence, a LSPX1 promoter (SEQ ID NO:66), a LSPX2 promoter (SEQ ID NO:67), a LTP1 promoter (SEQ ID NO:68), a LTP2 (SEQ ID NO:69) promoter, or a LTP3 (SEQ ID NO:70) promoter (see also Table 1).

### 5.3.N-Glycosylation, Tyrosine Sulfation, and O-Glycosylation

The amino acid sequence (primary sequence) of HuGlyFabs or HuPTM Fabs, HuPTMmAbs, and HuPTM scFvs disclosed herein each comprises at least one site at which N-glycosylation or tyrosine sulfation takes place (see exemplary FIG. 4) for glycosylation and/or sulfation positions within the amino acid sequences of the Fab fragments of the therapeutic antibodies). Post-translational modification also occurs in the Fc domain of full length antibodies, particularly at residue N297 (by EU numbering, see Table 6).

Alternatively, mutations may be introduced into the Fc domain to alter the glycosylation site at residue N297 (EU numbering, see Table 6), in particular substituting another amino acid for the asparagine at 297 or the threonine at 299 to remove the glycosylation site resulting in an aglycosylated Fc domain.

### 5.3.1. N-Glycosylation

Reverse Glycosylation Sites

The canonical N-glycosylation sequence is known in the art to be Asn-X-Ser (or Thr), wherein X can be any amino acid except Pro. However, it recently has been demonstrated that asparagine (Asn) residues of human antibodies can be glycosylated in the context of a reverse consensus motif, Ser(or Thr)-X-Asn, wherein X can be any amino acid except Pro. See Valliere-Douglass et al., 2009, J. Biol. Chem. 284:32493-32506; and Valliere-Douglass et al., 2010, J. Biol. Chem. 285:16012-16022. As disclosed herein, certain HuGlyFabs and HuPTM scFvs disclosed herein comprise such reverse consensus sequences.

Non-Consensus Glycosylation Sites

In addition to reverse N-glycosylation sites, it recently has been demonstrated that glutamine (Gln) residues of human antibodies can be glycosylated in the context of a non-consensus motif, Gln-Gly-Thr. See Valliere-Douglass et al., 2010, J. Biol. Chem. 285:16012-16022. Surprisingly, certain of the HuGlyFab fragments disclosed herein comprise such non-consensus sequences. In addition, O-glycosylation comprises the addition of N-acetyl-galactosamine to serine or threonine residues by the enzyme. It has been demonstrated that amino acid residues present in the hinge region of antibodies can be 0-glycosylated. The possibility of O-glycosylation confers another advantage to the therapeutic antibodies provided herein, as compared to, e.g., antigen-binding fragments produced in E. coli, again because the E. coli naturally does not contain machinery equivalent to that used in human O-glycosylation. (Instead, O-glycosylation in E. coli has been demonstrated only when the bacteria is modified to contain specific O-glycosylation machinery. See, e.g., Farid-Moayer et al., 2007, J. Bacteriol. 189:8088-8098.)

Engineered N-Glycosylation Sites

In certain embodiments, a nucleic acid encoding a HuPTM mAb, HuGlyFab or HuPTM scFv is modified to include 1, 2, 3, 4, 5, 6, 7, 8, 9, 10, or more N-glycosylation sites (including the canonical N-glycosylation consensus sequence, reverse N-glycosylation site, and non-consensus N-glycosylation sites) than would normally be associated with the HuPTM mAb, HuGlyFab or HuPTM scFv (e.g., relative to the number of N-glycosylation sites associated with the HuPTM mAb, HuGlyFab or HuPTM scFv in its unmodified state). In specific embodiments, introduction of glycosylation sites is accomplished by insertion of N-glycosylation sites (including the canonical N-glycosylation consensus sequence, reverse N-glycosylation site, and non-consensus N-glycosylation sites) anywhere in the primary structure of the antigen-binding fragment, so long as said introduction does not impact binding of the antibody or antigen-binding fragment to its antigen. Introduction of glycosylation sites can be accomplished by, e.g., adding new amino acids to the primary structure of the antigen-binding fragment, or the antibody from which the antigen-binding fragment is derived (e.g., the glycosylation sites are added, in full or in part), or by mutating existing amino acids in the antigen-binding fragment, or the antibody from which the antigen-binding fragment is derived, in order to generate the N-glycosylation sites (e.g., amino acids are not added to the antigen-binding fragment/antibody, but selected amino acids of the antigen-binding fragment/antibody are mutated so as to form N-glycosylation sites). Those of skill in the art will recognize that the amino acid sequence of a protein can be readily modified using approaches known in the art, e.g., recombinant approaches that include modification of the nucleic acid sequence encoding the protein.

In a specific embodiment, a HuGlyMab or antigen-binding fragment is modified such that, when expressed in mammalian cells, such as retina, CNS, liver or muscle cells, it can be hyperglycosylated. See Courtois et al., 2016, mAbs 8:99-112 which is incorporated by reference herein in its entirety.

N-Glycosylation of HuPTM mAbs and HuPTM Antigen-Binding Fragments

Unlike small molecule drugs, biologics usually comprise a mixture of many variants with different modifications or forms that could have a different potency, pharmacokinetics, and/or safety profile. It is not essential that every molecule produced either in the gene therapy or protein therapy approach be fully glycosylated and sulfated. Rather, the population of glycoproteins produced should have sufficient glycosylation (including 2,6-sialylation) and sulfation to demonstrate efficacy. The goal of gene therapy treatment provided herein can be, for example, to slow or arrest the progression of a disease or abnormal condition or to reduce the severity of one or more symptoms associated with the disease or abnormal condition.

When a HuPTM mAb, HuGlyFab or HuPTM scFv is expressed in a human cell, the N-glycosylation sites of the antigen-binding fragment can be glycosylated with various different glycans. N-glycans of antigen-binding fragments and the Fc domain have been characterized in the art. For example, Bondt et al., 2014, Mol. & Cell. Proteomics 13.11:3029-3039 (incorporated by reference herein in its entirety for its disclosure of Fab-associated N-glycans) characterizes glycans associated with Fabs, and demonstrates that Fab and Fc portions of antibodies comprise distinct glycosylation patterns, with Fab glycans being high in galactosylation, sialylation, and bisection (e.g., with bisecting GlcNAc) but low in fucosylation with respect to Fc glycans. Like Bondt, Huang et al., 2006, Anal. Biochem. 349:197-207 (incorporated by reference herein in its entirety for it disclosure of Fab-associated N-glycans) found that most glycans of Fabs are sialylated. However, in the Fab of the antibody examined by Huang (which was produced in a murine cell background), the identified sialic residues were N-Glycolylneuraminic acid (“Neu5Gc” or “NeuGc”) (which is not natural to humans) instead of N-acetylneuraminic acid (“Neu5Ac,” the predominant human sialic acid). In addition, Song et al., 2014, Anal. Chem. 86:5661-5666 (incorporated by reference herein in its entirety for its disclosure of Fab-associated N-glycans) describes a library of N-glycans associated with commercially available antibodies.

Glycosylation of the Fc domain has been characterized and is a single N-linked glycan at asparagine 297 (EU numbering; see Table 6). The glycan plays an integral structural and functional role, impacting antibody effector function, such as binding to Fc receptor (see, for example, Jennewein and Alter, 2017, Trends In Immunology 38:358 for a discussion of the role of Fc glycosylation in antibody function). Removal of the Fc region glycan almost completely ablates effector function (Jennewien and Alter at 362). The composition of the Fc glycan has been shown to impact effector function, for example hypergalactosylation and reduction in fucosylation have been shown to increase ADCC activity while sialylation correlates with anti-inflammatory effects (Id. at 364). Disease states, genetics and even diet can impact the composition of the Fc glycan in vivo. For recombinantly expressed antibodies, the glycan composition can differ significantly by the type of host cell used for recombinant expression and strategies are available to control and modify the composition of the glycan in therapeutic antibodies recombinantly expressed in cell culture, such as CHO to alter effector function (see, for example, US 2014/0193404 by Hansen et al.). Accordingly, the HuPTM mAbs provided herein may advantageously have a glycan at N297 that is more like the native, human glycan composition than antibodies expressed in non-human host cells.

Importantly, when the HuPTM mAb, HuGlyFab or HuPTM scFv are expressed in human cells, the need for in vitro production in prokaryotic host cells (e.g., E. coli) or eukaryotic host cells (e.g., CHO cells or NS0 cells) is circumvented. Instead, as a result of the methods described herein, N-glycosylation sites of the HuPTM mAb, HuGlyFab or HuPTM scFv are advantageously decorated with glycans relevant to and beneficial to treatment of humans. Such an advantage is unattainable when CHO cells, NS0 cells, or E. coli are utilized in antibody/antigen-binding fragment production, because e.g., CHO cells (1) do not express 2,6 sialyltransferase and thus cannot add 2,6 sialic acid during N-glycosylation; (2) can add Neu5Gc as sialic acid instead of Neu5Ac; and (3) can also produce an immunogenic glycan, the α-Gal antigen, which reacts with anti-α-Gal antibodies present in most individuals, which at high concentrations can trigger anaphylaxis; and because (4) E. coli does not naturally contain components needed for N-glycosylation.

Assays for determining the glycosylation pattern of antibodies, including antigen-binding fragments are known in the art. For example, hydrazinolysis can be used to analyze glycans. First, polysaccharides are released from their associated protein by incubation with hydrazine (the Ludger Liberate Hydrazinolysis Glycan Release Kit, Oxfordshire, UK can be used). The nucleophile hydrazine attacks the glycosidic bond between the polysaccharide and the carrier protein and allows release of the attached glycans. N-acetyl groups are lost during this treatment and have to be reconstituted by re-N-acetylation. Glycans may also be released using enzymes such as glycosidases or endoglycosidases, such as PNGase F and Endo H, which cleave cleanly and with fewer side reactions than hydrazines. The free glycans can be purified on carbon columns and subsequently labeled at the reducing end with the fluorophor 2-amino benzamide. The labeled polysaccharides can be separated on a GlycoSep-N column (GL Sciences) according to the HPLC protocol of Royle et al, Anal Biochem 2002, 304(1):70-90. The resulting fluorescence chromatogram indicates the polysaccharide length and number of repeating units. Structural information can be gathered by collecting individual peaks and subsequently performing MS/MS analysis. Thereby the monosaccharide composition and sequence of the repeating unit can be confirmed and additionally in homogeneity of the polysaccharide composition can be identified. Specific peaks of low or high molecular weight can be analyzed by MALDI-MS/MS and the result used to confirm the glycan sequence. Each peak in the chromatogram corresponds to a polymer, e.g., glycan, consisting of a certain number of repeat units and fragments, e.g., sugar residues, thereof. The chromatogram thus allows measurement of the polymer, e.g., glycan, length distribution. The elution time is an indication for polymer length, while fluorescence intensity correlates with molar abundance for the respective polymer, e.g., glycan. Other methods for assessing glycans associated with antigen-binding fragments include those described by Bondt et al., 2014, Mol. & Cell. Proteomics 13.11:3029-3039, Huang et al., 2006, Anal. Biochem. 349:197-207, and/or Song et al., 2014, Anal. Chem. 86:5661-5666.

Homogeneity or heterogeneity of the glycan patterns associated with antibodies (including antigen-binding fragments), as it relates to both glycan length or size and numbers glycans present across glycosylation sites, can be assessed using methods known in the art, e.g., methods that measure glycan length or size and hydrodynamic radius. HPLC, such as size exclusion, normal phase, reversed phase, and anion exchange HPLC, as well as capillary electrophoresis, allows the measurement of the hydrodynamic radius. Higher numbers of glycosylation sites in a protein lead to higher variation in hydrodynamic radius compared to a carrier with less glycosylation sites. However, when single glycan chains are analyzed, they may be more homogenous due to the more controlled length. Glycan length can be measured by hydrazinolysis, SDS PAGE, and capillary gel electrophoresis. In addition, homogeneity can also mean that certain glycosylation site usage patterns change to a broader/narrower range. These factors can be measured by Glycopeptide LC-MS/MS.

In certain embodiments, the HuPTM mAbs, or antigen binding fragments thereof, also do not contain detectable NeuGc and/or α-Gal. By “detectable NeuGc” or “detectable α-Gal” or “does not contain or does not have NeuGc or α-Gal” means herein that the HuPTM mAb or antigen-binding fragment, does not contain NeuGc or α-Gal moieties detectable by standard assay methods known in the art. For example, NeuGc may be detected by HPLC according to Hara et al., 1989, “Highly Sensitive Determination of N-Acetyl-and N-Glycolylneuraminic Acids in Human Serum and Urine and Rat Serum by Reversed-Phase Liquid Chromatography with Fluorescence Detection.” J. Chromatogr., B: Biomed. 377, 111-119, which is hereby incorporated by reference for the method of detecting NeuGc. Alternatively, NeuGc may be detected by mass spectrometry. The α-Gal may be detected using an ELISA, see, for example, Galili et al., 1998, “A sensitive assay for measuring α-Gal epitope expression on cells by a monoclonal anti-Gal antibody.” Transplantation. 65(8):1129-32, or by mass spectrometry, see, for example, Ayoub et al., 2013, “Correct primary structure assessment and extensive glyco-profiling of cetuximab by a combination of intact, middle-up, middle-down and bottom-up ESI and MALDI mass spectrometry techniques.” Landes Bioscience. 5(5):699-710. See also the references cited in Platts-Mills et al., 2015, “Anaphylaxis to the Carbohydrate Side-Chain Alpha-gal” Immunol Allergy Clin North Am. 35(2): 247-260.

Benefits of N-Glycosylation

N-glycosylation confers numerous benefits on the HuPTM mAb, HuGlyFab or HuPTM scFv described herein. Such benefits are unattainable by production of antigen-binding fragments in E. coli, because E. coli does not naturally possess components needed for N-glycosylation. Further, some benefits are unattainable through antibody production in, e.g., CHO cells (or murine cells such as NS0 cells), because CHO cells lack components needed for addition of certain glycans (e.g., 2,6 sialic acid and bisecting GlcNAc) and because either CHO or murine cell lines add N-N-Glycolylneuraminic acid (“Neu5Gc” or “NeuGc”) which is not natural to humans (and potentially immunogenic), instead of N-Acetylneuraminic acid (“Neu5Ac”) the predominant human sialic acid. See, e.g., Dumont et al., 2015, Crit. Rev. Biotechnol. 36(6):1110-1122; Huang et al., 2006, Anal. Biochem. 349:197-207 (NeuGc is the predominant sialic acid in murine cell lines such as SP2/0 and NS0); and Song et al., 2014, Anal. Chem. 86:5661-5666, each of which is incorporated by reference herein in its entirety). Moreover, CHO cells can also produce an immunogenic glycan, the α-Gal antigen, which reacts with anti-α-Gal antibodies present in most individuals, which at high concentrations can trigger anaphylaxis. See, e.g., Bosques, 2010, Nat. Biotech. 28:1153-1156. The human glycosylation pattern of the HuGlyFab of HuPTM scFv described herein should reduce immunogenicity of the transgene product and improve efficacy.

While non-canonical glycosylation sites usually result in low level glycosylation (e.g., 1-5%) of the antibody population, the functional benefits may be significant (See, e.g., van de Bovenkamp et al., 2016, J. Immunol. 196:1435-1441). For example, Fab glycosylation may affect the stability, half-life, and binding characteristics of an antibody. To determine the effects of Fab glycosylation on the affinity of the antibody for its target, any technique known to one of skill in the art may be used, for example, enzyme linked immunosorbent assay (ELISA), or surface plasmon resonance (SPR). To determine the effects of Fab glycosylation on the half-life of the antibody, any technique known to one of skill in the art may be used, for example, by measurement of the levels of radioactivity in the blood or organs in a subject to whom a radiolabelled antibody has been administered. To determine the effects of Fab glycosylation on the stability, for example, levels of aggregation or protein unfolding, of the antibody, any technique known to one of skill in the art may be used, for example, differential scanning calorimetry (DSC), high performance liquid chromatography (HPLC), e.g., size exclusion high performance liquid chromatography (SEC-HPLC), capillary electrophoresis, mass spectrometry, or turbidity measurement.

The presence of sialic acid on HuPTM mAb, HuGlyFab or HuPTM scFv used in the methods described herein can impact clearance rate of the HuPTM mAb, HuGlyFab or HuPTM scFv. Accordingly, sialic acid patterns of a HuPTM mAb, HuGlyFab or HuPTM scFv can be used to generate a therapeutic having an optimized clearance rate. Methods of assessing antigen-binding fragment clearance rate are known in the art. See, e.g., Huang et al., 2006, Anal. Biochem. 349:197-207.

In another specific embodiment, a benefit conferred by N-glycosylation is reduced aggregation. Occupied N-glycosylation sites can mask aggregation prone amino acid residues, resulting in decreased aggregation. Such N-glycosylation sites can be native to an antigen-binding fragment used herein or engineered into an antigen-binding fragment used herein, resulting in HuGlyFab or HuPTM scFv that is less prone to aggregation when expressed, e.g., expressed in human cells. Methods of assessing aggregation of antibodies are known in the art. See, e.g., Courtois et al., 2016, mAbs 8:99-112 which is incorporated by reference herein in its entirety.

In another specific embodiment, a benefit conferred by N-glycosylation is reduced immunogenicity. Such N-glycosylation sites can be native to an antigen-binding fragment used herein or engineered into an antigen-binding fragment used herein, resulting in HuPTM mAb, HuGlyFab or HuPTM scFv that is less prone to immunogenicity when expressed, e.g., expressed in human retinal cells, human CNS cells, human liver cells or human muscle cells.

In another specific embodiment, a benefit conferred by N-glycosylation is protein stability. N-glycosylation of proteins is well-known to confer stability on them, and methods of assessing protein stability resulting from N-glycosylation are known in the art. See, e.g., Sola and Griebenow, 2009, J Pharm Sci., 98(4): 1223-1245.

In another specific embodiment, a benefit conferred by N-glycosylation is altered binding affinity. It is known in the art that the presence of N-glycosylation sites in the variable domains of an antibody can increase the affinity of the antibody for its antigen. See, e.g., Bovenkamp et al., 2016, J. Immunol. 196:1435-1441. Assays for measuring antibody binding affinity are known in the art. See, e.g., Wright et al., 1991, EMBO J. 10:2717-2723; and Leibiger et al., 1999, Biochem. J. 338:529-538.

### 5.3.2 Tyrosine Sulfation

Tyrosine sulfation occurs at tyrosine (Y) residues with glutamate (E) or aspartate (D) within +5 to −5 position of Y, and where position −1 of Y is a neutral or acidic charged amino acid, but not a basic amino acid, e.g., arginine (R), lysine (K), or histidine (H) that abolishes sulfation. The HuGlyFabs and HuPTM scFvs described herein comprise tyrosine sulfation sites (see exemplary FIGS. 2A-2I).

Importantly, tyrosine-sulfated antigen-binding fragments cannot be produced in E. coli, which naturally does not possess the enzymes required for tyrosine-sulfation. Further, CHO cells are deficient for tyrosine sulfation—they are not secretory cells and have a limited capacity for post-translational tyrosine-sulfation. See, e.g., Mikkelsen & Ezban, 1991, Biochemistry 30: 1533-1537. Advantageously, the methods provided herein call for expression of HuPTM Fab in human cells that are secretory and have capacity for tyrosine sulfation.

Tyrosine sulfation is advantageous for several reasons. For example, tyrosine-sulfation of the antigen-binding fragment of therapeutic antibodies against targets has been shown to dramatically increase avidity for antigen and activity. See, e.g., Loos et al., 2015, PNAS 112: 12675-12680, and Choe et al., 2003, Cell 114: 161-170. Assays for detection tyrosine sulfation are known in the art. See, e.g., Yang et al., 2015, Molecules 20:2138-2164.

5.3.3 O-Glycosylation

O-glycosylation comprises the addition of N-acetyl-galactosamine to serine or threonine residues by the enzyme. It has been demonstrated that amino acid residues present in the hinge region of antibodies can be 0-glycosylated. In certain embodiments, the HuGlyFab comprise all or a portion of their hinge region, and thus are capable of being 0-glycosylated when expressed in human cells. The possibility of O-glycosylation confers another advantage to the HuGlyFab provided herein, as compared to, e.g., antigen-binding fragments produced in E. coli, again because the E. coli naturally does not contain machinery equivalent to that used in human O-glycosylation. (Instead, O-glycosylation in E. coli has been demonstrated only when the bacteria is modified to contain specific machinery. See, e.g., Farid-Moayer et al., 2007, J. Bacteriol. 189:8088-8098.) O-glycosylated HuGlyFab, by virtue of possessing glycans, shares advantageous characteristics with N-glycosylated HuGlyFab (as discussed above).

### 5.4 Anti-Factor XII HuPTM Constructs and Formulations for Angioedema, Thrombosis, and Hypercoagulation

Compositions and methods are described for the delivery of HuPTM mAbs and antigen-binding fragments thereof, such as HuPTM Fabs, that bind to factor XII, derived from an anti-factor XII antibody and indicated for treating angioedema, such as hereditary angioedema, thrombosis, and hypercoagulation. In certain embodiments, the HuPTM mAb has the amino acid sequence of garadacimab, AB042/AB043, AB054, DX-4012, Ab26036, Ab26048, Ab260489, Ab26076, 559C-M0071-F06, 559C-M0179-D04, 559C-M0181-C02, 559C-M0180-G03, 559C-M0184-B04, 620I-X0173-A11, 620I-X0173-C07, 620I-X0173-E07, or 620I-X0173-G11, or an antigen binding fragment thereof. The amino acid sequences of Fab fragments of anti-factor XII antibodies are provided in FIGS. 2A-2H and Table 7. Delivery may be accomplished via gene therapy—e.g., by administering a viral vector or other DNA expression construct encoding an factor XII-binding HuPTM mAb (or an antigen binding fragment and/or a hyperglycosylated derivative or other derivative, thereof) to patients (human subjects) diagnosed with angioedema, thrombosis, or hypercoagulation to create a permanent depot that continuously supplies the human PTM, e.g., human-glycosylated, transgene product.

Compositions and methods are also provided for the delivery of bi-specific HuPTM mAbs that bind to p-kallikrein (pKal) and factor XII, derived from an anti-pKal antibody and an anti-factor XII antibody and indicated for treating angioedema, such as hereditary angioedema, thrombosis, and hypercoagulation. In some embodiments, the bi-specific mAb comprises a heavy chain of a first antibody, the heavy chain comprising a heavy chain variable region and a heavy chain constant region fused to a single chain antibody, wherein the first antibody is an anti-pKal antibody and the second antibody is an anti-factor XII antibody. In a specific embodiment, the bi-specific HuPTM mAb has the amino acid sequence of 620I-X0177-A01 (SEQ ID NO: 9) provided in FIG. 2J and Table 7. Delivery may be accomplished via gene therapy—e.g., by administering a viral vector or other DNA expression construct encoding a bi-specific pKal and factor XII-binding HuPTM mAb (and/or a hyperglycosylated derivative or other derivative, thereof) to patients (human subjects) diagnosed with angioedema, thrombosis, or hypercoagulation to create a permanent depot that continuously supplies the human PTM, e.g., human-glycosylated, transgene product.

TABLE 7
Amino Acid Sequences of Heavy and Light Chain

| | Chain/ | |
|---|---|---|
| Ab | SEQ ID NO. | Sequence |
| Garadacimab | Heavy/ | EVQLLESGGGLVQPGGSLRLSCAASGFTFSKYIMQWVRQAPGKGLEWVSGIDIP |
| | SEQ ID | TKGTVYADSVKGRFTISRDNSKNTLYLOMNSLRAEDTAVYYCARALPRSGYLIS |
| | NO: 1 | PHYYYYALDVWGQGTTVTVSSASTKGPSVFPLAPCSRSTSESTAALGCLVKDYF |
| | | PEPVTVSWNSGALTSGVHTFPAVLQSSGLYSLSSVVTVPSSSLGTKTYTCNVDH |
| | | 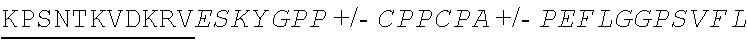 |
| Garadacimab | Light/ | QSVLTQPPSASGTPGQRVTISCSGSSSNIGRNYVYWYQQLPGTAPKLLIYSNNQ |
| | SEQ ID | RPSGVPDRFSGSKSGTSASLAISGLRSEDEADYYCAAWDASLRGVFGGGTKLTV |
| | NO: 2 | LGQPKAAPSVTLFPPSSEELQANKATLVCLISDFYPGAVTVAWKADSSPVKAGV |
| | | ETTTPSKQSNNKYAASSYLSLTPEQWKSHRSYSCOVTHEGSTVEKTVAPTECS |
| AB042/43 | Heavy/ | EVQLQQSGAELVRPGASVKLSCTASGFNIKDDYMHWVKQRPEQGLEWIGWIDPE |
| | SEQ ID | NGDTEYASKFQGKATITSDTSSNTAYLQLSSLTSEDTAVYYCTENYYGSSFFAY |
| | NO: 3 | WGQGTLVTVSA |
| AB042/43 | Light/ | DIVMTQATPSVPVTPGESVSISCRSSKSLLHSNGITYLYWFLQRPGQSPQRLIY |
| | SEQ ID | YMSNLASGVPDRESGRGSGTDFTLRISRVEAEDVGVYYCMQSLEYPYTFGGGTK |
| | NO:4 | LEIK |
| AB054 | Heavy/ | QVQLQESGPGLVKPSQTLSLTCTVSGYSITSGYSWHWIRQHPGKGLEWIGYIQY |
| | SEQ ID | SGNTNSNPSLKSRVTISRDTSKNQFSLKLSSVTAADTAVYYCARWGSFDYWGQG |
| | NO: 5 | TLVTVSSASTKGPSVFPLAPCSRSTSESTAALGCLVKDYFPEPVTVSWNSGALT |
| | | 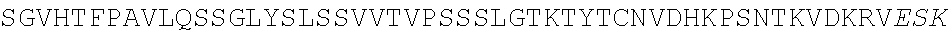 |
| | | 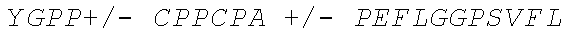 |
| AB054 | Light/ | EIVLTQSPATLSLSPGERATLSCRASSSVNYLHWYQQKPGQAPRRLIYDTSKLA |
| | SEQ ID | TGIPARFSGSGSGTDYTLTISSLEPEDFAVYYCQQWSGNPPTFGGGTKVEIKRT |
| | NO: 6 | VAAPSVFIFPPSDEQLKSGTASVVCLLNNFYPREAKVQWKVDNALQSGNSQESV |
| | | TEQDSKDSTYSLSSTLTLSKADYEKHKVYACEVTHQGLSSPVTKSENRGEC |
| DX-4012 | Heavy/ | EVQLLESGGGLVQPGGSLRLSCAASGFTFSQYVMHWVRQAPGKGLEWVSSIWPS |
| | SEQ ID | GGHTRYADSVKGRFTISRDNSKNTLYLQMNSLRAEDTAVYYCTRQRYRGPKYYY |
| | NO: 7 | YMDVWGKGTTVTVSSASTKGPSVFPLAPSSKSTSGGTAALGCLVKDYFPEPVTV |
| | | SWNSGALTSGVHTFPAVLQSSGLYSLSSVVTVPSSSLGTQTYICNVNHKPSNTK |
| | | 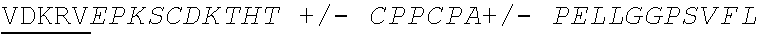 |
| DX-4012 | Light/ | EVQLLESGGGLVQPGGSLRLSCAASGFTFSQYVMHWVRQAPGKGLEWVSSIWPS |
| | SEQ ID | GGHTRYADSVKGRFTISRDNSKNTLYLQMNSLRAEDTAVYYCTRQRYRGPKYYY |
| | NO: 8 | YMDVWGKGTTVTVSS |
| 620I-X0177-A01 | scFv/ | EVQLLESGGGLVQPGGSLRLSCAASGFTFSHYIMMWVRQAPGKGLEWVSGIYSS |
| | SEQ ID | 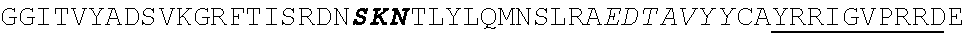 |
| | NO: 9 | FDIWGQGTMVTVSSASTKGPSVFPLAPSSKSTSGGTAALGCLVKDYFPEPVTVS |
| | | WNSGALTSGVHTFPAVLQSSGLYSLSSVVTVPSSSLGTQTYICNVNHKPSNTKV |
| | | 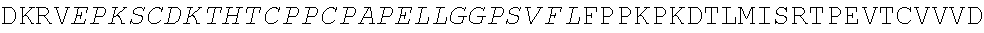 |
| | | 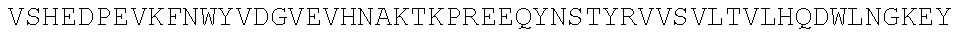 |
| | | 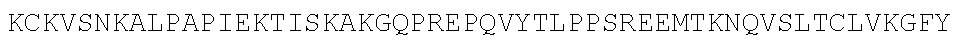 |
| | | 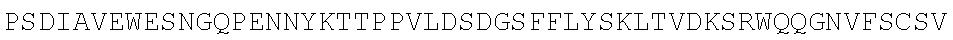 |
| | | 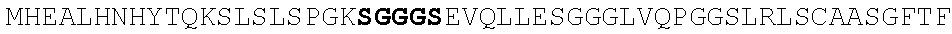 |
| | | SQYVMHWVRQAPGKCLEWVSSIWPSGGHTRYADSVKGRFTISRDNSKNTLYLQM |
| | | 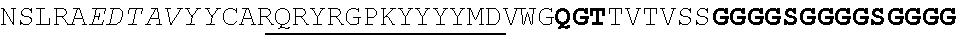 |
| | | SGGGGSDIVMTQSPLSLPVTPGEPASISCRSSQSLLHSNGYNYLDWYLQKPGQS |
| | | 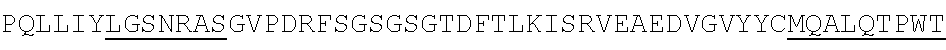 |
| | | FGCGTKVEIKR |
| Ab26036 | heavy/ | QVQLVQSGAEVKKPGASVTVSCKASGYTFTSYYLHWVRQAPGQGLEWMGIINPS |
| | SEQ ID | GGSTSYAQKFQGRVTMTRDTSTSTVYMELSSLRSEDTAVYYCARGVTTVTTYYH |
| | NO: 10 | YYNMDVWGQGTTVTVSS |
| Ab26036 | Light/ | DIQMTQSPSSLSASVGDRVTITCRASQGISNYLAWFQQKPGKAPKSLIYAASSL |
| | SEQ ID | QSGVPSKFSGSGSGTDFTLTISSLQPEDFATYYCQKYNTYPLTFGGGTKVEIK |
| | NO: 11 | |
| Ab26048 | Heavy/ | EVQLVESGGGLVQPRGSLRLSCVASGFTFSDYGMSWVRQAPGKGLEWVSVIGGA |
| | SEQ ID | GHGTYYADSVKGRFTISRDNSRNTLYLQMNSLRDEDTAVYYCAKKYYWNYVGGM |
| | NO: 12 | DVWGRGTTVTVSS |
| Ab26048 | Light/ | DIQMTQSPSSVSASVGDRVTITCRASQDINNWLAWYQQKPGKAPKLLISTASSL |
| | SEQ ID | QSGVPSRFSGSGSGTDFTLTISSLQPEDFATYYCQQANSLPFTFGPGTKVDIK |
| | NO: 13 | |
| Ab26049 | Heavy/ | EVQLVESGGDLVQPGGSLRLSCAASGFTENTYAMHWVRQAPGKGLEYISAIDTK |
| | SEQ ID | GGSTYYADSVKGRFTISRDNSKNAQYLQMDSLRVEDMAVYYCARGFGLDVWGQG |
| | NO: 14 | TTVTVSS |
| Ab26049 | Light/ | DVVMTQSPLSLAVTLGQPASISCRSSQSLGYSDGNTYLNWFQQRPGQSPRRLIY |
| | SEQ ID | KVSNRDSGVPDRFSGSGSGTDFTLKINRVETVDVGVYYCMQATHWPYTFGQGTK |
| | NO: 15 | LEIK |
| Ab26076 | Heavy/ | QVQLQQSGPGLVKPSQTLSLTCAISGDSVSSNSAAWNWIRQSPSRGLEWLGRTY |
| | SEQ ID | YRSKWENDYAVSVKSRIIINPDTTKNQFSLQVNSVTPEDTAVYYCARGEPARRG |
| | NO: 16 | EYFHHWGQGTLVTVSS |
| Ab26076 | Light/ | EIVLTQSPGTLSLSPGERATLSCRASQSVSSSYLAWYQQKPGQAPRLLIYGASS |
| | SEQ ID | RATGIPDRFSGSGSGTDFTLTISRLEPEDFAVYYCQQYGSSPWTFGQGKVEIK |
| | NO: 17 | |
| 620I-X0173-A11 | Heavy/ | EVQLLESGGGLVQPGGSLRLSCAASGFTFSQYVMHWVRQAPGKCLEWVSSIWPS |
| | SEQ ID | GGHTRYADSVKGRFTISRDNSKNTLYLQMNSLRAEDTAVYYCARQRYRGPKYYY |
| | NO: 18 | YMDVWGQGTTVTVSS |
| 620I-X0173-A11 | Light/ | DIVMTQSPLSLPVTPGEPASISCRSSQSLLHSNGYNYLDWYLQKPGQSPQLLIY |
| | SEQ ID | LGSNRASGVPDRESGSGSGTDFTLKISRVEAEDVGVYYCMQALQTPWTFGCGTK |
| | NO: 19 | VEIKR |
| 620I-X0173-C07 | Heavy/ | EVQLLESGGGLVQPGGSLRLSCAASGFTFSWYVMHWVRQAPGKCLEWVSSIYPS |
| | SEQ ID | GGKTSYADSVKGRFTISRDNSKNTLYLQMNSLRAEDTAVYYCARQRYRGPKYYY |
| | NO:20 | YMDVWGQGTTVTVSS |
| 620I-X0173-C07 | Light/ | DIVMTQSPLSLPVTPGEPASISCRSSQSLLHSNGYYLDWYLQKPGQSPQLLIYL |
| | SEQ ID | GSNRASGVPDRFSGSGSGTDFTLKISRVEAEDVGVYYCMQALQTPWTFGCGTKV |
| | NO:21 | EIKR |
| 620I-X0173-E07 | Heavy/ | EVQLLESGGGLVQPGGSLRLSCAASGFTFSWYSMHWVRQAPGKCLEWVSVIYPS |
| | SEQ ID | GGKTRYADSVKGRFTISRDNSKNTLYLQMNSLRAEDTAVYYCARQRYRGPKYYY |
| | NO:22 | YMDVWGQGTTVTVSS |
| 620I-X0173-E07 | Light/ | DIVMTQSPLSLPVTPGEPASISCRSSQSLLHSNGYYLDWYLQKPGQSPQLLIYL |
| | SEQ ID | GSNRASGVPDRFSGSGSGTDFTLKISRVEAEDVGVYYCMQALQTPWTFGCGTKV |
| | NO:23 | EIKR |
| 620I-X0173-G11 | Heavy/ | EVQLLESGGGLVQPGGSLRLSCAASGFTFSHYVMHWVRQAPGKCLEWVSSIYPS |
| | SEQ ID | GGLTKYADSVKGRFTISRDNSKNTLYLQMNSLRAEDTAVYYCARQRYRGPKYYY |
| | NO:24 | YMDVWGQGTTVTVSS |
| 620I-X0173-G11 | Light/ | DIVMTQSPLSLPVTPGEPASISCRSSQSLLHSNGYYLDWYLQKPGQSPQLLIYL |
| | SEQ ID | GSNRASGVPDRFSGSGSGTDFTLKISRVEAEDVGVYYCMQALQTPWTFGCGTKV |
| | NO:25 | EIKR |
| 559C-M0071- | Heavy/ | EVQLLESGGGLVQPGGSLRLSCAASGFTFSGYIMAWVRQAPGKGLEWVSYIYPS |
| F06 | SEQ ID | GGITVYADSVKGRFTISRDNSKNTLYLQMNSLRAEDTAVYYCTRQRYRGPKYYY |
| | NO:26 | YMDVWGKGTTVTVSS |
| 559C-M0071- | Light/ | DIQMTQSPLSLPVTPGEPASISCRSSQSLLHSNGYYLDWYLQKPGQSPQLLIYL |
| F06 | SEQ ID | GSNRASGVPDRFSGSGSGTDFTLKISRVEAEDVGVYYCMQALQTPWTFGQGTKV |
| | NO:27 | EIKR |
| 559C-M0179- | Heavy/ | EVQLLESGGGLVQPGGSLRLSCAASGFTFSGYIMAWVRQAPGKGLEWVSYIYPS |
| D04 | SEQ ID | GGITVYADSVKGRFTISRDNSKNTLYLQMNSLRAEDTAVYYCTRQRYRGPKYYY |
| | NO:26 | YMDVWGKGTTVTVSS |
| 559C-M0179- | Light/ | DIQMTQSPLSLSVAPGEPASISCRSSQSLLHRNGHNYLDWYLQKPGQSPQLLIY |
| D04 | SEQ ID | LGSNRASGVPERFSGSGSGTDFTLRISRVEAEDVGVYYCMQALQARTFGQGTKV |
| | NO:28 | EIKR |
| 559C-M0181- | Heavy/ | EVQLLESGGGLVQPGGSLRLSCAASGFTFSGYIMAWVRQAPGKGLEWVSYIYPS |
| C02 | SEQ ID | GGITVYADSVKGRFTISRDNSKNTLYLQMNSLRAEDTAVYYCTRQRYRGPKYYY |
| | NO:26 | YMDVWGKGTTVTVSS |
| 559C-M0181- | Light/ | DIQMTQSPLSLPVTPGEPASISCRSSQSLLHSNGYYLDWYLQKPGQSPQLLIYL |
| C02 | SEQ ID | GSNRASGVPDRFSGSGSGTDFTLKISRVEAEDVGVYYCMQALQTRTFGQGTKVE |
| | NO:29 | IKR |
| 559C-M0180- | Heavy/ | EVQLLESGGGLVQPGGSLRLSCAASGFTFSGYIMAWVRQAPGKGLEWVSYIYPS |
| G03 | SEQ ID | GGITVYADSVKGRFTISRDNSKNTLYLQMNSLRAEDTAVYYCTRQRYRGPKYYY |
| | NO:26 | YMDVWGKGTTVTVSS |
| 559C-M0180- | Light/ | DIQMTQSPLSLPVTPGEPASISCRSSQSLLHSNGYYLDWYLQKPGQSPQIMIYL |
| G03 | SEQ ID | GSNRASGVPDRFSGSGSGTDFTLKISRVEAEDVGVYYCMQALQTPRTFGQGTKV |
| | NO:30 | EIKR |
| 559C-M0184- | Heavy/ | EVQLLESGGGLVQPGGSLRLSCAASGFTFSFYSMHWVRQAPGKGLEWVSRIYPS |
| B04 | SEQ ID | GGVTKYADSVKGRFTISRDNSKNTLYLQMNSLRAEDTAVYYCTRQRYRGPKYYY |
| | NO:31 | YMDVWGKGTTVTVSS |
| 559C-M0184- | Light/ | DIQMTQSPLSLPVTPGEPASISCRSSQSLLHSNGYYLDWYLQKPGQSPQLLIYL |
| B04 | SEQ ID | GSNRASGVPDRESGSGSGTDFTLKISRVEAEDVGVYYCMQALQTPWTFGQGTKV |
| | NO:27 | EIKR |

TABLE 8
Nucleotide Sequences of Heavy and Light Chain

| | Chain/ | |
|---|---|---|
| | SEQ ID | |
| Ab | NO. | Sequence |
| Garadacimab | Heavy/ | GAGGTGCAGCTGCTGGAGAGCGGCGGCGGCCTGGTGCAGCCCGGCGGCAGCCTG |
| | SEQ ID | AGGCTGAGCTGCGCCGCCAGCGGCTTCACCTTCAGCAAGTACATCATGCAGTGG |
| | NO: 32 | GTGAGGCAGGCCCCCGGCAAGGGCCTGGAGTGGGTGAGCGGCATCGACATCCCC |
| | | ACCAAGGGCACCGTGTACGCCGACAGCGTGAAGGGCAGGTTCACCATCAGCAGG |
| | | GACAACAGCAAGAACACCCTGTACCTGCAGATGAACAGCCTGAGGGCCGAGGAC |
| | | ACCGCCGTGTACTACTGCGCCAGGGCCCTGCCCAGGAGCGGCTACCTGATCAGC |
| | | CCCCACTACTACTACTACGCCCTGGACGTGTGGGGCCAGGGCACCACCGTGACC |
| | | GTGAGCAGCGCCAGCACCAAGGGCCCCAGCGTGTTCCCCCTGGCCCCCTGCAGC |
| | | AGGAGCACCAGCGAGAGCACCGCCGCCCTGGGCTGCCTGGTGAAGGACTACTTC |
| | | CCCGAGCCCGTGACCGTGAGCTGGAACAGCGGCGCCCTGACCAGCGGCGTGCAC |
| | | ACCTTCCCCGCCGTGCTGCAGAGCAGCGGCCTGTACAGCCTGAGCAGCGTGGTG |
| | | ACCGTGCCCAGCAGCAGCCTGGGCACCAAGACCTACACCTGCAACGTGGACCAC |
| | | AAGCCCAGCAACACCAAGGTGGACAAGAGGGTGGAGAGCAAGTACGGCCCCCCC |
| | | TGCCCCCCCTGCCCCGCCCCCGAGTTCCTGGGCGGCCCCAGCGTGTTCCTG |
| Garadacimab | Light/SEQ | CAGAGCGTGCTGACCCAGCCCCCCAGCGCCAGCGGCACCCCCGGCCAGAGGGTG |
| | ID NO: 33 | ACCATCAGCTGCAGCGGCAGCAGCAGCAACATCGGCAGGAACTACGTGTACTGG |
| | | TACCAGCAGCTGCCCGGCACCGCCCCCAAGCTGCTGATCTACAGCAACAACCAG |
| | | AGGCCCAGCGGCGTGCCCGACAGGTTCAGCGGCAGCAAGAGCGGCACCAGCGCC |
| | | AGCCTGGCCATCAGCGGCCTGAGGAGCGAGGACGAGGCCGACTACTACTGCGCC |
| | | GCCTGGGACGCCAGCCTGAGGGGCGTGTTCGGCGGCGGCACCAAGCTGACCGTG |
| | | CTGGGCCAGCCCAAGGCCGCCCCCAGCGTGACCCTGTTCCCCCCCAGCAGCGAG |
| | | GAGCTGCAGGCCAACAAGGCCACCCTGGTGTGCCTGATCAGCGACTTCTACCCC |
| | | GGCGCCGTGACCGTGGCCTGGAAGGCCGACAGCAGCCCCGTGAAGGCC |
| | | GGCGTGGAGACCACCACCCCCAGCAAGCAGAGCAACAACAAGTACGCCGCCAGC |
| | | AGCTACCTGAGCCTGACCCCCGAGCAGTGGAAGAGCCACAGGAGCTACAGCTGC |
| | | CAGGTGACCCACGAGGGCAGCACCGTGGAGAAGACCGTGGCCCCCACCGAGTGC |
| | | AGC |
| AB042/43 | Heavy/ | GAGGTGCAGCTGCAGCAGAGCGGCGCCGAGCTGGTGAGGCCCGGCGCCAGCGTG |
| | SEQ ID | AAGCTGAGCTGCACCGCCAGCGGCTTCAACATCAAGGACGACTACATGCACTGG |
| | NO: 34 | GTGAAGCAGAGGCCCGAGCAGGGCCTGGAGTGGATCGGCTGGATCGACCCCGAG |
| | | AACGGCGACACCGAGTACGCCAGCAAGTTCCAGGGCAAGGCCACCATCACCAGC |
| | | GACACCAGCAGCAACACCGCCTACCTGCAGCTGAGCAGCCTGACCAGCGAGGAC |
| | | ACCGCCGTGTACTACTGCACCGAGAACTACTACGGCAGCAGCTTCTTCGCCTAC |
| | | TGGGGCCAGGGCACCCTGGTGACCGTGAGCGCC |
| AB042/43 | Light/SEQ | gatattgtgatgactcaggctacaccctctgtacctgtcactcctggagagtca |
| | ID NO: 35 | gtatccatctcctgcaggtctagtaagagtcttctgcatagtaatggcatcact |
| | | tacttgtattggttcctgcagaggccaggccagtctcctcagcgcctgatatat |
| | | tatatgtccaaccttgcctcaggagtcccagacaggttcagtggcagagggtca |
| | | ggaactgatttcacactgagaatcagtagagtggaggctgaggatgtgggtgtt |
| | | tattactgtatgcaaagtctagaatatccgtacacgttcggaggggggaccaag |
| | | ctggaaataaaa |
| AB054 | Heavy/ | caggtgcagc tccaggagag cggacccggt ctggtgaagc ccagccagac |
| | SEQ ID | cctgagcctg acctgcaccg tgagcggcta ctcaatcacc tctggctaca |
| | NO: 36 | gctggcactg gatcaggcag caccccggca agggcctgga gtggattggc |
| | | tatatccagt acagcggcaa caccaacagc aaccccagcc tcaagagcag |
| | | ggtgaccatc agcagggata caagcaagaa ccagttcagc ctgaagctga |
| | | gcagcgtgac cgccgctgac accgccgtgt actactgcgc caggtggggc |
| | | agcttcgact actggggcca gggcaccctg gtgaccgtgt cttctgctag |
| | | caccaagggc cccagcgtgt ttcctctcgc tccctgcagc cggagcacat |
| | | ccgagagcac cgctgctctg ggctgtctcg tgaaggacta cttccctgaa |
| | | cccgtcaccg tcagctggaa tagcggcgcc ctgacatccg gcgtccacac |
| | | attccccgct gtcctgcaga gcagcggcct gtacagcctg agctccgtgg |
| | | tcaccgtgcc tagcagcagc ctgggaacaa agacctacac ctgcaacgtg |
| | | gaccataagc cctccaacac caaggtggac aagcgggtgg aatccaagta |
| | | tggacccccc tgtcctcctt gccctgctcc tgaatttctc ggaggcccct |
| | | ccgtcttcct gtttcccccc aagcccaagg acaccctgat gatctcccgg |
| | | acacccgaag tcacctgcgt cgtggtggat gtcagccagg aagatcccga |
| | | ggtgcagttc aactggtacg tggacggagt ggaggtgcat aacgccaaaa |
| | | ccaagcccag ggaagagcag ttcaacagca cctatcgggt cgtgtccgtg |
| | | ctcaccgtcc tgcatcagga ttggctcaac ggcaaggagt acaagtgcaa |
| | | ggtgtccaac aagggcctgc cctcctccat cgagaagacc atctccaagg |
| | | ctaagggcca acctcgggag ccccaagtgt ataccctccc tcccagccag |
| | | gaggagatga ccaagaatca agtgagcctg acctgcctcg tgaagggatt |
| | | ttacccctcc gacatcgctg tggaatggga aagcaatggc caacctgaga |
| | | acaactacaa gaccacaccc cccgtgctgg actccgatgg ctccttcttc |
| | | ctgtacagca ggctgaccgt ggacaaatcc cggtggcaag agggaaacgt |
| | | gttcagctgc tccgtgatgc acgaggctct ccacaaccac tacacccaga |
| | | agagcctctc cctgagcctc ggctagtaa |
| AB054 | Light/ | gagatcgtgc tgacccagag cccagcaacc ctgagcttga gccccggtga |
| | SEQ ID | gagggccacc ctgtcatgca gggccagcag cagcgtgaac tacctgcact |
| | NO: 37 | ggtatcagca gaagcccggt caagccccca ggaggctgat ctacgacacc |
| | | agcaagctgg ccaccggcat ccccgccagg ttttccggca gcgggtcagg |
| | | caccgactac accctcacca taagcagcct ggagcccgag |
| | | gacttcgccg tgtactactg tcagcagtgg agcggcaacc cacctacctt |
| | | tggcggaggc actaaggtgg agatcaagcg gaccgtggcc gcccccagcg |
| | | tgttcatctt ccctcccagc gacgagcagc tgaagtctgg caccgccagc |
| | | gtggtgtgcc tgctgaacaa cttctacccc cgcgaggcca aggtgcagtg |
| | | gaaggtggac aacgccctgc agagcggcaa cagccaggag |
| | | agcgtgaccg agcaggactc caaggacagc acctacagcc tgagcagcac |
| | | cctgaccctg agcaaggccg actacgagaa gcacaaggtg tacgcctgcg |
| | | aggtgaccca ccagggactg tctagccccg tgaccaagag cttcaaccgg |
| | | ggcgagtgct aa |
| DX-4012 | Heavy/ | GAGGTGCAGCTGCTGGAGAGCGGCGGCGGCCTGGTGCAGCCCGGCGGCAGCCTG |
| | SEQ ID | AGGCTGAGCTGCGCCGCCAGCGGCTTCACCTTCAGCCAGTACGTGATGCACTGG |
| | NO: 38 | GTGAGGCAGGCCCCCGGCAAGGGCCTGGAGTGGGTGAGCAGCATCTGGCCCAGC |
| | | GGCGGCCACACCAGGTACGCCGACAGCGTGAAGGGCAGGTTCACCATCAGCAGG |
| | | GACAACAGCAAGAACACCCTGTACCTGCAGATGAACAGCCTGAGGGCCGAGGAC |
| | | ACCGCCGTGTACTACTGCACCAGGCAGAGGTACAGGGGCCCCAAGTACTACTAC |
| | | TACATGGACGTGTGGGGCAAGGGCACCACCGTGACCGTGAGCAGCGCCAGCACC |
| | | AAGGGCCCCAGCGTGTTCCCCCTGGCCCCCAGCAGCAAGAGCACCAGCGGCGGC |
| | | ACCGCCGCCCTGGGCTGCCTGGTGAAGGACTACTTCCCCGAGCCCGTGACCGTG |
| | | AGCTGGAACAGCGGCGCCCTGACCAGCGGCGTGCACACCTTCCCCGCCGTGCTG |
| | | CAGAGCAGCGGCCTGTACAGCCTGAGCAGCGTGGTGACCGTGCCCAGCAGCAGC |
| | | CTGGGCACCCAGACCTACATCTGCAACGTGAACCACAAGCCCAGCAACACCAAG |
| | | GTGGACAAGAGGGTGGAGCCCAAGAGCTGCGACAAGACCCACACCTGCCCCCCC |
| | | TGCCCCGCCCCCGAGCTGCTGGGCGGCCCCAGCGTGTTCCTG |
| DX-4012 | Light/SEQ | GAGGTGCAGCTGCTGGAGAGCGGCGGCGGCCTGGTGCAGCCCGGCGGCAGCCTG |
| | ID NO: 39 | AGGCTGAGCTGCGCCGCCAGCGGCTTCACCTTCAGCCAGTACGTGATGCACTGG |
| | | GTGAGGCAGGCCCCCGGCAAGGGCCTGGAGTGGGTGAGCAGCATCTGGCCCAGC |
| | | GGCGGCCACACCAGGTACGCCGACAGCGTGAAGGGCAGGTTCACCATCAGCAGG |
| | | GACAACAGCAAGAACACCCTGTACCTGCAGATGAACAGCCTGAGGGCCGAGGAC |
| | | ACCGCCGTGTACTACTGCACCAGGCAGAGGTACAGGGGCCCCAAGTACTACTAC |
| | | TACATGGACGTGTGGGGCAAGGGCACCACCGTGACCGTGAGCAGC |
| 620I-X0177-A01 | HC-scFv/ | GAGGTGCAGCTGCTGGAGAGCGGCGGCGGCCTGGTGCAGCCCGGCGGCAGCCTG |
| | SEQ ID | AGGCTGAGCTGCGCCGCCAGCGGCTTCACCTTCAGCCACTACATCATGATGTGG |
| | NO: 40 | GTGAGGCAGGCCCCCGGCAAGGGCCTGGAGTGGGTGAGCGGCATCTACAGCAGC |
| | | GGCGGCATCACCGTGTACGCCGACAGCGTGAAGGGCAGGTTCACCATCAGCAGG |
| | | GACAACAGCAAGAACACCCTGTACCTGCAGATGAACAGCCTGAGGGCCGAGGAC |
| | | ACCGCCGTGTACTACTGCGCCTACAGGAGGATCGGCGTGCCCAGGAGGGACGAG |
| | | TTCGACATCTGGGGCCAGGGCACCATGGTGACCGTGAGCAGCGCCAGCACCAAG |
| | | GGCCCCAGCGTGTTCCCCCTGGCCCCCAGCAGCAAGAGCACCAGCGGCGGCACC |
| | | GCCGCCCTGGGCTGCCTGGTGAAGGACTACTTCCCCGAGCCCGTGACCGTGAGC |
| | | TGGAACAGCGGCGCCCTGACCAGCGGCGTGCACACCTTCCCCGCCGTGCTGCAG |
| | | AGCAGCGGCCTGTACAGCCTGAGCAGCGTGGTGACCGTGCCCAGCAGCAGCCTG |
| | | GGCACCCAGACCTACATCTGCAACGTGAACCACAAGCCCAGCAACACCAAGGTG |
| | | GACAAGAGGGTGGAGCCCAAGAGCTGCGACAAGACCCACACCTGCCCCCCCTGC |
| | | CCCGCCCCCGAGCTGCTGGGCGGCCCCAGCGTGTTCCTGTTCCCCCCCAAGCCC |
| | | AAGGACACCCTGATGATCAGCAGGACCCCCGAGGTGACCTGCGTGGTGGTGGAC |
| | | GTGAGCCACGAGGACCCCGAGGTGAAGTTCAACTGGTACGTGGACGGCGTGGAG |
| | | GTGCACAACGCCAAGACCAAGCCCAGGGAGGAGCAGTACAACAGCACCTACAGG |
| | | GTGGTGAGCGTGCTGACCGTGCTGCACCAGGACTGGCTGAACGGCAAGGAGTAC |
| | | AAGTGCAAGGTGAGCAACAAGGCCCTGCCCGCCCCCATCGAGAAGACCATCAGC |
| | | AAGGCCAAGGGCCAGCCCAGGGAGCCCCAGGTGTACACCCTGCCCCCCAGCAGG |
| | | GAGGAGATGACCAAGAACCAGGTGAGCCTGACCTGCCTGGTGAAGGGCTTCTAC |
| | | CCCAGCGACATCGCCGTGGAGTGGGAGAGCAACGGCCAGCCCGAGAACAACTAC |
| | | AAGACCACCCCCCCCGTGCTGGACAGCGACGGCAGCTTCTTCCTGTACAGCAAG |
| | | CTGACCGTGGACAAGAGCAGGTGGCAGCAGGGCAACGTGTTCAGCTGCAGCGTG |
| | | ATGCACGAGGCCCTGCACAACCACTACACCCAGAAGAGCCTGAGCCTGAGCCCC |
| | | GGCAAGAGCGGCGGCGGCAGCGAGGTGCAGCTGCTGGAGAGCGGCGGCGGCCTG |
| | | GTGCAGCCCGGCGGCAGCCTGAGGCTGAGCTGCGCCGCCAGCGGCTTCACCTTC |
| | | AGCCAGTACGTGATGCACTGGGTGAGGCAGGCCCCCGGCAAGTGCCTGGAGTGG |
| | | GTGAGCAGCATCTGGCCCAGCGGCGGCCACACCAGGTACGCCGACAGCGTGAAG |
| | | GGCAGGTTCACCATCAGCAGGGACAACAGCAAGAACACCCTGTACCTGCAGATG |
| | | AACAGCCTGAGGGCCGAGGACACCGCCGTGTACTACTGCGCCAGGCAGAGGTAC |
| | | AGGGGCCCCAAGTACTACTACTACATGGACGTGTGGGGCCAGGGCACCACCGTG |
| | | ACCGTGAGCAGCGGCGGCGGCGGCAGCGGCGGCGGCGGCAGCGGCGGCGGCGGC |
| | | AGCGGCGGCGGCGGCAGCGACATCGTGATGACCCAGAGCCCCCTGAGCCTGCCC |
| | | GTGACCCCCGGCGAGCCCGCCAGCATCAGCTGCAGGAGCAGCCAGAGCCTGCTG |
| | | CACAGCAACGGCTACAACTACCTGGACTGGTACCTGCAGAAGCCCGGCCAGAGC |
| | | CCCCAGCTGCTGATCTACCTGGGCAGCAACAGGGCCAGCGGCGTGCCCGACAGG |
| | | TTCAGCGGCAGCGGCAGCGGCACCGACTTCACCCTGAAGATCAGCAGGGTGGAG |
| | | GCCGAGGACGTGGGCGTGTACTACTGCATGCAGGCCCTGCAGACCCCCTGGACC |
| | | TTCGGCTGCGGCACCAAGGTGGAGATCAAGAGG |
| Ab26036 | heavy/ | caggtgcagc tggtgcagtc tggggctgag gtgaagaagc ctggggcctc |
| | SEQ ID | agtgacggtt tcctgcaagg catctggata caccttcacc agctactatt |
| | NO: 41 | tgcactgggt gcgacaggcc cctggacaag gacttgagtg gatgggaata |
| | | atcaacccca gtggtggtag cacaagctac gcacagaagt tccagggcag |
| | | agtcaccatg accagggaca cgtccacgag cacagtctac atggagctga |
| | | gcagcctgag atctgaggac acggccgtgt attactgtgc gagaggcgtg |
| | | actacagtga ctacttacta ccactactac aatatggacg tctggggcca |
| | | agggaccacg gtcaccgtct cctca |
| Ab26036 | Light/ | gacatccaga tgacccagtc tccatcctca ctgtctgcat ctgtaggaga |
| | SEQ ID | cagagtcacc atcacttgtc gggcgagtca gggcattagc aattatttag |
| | NO: 42 | cctggtttca gcagaaacca gggaaagccc ctaagtccct gatctatgct |
| | | gcatccagtt tgcaaagtgg ggtcccatca aagttcagcg gcagtggatc |
| | | tgggacagat ttcactctca ccatcagcag cctgcagcct gaagattttg |
| | | caacttatta ctgccaaaag tataatactt accctctcac tttcggcgga |
| | | gggaccaagg tggagatcaa a |
| Ab26048 | Heavy/ | gaggtgcagc tggtggagtc tgggggaggc ttggtgcagc ctcgggggtc |
| | SEQ ID | cctgagactc tcctgtgtgg cctctggatt cacctttagc gactatggca |
| | NO: 43 | tgagttgggt ccgacaggct ccagggaagg ggctggagtg ggtctcagtt |
| | | attggtggtg ctggtcatgg cacatattac gctgactccg tgaagggccg |
| | | gttcaccatc tccagagaca attccaggaa cacgttgtat ctgcaaatga |
| | | acagcctgag agacgaggac acggccgtat attattgtgc gaaaaaatat |
| | | tactggaact acgtcggcgg tatggacgtc tggggccgag ggaccacggt |
| | | caccgtctcc tca |
| Ab26048 | Light/ | gacatccaga tgacccagtc tccatcttcc gtgtctgcat ctgtaggaga |
| | SEQ ID | cagagtcacc atcacttgtc gggcgagtca ggatattaac aactggttag |
| | NO: 44 | cctggtatca gcagaaacca gggaaagccc ctaaactcct gatctctact |
| | | gcatccagtt tgcaaagtgg ggtcccatca aggttcagcg gcagtggatc |
| | | tgggacagac ttcactctca ccatcagtag cctacagcct gaagattttg |
| | | caacttacta ttgtcaacag gctaacagtc tcccattcac tttcggccct |
| | | gggaccaaag tggatatcaa a |
| Ab26049 | Heavy/ | gaggtgcagc tggtggagtc tgggggagac ttggtccagc ctggggggtc |
| | SEQ ID | cctgagactc tcctgtgcag cctctggatt caccttcaat acctatgcta |
| | NO: 45 | tgcactgggt ccgccaggct ccagggaagg gactggaata tatttcagct |
| | | attgatacca aagggggttc cacatattat gcagactctg tgaagggcag |
| | | attcaccatt tccagagaca attccaagaa cgcgcagtat cttcaaatgg |
| | | acagcctgag agttgaagac atggctgttt attattgtgc gagagggttc |
| | | ggtctggacg tctggggcca agggaccacg gtcaccgtct cctca |
| Ab26049 | Light/ | gatgttgtga tgacccagtc tccactctcc ctggccgtca cccttggaca |
| | SEQ ID | gccggcctcc atctcctgca ggtctagtca aagcctcgga tacagtgatg |
| | NO: 46 | gaaacaccta cttgaattgg tttcaacaga ggccaggcca atctccaagg |
| | | cgcctaattt ataaggtttc taaccgggac tctggggtcc cagacagatt |
| | | cagcggcagt gggtcaggca ctgacttcac actgaaaatc aatagggtgg |
| | | agactgtgga tgttggggtt tactactgca tgcaagctac acactggccg |
| | | tacacttttg gccaggggac caagctggag atcaaa |
| Ab26076 | Heavy/ | caggtacagc tgcagcagtc aggtccagga ctggtgaagc cctcgcagac |
| | SEQ ID | cctctcactc acctgtgcca tctccgggga cagtgtctct agcaatagtg |
| | NO: 47 | ctgcttggaa ttggatcagg cagtccccct cgagaggcct tgagtggctg |
| | | ggaaggacat actacaggtc caagtggttt aatgattatg cagtgtctgt |
| | | gaaaagtcga ataatcatca acccagacac aaccaagaac cagttctccc |
| | | tgcaggtgaa ctctgtgact cccgaagaca cggctgtgta ttactgtgca |
| | | agaggagagc cagctcgtcg gggtgaatac ttccaccact ggggccaggg |
| | | caccctggtc accgtctcct ca |
| Ab26076 | Light/ | gaaattgtgt tgacgcagtc tccaggcacc ctgtctttgt ctccagggga |
| | SEQ ID | aagagccacc ctctcctgca gggccagtca gagtgttagc agcagctact |
| | NO: 48 | tagcctggta ccagcagaaa cctggccagg ctcccaggct cctcatctat |
| | | ggtgcatcca gcagggccac tggcatccca gacaggttca gtggcagtgg |
| | | gtctgggaca gacttcactc tcaccatcag cagactggag cctgaagatt |
| | | ttgcagtgta ttactgtcag cagtatggta gctcaccttg gacgttcggc |
| | | caagggacca aggtggaaat caaa |
| 620I-X0173-A11 | Heavy/ | GAGGTGCAGCTGCTGGAGAGCGGCGGCGGCCTGGTGCAGCCCGGCGGCAGCCTG |
| | SEQ ID | AGGCTGAGCTGCGCCGCCAGCGGCTTCACCTTCAGCCAGTACGTGATGCACTGG |
| | NO: 49 | GTGAGGCAGGCCCCCGGCAAGTGCCTGGAGTGGGTGAGCAGCATCTGGCCCAGC |
| | | GGCGGCCACACCAGGTACGCCGACAGCGTGAAGGGCAGGTTCACCATCAGCAGG |
| | | GACAACAGCAAGAACACCCTGTACCTGCAGATGAACAGCCTGAGGGCCGAGGAC |
| | | ACCGCCGTGTACTACTGCGCCAGGCAGAGGTACAGGGGCCCCAAGTACTACTAC |
| | | TACATGGACGTGTGGGGCCAGGGCACCACCGTGACCGTGAGCAGC |
| 620I-X0173-A11 | Light/ | GACATCGTGATGACCCAGAGCCCCCTGAGCCTGCCCGTGACCCCCGGCGAGCCC |
| | SEQ ID | GCCAGCATCAGCTGCAGGAGCAGCCAGAGCCTGCTGCACAGCAACGGCTACAAC |
| | NO: 50 | TACCTGGACTGGTACCTGCAGAAGCCCGGCCAGAGCCCCCAGCTGCTGATCTAC |
| | | CTGGGCAGCAACAGGGCCAGCGGCGTGCCCGACAGGTTCAGCGGCAGCGGCAGC |
| | | GGCACCGACTTCACCCTGAAGATCAGCAGGGTGGAGGCCGAGGACGTGGGCGTG |
| | | TACTACTGCATGCAGGCCCTGCAGACCCCCTGGACCTTCGGCTGCGGCACCAAG |
| | | GTGGAGATCAAGAGG |
| 620I-X0173-C07 | Heavy/ | GAGGTGCAGCTGCTGGAGAGCGGCGGCGGCCTGGTGCAGCCCGGCGGCAGCCTG |
| | SEQ ID | AGGCTGAGCTGCGCCGCCAGCGGCTTCACCTTCAGCTGGTACGTGATGCACTGG |
| | NO: 51 | GTGAGGCAGGCCCCCGGCAAGTGCCTGGAGTGGGTGAGCAGCATCTACCCCAGC |
| | | GGCGGCAAGACCAGCTACGCCGACAGCGTGAAGGGCAGGTTCACCATCAGCAGG |
| | | GACAACAGCAAGAACACCCTGTACCTGCAGATGAACAGCCTGAGGGCCGAGGAC |
| | | ACCGCCGTGTACTACTGCGCCAGGCAGAGGTACAGGGGCCCCAAGTACTACTAC |
| | | TACATGGACGTGTGGGGCCAGGGCACCACCGTGACCGTGAGCAGC |
| 620I-X0173-C07 | Light/ | GACATCGTGATGACCCAGAGCCCCCTGAGCCTGCCCGTGACCCCCGGCGAGCCC |
| | SEQ ID | GCCAGCATCAGCTGCAGGAGCAGCCAGAGCCTGCTGCACAGCAACGGCTACAAC |
| | NO: 50 | TACCTGGACTGGTACCTGCAGAAGCCCGGCCAGAGCCCCCAGCTGCTGATCTAC |
| | | CTGGGCAGCAACAGGGCCAGCGGCGTGCCCGACAGGTTCAGCGGCAGCGGCAGC |
| | | GGCACCGACTTCACCCTGAAGATCAGCAGGGTGGAGGCCGAGGACGTGGGCGTG |
| | | TACTACTGCATGCAGGCCCTGCAGACCCCCTGGACCTTCGGCTGCGGCACCAAG |
| | | GTGGAGATCAAGAGG |
| 620I-X0173-E07 | Heavy/ | GAGGTGCAGCTGCTGGAGAGCGGCGGCGGCCTGGTGCAGCCCGGCGGCAGCCTG |
| | SEQ ID | AGGCTGAGCTGCGCCGCCAGCGGCTTCACCTTCAGCTGGTACAGCATGCACTGG |
| | NO: 52 | GTGAGGCAGGCCCCCGGCAAGTGCCTGGAGTGGGTGAGCGTGATCTACCCCAGC |
| | | GGCGGCAAGACCAGGTACGCCGACAGCGTGAAGGGCAGGTTCACCATCAGCAGG |
| | | GACAACAGCAAGAACACCCTGTACCTGCAGATGAACAGCCTGAGGGCCGAGGAC |
| | | ACCGCCGTGTACTACTGCGCCAGGCAGAGGTACAGGGGCCCCAAGTACTACTAC |
| | | TACATGGACGTGTGGGGCCAGGGCACCACCGTGACCGTGAGCAGC |
| 620I-X0173-E07 | Light/ | GACATCGTGATGACCCAGAGCCCCCTGAGCCTGCCCGTGACCCCCGGCGAGCCC |
| | SEQ ID | GCCAGCATCAGCTGCAGGAGCAGCCAGAGCCTGCTGCACAGCAACGGCTACAAC |
| | NO: 50 | TACCTGGACTGGTACCTGCAGAAGCCCGGCCAGAGCCCCCAGCTGCTGATCTAC |
| | | CTGGGCAGCAACAGGGCCAGCGGCGTGCCCGACAGGTTCAGCGGCAGCGGCAGC |
| | | GGCACCGACTTCACCCTGAAGATCAGCAGGGTGGAGGCCGAGGACGTGGGCGTG |
| | | TACTACTGCATGCAGGCCCTGCAGACCCCCTGGACCTTCGGCTGCGGCACCAAG |
| | | GTGGAGATCAAGAGG |
| 620I-X0173-G11 | Heavy/ | GAGGTGCAGCTGCTGGAGAGCGGCGGCGGCCTGGTGCAGCCCGGCGGCAGCCTG |
| | SEQ ID | AGGCTGAGCTGCGCCGCCAGCGGCTTCACCTTCAGCCACTACGTGATGCACTGG |
| | NO: 53 | GTGAGGCAGGCCCCCGGCAAGTGCCTGGAGTGGGTGAGCAGCATCTACCCCAGC |
| | | GGCGGCCTGACCAAGTACGCCGACAGCGTGAAGGGCAGGTTCACCATCAGCAGG |
| | | GACAACAGCAAGAACACCCTGTACCTGCAGATGAACAGCCTGAGGGCCGAGGAC |
| | | ACCGCCGTGTACTACTGCGCCAGGCAGAGGTACAGGGGCCCCAAGTACTACTAC |
| | | TACATGGACGTGTGGGGCCAGGGCACCACCGTGACCGTGAGCAGC |
| 620I-X0173-G11 | Light/ | GACATCGTGATGACCCAGAGCCCCCTGAGCCTGCCCGTGACCCCCGGCGAGCCC |
| | SEQ ID | GCCAGCATCAGCTGCAGGAGCAGCCAGAGCCTGCTGCACAGCAACGGCTACAAC |
| | NO: 50 | TACCTGGACTGGTACCTGCAGAAGCCCGGCCAGAGCCCCCAGCTGCTGATCTAC |
| | | CTGGGCAGCAACAGGGCCAGCGGCGTGCCCGACAGGTTCAGCGGCAGCGGCAGC |
| | | GGCACCGACTTCACCCTGAAGATCAGCAGGGTGGAGGCCGAGGACGTGGGCGTG |
| | | TACTACTGCATGCAGGCCCTGCAGACCCCCTGGACCTTCGGCTGCGGCACCAAG |
| | | GTGGAGATCAAGAGG |
| 559C-M0071- | Heavy/ | GAGGTGCAGCTGCTGGAGAGCGGCGGCGGCCTGGTGCAGCCCGGCGGCAGCCTG |
| F06 | SEQ ID | AGGCTGAGCTGCGCCGCCAGCGGCTTCACCTTCAGCGGCTACATCATGGCCTGG |
| | NO: 54 | GTGAGGCAGGCCCCCGGCAAGGGCCTGGAGTGGGTGAGCTACATCTACCCCAGC |
| | | GGCGGCATCACCGTGTACGCCGACAGCGTGAAGGGCAGGTTCACCATCAGCAGG |
| | | GACAACAGCAAGAACACCCTGTACCTGCAGATGAACAGCCTGAGGGCCGAGGAC |
| | | ACCGCCGTGTACTACTGCACCAGGCAGAGGTACAGGGGCCCCAAGTACTACTAC |
| | | TACATGGACGTGTGGGGCAAGGGCACCACCGTGACCGTGAGCAGC |
| 559C-M0071- | Light/ | GACATCCAGATGACCCAGAGCCCCCTGAGCCTGCCCGTGACCCCCGGCGAGCCC |
| F06 | SEQ ID | GCCAGCATCAGCTGCAGGAGCAGCCAGAGCCTGCTGCACAGCAACGGCTACTAC |
| | NO: 55 | CTGGACTGGTACCTGCAGAAGCCCGGCCAGAGCCCCCAGCTGCTGATCTACCTG |
| | | GGCAGCAACAGGGCCAGCGGCGTGCCCGACAGGTTCAGCGGCAGCGGCAGCGGC |
| | | ACCGACTTCACCCTGAAGATCAGCAGGGTGGAGGCCGAGGACGTGGGCGTGTAC |
| | | TACTGCATGCAGGCCCTGCAGACCCCCTGGACCTTCGGCCAGGGCACCAAGGTG |
| | | GAGATCAAGAGG |
| 559C-M0179- | Heavy/ | GAGGTGCAGCTGCTGGAGAGCGGCGGCGGCCTGGTGCAGCCCGGCGGCAGCCTG |
| D04 | SEQ ID | AGGCTGAGCTGCGCCGCCAGCGGCTTCACCTTCAGCGGCTACATCATGGCCTGG |
| | NO: 54 | GTGAGGCAGGCCCCCGGCAAGGGCCTGGAGTGGGTGAGCTACATCTACCCCAGC |
| | | GGCGGCATCACCGTGTACGCCGACAGCGTGAAGGGCAGGTTCACCATCAGCAGG |
| | | GACAACAGCAAGAACACCCTGTACCTGCAGATGAACAGCCTGAGGGCCGAGGAC |
| | | ACCGCCGTGTACTACTGCACCAGGCAGAGGTACAGGGGCCCCAAGTACTACTAC |
| | | TACATGGACGTGTGGGGCAAGGGCACCACCGTGACCGTGAGCAGC |
| 559C-M0179- | Light/ | GACATCCAGATGACCCAGAGCCCCCTGAGCCTGAGCGTGGCCCCCGGCGAGCCC |
| D04 | SEQ ID | GCCAGCATCAGCTGCAGGAGCAGCCAGAGCCTGCTGCACAGGAACGGCCACAAC |
| | NO: 56 | TACCTGGACTGGTACCTGCAGAAGCCCGGCCAGAGCCCCCAGCTGCTGATCTAC |
| | | CTGGGCAGCAACAGGGCCAGCGGCGTGCCCGAGAGGTTCAGCGGCAGCGGCAGC |
| | | GGCACCGACTTCACCCTGAGGATCAGCAGGGTGGAGGCCGAGGACGTGGGCGTG |
| | | TACTACTGCATGCAGGCCCTGCAGGCCAGGACCTTCGGCCAGGGCACCAAGGTG |
| | | GAGATCAAGAGG |
| 559C-M0181- | Heavy/ | GAGGTGCAGCTGCTGGAGAGCGGCGGCGGCCTGGTGCAGCCCGGCGGCAGCCTG |
| C02 | SEQ ID | AGGCTGAGCTGCGCCGCCAGCGGCTTCACCTTCAGCGGCTACATCATGGCCTGG |
| | NO: 54 | GTGAGGCAGGCCCCCGGCAAGGGCCTGGAGTGGGTGAGCTACATCTACCCCAGC |
| | | GGCGGCATCACCGTGTACGCCGACAGCGTGAAGGGCAGGTTCACCATCAGCAGG |
| | | GACAACAGCAAGAACACCCTGTACCTGCAGATGAACAGCCTGAGGGCCGAGGAC |
| | | ACCGCCGTGTACTACTGCACCAGGCAGAGGTACAGGGGCCCCAAGTACTACTAC |
| | | TACATGGACGTGTGGGGCAAGGGCACCACCGTGACCGTGAGCAGC |
| 559C-M0181- | Light/ | GACATCCAGATGACCCAGAGCCCCCTGAGCCTGCCCGTGACCCCCGGCGAGCCC |
| C02 | SEQ ID | GCCAGCATCAGCTGCAGGAGCAGCCAGAGCCTGCTGCACAGCAACGGCTACTAC |
| | NO: 57 | CTGGACTGGTACCTGCAGAAGCCCGGCCAGAGCCCCCAGCTGCTGATCTACCTG |
| | | GGCAGCAACAGGGCCAGCGGCGTGCCCGACAGGTTCAGCGGCAGCGGCAGCGGC |
| | | ACCGACTTCACCCTGAAGATCAGCAGGGTGGAGGCCGAGGACGTGGGCGTGTAC |
| | | TACTGCATGCAGGCCCTGCAGACCAGGACCTTCGGCCAGGGCACCAAGGTGGAG |
| | | ATCAAGAGG |
| 559C-M0180- | Heavy/ | GAGGTGCAGCTGCTGGAGAGCGGCGGCGGCCTGGTGCAGCCCGGCGGCAGCCTG |
| G03 | SEQ ID | AGGCTGAGCTGCGCCGCCAGCGGCTTCACCTTCAGCGGCTACATCATGGCCTGG |
| | NO: 54 | GTGAGGCAGGCCCCCGGCAAGGGCCTGGAGTGGGTGAGCTACATCTACCCCAGC |
| | | GGCGGCATCACCGTGTACGCCGACAGCGTGAAGGGCAGGTTCACCATCAGCAGG |
| | | GACAACAGCAAGAACACCCTGTACCTGCAGATGAACAGCCTGAGGGCCGAGGAC |
| | | ACCGCCGTGTACTACTGCACCAGGCAGAGGTACAGGGGCCCCAAGTACTACTAC |
| | | TACATGGACGTGTGGGGCAAGGGCACCACCGTGACCGTGAGCAGC |
| 559C-M0180- | Light/ | GACATCCAGATGACCCAGAGCCCCCTGAGCCTGCCCGTGACCCCCGGCGAGCCC |
| G03 | SEQ ID | GCCAGCATCAGCTGCAGGAGCAGCCAGAGCCTGCTGCACAGCAACGGCTACTAC |
| | NO: 58 | CTGGACTGGTACCTGCAGAAGCCCGGCCAGAGCCCCCAGATCATGATCTACCTG |
| | | GGCAGCAACAGGGCCAGCGGCGTGCCCGACAGGTTCAGCGGCAGCGGCAGCGGC |
| | | ACCGACTTCACCCTGAAGATCAGCAGGGTGGAGGCCGAGGACGTGGGCGTGTAC |
| | | TACTGCATGCAGGCCCTGCAGACCCCCAGGACCTTCGGCCAGGGCACCAAGGTG |
| | | GAGATCAAGAGG |
| 559C-M0184- | Heavy/ | GAGGTGCAGCTGCTGGAGAGCGGCGGCGGCCTGGTGCAGCCCGGCGGCAGCCTG |
| B04 | SEQ ID | AGGCTGAGCTGCGCCGCCAGCGGCTTCACCTTCAGCTTCTACAGCATGCACTGG |
| | NO: 59 | GTGAGGCAGGCCCCCGGCAAGGGCCTGGAGTGGGTGAGCAGGATCTACCCCAGC |
| | | GGCGGCGTGACCAAGTACGCCGACAGCGTGAAGGGCAGGTTCACCATCAGCAGG |
| | | GACAACAGCAAGAACACCCTGTACCTGCAGATGAACAGCCTGAGGGCCGAGGAC |
| | | ACCGCCGTGTACTACTGCACCAGGCAGAGGTACAGGGGCCCCAAGTACTACTAC |
| | | TACATGGACGTGTGGGGCAAGGGCACCACCGTGACCGTGAGCAGC |
| 559C-M0184- | Light/ | GACATCCAGATGACCCAGAGCCCCCTGAGCCTGCCCGTGACCCCCGGCGAGCCC |
| B04 | SEQ ID | GCCAGCATCAGCTGCAGGAGCAGCCAGAGCCTGCTGCACAGCAACGGCTACTAC |
| | NO: 55 | CTGGACTGGTACCTGCAGAAGCCCGGCCAGAGCCCCCAGCTGCTGATCTACCTG |
| | | GGCAGCAACAGGGCCAGCGGCGTGCCCGACAGGTTCAGCGGCAGCGGCAGCGGC |
| | | ACCGACTTCACCCTGAAGATCAGCAGGGTGGAGGCCGAGGACGTGGGCGTGTAC |
| | | TACTGCATGCAGGCCCTGCAGACCCCCTGGACCTTCGGCCAGGGCACCAAGGTG |
| | | GAGATCAAGAGG |

Transgenes

Provided are recombinant vectors containing a transgene encoding a HuPTM mAb or HuPTM Fab (or other antigen binding fragment of the HuPTM mAb) that binds to factor XII 1 that can be administered to deliver the HuPTM mAb or antigen binding fragment in a patient. The transgene is a nucleic acid comprising the nucleotide sequences encoding an antigen binding fragment of an antibody that binds to factor XII, such as garadacimab, AB042/AB043, AB054, DX-4012, Ab26036, Ab26048, Ab260489, Ab26076, 559C-M0071-F06, 559C-M0179-D04, 559C-M0181-C02, 559C-M0180-G03, 559C-M0184-B04, 620I-X0173-A11, 620I-X0173-C07, 620I-X0173-E07, or 620I-X0173-G11, or variants thereof as detailed herein. The transgene may also encode an anti-factor XII antigen binding fragment that contains additional glycosylation sites (e.g., see Courtois et al.).

Provided are also recombinant vectors containing a transgene encoding a bi-specific HuPTM antibody that binds to pKal and factor XII that can be administered to deliver the HuPTM antibody in a patient. The transgene is a nucleic acid comprising a first and a second nucleotide sequence encoding the heavy or light chain (including variable and constant regions) of an anti-pKal antibody (e.g. DX-2930) and a single chain format of an anti-factor XII antibody, such as garadacimab, AB042/AB043, AB054, DX-4012, Ab26036, Ab26048, Ab260489, Ab26076, 559C-M0071-F06, 559C-M0179-D04, 559C-M0181-C02, 559C-M0180-G03, 559C-M0184-B04, 620I-X0173-A11, 620I-X0173-C07, 620I-X0173-E07, or 620I-X0173-G11, wherein the anti-factor XII antibody is fused to the C-terminus of the heavy or light chain of the anti-pKal antibody, or variants thereof as detailed herein. Alternatively, the anti-pKal can be the single chain format fused to the C-terminus of the heavy or light chain of the anti-Factor XII antibody. The transgene may also encode a bi-specific antibody that contains additional glycosylation sites (e.g., see Courtois et al.). Each antibody portion in the bispecific antibody as described herein can be an antibody in any form, including, but not limited to, intact (i.e., full-length) antibodies, or an antigen-binding fragments thereof (such as Fab, Fab′, F(ab′)2, Fv), single chain antibodies (scFv antibodies), and tetravalent antibodies.

In certain embodiments, the anti-factor XII antigen-binding fragment transgene comprises the nucleotide sequences encoding the heavy and light chains of the Fab portion of garadacimab (having amino acid sequences of SEQ ID NOs: 1 and 2, respectively, see Table 7 and FIG. 2A). The nucleotide sequences may be codon optimized for expression in human cells. Nucleotide sequences may, for example, comprise the nucleotide sequences of SEQ ID NO:32 (encoding the garadacimab heavy chain Fab portion) and SEQ ID NO:33 (encoding the garadacimab light chain Fab portion) as set forth in Table 7. The heavy and light chain sequences both have a signal or leader sequence at the N-terminus appropriate for expression and secretion in human cells, in particular, human liver cells (e.g., hepatocytes) or muscle cells. The signal sequence may have the amino acid sequence of MYRMQLLLLIALSLALVTNS (SEQ ID NO:103). Alternatively, the signal sequence may have an amino acid sequence selected from any one of the signal sequences set forth in Table 2 or 3 that correspond to the proteins secreted by myocytes or hepatocytes, respectively.

In addition to the heavy and light chain variable domain and CH1 and CL domain sequences, the transgenes may comprise, at the C-terminus of the heavy chain CH1 domain sequence, all or a portion of the hinge region. In specific embodiments, the anti-factor XII-antigen binding domain has a heavy chain Fab domain of SEQ ID NO: 2 with additional hinge region sequence starting after the C-terminal valine (V), contains all or a portion of the amino acid sequence ESKYGPPCPPCPAPEFLGG (SEQ ID NO:184), and specifically, ESKYGPPCPPCPA (SEQ ID NO:185), ESKYGPPCPSCPA (SEQ ID NO:186), ESKYGPPCPSCPAPEFLGGPSVFL (SEQ ID NO:187), or ESKYGPPCPPCPAPEFLGGPSVFL (SEQ ID NO:188) as set forth in Table 5 and FIG. 2A. These hinge regions may be encoded by nucleotide sequences at the 3′ end of SEQ ID NO:32 by the hinge region encoding sequences set forth in Table 7. In another embodiment, the transgenes comprise the amino acid sequences encoding the full length (or substantially full length) heavy and light chains of the antibody, comprising the Fc domain at the C terminus of the heavy chain, e.g. having an amino acid sequence of SEQ ID NO:63 (Table 6) or an IgG4 Fc domain, such as SEQ ID NO:62 or as depicted in Table 6, or a mutant or variant thereof. The Fc domain may be engineered for altered binding to one or more Fc receptors and/or effector function as disclosed in Section 5.1.9, infra.

In certain embodiments, the anti-factor XII antigen-binding fragment transgene encodes an factor XII antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO:2. In certain embodiments, the anti-factor XII antigen-binding fragment transgene encodes an factor XII antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO:1. In certain embodiments, the anti-factor XII antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO:2 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO:1. In specific embodiments, the factor XII antigen binding fragment comprises a heavy chain comprising an amino acid sequence of SEQ ID NO:1 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 2A). In specific embodiments, the factor XII antigen binding fragment comprises a light chain comprising an amino acid sequence of SEQ ID NO:2 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 2A).

In certain embodiments, the anti-factor XII antigen-binding fragment transgene encodes an antigen-binding fragment and comprises the nucleotide sequences encoding the six garadacimab CDRs which are underlined in the heavy and light chain variable domain sequences of FIG. 2A which are spaced between framework regions, generally human framework regions, and associated with constant domains depending upon the form of the antigen-binding molecule, as is known in the art to form the heavy and/or light chain variable domain of an anti-factor XII antibody or antigen-binding fragment thereof.

In certain embodiments, the anti-factor XII antigen-binding fragment transgene comprises the nucleotide sequences encoding the heavy and light chains of the Fab portion of AB054 (having amino acid sequences of SEQ ID NOs:5 and 6, respectively, see Table 7 and FIG. 2C). The nucleotide sequences may be codon optimized for expression in human cells. Nucleotide sequences may, for example, comprise the nucleotide sequences of SEQ ID NO:36 (encoding the AB054 heavy chain Fab portion) and SEQ ID NO:37 (encoding the AB054 light chain Fab portion) as set forth in Table 7. The heavy and light chain sequences both have a signal or leader sequence at the N-terminus appropriate for expression and secretion in human cells, in particular, human liver cells (e.g., hepatocytes) or muscle cells. The signal sequence may have the amino acid sequence of MYRMQLLLLIALSLALVTNS (SEQ ID NO:103). Alternatively, the signal sequence may have an amino acid sequence selected from any one of the signal sequences set forth in Table 2 or 3 that correspond to the proteins secreted by myocytes or hepatocytes, respectively.

In addition to the heavy and light chain variable domain and CH1 and CL domain sequences, the transgenes may comprise, at the C-terminus of the heavy chain CH1 domain sequence, all or a portion of the hinge region. In specific embodiments, the anti-factor XII-antigen binding domain has a heavy chain Fab domain of SEQ ID NO:5 with additional hinge region sequence starting after the C-terminal valine (V), contains all or a portion of the amino acid sequence ESKYGPPCPPCPAPEFLGG (SEQ ID NO:184), and specifically, ESKYGPPCPPCPA (SEQ ID NO:185), ESKYGPPCPSCPA (SEQ ID NO:186), ESKYGPPCPSCPAPEFLGGPSVFL (SEQ ID NO:187), or ESKYGPPCPPCPAPEFLGGPSVFL (SEQ ID NO:188) as set forth in Table 5 and FIG. 2C. These hinge regions may be encoded by nucleotide sequences at the 3′ end of SEQ ID NO:36 by the hinge region encoding sequences set forth in Table 7. In another embodiment, the transgenes comprise the amino acid sequences encoding the full length (or substantially full length) heavy and light chains of the antibody, comprising the Fc domain at the C terminus of the heavy chain, e.g. having an amino acid sequence of SEQ ID NO:64 (Table 6) or an IgG4 Fc domain, such as SEQ ID NO:62 or as depicted in Table 6, or a mutant or variant thereof. The Fc domain may be engineered for altered binding to one or more Fc receptors and/or effector function as disclosed in Section 5.1.9, infra.

In certain embodiments, the anti-factor XII antigen-binding fragment transgene encodes an factor XII antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO:6. In certain embodiments, the anti-factor XII antigen-binding fragment transgene encodes an factor XII antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO:5. In certain embodiments, the anti-factor XII antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO:6 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO:5. In specific embodiments, the factor XII antigen binding fragment comprises a heavy chain comprising an amino acid sequence of SEQ ID NO:1 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 2C). In specific embodiments, the factor XII antigen binding fragment comprises a light chain comprising an amino acid sequence of SEQ ID NO:6 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 2C).

In certain embodiments, the anti-factor XII antigen-binding fragment transgene encodes an antigen-binding fragment and comprises the nucleotide sequences encoding the six AB054 CDRs which are underlined in the heavy and light chain variable domain sequences of FIG. 2C which are spaced between framework regions, generally human framework regions, and associated with constant domains depending upon the form of the antigen-binding molecule, as is known in the art to form the heavy and/or light chain variable domain of an anti-factor XII antibody or antigen-binding fragment thereof.

In certain embodiments, the anti-factor XII antigen-binding fragment transgene comprises the nucleotide sequences encoding the heavy and light chains of the Fab portion of DX-4012 (having amino acid sequences of SEQ ID NOs:7 and 8, respectively, see Table 7 and FIG. 2D). The nucleotide sequences may be codon optimized for expression in human cells. Nucleotide sequences may, for example, comprise the nucleotide sequences of SEQ ID NO:38 (encoding the DX-4012 heavy chain Fab portion) and SEQ ID NO:39 (encoding the DX-4012 light chain Fab portion) as set forth in Table 7. The heavy and light chain sequences both have a signal or leader sequence at the N-terminus appropriate for expression and secretion in human cells, in particular, human liver cells (e.g., hepatocytes) or muscle cells. The signal sequence may have the amino acid sequence of MYRMQLLLLIALSLALVTNS (SEQ ID NO:103). Alternatively, the signal sequence may have an amino acid sequence selected from any one of the signal sequences set forth in Table 2 or 3 that correspond to the proteins secreted by myocytes or hepatocytes, respectively.

In addition to the heavy and light chain variable domain and CH1 and CL domain sequences, the transgenes may comprise, at the C-terminus of the heavy chain CH1 domain sequence, all or a portion of the hinge region. In specific embodiments, the anti-factor XII-antigen binding domain has a heavy chain Fab domain of SEQ ID NO:7 with additional hinge region sequence starting after the C-terminal valine (V), contains all or a portion of the amino acid sequence EPKSCDKTHTCPPCPAPELLGG (SEQ ID NO:171), and specifically, EPKSCDKTHL (SEQ ID NO:173), EPKSCDKTHT (SEQ ID NO:174), EPKSCDKTHTCPPCPA (SEQ ID NO:175), EPKSCDKTHLCPPCPA (SEQ ID NO:176), EPKSCDKTHTCPPCPAPELLGGPSVFL (SEQ ID NO:177) or EPKSCDKTHLCPPCPAPELLGGPSVFL (SEQ ID NO:178) as set forth in Table 5 and FIG. 2D. These hinge regions may be encoded by nucleotide sequences at the 3′ end of SEQ ID NO:38 by the hinge region encoding sequences set forth in Table 7. In another embodiment, the transgenes comprise the amino acid sequences encoding the full length (or substantially full length) heavy and light chains of the antibody, comprising the Fc domain at the C terminus of the heavy chain, e.g. having an amino acid sequence of SEQ ID NO:65 (Table 6) or an IgG1 Fc domain, such as SEQ ID NO:60 or as depicted in Table 6, or a mutant or variant thereof. The Fc domain may be engineered for altered binding to one or more Fc receptors and/or effector function as disclosed in Section 5.1.9, infra.

In certain embodiments, the anti-factor XII antigen-binding fragment transgene encodes an factor XII antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO:8. In certain embodiments, the anti-factor XII antigen-binding fragment transgene encodes an factor XII antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO:7. In certain embodiments, the anti-factor XII antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 8 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO:7. In specific embodiments, the factor XII antigen binding fragment comprises a heavy chain comprising an amino acid sequence of SEQ ID NO:1 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 2D). In specific embodiments, the factor XII antigen binding fragment comprises a light chain comprising an amino acid sequence of SEQ ID NO:8 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 2D).

In certain embodiments, the anti-factor XII antigen-binding fragment transgene encodes an antigen-binding fragment and comprises the nucleotide sequences encoding the six DX-4012 CDRs which are underlined in the heavy and light chain variable domain sequences of FIG. 2D which are spaced between framework regions, generally human framework regions, and associated with constant domains depending upon the form of the antigen-binding molecule, as is known in the art to form the heavy and/or light chain variable domain of an anti-factor XII antibody or antigen-binding fragment thereof.

In certain embodiments, the anti-factor XII antigen-binding fragment transgene comprises the nucleotide sequences encoding the heavy and light chain variable domains of AB042/AB043 (having amino acid sequences of SEQ ID NOs:3 and 4, respectively, see Table 7 and FIG. 2B). The nucleotide sequences may be codon optimized for expression in human cells. Nucleotide sequences may, for example, comprise the nucleotide sequences of SEQ ID NO:34 (encoding the AB042/043 heavy chain variable domain) and SEQ ID NO:35 (encoding the AB042/43 light chain variable domain) as set forth in Table 7. The heavy and light chain sequences both have a signal or leader sequence at the N-terminus appropriate for expression and secretion in human cells, in particular, human liver cells (e.g., hepatocytes) or muscle cells. The signal sequence may have the amino acid sequence of MYRMQLLLLIALSLALVTNS (SEQ ID NO:103). Alternatively, the signal sequence may have an amino acid sequence selected from any one of the signal sequences set forth in Table 2 or 3 that correspond to the proteins secreted by myocytes or hepatocytes, respectively. The anti-factor XII antigen-binding fragment will also comprise a CH1 domain at the C-terminus of the heavy chain variable domain and a CL domain at the C-terminus of the light chain variable domain (e.g. see Table 7 for specific sequences).

In addition to the heavy and light chain variable domain and CH1 and CL domain sequences, the transgenes may comprise, at the C-terminus of the heavy chain CH1 domain sequence, all or a portion of the hinge region. In specific embodiments, the anti-factor XII-antigen binding domain has a heavy chain variable domain of SEQ ID NO:3 and a CH1 domain, with additional hinge region sequence as set forth in Table 5. In another embodiment, the transgenes comprise the amino acid sequences encoding the full length (or substantially full length) heavy and light chains of the antibody, comprising the Fc domain at the C terminus of the heavy chain, e.g. having an amino acid sequence of an IgG1, IgG2, or IgG4 Fc domain, such as SEQ ID NO:60-62 or as depicted in Table 6, or a mutant or variant thereof. The Fc domain may be engineered for altered binding to one or more Fc receptors and/or effector function as disclosed in Section 5.1.9, infra.

In certain embodiments, the anti-factor XII antigen-binding fragment transgene encodes an factor XII antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO:4. In certain embodiments, the anti-factor XII antigen-binding fragment transgene encodes an factor XII antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO:3. In certain embodiments, the anti-factor XII antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO:4 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO:3. In specific embodiments, the factor XII antigen binding fragment comprises a heavy chain comprising an amino acid sequence of SEQ ID NO:3 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 2B). In specific embodiments, the factor XII antigen binding fragment comprises a light chain comprising an amino acid sequence of SEQ ID NO:4 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 2B).

In certain embodiments, the anti-factor XII antigen-binding fragment transgene encodes an antigen-binding fragment and comprises the nucleotide sequences encoding the six AB042/43 CDRs which are underlined in the heavy and light chain variable domain sequences of FIG. 2B which are spaced between framework regions, generally human framework regions, and associated with constant domains depending upon the form of the antigen-binding molecule, as is known in the art to form the heavy and/or light chain variable domain of an anti-factor XII antibody or antigen-binding fragment thereof.

In certain embodiments, the anti-factor XII antigen-binding fragment transgene comprises the nucleotide sequences encoding the heavy and light chain variable domains of ab26036 (having amino acid sequences of SEQ ID NOs:10 and 11, respectively, see Table 7 and FIG. 2E). The nucleotide sequences may be codon optimized for expression in human cells. Nucleotide sequences may, for example, comprise the nucleotide sequences of SEQ ID NO:41 (encoding the ab26036 heavy chain variable domain) and SEQ ID NO:42 (encoding the ab26036 light chain variable domain) as set forth in Table 7. The heavy and light chain sequences both have a signal or leader sequence at the N-terminus appropriate for expression and secretion in human cells, in particular, human liver cells (e.g., hepatocytes) or muscle cells. The signal sequence may have the amino acid sequence of MYRMQLLLLIALSLALVTNS (SEQ ID NO:103). Alternatively, the signal sequence may have an amino acid sequence selected from any one of the signal sequences set forth in Table 2 or 3 that correspond to the proteins secreted by myocytes or hepatocytes, respectively. The anti-factor XII antigen-binding fragment will also comprise a CH1 domain at the C-terminus of the heavy chain variable domain and a CL domain at the C-terminus of the light chain variable domain (e.g. see Table 7 for specific sequences).

In addition to the heavy and light chain variable domain and CH1 and CL domain sequences, the transgenes may comprise, at the C-terminus of the heavy chain CH1 domain sequence, all or a portion of the hinge region. In specific embodiments, the anti-factor XII-antigen binding domain has a heavy chain variable domain of SEQ ID NO:10 and a CH1 domain, with additional hinge region sequence as set forth in Table 5. In another embodiment, the transgenes comprise the amino acid sequences encoding the full length (or substantially full length) heavy and light chains of the antibody, comprising the Fc domain at the C terminus of the heavy chain, e.g. having an amino acid sequence of an IgG1, IgG2, or IgG4 Fc domain, such as SEQ ID NOs:60-62 or as depicted in Table 6, or a mutant or variant thereof. The Fc domain may be engineered for altered binding to one or more Fc receptors and/or effector function as disclosed in Section 5.1.9, infra.

In certain embodiments, the anti-factor XII antigen-binding fragment transgene encodes an factor XII antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO:11. In certain embodiments, the anti-factor XII antigen-binding fragment transgene encodes an factor XII antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO:10. In certain embodiments, the anti-factor XII antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO:11 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO:10. In specific embodiments, the factor XII antigen binding fragment comprises a heavy chain comprising an amino acid sequence of SEQ ID NO:10 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 2E). In specific embodiments, the factor XII antigen binding fragment comprises a light chain comprising an amino acid sequence of SEQ ID NO:11 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 2E).

In certain embodiments, the anti-factor XII antigen-binding fragment transgene encodes an antigen-binding fragment and comprises the nucleotide sequences encoding the six ab26036 CDRs which are underlined in the heavy and light chain variable domain sequences of FIG. 2E which are spaced between framework regions, generally human framework regions, and associated with constant domains depending upon the form of the antigen-binding molecule, as is known in the art to form the heavy and/or light chain variable domain of an anti-factor XII antibody or antigen-binding fragment thereof.

In certain embodiments, the anti-factor XII antigen-binding fragment transgene comprises the nucleotide sequences encoding the heavy and light chain variable domains of ab26048 (having amino acid sequences of SEQ ID NOs:12 and 13, respectively, see Table 7 and FIG. 2F). The nucleotide sequences may be codon optimized for expression in human cells. Nucleotide sequences may, for example, comprise the nucleotide sequences of SEQ ID NO:43 (encoding the ab26048 heavy chain variable domain) and SEQ ID NO:44 (encoding the ab26048 light chain variable domain) as set forth in Table 7. The heavy and light chain sequences both have a signal or leader sequence at the N-terminus appropriate for expression and secretion in human cells, in particular, human liver cells (e.g., hepatocytes) or muscle cells. The signal sequence may have the amino acid sequence of MYRMQLLLLIALSLALVTNS (SEQ ID NO:103). Alternatively, the signal sequence may have an amino acid sequence selected from any one of the signal sequences set forth in Table 2 or 3 that correspond to the proteins secreted by myocytes or hepatocytes, respectively. The anti-factor XII antigen-binding fragment will also comprise a CH1 domain at the C-terminus of the heavy chain variable domain and a CL domain at the C-terminus of the light chain variable domain (e.g. see Table 7 for specific sequences).

In addition to the heavy and light chain variable domain and CH1 and CL domain sequences, the transgenes may comprise, at the C-terminus of the heavy chain CH1 domain sequence, all or a portion of the hinge region. In specific embodiments, the anti-factor XII-antigen binding domain has a heavy chain variable domain of SEQ ID NO:12 and a CH1 domain, with additional hinge region sequence as set forth in Table 5. In another embodiment, the transgenes comprise the amino acid sequences encoding the full length (or substantially full length) heavy and light chains of the antibody, comprising the Fc domain at the C terminus of the heavy chain, e.g. having an amino acid sequence of an IgG1, IgG2, or IgG4 Fc domain, such as SEQ ID NOs:60-62 or as depicted in Table 6, or a mutant or variant thereof. The Fc domain may be engineered for altered binding to one or more Fc receptors and/or effector function as disclosed in Section 5.1.9, infra.

In certain embodiments, the anti-factor XII antigen-binding fragment transgene encodes an factor XII antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO:13. In certain embodiments, the anti-factor XII antigen-binding fragment transgene encodes an factor XII antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO:12. In certain embodiments, the anti-factor XII antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO:13 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO:12. In specific embodiments, the factor XII antigen binding fragment comprises a heavy chain comprising an amino acid sequence of SEQ ID NO: 12 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 2F). In specific embodiments, the factor XII antigen binding fragment comprises a light chain comprising an amino acid sequence of SEQ ID NO:13 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 2F).

In certain embodiments, the anti-factor XII antigen-binding fragment transgene encodes an antigen-binding fragment and comprises the nucleotide sequences encoding the six ab26048 CDRs which are underlined in the heavy and light chain variable domain sequences of FIG. 2F which are spaced between framework regions, generally human framework regions, and associated with constant domains depending upon the form of the antigen-binding molecule, as is known in the art to form the heavy and/or light chain variable domain of an anti-factor XII antibody or antigen-binding fragment thereof.

In certain embodiments, the anti-factor XII antigen-binding fragment transgene comprises the nucleotide sequences encoding the heavy and light chain variable domains of ab26049 (having amino acid sequences of SEQ ID NOs:14 and 15, respectively, see Table 7 and FIG. 2G). The nucleotide sequences may be codon optimized for expression in human cells. Nucleotide sequences may, for example, comprise the nucleotide sequences of SEQ ID NO:45 (encoding the ab26049 heavy chain variable domain) and SEQ ID NO:46 (encoding the ab26049 light chain variable domain) as set forth in Table 7. The heavy and light chain sequences both have a signal or leader sequence at the N-terminus appropriate for expression and secretion in human cells, in particular, human liver cells (e.g., hepatocytes) or muscle cells. The signal sequence may have the amino acid sequence of MYRMQLLLLIALSLALVTNS (SEQ ID NO:103). Alternatively, the signal sequence may have an amino acid sequence selected from any one of the signal sequences set forth in Table 2 or 3 that correspond to the proteins secreted by myocytes or hepatocytes, respectively. The anti-factor XII antigen-binding fragment will also comprise a CH1 domain at the C-terminus of the heavy chain variable domain and a CL domain at the C-terminus of the light chain variable domain (e.g. see Table 7 for specific sequences).

In addition to the heavy and light chain variable domain and CH1 and CL domain sequences, the transgenes may comprise, at the C-terminus of the heavy chain CH1 domain sequence, all or a portion of the hinge region. In specific embodiments, the anti-factor XII-antigen binding domain has a heavy chain variable domain of SEQ ID NO:14 and a CH1 domain, with additional hinge region sequence as set forth in Table 5. In another embodiment, the transgenes comprise the amino acid sequences encoding the full length (or substantially full length) heavy and light chains of the antibody, comprising the Fc domain at the C terminus of the heavy chain, e.g. having an amino acid sequence of an IgG1, IgG2, or IgG4 Fc domain, such as SEQ ID NOs:60-62 or as depicted in Table 6, or a mutant or variant thereof. The Fc domain may be engineered for altered binding to one or more Fc receptors and/or effector function as disclosed in Section 5.1.9, infra.

In certain embodiments, the anti-factor XII antigen-binding fragment transgene encodes an factor XII antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO:15. In certain embodiments, the anti-factor XII antigen-binding fragment transgene encodes an factor XII antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO:14. In certain embodiments, the anti-factor XII antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 15 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO:14. In specific embodiments, the factor XII antigen binding fragment comprises a heavy chain comprising an amino acid sequence of SEQ ID NO:14 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 2G). In specific embodiments, the factor XII antigen binding fragment comprises a light chain comprising an amino acid sequence of SEQ ID NO:15 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 2G).

In certain embodiments, the anti-factor XII antigen-binding fragment transgene encodes an antigen-binding fragment and comprises the nucleotide sequences encoding the six ab26049 CDRs which are underlined in the heavy and light chain variable domain sequences of FIG. 2G which are spaced between framework regions, generally human framework regions, and associated with constant domains depending upon the form of the antigen-binding molecule, as is known in the art to form the heavy and/or light chain variable domain of an anti-factor XII antibody or antigen-binding fragment thereof.

In certain embodiments, the anti-factor XII antigen-binding fragment transgene comprises the nucleotide sequences encoding the heavy and light chain variable domains of ab26076 (having amino acid sequences of SEQ ID NOs:16 and 17, respectively, see Table 7 and FIG. 2H). The nucleotide sequences may be codon optimized for expression in human cells. Nucleotide sequences may, for example, comprise the nucleotide sequences of SEQ ID NO:47 (encoding the ab26076 heavy chain variable domain) and SEQ ID NO:48 (encoding the ab26076 light chain variable domain) as set forth in Table 7. The heavy and light chain sequences both have a signal or leader sequence at the N-terminus appropriate for expression and secretion in human cells, in particular, human liver cells (e.g., hepatocytes) or muscle cells. The signal sequence may have the amino acid sequence of MYRMQLLLLIALSLALVTNS (SEQ ID NO:103). Alternatively, the signal sequence may have an amino acid sequence selected from any one of the signal sequences set forth in Table 2 or 3 that correspond to the proteins secreted by myocytes or hepatocytes, respectively. The anti-factor XII antigen-binding fragment will also comprise a CH1 domain at the C-terminus of the heavy chain variable domain and a CL domain at the C-terminus of the light chain variable domain (e.g. see Table 7 for specific sequences).

In addition to the heavy and light chain variable domain and CH1 and CL domain sequences, the transgenes may comprise, at the C-terminus of the heavy chain CH1 domain sequence, all or a portion of the hinge region. In specific embodiments, the anti-factor XII-antigen binding domain has a heavy chain variable domain of SEQ ID NO:16 and a CH1 domain, with additional hinge region sequence as set forth in Table 5. In another embodiment, the transgenes comprise the amino acid sequences encoding the full length (or substantially full length) heavy and light chains of the antibody, comprising the Fc domain at the C terminus of the heavy chain, e.g. having an amino acid sequence of an IgG1, IgG2, or IgG4 Fc domain, such as SEQ ID NOs:60-62 or as depicted in Table 6, or a mutant or variant thereof. The Fc domain may be engineered for altered binding to one or more Fc receptors and/or effector function as disclosed in Section 5.1.9, infra.

In certain embodiments, the anti-factor XII antigen-binding fragment transgene encodes an factor XII antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO:17. In certain embodiments, the anti-factor XII antigen-binding fragment transgene encodes an factor XII antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO:16. In certain embodiments, the anti-factor XII antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO:17 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO:16. In specific embodiments, the factor XII antigen binding fragment comprises a heavy chain comprising an amino acid sequence of SEQ ID NO:16 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 2H). In specific embodiments, the factor XII antigen binding fragment comprises a light chain comprising an amino acid sequence of SEQ ID NO:17 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 2H).

In certain embodiments, the anti-factor XII antigen-binding fragment transgene encodes an antigen-binding fragment and comprises the nucleotide sequences encoding the six ab26076 CDRs which are underlined in the heavy and light chain variable domain sequences of FIG. 2G which are spaced between framework regions, generally human framework regions, and associated with constant domains depending upon the form of the antigen-binding molecule, as is known in the art to form the heavy and/or light chain variable domain of an anti-factor XII antibody or antigen-binding fragment thereof.

In certain embodiments, the anti-factor XII antigen-binding fragment transgene comprises the nucleotide sequences encoding the heavy and light chain variable domains of 620I-X0173-A11 (having amino acid sequences of SEQ ID NOs:18 and 19, respectively, see Table 7 and FIG. 2I). The nucleotide sequences may be codon optimized for expression in human cells. Nucleotide sequences may, for example, comprise the nucleotide sequences of SEQ ID NO:49 (encoding the 620I-X0173-A11 heavy chain variable domain) and SEQ ID NO:50 (encoding the 620I-X0173-A11 light chain variable domain) as set forth in Table 7. The heavy and light chain sequences both have a signal or leader sequence at the N-terminus appropriate for expression and secretion in human cells, in particular, human liver cells (e.g., hepatocytes) or muscle cells. The signal sequence may have the amino acid sequence of MYRMQLLLLIALSLALVTNS (SEQ ID NO:103). Alternatively, the signal sequence may have an amino acid sequence selected from any one of the signal sequences set forth in Table 2 or 3 that correspond to the proteins secreted by myocytes or hepatocytes, respectively. The anti-factor XII antigen-binding fragment will also comprise a CH1 domain at the C-terminus of the heavy chain variable domain and a CL domain at the C-terminus of the light chain variable domain (e.g. see Table 7 for specific sequences).

In addition to the heavy and light chain variable domain and CH1 and CL domain sequences, the transgenes may comprise, at the C-terminus of the heavy chain CH1 domain sequence, all or a portion of the hinge region. In specific embodiments, the anti-factor XII-antigen binding domain has a heavy chain variable domain of SEQ ID NO:18 and a CH1 domain, with additional hinge region sequence as set forth in Table 5. In another embodiment, the transgenes comprise the amino acid sequences encoding the full length (or substantially full length) heavy and light chains of the antibody, comprising the Fc domain at the C terminus of the heavy chain, e.g. having an amino acid sequence of an IgG1, IgG2, or IgG4 Fc domain, such as SEQ ID NOs:60-62 or as depicted in Table 6, or a mutant or variant thereof. The Fc domain may be engineered for altered binding to one or more Fc receptors and/or effector function as disclosed in Section 5.1.9, infra.

In certain embodiments, the anti-factor XII antigen-binding fragment transgene encodes an factor XII antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO:19. In certain embodiments, the anti-factor XII antigen-binding fragment transgene encodes an factor XII antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO:18. In certain embodiments, the anti-factor XII antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO:19 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO:16. In specific embodiments, the factor XII antigen binding fragment comprises a heavy chain comprising an amino acid sequence of SEQ ID NO:18 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 2I). In specific embodiments, the factor XII antigen binding fragment comprises a light chain comprising an amino acid sequence of SEQ ID NO:19 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 2I).

In certain embodiments, the anti-factor XII antigen-binding fragment transgene encodes an antigen-binding fragment and comprises the nucleotide sequences encoding the six 620I-X0173-A11 CDRs which are underlined in the heavy and light chain variable domain sequences of FIG. 2I which are spaced between framework regions, generally human framework regions, and associated with constant domains depending upon the form of the antigen-binding molecule, as is known in the art to form the heavy and/or light chain variable domain of an anti-factor XII antibody or antigen-binding fragment thereof.

Gene Therapy Methods

Provided are methods of treating human subjects for angioedema by administration of a viral vector containing a transgene encoding an anti-Factor XII antibody, or antigen binding fragment thereof. The antibody may be garadacimab, AB042/AB043, AB054, DX-4012, Ab26036, Ab26048, Ab260489, Ab26076, 559C-M0071-F06, 559C-M0179-D04, 559C-M0181-C02, 559C-M0180-G03, 559C-M0184-B04, 620I-X0173-A11, 620I-X0173-C07, 620I-X0173-E07, or 620I-X0173-G11 and is, e.g., a full length or substantially full length antibody or Fab fragment thereof, or other antigen-binding fragment thereof. In embodiments, the patient has been diagnosed with and/or has symptoms associated with angioedema. In other embodiments, the patient has hypercoagulation or thrombosis or thrombotic disease. Recombinant vectors used for delivering the transgene are described in above and in Section 5.1 and exemplary transgenes are provided above. Such vectors should have a tropism for human liver or muscle cells and can include non-replicating rAAV, particularly those bearing an AAV8 capsid. The recombinant vectors, such as shown in FIGS. 2A-J, can be administered in any manner such that the recombinant vector enters the liver tissue, e.g., by introducing the recombinant vector into the bloodstream, for example by intravenous or intramuscular administration. See Section for details regarding the methods of treatment.

The example provide results of serum levels of lanadelumab in mice, rats and non-human primates administered AAV vectors encoding full length lanadelumab to assess different promoters and other regulatory elements, linkers, AAV types, modes of administration, etc. Such results inform dosage of a recombinant AAV vector encoding an anti-Factor XII antibody to achieve serum levels, particularly, steady state serum levels, sufficient for therapeutic efficacy. Steady state serum levels of sufficient therapeutic efficacy may be determined through clinical studies or in animal studies, for example, as disclosed in the examples herein. In particular embodiments, the AAV8 Factor XII antibody vector is administered to a patient in need thereof, for example, a patient diagnosed with or suffering from HAE or hypercoagulation or thrombosis, at a dosage (vector genomes) sufficient for to expression of therapeutically effective levels of anti-Factor XII antibody in the patient serum while minimizing side effects such as transaminitis or the development of anti-drug antibodies. In particular embodiments, the dosage s 1E11 vg/kg to 1E14 vg/kg, including 1E11 vg/kg, 1E12 vg/kg, 1E13 vg/kg, or 1E14 vg/kg. The methods of treatment provided herein reduce the incidence or severity of angioedema occurrences or attacks. In particular embodiments, the angioedema occurs in the skin, the gastrointestinal tract or the upper airway. In addition, the methods of treatment disclosed herein reduce hypercoagulation or thrombosis. The therapeutically effective amount may be determined as the amount that reduces the incidence or severity of angioedema occurrences or attacks, reduces the coagulability of a subject's blood or reduces the incidence of thrombosis in a subject.

Subjects to whom such gene therapy is administered can be those responsive to anti-Factor XII therapy. In certain embodiments, the methods encompass treating patients who have been diagnosed with angioedema or hypercoagulation or thrombosis, or have one or more symptoms associated therewith, and identified as responsive to treatment with an anti-Factor XII antibody or considered a good candidate for therapy with an anti-Factor XII antibody. In specific embodiments, the patients have previously been treated with anti-Factor XII antibody, and have been found to be responsive to anti-Factor XII antibody. To determine responsiveness, the anti-Factor XII antibody or antigen-binding fragment transgene product (e.g., produced in cell culture, bioreactors, etc.) may be administered directly to the subject.

Human Post Translationally Modified Antibodies

The production of the anti-Factor XII HuPTM mAb or HuPTM Fab, should result in a “biobetter” molecule for the treatment of angioedema, hypercoagulation or thrombosis accomplished via gene therapy—e.g., by administering a viral vector or other DNA expression construct encoding the anti-Factor XII HuPTM Fab, intravenously to human subjects (patients) diagnosed with or having one or more symptoms of angioedema, hypercoagulation or thrombosis, to create a permanent depot in the liver or muscle tissue that continuously supplies the fully-human post-translationally modified, e.g., human-glycosylated, sulfated transgene product produced by transduced liver or muscle cells.

In specific embodiments, the anti-Factor II HuPTM mAb or antigen-binding fragment thereof has heavy and light chains with the amino acid sequences of the heavy and light chain Fab portions of garadacimab, AB042/AB043, AB054, DX-4012, Ab26036, Ab26048, Ab260489, Ab26076, 559C-M0071-F06, 559C-M0179-D04, 559C-M0181-C02, 559C-M0180-G03, 559C-M0184-B04, 620I-X0173-A11, 620I-X0173-C07, 620I-X0173-E07, or 620I-X0173-G11, as set forth in FIGS. 2A-J. The figures indicate the glutamine (Q) glycosylation sites; asparaginal (N) glycosylation sites, non-consensus asparaginal (N) glycosylation sites; and tyrosine-O-sulfation sites (Y) are as indicated in the legend. In embodiments, the anti-Factor XII HuPTM mAb or antigen-binding fragment thereof does not contain detectable NeuGc moieties and/or does not contain detectable alpha-Gal moieties. In certain embodiments, the HuPTM mAb is a full length or substantially full length mAb with an Fc region.

In certain embodiments, the HuPTM mAb or Fab (or a hyperglycosylated derivative of either) is therapeutically effective and is at least 0.5%, 1% or 2% glycosylated and/or sulfated and may be at least 5%, 10% or even 50% or 100% glycosylated and/or sulfated. The goal of gene therapy treatment provided herein is to slow or arrest the progression of angioedema, reduce the levels of pain or discomfort for the patient, or reduce levels of autoreactive B cells and immunoglobulin producing plasma cells or reduce coagulation or thrombosis. Efficacy may be monitored by scoring the function, symptoms, or degree of inflammation in the affected tissue or area of the body, e.g., such as the skin, joints, kidneys, lungs, blood cells, heart, and brain. For example, efficacy can be monitored by assessing changes in attack severity or frequency or reduction in coagulation or thrombosis formation.

Combinations of delivery of the anti-Factor XII HuPTM mAb or antigen-binding fragment thereof, to the liver or muscle accompanied by delivery of other available treatments are encompassed by the methods provided herein. The additional treatments may be administered before, concurrently, or subsequent to the gene therapy treatment. Available treatments for angioedema that could be combined with the gene therapy provided herein include but are not limited to danazol, bradykinin receptor antagonist (e.g., icatibant), plasma kallikrein inhibitor (e.g., ecallantide), C1 esterase inhibitor, conestat alfa, anti-fibrinolytic agents (e.g., tranexamic acid), omalizumab, and fresh frozen plasma transfusions, antihistamines, and corticosteroids and administration with anti-Factor XII antibody agents.

5.4.1 Dose Administration of Anti-Factor XII Antibodies

Section 5.1. describes recombinant vectors that contain a transgene encoding a HuPTM mAb or HuPTM Fab (or other antigen binding fragment of the HuPTM mAb) that binds to Factor XII. Therapeutically effective doses of any such recombinant vector should be administered in any manner such that the recombinant vector enters the liver or muscle (e.g., skeletal muscle), e.g. by introducing the recombinant vector into the bloodstream. Alternatively, the vector may be administered directly to the liver through hepatic blood flow, e.g., via the suprahepatic veins or via the hepatic artery. In specific, embodiments, the vector is administered subcutaneously, intramuscularly or intravenously. Intramuscular, subcutaneous, intravenous or hepatic administration should result in expression of the soluble transgene product in cells of the liver or muscle. Alternatively, the vector may be administered directly to the liver through hepatic blood flow, e.g., via the suprahepatic veins or via the hepatic artery. The expression of the transgene encoding an anti-Factor XII antibody creates a permanent depot in liver and/or muscle of the patient that continuously supplies the anti-Factor XII HuPTM mAb, or antigen binding fragment of the anti-Factor XII mAb to the circulation of the subject.

In certain embodiments, the range of a therapeutically or prophylactically effective amount of an AAV gene therapy vector or pharmaceutical composition is 1E11 to 1E14 genome copies (gc)/kg, preferably between 1E11 to 1E13, and even more preferably 1E12. The dose ranges described herein are exemplary only and do not limit the dose ranges that can be selected.

In certain embodiments, intravenous administration of an AAV gene therapy vector encoding an anti-Factor XII antibody results in expression of a therapeutically effective level of transgene product expression in human serum at least 20, 30, 40, 50 or 60 days after administration.

However, in all cases because the transgene product is continuously produced, maintenance of lower concentrations can be effective. Notwithstanding, because the transgene product is continuously produced, maintenance of lower concentrations can be effective. The concentration of the transgene product can be measured in patient blood serum samples.

Pharmaceutical compositions suitable for intravenous, intramuscular, subcutaneous or hepatic administration comprise a suspension of the recombinant vector comprising the transgene encoding the anti-Factor XII antibody, or antigen-binding fragment thereof, in a formulation buffer comprising a physiologically compatible aqueous buffer. The formulation buffer can comprise one or more of a polysaccharide, a surfactant, polymer, or oil.

### 6. EXAMPLES

### Example 1: Garadacimab Fab cDNA-Based Vector

A garadacimab Fab cDNA-based vector is constructed comprising a transgene comprising nucleotide sequences encoding the Fab portion of the heavy and light chain sequences of garadacimab (amino acid sequences being SEQ ID NOs:1 and 2, respectively). The nucleotide sequence coding for the Fab portion of the heavy and light chain may be the nucleotide sequence of SEQ ID NOs:32 and 33, respectively. The transgene also comprises nucleotide sequences that encodes a signal peptide, e.g., MYRMQLLLLIALSLALVTNS (SEQ ID NO:103). The nucleotide sequences encoding the light chain and heavy chain are separated by IRES elements or 2A cleavage sites (See Table 4, particularly, SEQ ID NO:156 or 157) to create a bicistronic vector. The vector additionally includes a constitutive promoter, such as CB7, a tissue-specific promoter, such as a liver specific promoter, particularly ApoE.hAAT promoter (SEQ ID NO:78) an inducible promoter, such as a hypoxia-inducible promoter.

### Example 2: AB042/AB043 scFv cDNA-Based Vector

An AB042/AB043 svFv cDNA-based vector is constructed comprising a transgene comprising nucleotide sequences encoding the heavy chain variable domain and light chain variable domain sequences of AB042/AB043 (amino acid sequences being SEQ ID NOs:3 and 4, respectively). The nucleotide sequence coding for the variable domain of the heavy and light chain may be the nucleotide sequence of SEQ ID NOs:34 and 35, respectively. The transgene also comprises nucleotide sequences that encodes a signal peptide, e.g., MYRMQLLLLIALSLALVTNS (SEQ ID NO:103). The nucleotide sequences encoding the light chain and heavy chain are separated by IRES elements or 2A cleavage sites (See Table 4, particularly, SEQ ID NO:156 or 158) to create a bicistronic vector. The vector additionally includes a constitutive promoter, such as CB7, a tissue-specific promoter, such as a liver specific promoter, particularly ApoE.hAAT promoter (SEQ ID NO:78) an inducible promoter, such as a hypoxia-inducible promoter.

### Example 3: AB054 Fab cDNA-Based Vector

A AB054 Fab cDNA-based vector is constructed comprising a transgene comprising nucleotide sequences encoding the Fab portion of the heavy and light chain sequences of AB054 (amino acid sequences being SEQ ID NOs:5 and 6, respectively). The nucleotide sequence coding for the Fab portion of the heavy and light chain may be the nucleotide sequence of SEQ ID NOs:36 and 37, respectively. The transgene also comprises nucleotide sequences that encodes a signal peptide, e.g., MYRMQLLLLIALSLALVTNS (SEQ ID NO:103). The nucleotide sequences encoding the light chain and heavy chain are separated by IRES elements or 2A cleavage sites (See Table 4, particularly, SEQ ID NO:156 or 158) to create a bicistronic vector. The vector additionally includes a constitutive promoter, such as CB7, a tissue-specific promoter, such as a liver specific promoter, particularly ApoE.hAAT promoter (SEQ ID NO:78) an inducible promoter, such as a hypoxia-inducible promoter.

### Example 4: DX-4012 Fab cDNA-Based Vector

ADX-4012 Fab cDNA-based vector is constructed comprising a transgene comprising nucleotide sequences encoding the Fab portion of the heavy and light chain sequences of DX-4012 (amino acid sequences being SEQ ID NOs:7 and 8, respectively). The nucleotide sequence coding for the Fab portion of the heavy and light chain may be the nucleotide sequence of SEQ ID NOs:38 and 39, respectively. The transgene also comprises nucleotide sequences that encodes a signal peptide, e.g., MYRMQLLLLIALSLALVTNS (SEQ ID NO:103). The nucleotide sequences encoding the light chain and heavy chain are separated by IRES elements or 2A cleavage sites (See Table 4, particularly, SEQ ID NO:156 or 158) to create a bicistronic vector. The vector additionally includes a constitutive promoter, such as CB7, a tissue-specific promoter, such as a liver specific promoter, particularly ApoE.hAAT promoter (SEQ ID NO:78) an inducible promoter, such as a hypoxia-inducible promoter.

### Example 5: Ab26036 scFv cDNA-Based Vector

An ab26036 svFv cDNA-based vector is constructed comprising a transgene comprising nucleotide sequences encoding the heavy chain variable domain and light chain variable domain sequences of ab26036 (amino acid sequences being SEQ ID NOs:10 and 11, respectively). The nucleotide sequence coding for the variable domain of the heavy and light chain may be the nucleotide sequence of SEQ ID NOs:41 and 42, respectively. The transgene also comprises nucleotide sequences that encodes a signal peptide, e.g., MYRMQLLLLIALSLALVTNS (SEQ ID NO:103). The nucleotide sequences encoding the light chain and heavy chain are separated by IRES elements or 2A cleavage sites (See Table 4, particularly, SEQ ID NO:156 or 158) to create a bicistronic vector. The vector additionally includes a constitutive promoter, such as CB7, a tissue-specific promoter, such as a liver specific promoter, particularly ApoE.hAAT promoter (SEQ ID NO:78) an inducible promoter, such as a hypoxia-inducible promoter.

### Example 6: Ab26048 scFv cDNA-Based Vector

An ab26048 svFv cDNA-based vector is constructed comprising a transgene comprising nucleotide sequences encoding the heavy chain variable domain and light chain variable domain sequences of ab26048 (amino acid sequences being SEQ ID NOs:12 and 13, respectively). The nucleotide sequence coding for the variable domain of the heavy and light chain may be the nucleotide sequence of SEQ ID NOs:43 and 44, respectively. The transgene also comprises nucleotide sequences that encodes a signal peptide, e.g., MYRMQLLLLIALSLALVTNS (SEQ ID NO:103). The nucleotide sequences encoding the light chain and heavy chain are separated by IRES elements or 2A cleavage sites (See Table 4, particularly, SEQ ID NO:156 or 158) to create a bicistronic vector. The vector additionally includes a constitutive promoter, such as CB7, a tissue-specific promoter, such as a liver specific promoter, particularly ApoE.hAAT promoter (SEQ ID NO:78) an inducible promoter, such as a hypoxia-inducible promoter.

### Example 7: Ab26049 scFv cDNA-Based Vector

An ab26049 svFv cDNA-based vector is constructed comprising a transgene comprising nucleotide sequences encoding the heavy chain variable domain and light chain variable domain sequences of ab26049 (amino acid sequences being SEQ ID NOs:14 and 15, respectively). The nucleotide sequence coding for the variable domain of the heavy and light chain may be the nucleotide sequence of SEQ ID NOs:45 and 46, respectively. The transgene also comprises nucleotide sequences that encodes a signal peptide, e.g., MYRMQLLLLIALSLALVTNS (SEQ ID NO:103). The nucleotide sequences encoding the light chain and heavy chain are separated by IRES elements or 2A cleavage sites (See Table 4, particularly, SEQ ID NO:156 or 158) to create a bicistronic vector. The vector additionally includes a constitutive promoter, such as CB7, a tissue-specific promoter, such as a liver specific promoter, particularly ApoE.hAAT promoter (SEQ ID NO:78) an inducible promoter, such as a hypoxia-inducible promoter.

### Example 8: Ab26076 scFv cDNA-Based Vector

An ab26076 svFv cDNA-based vector is constructed comprising a transgene comprising nucleotide sequences encoding the heavy chain variable domain and light chain variable domain sequences of ab26076 (amino acid sequences being SEQ ID NOs:16 and 17, respectively). The nucleotide sequence coding for the variable domain of the heavy and light chain may be the nucleotide sequence of SEQ ID NOs:47 and 48, respectively. The transgene also comprises nucleotide sequences that encodes a signal peptide, e.g., MYRMQLLLLIALSLALVTNS (SEQ ID NO:103). The nucleotide sequences encoding the light chain and heavy chain are separated by IRES elements or 2A cleavage sites (See Table 4, particularly, SEQ ID NO:156 or 158) to create a bicistronic vector. The vector additionally includes a constitutive promoter, such as CB7, a tissue-specific promoter, such as a liver specific promoter, particularly ApoE.hAAT promoter (SEQ ID NO:78) an inducible promoter, such as a hypoxia-inducible promoter.

### Example 9: 620I-X0173-A01 scFv cDNA-Based Vector

An 620I-X0173-A01 svFv cDNA-based vector is constructed comprising a transgene comprising nucleotide sequences encoding the heavy chain variable domain and light chain variable domain sequences of 620I-X0173-A01 (amino acid sequences being SEQ ID NOs:18 and 19, respectively). The nucleotide sequence coding for the variable domain of the heavy and light chain may be the nucleotide sequence of SEQ ID NOs:49 and 50, respectively. The transgene also comprises nucleotide sequences that encodes a signal peptide, e.g., MYRMQLLLLIALSLALVTNS (SEQ ID NO:103). The nucleotide sequences encoding the light chain and heavy chain are separated by IRES elements or 2A cleavage sites (See Table 4, particularly, SEQ ID NO:156 or 158) to create a bicistronic vector. The vector additionally includes a constitutive promoter, such as CB7, a tissue-specific promoter, such as a liver specific promoter, particularly ApoE.hAAT promoter (SEQ ID NO:78) an inducible promoter, such as a hypoxia-inducible promoter.

### Example 10: Protein Expression Analysis of Lanadelumab in Cell Lysates and Supernatant

Cell culture studies were performed to assess the expression of full length mAb sequences (containing Fc region) from AAV constructs in human cells.

### Methods

A lanadelumab cDNA-based vector was constructed comprising a transgene comprising a nucleotide sequence encoding the heavy and light chain sequences of lanadelumab (amino acid sequences being SEQ ID NOs. 204 and 205, respectively). The nucleotide sequence coding for the heavy and light chain of lanadelumab was codon optimized to generate the three nucleotide sequences provided in Table 9 below, L01 (SEQ ID NO:218), L02 (SEQ ID NO:219), and L03 (SEQ ID NO:220). L02 and L03 also have reduced incidence of CpG dimers in the sequence. The transgene also comprised a nucleotide sequence that encodes the signal peptide MYRMQLLLLIALSLALVTNS (SEQ ID NO:103). The nucleotide sequences encoding the light chain and heavy chain were separated by a Furin-F2A linker (SEQ ID NOS:157 or 158) or a Furin T2A linker (SEQ ID NOS:155 or 156) to create a bicistronic vector. The vector additionally included a constitutive CAG promoter (SEQ ID NO:89). Sequences of the constructs are provided in Table 9 (SEQ ID NOS:221-229).

Table 1 above provides the sequences of composite nucleic acid regulatory sequences that may be incorporated into expression cassettes and be operably linked to the transgene to promote liver-specific expression (LSPX1, LSPX2, LTP1, LTP2, or LTP3, SEQ ID NOS:66-70, respectively) and liver and muscle expression (LMTP6, LMTP13, LMTP15, LMTP18, LMTP19 or LMTP20, SEQ ID NOS:71-76 respectively). Other promoter sequences provided, include the ApoE.hAAT (SEQ ID NO:78, Table 1 above) promoter, wherein four copies of the liver-specific apolipoprotein E (ApoE) enhancer were placed upstream of the human alpha 1-antitrypsin (hAAT) promoter).

TABLE 9

| Name/ | |
|---|---|
| SEQ ID NO. | Sequence |
| Lanadelumab | EVOLLESGGG LVQPGGSLRL SCAASGFTFS HYIMMWVRQA PGKGLEWVSG |
| Heavy/SEQ ID | IYSSGGITVY ADSVKGRFTI SRDNSKNTLY LOMNSLRAED TAVYYCAYRR |
| NO: 214 | IGVPRRDEFD IWGQGTMVTV SSASTKGPSV FPLAPSSKST SGGTAALGCL |
| | VKDYFPEPVT VSWNSGALTS GVHTFPAVLQ SSGLYSLSSV VTVPSSSLGT |
| | QTYICNVNHK PSNTKVDKRV EPKSCD +/− KTHT (or KTHL) +/− CPPCPA |
| | +/− PELLGGPSVFL |
| Lanadelumab | DIQMTQSPST LSASVGDRVT ITCRASQSIS SWLAWYQQKP GKAPKLLIYK |
| Light/SEQ ID | ASTLESGVPS RFSGSGSGTE FTLTISSLOP DDFATYYCQQ YNTYWTFGQG |
| NO: 215 | TKVEIKRTVA APSVFIFPPS DEQLKSGTAS VVCLLNNFYP REAKVQWKVD |
| | NALQSGNSQE SVTEQDSKDS TYSLSSTLTL SKADYEKHKV YACEVTHQGL |
| | SSPVTKSENR GEC |
| Lanadelumab | GAGGTGCAGCTGCTGGAGAGCGGCGGCGGCCTGGTGCAGCCCGGCGGCAGCCTGAGGCTG |
| Heavy/SEQ ID | AGCTGCGCCGCCAGCGGCTTCACCTTCAGCCACTACATCATGATGTGGGTGAGGCAGGCC |
| NO 216 | CCCGGCAAGGGCCTGGAGTGGGTGAGCGGCATCTACAGCAGCGGCGGCATCACCGTGTAC |
| | GCCGACAGCGTGAAGGGCAGGTTCACCATCAGCAGGGACAACAGCAAGAACACCCTGTAC |
| | CTGCAGATGAACAGCCTGAGGGCCGAGGACACCGCCGTGTACTACTGCGCCTACAGGAGG |
| | ATCGGCGTGCCCAGGAGGGACGAGTTCGACATCTGGGGCCAGGGCACCATGGTGACCGTG |
| | AGCAGCGCCAGCACCAAGGGCCCCAGCGTGTTCCCCCTGGCCCCCAGCAGCAAGAGCACC |
| | AGCGGCGGCACCGCCGCCCTGGGCTGCCTGGTGAAGGACTACTTCCCCGAGCCCGTGACC |
| | GTGAGCTGGAACAGCGGCGCCCTGACCAGCGGCGTGCACACCTTCCCCGCCGTGCTGCAG |
| | AGCAGCGGCCTGTACAGCCTGAGCAGCGTGGTGACCGTGCCCAGCAGCAGCCTGGGCACC |
| | CAGACCTACATCTGCAACGTGAACCACAAGCCCAGCAACACCAAGGTGGACAAGAGGGTG |
| | GAGCCCAAGAGCTGCGAC+/− AAGACCCACACC (or AAGACCCACCTG) +/− |
| | TGCCCCCCCTGCCCCGCC+/− CCCGAGCTGCTGGGCGGCCCCAGCGTGT TCCTG |
| Lanadelumab | GACATCCAGATGACCCAGAGCCCCAGCACCCTGAGCGCCAGCGTGGGCGACAGGGTGACC |
| Light/SEQ ID | ATCACCTGCAGGGCCAGCCAGAGCATCAGCAGCTGGCTGGCCTGGTACCAGCAGAAGCCC |
| NO: 217 | GGCAAGGCCCCCAAGCTGCTGATCTACAAGGCCAGCACCCTGGAGAGCGGCGTGCCCAGC |
| | AGGTTCAGCGGCAGCGGCAGCGGCACCGAGTTCACCCTGACCATCAGCAGCCTGCAGCCC |
| | GACGACTTCGCCACCTACTACTGCCAGCAGTACAACACCTACTGGACCTTCGGCCAGGGC |
| | ACCAAGGTGGAGATCAAGAGGACCGTGGCCGCCCCCAGCGTGTTCATCTTCCCCCCCAGC |
| | GACGAGCAGCTGAAGAGCGGCACCGCCAGCGTGGTGTGCCTGCTGAACAACTTCTACCCC |
| | AGGGAGGCCAAGGTGCAGTGGAAGGTGGACAACGCCCTGCAGAGCGGCAACAGCCAGGAG |
| | AGCGTGACCGAGCAGGACAGCAAGGACAGCACCTACAGCCTGAGCAGCACCCTGACCCTG |
| | AGCAAGGCCGACTACGAGAAGCACAAGGTGTACGCCTGCGAGGTGACCCACCAGGGCCTG |
| | AGCAGCCCCGTGACCAAGAGCTTCAACAGGGGCGAGTGC |
| L01 | ATGTACCGGATGCAGCTGCTGCTGCTCATTGCCCTGTCTCTGGCCCTGGTCACCAATAGC |
| SEQ ID NO: 218 | GAAGTCCAGCTGCTGGAATCTGGCGGCGGACTTGTTCAACCTGGCGGCTCTCTGAGACTG |
| | AGCTGTGCCGCTTCCGGCTTCACCTTCAGCCACTATATCATGATGTGGGTCCGACAGGCC |
| | CCTGGCAAAGGCCTTGAATGGGTGTCCGGCATCTATAGCAGCGGCGGCATCACAGTGTAC |
| | GCCGACTCTGTGAAGGGCAGATTCACCATCAGCCGGGACAACAGCAAGAACACCCTGTAC |
| | CTGCAGATGAACAGCCTGAGAGCCGAGGACACCGCCGTGTACTACTGCGCCTATAGAAGA |
| | ATCGGCGTGCCCAGACGGGACGAGTTCGATATTTGGGGCCAGGGCACCATGGTCACCGTG |
| | TCTAGCGCCTCTACAAAGGGCCCTAGCGTGTTCCCTCTGGCTCCTAGCAGCAAGAGCACA |
| | AGCGGAGGAACAGCCGCTCTGGGCTGTCTGGTCAAGGACTACTTTCCCGAGCCTGTGACC |
| | GTGTCCTGGAATAGCGGAGCACTGACAAGCGGCGTGCACACCTTTCCAGCTGTGCTGCAA |
| | AGCAGCGGCCTGTACTCTCTGAGCAGCGTGGTCACAGTGCCAAGCTCTAGCCTGGGCACC |
| | CAGACCTACATCTGCAATGTGAACCACAAGCCTAGCAACACCAAGGTGGACAAGAGAGTG |
| | GAACCCAAGAGCTGCGACAAGACCCACACCTGTCCTCCATGTCCTGCTCCAGAACTGCTC |
| | GGCGGACCTTCCGTGTTCCTGTTTCCTCCAAAGCCTAAGGACACCCTGATGATCAGCAGA |
| | ACCCCTGAAGTGACCTGCGTGGTGGTGGATGTGTCCCACGAGGATCCCGAAGTGAAGTTC |
| | AATTGGTACGTGGACGGCGTGGAAGTGCACAACGCCAAGACCAAGCCTAGAGAGGAACAG |
| | TACAACAGCACCTACAGAGTGGTGTCCGTGCTGACCGTGCTGCACCAGGATTGGCTGAAC |
| | GGCAAAGAGTACAAGTGCAAGGTGTCCAACAAGGCCCTGCCTGCTCCTATCGAGAAAACC |
| | ATCAGCAAGGCCAAGGGCCAGCCTAGGGAACCCCAGGTTTACACACTGCCTCCAAGCCGG |
| | GAAGAGATGACCAAGAACCAGGTGTCCCTGACCTGCCTCGTGAAGGGCTTCTACCCTTCC |
| | GATATCGCCGTGGAATGGGAGAGCAATGGCCAGCCAGAGAACAACTACAAGACAACCCCT |
| | CCTGTGCTGGACAGCGACGGCTCATTCTTCCTGTACAGCAAGCTGACAGTGGACAAGTCC |
| | AGATGGCAGCAGGGCAACGTGTTCAGCTGCAGCGTGATGCACGAGGCCCTGCACAACCAC |
| | TACACCCAGAAGAGTCTGAGCCTGTCTCCAGGCCTGCGGAAGAGAAGGGCTCCTGTGAAG |
| | CAGACCCTGAACTTCGACCTGCTGAAACTGGCCGGCGACGTGGAAAGCAACCCCGGACCT |
| | ATGTATAGAATGCAGCTCCTCCTGCTGATCGCACTGAGCCTGGCTCTCGTGACCAACAGC |
| | GACATCCAGATGACACAGAGCCCCAGCACACTGTCTGCCAGCGTGGGAGACAGAGTGACC |
| | ATCACCTGTAGAGCCAGCCAGTCCATCTCCTCTTGGCTGGCCTGGTATCAGCAAAAGCCT |
| | GGCAAGGCCCCTAAGCTGCTGATCTACAAGGCCTCCACACTGGAAAGCGGGGTGCCCTCC |
| | AGATTTTCTGGCAGCGGATCTGGCACCGAGTTCACCCTGACCATCAGTAGCCTGCAGCCT |
| | GACGACTTCGCCACCTACTACTGCCAGCAGTACAATACCTACTGGACCTTCGGCCAGGGA |
| | ACAAAGGTGGAAATCAAGCGGACTGTGGCCGCTCCAAGCGTGTTCATCTTTCCACCTAGC |
| | GACGAGCAGCTGAAGTCCGGCACAGCCTCTGTTGTGTGCCTGCTGAACAACTTCTACCCC |
| | AGAGAAGCCAAGGTGCAGTGGAAAGTGGACAATGCCCTGCAGAGCGGCAACTCCCAAGAG |
| | AGCGTGACAGAGCAGGACTCCAAGGATTCCACCTACAGCCTGTCTAGCACCCTGACACTG |
| | AGCAAGGCCGACTACGAGAAGCACAAAGTGTATGCCTGCGAAGTGACCCACCAGGGCCTT |
| | AGCTCTCCAGTGACCAAGAGCTTCAACCGGGGCGAGTGTTGATAA |
| L02 | ATGTACAGAATGCAGCTGCTGCTGCTCATTGCCCTGTCTCTGGCCCTGGTCACCAATTCT |
| SEQ ID NO: 219 | GAGGTCCAGCTGCTTGAGAGTGGTGGTGGACTGGTTCAGCCTGGTGGCAGCCTGAGACTG |
| | TCTTGTGCTGCCTCTGGCTTCACCTTCAGCCACTATATCATGATGTGGGTCAGACAGGCC |
| | CCTGGCAAAGGCCTGGAATGGGTGTCAGGCATCTACAGCAGTGGTGGCATCACAGTGTAT |
| | GCTGACTCTGTGAAGGGCAGATTCACCATCAGCAGAGACAACAGCAAGAACACCCTGTAC |
| | CTGCAGATGAACTCCCTGAGAGCTGAGGACACAGCAGTGTACTACTGTGCCTATAGAAGA |
| | ATTGGGGTGCCCAGAAGGGATGAGTTTGACATCTGGGGCCAGGGCACCATGGTTACAGTG |
| | TCCTCTGCCAGCACAAAGGGCCCCTCTGTTTTTCCACTGGCTCCCAGCAGCAAGAGCACC |
| | AGTGGTGGAACAGCTGCCCTG |
| | GGCTGTCTGGTCAAGGATTACTTCCCTGAGCCTGTGACTGTGTCCTGGAACTCTGGGGCT |
| | CTGACTTCTGGGGTGCACACCTTTCCAGCTGTGCTGCAGTCCTCTGGCCTGTACTCTCTG |
| | TCCTCTGTGGTCACAGTGCCTAGCTCTAGCCTGGGCACACAGACCTACATCTGCAATGTG |
| | AACCACAAGCCTAGCAACACCAAGGTGGACAAGAGAGTGGAACCCAAGAGCTGTGACAAG |
| | ACCCACACCTGTCCTCCATGTCCTGCTCCAGAACTGCTTGGAGGCCCTTCTGTGTTCCTG |
| | TTTCCTCCAAAGCCTAAGGACACCCTGATGATCAGCAGAACCCCTGAAGTGACCTGTGTG |
| | GTGGTTGATGTGTCCCATGAGGACCCAGAAGTGAAGTTCAATTGGTATGTGGATGGGGTT |
| | GAAGTGCACAATGCCAAGACCAAGCCTAGAGAGGAACAGTACAACAGCACCTACAGAGTG |
| | GTTTCTGTGCTGACAGTGCTGCACCAGGACTGGCTGAATGGCAAAGAGTACAAGTGCAAG |
| | GTGTCCAACAAGGCCCTGCCTGCTCCTATTGAGAAAACCATCTCCAAGGCCAAGGGCCAG |
| | CCAAGAGAACCCCAGGTTTACACCCTGCCACCTAGCAGAGAAGAGATGACCAAGAACCAG |
| | GTGTCCCTGACCTGCCTGGTTAAGGGCTTCTACCCCTCTGACATTGCTGTGGAATGGGAG |
| | AGCAATGGCCAGCCTGAAAACAACTACAAGACAACCCCTCCTGTGCTGGACTCTGATGGC |
| | TCATTCTTCCTGTACAGCAAGCTGACTGTGGACAAGTCCAGATGGCAGCAGGGAAATGTG |
| | TTCAGCTGCTCTGTGATGCATGAGGCCCTGCACAACCACTACACCCAGAAAAGTCTGTCT |
| | CTGTCCCCTGGCAGAAAGAGAAGAGGCTCTGGAGAAGGCAGAGGCTCCCTGCTGACATGT |
| | GGGGATGTTGAAGAGAATCCTGGGCCTATGTATAGGATGCAACTGCTCCTCCTGATTGCT |
| | CTGAGCCTGGCTCTTGTGACCAACTCTGACATCCAGATGACACAGAGCCCCTCCACACTG |
| | TCTGCATCTGTGGGAGACAGAGTGACCATCACCTGTAGAGCCAGCCAGTCTATCTCTAGC |
| | TGGCTGGCCTGGTATCAGCAAAAGCCTGGCAAGGCCCCTAAGCTGCTGATCTACAAGGCC |
| | AGCACACTTGAGTCAGGGGTGCCCTCCAGATTTTCTGGCTCTGGATCTGGCACAGAGTTC |
| | ACCCTGACCATCAGCTCCCTGCAGCCAGATGACTTTGCCACCTACTACTGCCAGCAGTAC |
| | AATACCTACTGGACCTTTGGCCAGGGAACAAAGGTGGAAATCAAGAGAACAGTGGCTGCC |
| | CCATCTGTGTTCATCTTCCCACCATCTGATGAACAGCTGAAGTCTGGCACTGCCTCTGTT |
| | GTGTGCCTGCTGAACAACTTTTACCCCAGAGAAGCCAAGGTGCAGTGGAAAGTGGATAAT |
| | GCCCTGCAGTCTGGCAATAGCCAAGAATCTGTGACAGAGCAGGACTCCAAGGATTCCACC |
| | TACAGCCTGAGCAGCACCCTGACACTGAGCAAGGCTGACTATGAGAAGCACAAAGTGTAT |
| | GCCTGTGAAGTGACACACCAGGGACTGAGCAGCCCAGTGACCAAGAGCTTCAACAGGGGA |
| | GAGTGCTGATAA |
| L03 | ATGTACAGAATGCAGCTGCTGCTGCTCATTGCCCTGTCTCTGGCCCTGGTCACCAATTCT |
| SEQ ID NO: 220 | GAGGTCCAGCTGCTTGAGAGTGGTGGTGGACTGGTTCAGCCTGGTGGCAGCCTGAGACTG |
| | TCTTGTGCTGCCTCTGGCTTCACCTTCAGCCACTATATCATGATGTGGGTCAGACAGGCC |
| | CCTGGCAAAGGCCTGGAATGGGTGTCAGGCATCTACAGCAGTGGTGGCATCACAGTGTAT |
| | GCTGACTCTGTGAAGGGCAGATTCACCATCAGCAGAGACAACAGCAAGAACACCCTGTAC |
| | CTGCAGATGAACTCCCTGAGAGCTGAGGACACAGCAGTGTACTACTGTGCCTATAGAAGA |
| | ATTGGGGTGCCCAGAAGGGATGAGTTTGACATCTGGGGCCAGGGCACCATGGTTACAGTG |
| | TCCTCTGCCAGCACAAAGGGCCCCTCTGTTTTTCCACTGGCTCCCAGCAGCAAGAGCACC |
| | AGTGGTGGAACAGCTGCCCTGGGCTGTCTGGTCAAGGATTACTTCCCTGAGCCTGTGACT |
| | GTGTCCTGGAACTCTGGGGCTCTGACCTCTGGGGTGCACACCTTTCCAGCTGTGCTGCAG |
| | TCCTCTGGCCTGTACTCTCTGTCCTCTGTGGTCACAGTGCCTAGCTCTAGCCTGGGCACA |
| | CAGACCTACATCTGCAATGTGAACCACAAGCCTAGCAACACCAAGGTGGACAAGAGAGTG |
| | GAACCCAAGAGCTGTGACAAGACCCACACCTGTCCTCCATGTCCTGCTCCAGAACTGCTT |
| | GGAGGCCCTTCTGTGTTCCTGTTTCCTCCAAAGCCTAAGGACACCCTGATGATCAGCAGA |
| | ACCCCTGAAGTGACCTGTGTGGTGGTTGATGTGTCCCATGAGGACCCAGAAGTGAAGTTC |
| | AATTGGTATGTGGATGGGGTTGAAGTGCACAATGCCAAGACCAAGCCTAGAGAGGAACAG |
| | TACAACAGCACCTACAGAGTGGTTTCTGTGCTGACAGTGCTGCACCAGGACTGGCTGAAT |
| | GGCAAAGAGTACAAGTGCAAGGTGTCCAACAAGGCCCTGCCTGCTCCTATTGAGAAAACC |
| | ATCTCCAAGGCCAAGGGCCAGCCAAGAGAACCCCAGGTTTACACCCTGCCACCTAGCAGA |
| | GAAGAGATGACCAAGAACCAGGTGTCCCTGACCTGCCTGGTTAAGGGCTTCTACCCCTCT |
| | GACATTGCTGTGGAATGGGAGAGCAATGGCCAGCCTGAAAACAACTACAAGACAACCCCT |
| | CCTGTGCTGGACTCTGATGGCTCATTCTTCCTGTACAGCAAGCTGACTGTGGACAAGTCC |
| | AGATGGCAGCAGGGAAATGTGTTCAGCTGCTCTGTGATGCATGAGGCCCTGCACAACCAC |
| | TACACCCAGAAAAGTCTGTCTCTGTCCCCTGGCAGAAAGAGAAGAGGATCAGGGGCCCCA |
| | GTGAAGCAGACCCTGAACTTTGATCTGCTGAAGCTGGCTGGGGATGTTGAGAGCAACCCT |
| | GGACCTATGTATAGGATGCAACTGCTCCTCCTGATTGCTCTGAGCCTGGCTCTTGTGACC |
| | AACTCTGACATCCAGATGACACAGAGCCCCTCCACACTGTCTGCATCTGTGGGAGACAGA |
| | GTGACCATCACCTGTAGAGCCAGCCAGTCTATCTCTTCCTGGCTGGCCTGGTATCAGCAA |
| | AAGCCTGGCAAGGCCCCTAAGCTGCTGATCTACAAGGCCAGCACACTTGAGTCAGGGGTG |
| | CCCTCCAGATTTTCTGGCTCTGGATCTGGCACAGAGTTCACCCTGACCATCAGCTCCCTG |
| | CAGCCAGATGACTTTGCCACCTACTACTGCCAGCAGTACAATACCTACTGGACCTTTGGC |
| | CAGGGAACAAAGGTGGAAATCAAGAGAACAGTGGCTGCCCCATCTGTGTTCATCTTCCCA |
| | CCATCTGATGAACAGCTGAAGTCTGGCACTGCCTCTGTTGTGTGCCTGCTGAACAACTTT |
| | TACCCTAGAGAAGCCAAGGTGCAGTGGAAAGTGGATAATGCCCTGCAGTCTGGCAATAGC |
| | CAAGAATCTGTGACAGAGCAGGACTCCAAGGATTCCACCTACAGCCTGAGCAGCACCCTG |
| | ACACTGAGCAAGGCTGACTATGAGAAGCACAAAGTGTATGCCTGTGAAGTGACACACCAG |
| | GGACTGAGCAGCCCAGTGACCAAGAGCTTCAACAGGGGAGAGTGCTGATAA |
| CAG.LAN.F2A | ctagtcgacattgattattgactagttattaatagtaatcaattacggggtcattagttc |
| (CAG.L01) | atagcccatatatggagttccgcgttacataacttacggtaaatggcccgcctggctgac |
| SEQ ID NO: 221 | cgcccaacgacccccgcccattgacgtcaataatgacgtatgttcccatagtaacgccaa |
| | tagggactttccattgacgtcaatgggtggagtatttacggtaaactgcccacttggcag |
| | tacatcaagtgtatcatatgccaagtacgccccctattgacgtcaatgacggtaaatggc |
| | ccgcctggcattatgcccagtacatgaccttatgggactttcctacttggcagtacatct |
| | acgtattagtcatcgctattaccatggtcgaggtgagccccacgttctgcttcactctcc |
| | ccatctcccccccctccccacccccaattttgtatttatttattttttaattattttgtg |
| | cagcgatgggggcggggggggggggggggcgcgcgccaggcggggcggggcggggcgagg |
| | ggcggggcggggcgaggcggagaggtgcggcggcagccaatcagagcggcgcgctccgaa |
| | agtttccttttatggcgaggcggcggcggcggcggccctataaaaagcgaagcgcgcggc |
| | gggcgggagtcgctgcgcgctgccttcgccccgtgccccgctccgccgccgcctcgcgcc |
| | gcccgccccggctctgactgaccgcgttactcccacaggtgagcgggcgggacggccctt |
| | ctcctccgggctgtaattagcgcttggtttaatgacggcttgtttcttttctgtggctgc |
| | gtgaaagccttgaggggctccgggagggccctttgtgcggggggagcggctcggggggtg |
| | cgtgcgtgtgtgtgtgcgtggggagcgccgcgtgcggctccgcgctgcccggcggctgtg |
| | agcgctgcgggcgcggcgcggggctttgtgcgctccgcagtgtgcgcgaggggagcgcgg |
| | ccgggggcggtgccccgcggtgcggggggggctgcgaggggaacaaaggctgcgtgcggg |
| | gtgtgtgcgtgggggggtgagcagggggtgtgggcgcgtcggtcgggctgcaaccccccc |
| | tgcacccccctccccgagttgctgagcacggcccggcttcgggtgcggggctccgtacgg |
| | ggcgtggcgcggggctcgccgtgccgggcggggggtggcggcaggtgggggtgccgggcg |
| | gggcggggccgcctcgggccggggagggctcgggggaggggcgcggcggcccccggagcg |
| | ccggcggctgtcgaggcgcggcgagccgcagccattgccttttatggtaatcgtgcgaga |
| | gggcgcagggacttcctttgtcccaaatctgtgcggagccgaaatctgggaggcgccgcc |
| | gcaccccctctagcgggcgcggggcgaagcggtgcggcgccggcaggaaggaaatgggcg |
| | gggagggccttcgtgcgtcgccgcgccgccgtccccttctccctctccagcctcggggct |
| | gtccgcggggggacggctgccttcgggggggacggggcagggcggggttcggcttctggc |
| | gtgtgaccggcggctctagagcctctgctaaccatgttcatgccttcttctttttcctac |
| | agctcctgggcaacgtgctggttattgtgctgtctcatcattttggcaaagaattcgcta |
| | gcgggcactttgcactggaacttacaacacccgagcaaggacgcgactctccaccatgta |
| | ccggatgcagctgctgctgctcattgccctgtctctggccctggtcaccaatagcgaagt |
| | ccagctgctggaatctggcggcggacttgttcaacctggcggctctctgagactgagctg |
| | tgccgcttccggcttcaccttcagccactatatcatgatgtgggtccgacaggcccctgg |
| | caaaggccttgaatgggtgtccggcatctatagcagcggcggcatcacagtgtacgccga |
| | ctctgtgaagggcagattcaccatcagccgggacaacagcaagaacaccctgtacctgca |
| | gatgaacagcctgagagccgaggacaccgccgtgtactactgcgcctatagaagaatcgg |
| | cgtgcccagacgggacgagttcgatatttggggccagggcaccatggtcaccgtgtctag |
| | cgcctctacaaagggccctagcgtgttccctctggctcctagcagcaagagcacaagcgg |
| | aggaacagccgctctgggctgtctggtcaaggactactttcccgagcctgtgaccgtgtc |
| | ctggaatagcggagcactgacaagcggcgtgcacacctttccagctgtgctgcaaagcag |
| | cggcctgtactctctgagcagcgtggtcacagtgccaagctctagcctgggcacccagac |
| | ctacatctgcaatgtgaaccacaagcctagcaacaccaaggtggacaagagagtggaacc |
| | caagagctgcgacaagacccacacctgtcctccatgtcctgctccagaactgctcggcgg |
| | accttccgtgttcctgtttcctccaaagcctaaggacaccctgatgatcagcagaacccc |
| | tgaagtgacctgcgtggtggtggatgtgtcccacgaggatcccgaagtgaagttcaattg |
| | gtacgtggacggcgtggaagtgcacaacgccaagaccaagcctagagaggaacagtacaa |
| | cagcacctacagagtggtgtccgtgctgaccgtgctgcaccaggattggctgaacggcaa |
| | agagtacaagtgcaaggtgtccaacaaggccctgcctgctcctatcgagaaaaccatcag |
| | caaggccaagggccagcctagggaaccccaggtttacacactgcctccaagccgggaaga |
| | gatgaccaagaaccaggtgtccctgacctgcctcgtgaagggcttctacccttccgatat |
| | cgccgtggaatgggagagcaatggccagccagagaacaactacaagacaacccctcctgt |
| | gctggacagcgacggctcattcttcctgtacagcaagctgacagtggacaagtccagatg |
| | gcagcagggcaacgtgttcagctgcagcgtgatgcacgaggccctgcacaaccactacac |
| | ccagaagagtctgagcctgtctccaggcctgcggaagagaagggctcctgtgaagcagac |
| | cctgaacttcgacctgctgaaactggccggcgacgtggaaagcaaccccggacctatgta |
| | tagaatgcagctcctcctgctgatcgcactgagcctggctctcgtgaccaacagcgacat |
| | ccagatgacacagagccccagcacactgtctgccagcgtgggagacagagtgaccatcac |
| | ctgtagagccagccagtccatctcctcttggctggcctggtatcagcaaaagcctggcaa |
| | ggcccctaagctgctgatctacaaggcctccacactggaaagcggggtgccctccagatt |
| | ttctggcagcggatctggcaccgagttcaccctgaccatcagtagcctgcagcctgacga |
| | cttcgccacctactactgccagcagtacaatacctactggaccttcggccagggaacaaa |
| | ggtggaaatcaagcggactgtggccgctccaagcgtgttcatctttccacctagcgacga |
| | gcagctgaagtccggcacagcctctgttgtgtgcctgctgaacaacttctaccccagaga |
| | agccaaggtgcagtggaaagtggacaatgccctgcagagcggcaactcccaagagagcgt |
| | gacagagcaggactccaaggattccacctacagcctgtctagcaccctgacactgagcaa |
| | ggccgactacgagaagcacaaagtgtatgcctgcgaagtgacccaccagggccttagctc |
| | tccagtgaccaagagcttcaaccggggcgagtgttgataaagcggccgcggtacctctag |
| | agtcgacccgggcggcctcgaggacggggtgaactacgcctgaggatccgatctttttcc |
| | ctctgccaaaaattatggggacatcatgaagccccttgagcatctgacttctggctaata |
| | aaggaaatttattttcattgcaatagtgtgttggaattttttgtgtctctcactcg |
| CAG.LANv2.F2 | gacattgattattgactagttattaatagtaatcaattacggggtcattagttcatagcc |
| A (CAG.L03) | catatatggagttccgcgttacataacttacggtaaatggcccgcctggctgaccgccca |
| SEQ ID NO: 222 | acgacccccgcccattgacgtcaataatgacgtatgttcccatagtaacgccaataggga |
| | ctttccattgacgtcaatgggtggagtatttacggtaaactgcccacttggcagtacatc |
| | aagtgtatcatatgccaagtacgccccctattgacgtcaatgacggtaaatggcccgcct |
| | ggcattatgcccagtacatgaccttatgggactttcctacttggcagtacatctacgtat |
| | tagtcatcgctattaccatggtcgaggtgagccccacgttctgcttcactctccccatct |
| | cccccccctccccacccccaattttgtatttatttattttttaattattttgtgcagcga |
| | tgggggcggggggggggggggggcgcgcgccaggcggggcggggcggggcgaggggcggg |
| | gcggggcgaggcggagaggtgcggcggcagccaatcagagcggcgcgctccgaaagtttc |
| | cttttatggcgaggcggcggcggcggcggccctataaaaagcgaagcgcgcggcgggcgg |
| | gagtcgctgcgcgctgccttcgccccgtgccccgctccgccgccgcctcgcgccgcccgc |
| | cccggctctgactgaccgcgttactcccacaggtgagcgggcgggacggcccttctcctc |
| | cgggctgtaattagcgcttggtttaatgacggcttgtttcttttctgtggctgcgtgaaa |
| | gccttgaggggctccgggagggccctttgtgcggggggagcggctcggggggtgcgtgcg |
| | tgtgtgtgtgcgtggggagcgccgcgtgcggctccgcgctgcccggcggctgtgagcgct |
| | gcgggcgcggcgcggggctttgtgcgctccgcagtgtgcgcgaggggagcgcggccgggg |
| | gcggtgccccgcggtgcggggggggctgcgaggggaacaaaggctgcgtgcggggtgtgt |
| | gcgtgggggggtgagcagggggtgtgggcgcgtcggtcgggctgcaaccccccctgcacc |
| | cccctccccgagttgctgagcacggcccggcttcgggtgcggggctccgtacggggcgtg |
| | gcgcggggctcgccgtgccgggcggggggtggcggcaggtgggggtgccgggcggggcgg |
| | ggccgcctcgggccggggagggctcgggggaggggcgcggcggcccccggagcgccggcg |
| | gctgtcgaggcgcggcgagccgcagccattgccttttatggtaatcgtgcgagagggcgc |
| | agggacttcctttgtcccaaatctgtgcggagccgaaatctgggaggcgccgccgcaccc |
| | cctctagcgggcgcggggcgaagcggtgcggcgccggcaggaaggaaatgggcggggagg |
| | gccttcgtgcgtcgccgcgccgccgtccccttctccctctccagcctcggggctgtccgc |
| | ggggggacggctgccttcgggggggacggggcagggcggggttcggcttctggcgtgtga |
| | ccggcggctctagagcctctgctaaccatgttcatgccttcttctttttcctacagctcc |
| | tgggcaacgtgctggttattgtgctgtctcatcattttggcaaagaattcgccgccacca |
| | tgtacagaatgcagctgctgctgctcattgccctgtctctggccctggtcaccaattctg |
| | aggtccagctgcttgagagtggtggtggactggttcagcctggtggcagcctgagactgt |
| | cttgtgctgcctctggcttcaccttcagccactatatcatgatgtgggtcagacaggccc |
| | ctggcaaaggcctggaatgggtgtcaggcatctacagcagtggtggcatcacagtgtatg |
| | ctgactctgtgaagggcagattcaccatcagcagagacaacagcaagaacaccctgtacc |
| | tgcagatgaactccctgagagctgaggacacagcagtgtactactgtgcctatagaagaa |
| | ttggggtgcccagaagggatgagtttgacatctggggccagggcaccatggttacagtgt |
| | cctctgccagcacaaagggcccctctgtttttccactggctcccagcagcaagagcacca |
| | gtggtggaacagctgccctgggctgtctggtcaaggattacttccctgagcctgtgactg |
| | tgtcctggaactctggggctctgacctctggggtgcacacctttccagctgtgctgcagt |
| | cctctggcctgtactctctgtcctctgtggtcacagtgcctagctctagcctgggcacac |
| | agacctacatctgcaatgtgaaccacaagcctagcaacaccaaggtggacaagagagtgg |
| | aacccaagagctgtgacaagacccacacctgtcctccatgtcctgctccagaactgcttg |
| | gaggcccttctgtgttcctgtttcctccaaagcctaaggacaccctgatgatcagcagaa |
| | cccctgaagtgacctgtgtggtggttgatgtgtcccatgaggacccagaagtgaagttca |
| | attggtatgtggatggggttgaagtgcacaatgccaagaccaagcctagagaggaacagt |
| | acaacagcacctacagagtggtttctgtgctgacagtgctgcaccaggactggctgaatg |
| | gcaaagagtacaagtgcaaggtgtccaacaaggccctgcctgctcctattgagaaaacca |
| | tctccaaggccaagggccagccaagagaaccccaggtttacaccctgccacctagcagag |
| | aagagatgaccaagaaccaggtgtccctgacctgcctggttaagggcttctacccctctg |
| | acattgctgtggaatgggagagcaatggccagcctgaaaacaactacaagacaacccctc |
| | ctgtgctggactctgatggctcattcttcctgtacagcaagctgactgtggacaagtcca |
| | gatggcagcagggaaatgtgttcagctgctctgtgatgcatgaggccctgcacaaccact |
| | acacccagaaaagtctgtctctgtcccctggcagaaagagaagaggatcaggggccccag |
| | tgaagcagaccctgaactttgatctgctgaagctggctggggatgttgagagcaaccctg |
| | gacctatgtataggatgcaactgctcctcctgattgctctgagcctggctcttgtgacca |
| | actctgacatccagatgacacagagcccctccacactgtctgcatctgtgggagacagag |
| | tgaccatcacctgtagagccagccagtctatctcttcctggctggcctggtatcagcaaa |
| | agcctggcaaggcccctaagctgctgatctacaaggccagcacacttgagtcaggggtgc |
| | cctccagattttctggctctggatctggcacagagttcaccctgaccatcagctccctgc |
| | agccagatgactttgccacctactactgccagcagtacaatacctactggacctttggcc |
| | agggaacaaaggtggaaatcaagagaacagtggctgccccatctgtgttcatcttcccac |
| | catctgatgaacagctgaagtctggcactgcctctgttgtgtgcctgctgaacaactttt |
| | accctagagaagccaaggtgcagtggaaagtggataatgccctgcagtctggcaatagcc |
| | aagaatctgtgacagagcaggactccaaggattccacctacagcctgagcagcaccctga |
| | cactgagcaaggctgactatgagaagcacaaagtgtatgcctgtgaagtgacacaccagg |
| | gactgagcagcccagtgaccaagagcttcaacaggggagagtgctgataactcgaggacg |
| | gggtgaactacgcctgaggatccgatctttttccctctgccaaaaattatggggacatca |
| | tgaagccccttgagcatctgacttctggctaataaaggaaatttattttcattgcaatag |
| | tgtgttggaattttttgtgtctctca |
| CAG.LANv2. | gacattgattattgactagttattaatagtaatcaattacggggtcattagttcatagcc |
| T2A | catatatggagttccgcgttacataacttacggtaaatggcccgcctggctgaccgccca |
| (CAG.L02) | acgacccccgcccattgacgtcaataatgacgtatgttcccatagtaacgccaataggga |
| SEQ ID NO: 223 | ctttccattgacgtcaatgggtggagtatttacggtaaactgcccacttggcagtacatc |
| | aagtgtatcatatgccaagtacgccccctattgacgtcaatgacggtaaatggcccgcct |
| | ggcattatgcccagtacatgaccttatgggactttcctacttggcagtacatctacgtat |
| | tagtcatcgctattaccatggtcgaggtgagccccacgttctgcttcactctccccatct |
| | cccccccctccccacccccaattttgtatttatttattttttaattattttgtgcagcga |
| | tgggggcggggggggggggggggcgcgcgccaggcggggcggggcggggcgaggggcggg |
| | gcggggcgaggcggagaggtgcggcggcagccaatcagagcggcgcgctccgaaagtttc |
| | cttttatggcgaggcggcggcggcggcggccctataaaaagcgaagcgcgcggcgggcgg |
| | gagtcgctgcgcgctgccttcgccccgtgccccgctccgccgccgcctcgcgccgcccgc |
| | cccggctctgactgaccgcgttactcccacaggtgagcgggcgggacggcccttctcctc |
| | cgggctgtaattagcgcttggtttaatgacggcttgtttcttttctgtggctgcgtgaaa |
| | gccttgaggggctccgggagggccctttgtgcggggggagcggctcggggggtgcgtgcg |
| | tgtgtgtgtgcgtggggagcgccgcgtgcggctccgcgctgcccggcggctgtgagcgct |
| | gcgggcgcggcgcggggctttgtgcgctccgcagtgtgcgcgaggggagcgcggccgggg |
| | gcggtgccccgcggtgcggggggggctgcgaggggaacaaaggctgcgtgcggggtgtgt |
| | gcgtgggggggtgagcagggggtgtgggcgcgtcggtcgggctgcaaccccccctgcacc |
| | cccctccccgagttgctgagcacggcccggcttcgggtgcggggctccgtacggggcgtg |
| | gcgcggggctcgccgtgccgggcggggggtggcggcaggtgggggtgccgggcggggcgg |
| | ggccgcctcgggccggggagggctcgggggaggggcgcggcggcccccggagcgccggcg |
| | gctgtcgaggcgcggcgagccgcagccattgccttttatggtaatcgtgcgagagggcgc |
| | agggacttcctttgtcccaaatctgtgcggagccgaaatctgggaggcgccgccgcaccc |
| | cctctagcgggcgcggggcgaagcggtgcggcgccggcaggaaggaaatgggcggggagg |
| | gccttcgtgcgtcgccgcgccgccgtccccttctccctctccagcctcggggctgtccgc |
| | ggggggacggctgccttcgggggggacggggcagggcggggttcggcttctggcgtgtga |
| | ccggcggctctagagcctctgctaaccatgttcatgccttcttctttttcctacagctcc |
| | tgggcaacgtgctggttattgtgctgtctcatcattttggcaaagaattcgccgccacca |
| | tgtacagaatgcagctgctgctgctcattgccctgtctctggccctggtcaccaattctg |
| | aggtccagctgcttgagagtggtggtggactggttcagcctggtggcagcctgagactgt |
| | cttgtgctgcctctggcttcaccttcagccactatatcatgatgtgggtcagacaggccc |
| | ctggcaaaggcctggaatgggtgtcaggcatctacagcagtggtggcatcacagtgtatg |
| | ctgactctgtgaagggcagattcaccatcagcagagacaacagcaagaacaccctgtacc |
| | tgcagatgaactccctgagagctgaggacacagcagtgtactactgtgcctatagaagaa |
| | ttggggtgcccagaagggatgagtttgacatctggggccagggcaccatggttacagtgt |
| | cctctgccagcacaaagggcccctctgtttttccactggctcccagcagcaagagcacca |
| | gtggtggaacagctgccctgggctgtctggtcaaggattacttccctgagcctgtgactg |
| | tgtcctggaactctggggctctgacttctggggtgcacacctttccagctgtgctgcagt |
| | cctctggcctgtactctctgtcctctgtggtcacagtgcctagctctagcctgggcacac |
| | agacctacatctgcaatgtgaaccacaagcctagcaacaccaaggtggacaagagagtgg |
| | aacccaagagctgtgacaagacccacacctgtcctccatgtcctgctccagaactgcttg |
| | gaggcccttctgtgttcctgtttcctccaaagcctaaggacaccctgatgatcagcagaa |
| | cccctgaagtgacctgtgtggtggttgatgtgtcccatgaggacccagaagtgaagttca |
| | attggtatgtggatggggttgaagtgcacaatgccaagaccaagcctagagaggaacagt |
| | acaacagcacctacagagtggtttctgtgctgacagtgctgcaccaggactggctgaatg |
| | gcaaagagtacaagtgcaaggtgtccaacaaggccctgcctgctcctattgagaaaacca |
| | tctccaaggccaagggccagccaagagaaccccaggtttacaccctgccacctagcagag |
| | aagagatgaccaagaaccaggtgtccctgacctgcctggttaagggcttctacccctctg |
| | acattgctgtggaatgggagagcaatggccagcctgaaaacaactacaagacaacccctc |
| | ctgtgctggactctgatggctcattcttcctgtacagcaagctgactgtggacaagtcca |
| | gatggcagcagggaaatgtgttcagctgctctgtgatgcatgaggccctgcacaaccact |
| | acacccagaaaagtctgtctctgtcccctggcagaaagagaagaggctctggagaaggca |
| | gaggctccctgctgacatgtggggatgttgaagagaatcctgggcctatgtataggatgc |
| | aactgctcctcctgattgctctgagcctggctcttgtgaccaactctgacatccagatga |
| | cacagagcccctccacactgtctgcatctgtgggagacagagtgaccatcacctgtagag |
| | ccagccagtctatctctagctggctggcctggtatcagcaaaagcctggcaaggccccta |
| | agctgctgatctacaaggccagcacacttgagtcaggggtgccctccagattttctggct |
| | ctggatctggcacagagttcaccctgaccatcagctccctgcagccagatgactttgcca |
| | cctactactgccagcagtacaatacctactggacctttggccagggaacaaaggtggaaa |
| | tcaagagaacagtggctgccccatctgtgttcatcttcccaccatctgatgaacagctga |
| | agtctggcactgcctctgttgtgtgcctgctgaacaacttttaccccagagaagccaagg |
| | tgcagtggaaagtggataatgccctgcagtctggcaatagccaagaatctgtgacagagc |
| | aggactccaaggattccacctacagcctgagcagcaccctgacactgagcaaggctgact |
| | atgagaagcacaaagtgtatgcctgtgaagtgacacaccagggactgagcagcccagtga |
| | ccaagagcttcaacaggggagagtgctgataactcgaggacggggtgaactacgcctgag |
| | gatccgatctttttccctctgccaaaaattatggggacatcatgaagccccttgagcatc |
| | tgacttctggctaataaaggaaatttattttcattgcaatagtgtgttggaattttttgt |
| | gtctctcactcg |
| TBG.LANv2. | aggttaatttttaaaaagcagtcaaaagtccaagtggcccttggcagcatttactctctc |
| T2A | tgtttgctctggttaataatctcaggagcacaaacattccagatccaggttaatttttaa |
| (TBG.L02) | aaagcagtcaaaagtccaagtggcccttggcagcatttactctctctgtttgctctggtt |
| SEQ ID NO: 224 | aataatctcaggagcacaaacattccagatccggcgcgccagggctggaagctacctttg |
| | acatcatttcctctgcgaatgcatgtataatttctacagaacctattagaaaggatcacc |
| | cagcctctgcttttgtacaactttcccttaaaaaactgccaattccactgctgtttggcc |
| | caatagtgagaactttttcctgctgcctcttggtgcttttgcctatggcccctattctgc |
| | ctgctgaagacactcttgccagcatggacttaaacccctccagctctgacaatcctcttt |
| | ctcttttgttttacatgaagggtctggcagccaaagcaatcactcaaagttcaaacctta |
| | tcattttttgctttgttcctcttggccttggttttgtacatcagctttgaaaataccatc |
| | ccagggttaatgctggggttaatttataactaagagtgctctagttttgcaatacaggac |
| | atgctataaaaatggaaagatgttgctttctgagagacagctttattgcggtagtttatc |
| | acagttaaattgctaacgcagtcagtgcttctgacacaacagtctcgaacttaagctgca |
| | gaagttggtcgtgaggcactgggcaggtaagtatcaaggttacaagacaggtttaaggag |
| | accaatagaaactgggcttgtcgagacagagaagactcttgcgtttctgataggcaccta |
| | ttggtcttactgacatccactttgcctttctctccacaggtgtccactcccagttcaatt |
| | acagctcttaaggctagagtacttaatacgactcactataggctagcctcgagaattcac |
| | gcgtgccgccaccatgtacagaatgcagctgctgctgctcattgccctgtctctggccct |
| | ggtcaccaattctgaggtccagctgcttgagagtggtggtggactggttcagcctggtgg |
| | cagcctgagactgtcttgtgctgcctctggcttcaccttcagccactatatcatgatgtg |
| | ggtcagacaggcccctggcaaaggcctggaatgggtgtcaggcatctacagcagtggtgg |
| | catcacagtgtatgctgactctgtgaagggcagattcaccatcagcagagacaacagcaa |
| | gaacaccctgtacctgcagatgaactccctgagagctgaggacacagcagtgtactactg |
| | tgcctatagaagaattggggtgcccagaagggatgagtttgacatctggggccagggcac |
| | catggttacagtgtcctctgccagcacaaagggcccctctgtttttccactggctcccag |
| | cagcaagagcaccagtggtggaacagctgccctgggctgtctggtcaaggattacttccc |
| | tgagcctgtgactgtgtcctggaactctggggctctgacttctggggtgcacacctttcc |
| | agctgtgctgcagtcctctggcctgtactctctgtcctctgtggtcacagtgcctagctc |
| | tagcctgggcacacagacctacatctgcaatgtgaaccacaagcctagcaacaccaaggt |
| | ggacaagagagtggaacccaagagctgtgacaagacccacacctgtcctccatgtcctgc |
| | tccagaactgcttggaggcccttctgtgttcctgtttcctccaaagcctaaggacaccct |
| | gatgatcagcagaacccctgaagtgacctgtgtggtggttgatgtgtcccatgaggaccc |
| | agaagtgaagttcaattggtatgtggatggggttgaagtgcacaatgccaagaccaagcc |
| | tagagaggaacagtacaacagcacctacagagtggtttctgtgctgacagtgctgcacca |
| | ggactggctgaatggcaaagagtacaagtgcaaggtgtccaacaaggccctgcctgctcc |
| | tattgagaaaaccatctccaaggccaagggccagccaagagaaccccaggtttacaccct |
| | gccacctagcagagaagagatgaccaagaaccaggtgtccctgacctgcctggttaaggg |
| | cttctacccctctgacattgctgtggaatgggagagcaatggccagcctgaaaacaacta |
| | caagacaacccctcctgtgctggactctgatggctcattcttcctgtacagcaagctgac |
| | tgtggacaagtccagatggcagcagggaaatgtgttcagctgctctgtgatgcatgaggc |
| | cctgcacaaccactacacccagaaaagtctgtctctgtcccctggcagaaagagaagagg |
| | ctctggagaaggcagaggctccctgctgacatgtggggatgttgaagagaatcctgggcc |
| | tatgtataggatgcaactgctcctcctgattgctctgagcctggctcttgtgaccaactc |
| | tgacatccagatgacacagagcccctccacactgtctgcatctgtgggagacagagtgac |
| | catcacctgtagagccagccagtctatctctagctggctggcctggtatcagcaaaagcc |
| | tggcaaggcccctaagctgctgatctacaaggccagcacacttgagtcaggggtgccctc |
| | cagattttctggctctggatctggcacagagttcaccctgaccatcagctccctgcagcc |
| | agatgactttgccacctactactgccagcagtacaatacctactggacctttggccaggg |
| | aacaaaggtggaaatcaagagaacagtggctgccccatctgtgttcatcttcccaccatc |
| | tgatgaacagctgaagtctggcactgcctctgttgtgtgcctgctgaacaacttttaccc |
| | cagagaagccaaggtgcagtggaaagtggataatgccctgcagtctggcaatagccaaga |
| | atctgtgacagagcaggactccaaggattccacctacagcctgagcagcaccctgacact |
| | gagcaaggctgactatgagaagcacaaagtgtatgcctgtgaagtgacacaccagggact |
| | gagcagcccagtgaccaagagcttcaacaggggagagtgctgataagtcgacccgggcgg |
| | cctcgaggacggggtgaactacgcctgaggatccgatctttttccctctgccaaaaatta |
| | tggggacatcatgaagccccttgagcatctgacttctggctaataaaggaaatttatttt |
| | cattgcaatagtgtgttggaattttttgtgtctctca |
| ApoE.hAAT.LA | ggctcagaggcacacaggagtttctgggctcaccctgcccccttccaacccctcagttcc |
| Nv2.T2A | catcctccagcagctgtttgtgtgctgcctctgaagtccacactgaacaaacttcagcct |
| (ApoE.hAAT. | actcatgtccctaaaatgggcaaacattgcaagcagcaaacagcaaacacacagccctcc |
| L02) | ctgcctgctgaccttggagctggggcagaggtcagagacctctctgggcccatgccacct |
| SEQ ID NO: 225 | ccaacatccactcgaccccttggaatttcggtggagaggagcagaggttgtcctggcgtg |
| | gtttaggtagtgtgagaggggtacccggggatcttgctaccagtggaacagccactaagg |
| | attctgcagtgagagcagagggccagctaagtggtactctcccagagactgtctgactca |
| | cgccaccccctccaccttggacacaggacgctgtggtttctgagccaggtacaatgactc |
| | ctttcggtaagtgcagtggaagctgtacactgcccaggcaaagcgtccgggcagcgtagg |
| | cgggcgactcagatcccagccagtggacttagcccctgtttgctcctccgataactgggg |
| | tgaccttggttaatattcaccagcagcctcccccgttgcccctctggatccactgcttaa |
| | atacggacgaggacagggccctgtctcctcagcttcaggcaccaccactgacctgggaca |
| | gtcaggtaagtatcaaggttacaagacaggtttaaggagaccaatagaaactgggcttgt |
| | cgagacagagaagactcttgcgtttctgataggcacctattggtcttactgacatccact |
| | ttgcctttctctccacaggttcgaattcgccgccaccatgtacagaatgcagctgctgct |
| | gctcattgccctgtctctggccctggtcaccaattctgaggtccagctgcttgagagtgg |
| | tggtggactggttcagcctggtggcagcctgagactgtcttgtgctgcctctggcttcac |
| | cttcagccactatatcatgatgtgggtcagacaggcccctggcaaaggcctggaatgggt |
| | gtcaggcatctacagcagtggtggcatcacagtgtatgctgactctgtgaagggcagatt |
| | caccatcagcagagacaacagcaagaacaccctgtacctgcagatgaactccctgagagc |
| | tgaggacacagcagtgtactactgtgcctatagaagaattggggtgcccagaagggatga |
| | gtttgacatctggggccagggcaccatggttacagtgtcctctgccagcacaaagggccc |
| | ctctgtttttccactggctcccagcagcaagagcaccagtggtggaacagctgccctggg |
| | ctgtctggtcaaggattacttccctgagcctgtgactgtgtcctggaactctggggctct |
| | gacttctggggtgcacacctttccagctgtgctgcagtcctctggcctgtactctctgtc |
| | ctctgtggtcacagtgcctagctctagcctgggcacacagacctacatctgcaatgtgaa |
| | ccacaagcctagcaacaccaaggtggacaagagagtggaacccaagagctgtgacaagac |
| | ccacacctgtcctccatgtcctgctccagaactgcttggaggcccttctgtgttcctgtt |
| | tcctccaaagcctaaggacaccctgatgatcagcagaacccctgaagtgacctgtgtggt |
| | ggttgatgtgtcccatgaggacccagaagtgaagttcaattggtatgtggatggggttga |
| | agtgcacaatgccaagaccaagcctagagaggaacagtacaacagcacctacagagtggt |
| | ttctgtgctgacagtgctgcaccaggactggctgaatggcaaagagtacaagtgcaaggt |
| | gtccaacaaggccctgcctgctcctattgagaaaaccatctccaaggccaagggccagcc |
| | aagagaaccccaggtttacaccctgccacctagcagagaagagatgaccaagaaccaggt |
| | gtccctgacctgcctggttaagggcttctacccctctgacattgctgtggaatgggagag |
| | caatggccagcctgaaaacaactacaagacaacccctcctgtgctggactctgatggctc |
| | attcttcctgtacagcaagctgactgtggacaagtccagatggcagcagggaaatgtgtt |
| | cagctgctctgtgatgcatgaggccctgcacaaccactacacccagaaaagtctgtctct |
| | gtcccctggcagaaagagaagaggctctggagaaggcagaggctccctgctgacatgtgg |
| | ggatgttgaagagaatcctgggcctatgtataggatgcaactgctcctcctgattgctct |
| | gagcctggctcttgtgaccaactctgacatccagatgacacagagcccctccacactgtc |
| | tgcatctgtgggagacagagtgaccatcacctgtagagccagccagtctatctctagctg |
| | gctggcctggtatcagcaaaagcctggcaaggcccctaagctgctgatctacaaggccag |
| | cacacttgagtcaggggtgccctccagattttctggctctggatctggcacagagttcac |
| | cctgaccatcagctccctgcagccagatgactttgccacctactactgccagcagtacaa |
| | tacctactggacctttggccagggaacaaaggtggaaatcaagagaacagtggctgcccc |
| | atctgtgttcatcttcccaccatctgatgaacagctgaagtctggcactgcctctgttgt |
| | gtgcctgctgaacaacttttaccccagagaagccaaggtgcagtggaaagtggataatgc |
| | cctgcagtctggcaatagccaagaatctgtgacagagcaggactccaaggattccaccta |
| | cagcctgagcagcaccctgacactgagcaaggctgactatgagaagcacaaagtgtatgc |
| | ctgtgaagtgacacaccagggactgagcagcccagtgaccaagagcttcaacaggggaga |
| | gtgctgataactcgaggacggggtgaactacgcctgaggatccgatctttttccctctgc |
| | caaaaattatggggacatcatgaagccccttgagcatctgacttctggctaataaaggaa |
| | atttattttcattgcaatagtgtgttggaattttttgtgtctctca |
| LSPX1.LANv2. | aggttaatttttaaaaagcagtcaaaagtccaagtggcccttggcagcatttactctctc |
| T2A | tgtttgctctggttaataatctcaggagcacaaacattccagatccaggttaatttttaa |
| (LSPX.L02) | aaagcagtcaaaagtccaagtggcccttggcagcatttactctctctgtttgctctggtt |
| SEQ ID NO: 226 | aataatctcaggagcacaaacattccagatccggcgcgccagggctggaagctacctttg |
| | tctagaaggctcagaggcacacaggagtttctgggctcaccctgcccccttccaacccct |
| | cagttcccatcctccagcagctgtttgtgtgctgcctctgaagtccacactgaacaaact |
| | tcagcctactcatgtccctaaaatgggcaaacattgcaagcagcaaacagcaaacacaca |
| | gccctccctgcctgctgaccttggagctggggcagaggtcagagacctctctgggcccat |
| | gccacctccaacatccactcgaccccttggaatttcggtggagaggagcagaggttgtcc |
| | tggcgtggtttaggtagtgtgagaggggtacccggggatcttgctaccagtggaacagcc |
| | actaaggattctgcagtgagagcagagggccagctaagtggtactctcccagagactgtc |
| | tgactcacgccaccccctccaccttggacacaggacgctgtggtttctgagccaggtaca |
| | atgactcctttcggtaagtgcagtggaagctgtacactgcccaggcaaagcgtccgggca |
| | gcgtaggcgggcgactcagatcccagccagtggacttagcccctgtttgctcctccgata |
| | actggggtgaccttggttaatattcaccagcagcctcccccgttgcccctctggatccac |
| | tgcttaaatacggacgaggacagggccctgtctcctcagcttcaggcaccaccactgacc |
| | tgggacagtcaggtaagtatcaaggttacaagacaggtttaaggagaccaatagaaactg |
| | ggcttgtcgagacagagaagactcttgcgtttctgataggcacctattggtcttactgac |
| | atccactttgcctttctctccacaggttgaattcgccgccaccatgtacagaatgcagct |
| | gctgctgctcattgccctgtctctggccctggtcaccaattctgaggtccagctgcttga |
| | gagtggtggtggactggttcagcctggtggcagcctgagactgtcttgtgctgcctctgg |
| | cttcaccttcagccactatatcatgatgtgggtcagacaggcccctggcaaaggcctgga |
| | atgggtgtcaggcatctacagcagtggtggcatcacagtgtatgctgactctgtgaaggg |
| | cagattcaccatcagcagagacaacagcaagaacaccctgtacctgcagatgaactccct |
| | gagagctgaggacacagcagtgtactactgtgcctatagaagaattggggtgcccagaag |
| | ggatgagtttgacatctggggccagggcaccatggttacagtgtcctctgccagcacaaa |
| | gggcccctctgtttttccactggctcccagcagcaagagcaccagtggtggaacagctgc |
| | cctgggctgtctggtcaaggattacttccctgagcctgtgactgtgtcctggaactctgg |
| | ggctctgacttctggggtgcacacctttccagctgtgctgcagtcctctggcctgtactc |
| | tctgtcctctgtggtcacagtgcctagctctagcctgggcacacagacctacatctgcaa |
| | tgtgaaccacaagcctagcaacaccaaggtggacaagagagtggaacccaagagctgtga |
| | caagacccacacctgtcctccatgtcctgctccagaactgcttggaggcccttctgtgtt |
| | cctgtttcctccaaagcctaaggacaccctgatgatcagcagaacccctgaagtgacctg |
| | tgtggtggttgatgtgtcccatgaggacccagaagtgaagttcaattggtatgtggatgg |
| | ggttgaagtgcacaatgccaagaccaagcctagagaggaacagtacaacagcacctacag |
| | agtggtttctgtgctgacagtgctgcaccaggactggctgaatggcaaagagtacaagtg |
| | caaggtgtccaacaaggccctgcctgctcctattgagaaaaccatctccaaggccaaggg |
| | ccagccaagagaaccccaggtttacaccctgccacctagcagagaagagatgaccaagaa |
| | ccaggtgtccctgacctgcctggttaagggcttctacccctctgacattgctgtggaatg |
| | ggagagcaatggccagcctgaaaacaactacaagacaacccctcctgtgctggactctga |
| | tggctcattcttcctgtacagcaagctgactgtggacaagtccagatggcagcagggaaa |
| | tgtgttcagctgctctgtgatgcatgaggccctgcacaaccactacacccagaaaagtct |
| | gtctctgtcccctggcagaaagagaagaggctctggagaaggcagaggctccctgctgac |
| | atgtggggatgttgaagagaatcctgggcctatgtataggatgcaactgctcctcctgat |
| | tgctctgagcctggctcttgtgaccaactctgacatccagatgacacagagcccctccac |
| | actgtctgcatctgtgggagacagagtgaccatcacctgtagagccagccagtctatctc |
| | tagctggctggcctggtatcagcaaaagcctggcaaggcccctaagctgctgatctacaa |
| | ggccagcacacttgagtcaggggtgccctccagattttctggctctggatctggcacaga |
| | gttcaccctgaccatcagctccctgcagccagatgactttgccacctactactgccagca |
| | gtacaatacctactggacctttggccagggaacaaaggtggaaatcaagagaacagtggc |
| | tgccccatctgtgttcatcttcccaccatctgatgaacagctgaagtctggcactgcctc |
| | tgttgtgtgcctgctgaacaacttttaccccagagaagccaaggtgcagtggaaagtgga |
| | taatgccctgcagtctggcaatagccaagaatctgtgacagagcaggactccaaggattc |
| | cacctacagcctgagcagcaccctgacactgagcaaggctgactatgagaagcacaaagt |
| | gtatgcctgtgaagtgacacaccagggactgagcagcccagtgaccaagagcttcaacag |
| | gggagagtgctgataactcgaggacggggtgaactacgcctgaggatccgatctttttcc |
| | ctctgccaaaaattatggggacatcatgaagccccttgagcatctgacttctggctaata |
| | aaggaaatttattttcattgcaatagtgtgttggaattttttgtgtctctca |
| LSPX2.LANv2. | aggctcagaggcacacaggagtttctgggctcaccctgcccccttccaacccctcagttc |
| T2A | ccatcctccagcagctgtttgtgtgctgcctctgaagtccacactgaacaaacttcagcc |
| (LSPX2.L02) | tactcatgtccctaaaatgggcaaacattgcaagcagcaaacagcaaacacacagccctc |
| SEQ ID NO: 227 | cctgcctgctgaccttggagctggggcagaggtcagagacctctctgggcccatgccacc |
| | tccaacatccactcgaccccttggaatttcggtggagaggagcagaggttgtcctggcgt |
| | ggtttaggtagtgtgagagggtctagaaggctcagaggcacacaggagtttctgggctca |
| | ccctgcccccttccaacccctcagttcccatcctccagcagctgtttgtgtgctgcctct |
| | gaagtccacactgaacaaacttcagcctactcatgtccctaaaatgggcaaacattgcaa |
| | gcagcaaacagcaaacacacagccctccctgcctgctgaccttggagctggggcagaggt |
| | cagagacctctctgggcccatgccacctccaacatccactcgaccccttggaatttcggt |
| | ggagaggagcagaggttgtcctggcgtggtttaggtagtgtgagaggggtacccggggat |
| | cttgctaccagtggaacagccactaaggattctgcagtgagagcagagggccagctaagt |
| | ggtactctcccagagactgtctgactcacgccaccccctccaccttggacacaggacgct |
| | gtggtttctgagccaggtacaatgactcctttcggtaagtgcagtggaagctgtacactg |
| | cccaggcaaagcgtccgggcagcgtaggcgggcgactcagatcccagccagtggacttag |
| | cccctgtttgctcctccgataactggggtgaccttggttaatattcaccagcagcctccc |
| | ccgttgcccctctggatccactgcttaaatacggacgaggacagggccctgtctcctcag |
| | cttcaggcaccaccactgacctgggacagtcaggtaagtatcaaggttacaagacaggtt |
| | taaggagaccaatagaaactgggcttgtcgagacagagaagactcttgcgtttctgatag |
| | gcacctattggtcttactgacatccactttgcctttctctccacaggttgaattcgccgc |
| | caccatgtacagaatgcagctgctgctgctcattgccctgtctctggccctggtcaccaa |
| | ttctgaggtccagctgcttgagagtggtggtggactggttcagcctggtggcagcctgag |
| | actgtcttgtgctgcctctggcttcaccttcagccactatatcatgatgtgggtcagaca |
| | ggcccctggcaaaggcctggaatgggtgtcaggcatctacagcagtggtggcatcacagt |
| | gtatgctgactctgtgaagggcagattcaccatcagcagagacaacagcaagaacaccct |
| | gtacctgcagatgaactccctgagagctgaggacacagcagtgtactactgtgcctatag |
| | aagaattggggtgcccagaagggatgagtttgacatctggggccagggcaccatggttac |
| | agtgtcctctgccagcacaaagggcccctctgtttttccactggctcccagcagcaagag |
| | caccagtggtggaacagctgccctgggctgtctggtcaaggattacttccctgagcctgt |
| | gactgtgtcctggaactctggggctctgacttctggggtgcacacctttccagctgtgct |
| | gcagtcctctggcctgtactctctgtcctctgtggtcacagtgcctagctctagcctggg |
| | cacacagacctacatctgcaatgtgaaccacaagcctagcaacaccaaggtggacaagag |
| | agtggaacccaagagctgtgacaagacccacacctgtcctccatgtcctgctccagaact |
| | gcttggaggcccttctgtgttcctgtttcctccaaagcctaaggacaccctgatgatcag |
| | cagaacccctgaagtgacctgtgtggtggttgatgtgtcccatgaggacccagaagtgaa |
| | gttcaattggtatgtggatggggttgaagtgcacaatgccaagaccaagcctagagagga |
| | acagtacaacagcacctacagagtggtttctgtgctgacagtgctgcaccaggactggct |
| | gaatggcaaagagtacaagtgcaaggtgtccaacaaggccctgcctgctcctattgagaa |
| | aaccatctccaaggccaagggccagccaagagaaccccaggtttacaccctgccacctag |
| | cagagaagagatgaccaagaaccaggtgtccctgacctgcctggttaagggcttctaccc |
| | ctctgacattgctgtggaatgggagagcaatggccagcctgaaaacaactacaagacaac |
| | ccctcctgtgctggactctgatggctcattcttcctgtacagcaagctgactgtggacaa |
| | gtccagatggcagcagggaaatgtgttcagctgctctgtgatgcatgaggccctgcacaa |
| | ccactacacccagaaaagtctgtctctgtcccctggcagaaagagaagaggctctggaga |
| | aggcagaggctccctgctgacatgtggggatgttgaagagaatcctgggcctatgtatag |
| | gatgcaactgctcctcctgattgctctgagcctggctcttgtgaccaactctgacatcca |
| | gatgacacagagcccctccacactgtctgcatctgtgggagacagagtgaccatcacctg |
| | tagagccagccagtctatctctagctggctggcctggtatcagcaaaagcctggcaaggc |
| | ccctaagctgctgatctacaaggccagcacacttgagtcaggggtgccctccagattttc |
| | tggctctggatctggcacagagttcaccctgaccatcagctccctgcagccagatgactt |
| | tgccacctactactgccagcagtacaatacctactggacctttggccagggaacaaaggt |
| | ggaaatcaagagaacagtggctgccccatctgtgttcatcttcccaccatctgatgaaca |
| | gctgaagtctggcactgcctctgttgtgtgcctgctgaacaacttttaccccagagaagc |
| | caaggtgcagtggaaagtggataatgccctgcagtctggcaatagccaagaatctgtgac |
| | agagcaggactccaaggattccacctacagcctgagcagcaccctgacactgagcaaggc |
| | tgactatgagaagcacaaagtgtatgcctgtgaagtgacacaccagggactgagcagccc |
| | agtgaccaagagcttcaacaggggagagtgctgataactcgaggacggggtgaactacgc |
| | ctgaggatccgatctttttccctctgccaaaaattatggggacatcatgaagccccttga |
| | gcatctgacttctggctaataaaggaaatttattttcattgcaatagtgtgttggaattt |
| | tttgtgtctctca |
| LTP1.LANv2. | aggttaatttttaaaaagcagtcaaaagtccaagtggcccttggcagcatttactctctc |
| T2A | tgtttgctctggttaataatctcaggagcacaaacattccagatccaggttaatttttaa |
| (LTP1.L02) | aaagcagtcaaaagtccaagtggcccttggcagcatttactctctctgtttgctctggtt |
| SEQ ID NO: 228 | aataatctcaggagcacaaacattccagatccggcgcgccagggctggaagctacctttg |
| | acatcatttcctctgcgaatgcatgtataatttctacagaacctattagaaaggatcacc |
| | cagcctctgcttttgtacaactttcccttaaaaaactgccaattccactgctgtttggcc |
| | caatagtgagaactttttcctgctgcctcttggtgcttttgcctatggcccctattctgc |
| | ctgctgaagacactcttgccagcatggacttaaacccctccagctctgacaatcctcttt |
| | ctcttttgttttacatgaagggtctggcagccaaagcaatcactcaaagttcaaacctta |
| | tcattttttgctttgttcctcttggccttggttttgtacatcagctttgaaaataccatc |
| | ccagggttaatgctggggttaatttataactaagagtgctctagttttgcaatacaggac |
| | atgctataaaaatggaaagatgttgctttctgagaggatcttgctaccagtggaacagcc |
| | actaaggattctgcagtgagagcagagggccagctaagtggtactctcccagagactgtc |
| | tgactcacgccaccccctccaccttggacacaggacgctgtggtttctgagccaggtaca |
| | gtgactcctttcggtaagtgcagtggaagctgtacactgcccaggcaaagcgtccgggca |
| | gcgtaggcgggcgactcagatcccagccagtggacttagcccctgtttgctcctccgata |
| | actggggtgaccttggttaatattcaccagcagcctcccccgttgcccctctggatccac |
| | tgcttaaatacggacgaggacagggccctgtctcctcagcttcaggcaccaccactgacc |
| | tgggacagtcaggtaagtatcaaggttacaagacaggtttaaggagaccaatagaaactg |
| | ggcttgtcgagacagagaagactcttgcgtttctgataggcacctattggtcttactgac |
| | atccactttgcctttctctccacaggttgaattcgccgccaccatgtacagaatgcagct |
| | gctgctgctcattgccctgtctctggccctggtcaccaattctgaggtccagctgcttga |
| | gagtggtggtggactggttcagcctggtggcagcctgagactgtcttgtgctgcctctgg |
| | cttcaccttcagccactatatcatgatgtgggtcagacaggcccctggcaaaggcctgga |
| | atgggtgtcaggcatctacagcagtggtggcatcacagtgtatgctgactctgtgaaggg |
| | cagattcaccatcagcagagacaacagcaagaacaccctgtacctgcagatgaactccct |
| | gagagctgaggacacagcagtgtactactgtgcctatagaagaattggggtgcccagaag |
| | ggatgagtttgacatctggggccagggcaccatggttacagtgtcctctgccagcacaaa |
| | gggcccctctgtttttccactggctcccagcagcaagagcaccagtggtggaacagctgc |
| | cctgggctgtctggtcaaggattacttccctgagcctgtgactgtgtcctggaactctgg |
| | ggctctgacttctggggtgcacacctttccagctgtgctgcagtcctctggcctgtactc |
| | tctgtcctctgtggtcacagtgcctagctctagcctgggcacacagacctacatctgcaa |
| | tgtgaaccacaagcctagcaacaccaaggtggacaagagagtggaacccaagagctgtga |
| | caagacccacacctgtcctccatgtcctgctccagaactgcttggaggcccttctgtgtt |
| | cctgtttcctccaaagcctaaggacaccctgatgatcagcagaacccctgaagtgacctg |
| | tgtggtggttgatgtgtcccatgaggacccagaagtgaagttcaattggtatgtggatgg |
| | ggttgaagtgcacaatgccaagaccaagcctagagaggaacagtacaacagcacctacag |
| | agtggtttctgtgctgacagtgctgcaccaggactggctgaatggcaaagagtacaagtg |
| | caaggtgtccaacaaggccctgcctgctcctattgagaaaaccatctccaaggccaaggg |
| | ccagccaagagaaccccaggtttacaccctgccacctagcagagaagagatgaccaagaa |
| | ccaggtgtccctgacctgcctggttaagggcttctacccctctgacattgctgtggaatg |
| | ggagagcaatggccagcctgaaaacaactacaagacaacccctcctgtgctggactctga |
| | tggctcattcttcctgtacagcaagctgactgtggacaagtccagatggcagcagggaaa |
| | tgtgttcagctgctctgtgatgcatgaggccctgcacaaccactacacccagaaaagtct |
| | gtctctgtcccctggcagaaagagaagaggctctggagaaggcagaggctccctgctgac |
| | atgtggggatgttgaagagaatcctgggcctatgtataggatgcaactgctcctcctgat |
| | tgctctgagcctggctcttgtgaccaactctgacatccagatgacacagagcccctccac |
| | actgtctgcatctgtgggagacagagtgaccatcacctgtagagccagccagtctatctc |
| | tagctggctggcctggtatcagcaaaagcctggcaaggcccctaagctgctgatctacaa |
| | ggccagcacacttgagtcaggggtgccctccagattttctggctctggatctggcacaga |
| | gttcaccctgaccatcagctccctgcagccagatgactttgccacctactactgccagca |
| | gtacaatacctactggacctttggccagggaacaaaggtggaaatcaagagaacagtggc |
| | tgccccatctgtgttcatcttcccaccatctgatgaacagctgaagtctggcactgcctc |
| | tgttgtgtgcctgctgaacaacttttaccccagagaagccaaggtgcagtggaaagtgga |
| | taatgccctgcagtctggcaatagccaagaatctgtgacagagcaggactccaaggattc |
| | cacctacagcctgagcagcaccctgacactgagcaaggctgactatgagaagcacaaagt |
| | gtatgcctgtgaagtgacacaccagggactgagcagcccagtgaccaagagcttcaacag |
| | gggagagtgctgataactcgaggacggggtgaactacgcctgaggatccgatctttttcc |
| | ctctgccaaaaattatggggacatcatgaagccccttgagcatctgacttctggctaata |
| | aaggaaatttattttcattgcaatagtgtgttggaattttttgtgtctctca |
| LMTP6.LANv2. | aggctcagaggcacacaggagtttctgggctcaccctgcccccttccaacccctcagttc |
| T2A | ccatcctccagcagctgtttgtgtgctgcctctgaagtccacactgaacaaacttcagcc |
| (LMTP6.L02) | tactcatgtccctaaaatgggcaaacattgcaagcagcaaacagcaaacacacagccctc |
| SEQ ID NO: 229 | cctgcctgctgaccttggagctggggcagaggtcagagacctctctgggcccatgccacc |
| | tccaacatccactcgaccccttggaatttcggtggagaggagcagaggttgtcctggcgt |
| | ggtttaggtagtgtgagagggccactacgggtttaggctgcccatgtaaggaggcaaggc |
| | ctggggacacccgagatgcctggttataattaacccagacatgtggctgccccccccccc |
| | cccaacacctgctgcctctaaaaataaccctgtccctggtggatcccactacgggtttag |
| | gctgcccatgtaaggaggcaaggcctggggacacccgagatgcctggttataattaaccc |
| | agacatgtggctgccccccccccccccaacacctgctgcctctaaaaataaccctgtccc |
| | tggtggatcccactacgggtttaggctgcccatgtaaggaggcaaggcctggggacaccc |
| | gagatgcctggttataattaacccagacatgtggctgccccccccccccccaacacctgc |
| | tgcctctaaaaataaccctgtccctggtggatcccctgcatgcgaagatcttcgaacaag |
| | gctgtgggggactgagggcaggctgtaacaggcttgggggccagggcttatacgtgcctg |
| | ggactcccaaagtattactgttccatgttcccggcgaagggccagctgtcccccgccagc |
| | tagactcagcacttagtttaggaaccagtgagcaagtcagcccttggggcagcccataca |
| | aggccatggggctgggcaagctgcacgcctgggtccggggtgggcacggtgcccgggcaa |
| | cgagctgaaagctcatctgctctcaggggcccctccctggggacagcccctcctggctag |
| | tcacaccctgtaggctcctctatataacccaggggcacaggggctgccctcattctacca |
| | ccacctccacagcacagacagacactcaggagccagccagcgtcgagatcttgctaccag |
| | tggaacagccactaaggattctgcagtgagagcagagggccagctaagtggtactctccc |
| | agagactgtctgactcacgccaccccctccaccttggacacaggacgctgtggtttctga |
| | gccaggtacagtgactcctttcggtaagtgcagtggaagctgtacactgcccaggcaaag |
| | cgtccgggcagcgtaggcgggcgactcagatcccagccagtggacttagcccctgtttgc |
| | tcctccgataactggggtgaccttggttaatattcaccagcagcctcccccgttgcccct |
| | ctggatccactgcttaaatacggacgaggacagggccctgtctcctcagcttcaggcacc |
| | accactgacctgggacagtcaggtaagtatcaaggttacaagacaggtttaaggagacca |
| | atagaaactgggcttgtcgagacagagaagactcttgcgtttctgataggcacctattgg |
| | tcttactgacatccactttgcctttctctccacaggttcgaattcgccgccaccatgtac |
| | agaatgcagctgctgctgctcattgccctgtctctggccctggtcaccaattctgaggtc |
| | cagctgcttgagagtggtggtggactggttcagcctggtggcagcctgagactgtcttgt |
| | gctgcctctggcttcaccttcagccactatatcatgatgtgggtcagacaggcccctggc |
| | aaaggcctggaatgggtgtcaggcatctacagcagtggtggcatcacagtgtatgctgac |
| | tctgtgaagggcagattcaccatcagcagagacaacagcaagaacaccctgtacctgcag |
| | atgaactccctgagagctgaggacacagcagtgtactactgtgcctatagaagaattggg |
| | gtgcccagaagggatgagtttgacatctggggccagggcaccatggttacagtgtcctct |
| | gccagcacaaagggcccctctgtttttccactggctcccagcagcaagagcaccagtggt |
| | ggaacagctgccctgggctgtctggtcaaggattacttccctgagcctgtgactgtgtcc |
| | tggaactctggggctctgacttctggggtgcacacctttccagctgtgctgcagtcctct |
| | ggcctgtactctctgtcctctgtggtcacagtgcctagctctagcctgggcacacagacc |
| | tacatctgcaatgtgaaccacaagcctagcaacaccaaggtggacaagagagtggaaccc |
| | aagagctgtgacaagacccacacctgtcctccatgtcctgctccagaactgcttggaggc |
| | ccttctgtgttcctgtttcctccaaagcctaaggacaccctgatgatcagcagaacccct |
| | gaagtgacctgtgtggtggttgatgtgtcccatgaggacccagaagtgaagttcaattgg |
| | tatgtggatggggttgaagtgcacaatgccaagaccaagcctagagaggaacagtacaac |
| | agcacctacagagtggtttctgtgctgacagtgctgcaccaggactggctgaatggcaaa |
| | gagtacaagtgcaaggtgtccaacaaggccctgcctgctcctattgagaaaaccatctcc |
| | aaggccaagggccagccaagagaaccccaggtttacaccctgccacctagcagagaagag |
| | atgaccaagaaccaggtgtccctgacctgcctggttaagggcttctacccctctgacatt |
| | gctgtggaatgggagagcaatggccagcctgaaaacaactacaagacaacccctcctgtg |
| | ctggactctgatggctcattcttcctgtacagcaagctgactgtggacaagtccagatgg |
| | cagcagggaaatgtgttcagctgctctgtgatgcatgaggccctgcacaaccactacacc |
| | cagaaaagtctgtctctgtcccctggcagaaagagaagaggctctggagaaggcagaggc |
| | tccctgctgacatgtggggatgttgaagagaatcctgggcctatgtataggatgcaactg |
| | ctcctcctgattgctctgagcctggctcttgtgaccaactctgacatccagatgacacag |
| | agcccctccacactgtctgcatctgtgggagacagagtgaccatcacctgtagagccagc |
| | cagtctatctctagctggctggcctggtatcagcaaaagcctggcaaggcccctaagctg |
| | ctgatctacaaggccagcacacttgagtcaggggtgccctccagattttctggctctgga |
| | tctggcacagagttcaccctgaccatcagctccctgcagccagatgactttgccacctac |
| | tactgccagcagtacaatacctactggacctttggccagggaacaaaggtggaaatcaag |
| | agaacagtggctgccccatctgtgttcatcttcccaccatctgatgaacagctgaagtct |
| | ggcactgcctctgttgtgtgcctgctgaacaacttttaccccagagaagccaaggtgcag |
| | tggaaagtggataatgccctgcagtctggcaatagccaagaatctgtgacagagcaggac |
| | tccaaggattccacctacagcctgagcagcaccctgacactgagcaaggctgactatgag |
| | aagcacaaagtgtatgcctgtgaagtgacacaccagggactgagcagcccagtgaccaag |
| | agcttcaacaggggagagtgctgataactcgaggacggggtgaactacgcctgaggatcc |
| | gatctttttccctctgccaaaaattatggggacatcatgaagccccttgagcatctgact |
| | tctggctaataaaggaaatttattttcattgcaatagtgtgttggaattttttgtgtctc |
| | tca |

HEK293 cells were plated at a density of 7.5×10⁵ cells/well in each well of a standard 6-well dish containing Dulbecco's modified eagle medium (DMEM) supplied with 10% fetal bovine serum (FBS). The next day, cells were transfected with CAG.L01 (SEQ ID NO:221), CAG.L02 (SEQ ID NO:223), and CAG.L03 (SEQ ID NO:222) AAV constructs using Lifpofectamine 2000 (Invitrogen) according the manufacturer's protocol). Non-transfected cells were used as negative control. Cell culture medium was changed 24 hours post-transfection to opti-mem I reduced serum media (2 mL/well). Cell culture supernatant was harvested at 48 hours post-transfection, and cell lysates were harvested with RIPA buffer (Pierce) supplemented with EDTA-free protease inhibitor tablets (Pierce). Supernatant and lysates samples were stored at −80 C.

Proteins from supernatant or cell lysate samples were separated via the NuPAGE electrophoresis system (Thermo Fisher Scientific). For samples derived from cell lysates, 40 μg of protein was loaded unless indicated otherwise. Purified human IgG or Lanadelumab IgG (produced by Genscript) were used as loading controls (50-100 ng). Samples were heated with LDS sample buffer and NuPAGE reducing agent at 70 C for 10 minutes and then loaded into NuPAGE 4-12% Bis-Tris protein gels. Separated proteins were transferred to PVDF membranes using the iBlot2 dry blotting system according to manufacturer's instructions (P3 default setting was used for the protein transfer). Membranes were immediately washed in phosphate buffer saline with 0.1% v/v Tween-20 (PBST). Membranes were then incubated in blocking solution containing PBST and 1% Clear Milk Blocking Buffer (Thermo Scientific) for 1 hour at room temperature. Membranes were then incubated in fresh blocking solution supplemented with goat anti-human kappa light chain-HRP antibody (Bethyl Laboratories; 1:2000 dilution) and goat anti-human IgG Fc-HRP antibody (1:2000 dilution). Following antibody incubation, membranes were washed three times in PB ST for 5 minutes per wash. Finally, membranes were incubated in SuperSignal West Pico PLUS chemiluminescent substrate for 5 minutes and imaged on the BioRad Universal Hood II gel doc system for detection of horseradish peroxidase (HRP) signal.

### Results

Expression analysis of reporter transgene (eGFP) following transfection of different plasmid quantities (4 μg-nontransfected) showed a dose dependent increase in eGFP levels. Protein expression analysis of lanadelumab in the cell lysate and in the cell supernatant showed dose-dependent levels of lanadelumab in cell lysates and supernatant. Transfection with the construct containing the L02 transgene (SEQ ID NO:219, CAG.L02 (SEQ ID NO: 223)), a codon-optimized and depleted of CpG dinucleotide sequences construction, resulted in higher expression levels compared to L01 transgene (SEQ ID NO: 218, CAG.L01 SEQ ID NO: 221). Transfection of CAG.L02 (SEQ ID NO:223) and CAG.L03 (SEQ ID NO:222) resulted in similar expression levels.

### Example 11: Serum Expression of Lanadelumab in Mice

### Methods

A. Mouse experiments were performed with either AAV8 or AAV9 containing an AAV construct comprising the L01 sequence (SEQ ID NO:218), which contains the Furin and F2A sequence (SEQ ID NO:213). AAV8 and AAV9 vectors (n=5 mice per group; 2e11 genome copies (gc)) were administered to immunocompromised NSG mice via either intravenous (IV) or intramuscular (IM) routes. IV administrations were into the tail vein and IM administrations were bilateral into the gastrocnemius muscles. Mice injected with vehicle were included as controls. Seven weeks post administration mice were sacrificed and serum human antibody levels were determined by enzyme-linked immunosorbent assay (ELISA).

Lanadelumab levels in NSG mouse serum was assessed by ELISA. Briefly, mouse serum was obtained before treatment and at 1, 3, 5 and 7 weeks post in vivo gene transfection and stored at −80° C. 96-well plate was coated with 1 μg/ml human IgG-Fc fragment antibody (Bethyl, Montgomery, TX) in carbonate bicarbonate buffer (0.05M, pH 9.6, Sigma-Aldrich, St. Louis, MO) and incubated overnight at 4° C. After washing with Tween 20 washing buffer (PBST, 0.05%, Alfa Aesar, Haverhill, MA), plate was incubated with blocking buffer (3% BSA in PBS, ThermoFisher Scientific, Waltham, MA) for 1 h at 37° C. followed by washing. Mouse serum samples diluted in sample dilution buffer (0.1% Tween 20 and 3% BSA in PBS) was added to the plate (50 μl/well) and incubated for 2 h at 37° C. A standard curve of known lanadelumab concentrations ranging from 360 to 0.001 ng/mL was included in each plate. Plate was washed with PBST for five times after incubation. The levels of lanadelumab was detected by incubation with horseradish peroxidase-conjugated goat anti-human IgG (H+L) (200 ng/mL; Bethyl, Montgomery, TX) for 1 h at 37° C. The optical density was assessed using KPL TMB Microwell Peroxidase Substrate System (Seracare, Milford, MA) following the manufacturer's specifications. Data analysis was performed with SoftMax Pro version 7.0.2 software (Molecular Devices, Sunnyvale, CA).

### Results

A. Results from a representative experiment are shown in FIG. 5. Serum analysis of AAV8-, AAV9-injected and control (vehicle) NSG mice at 7 weeks post gene transfer showed expression and serum accumulation of Lanadelumab following AAV9 delivery (2E″ gc). Serum Lanadelumab concentration was 100-fold higher in AAV9-injected mice compared to AAV8-injected mice and slightly higher in IV-AAV9-injected compared to IM-AAV9-injected mice. Serum human antibody levels in control mice were undetectable at 7 week time point.

B. In an analogous experiment, a time course of lanadelumab serum levels in NSG mice post-AAV9 administration (n=5 per group) was performed. AAV9 vectors (2E″ gc) were injected either IV or IM (as above, in experiment A), and serum antibody levels were determined by ELISA at day 7 (D7), day 21 (D21), day 35 (D35), and day 49 (D49).

Serum Lanadelumab expression is detectable as early as 1 week (D7) after AAV9 administration in NSG mice. The expression levels increased at 3 weeks (D2), peaked at 5 weeks (D35) and then sustained up to 7 week post-injection (D49). It was observed that serum lanadelumab concentration is higher in IV vs. IM injected mice over the entire time course. See FIG. 6.

C. In an analogous experiment, a time course of lanadelumab serum levels in C/57BL6 mice post AAV8 administration was performed. The optimized expression cassette containing a liver-specific promoter and a codon optimized and CpG depleted transgene with a modified furin-2A processing signal resulted in robust serum antibody concentration when delivered intravenously using an AAV8 vector. Very high (>1 mg/ml) and sustained levels of functional anti-kallikrein antibody were achieved in the serum of C57BL/6 mice following IV vector administration at a dose of 1E¹³ gc/kg.

### Example 12: Analysis of In Vitro Transduction and Expression of Tandem Liver- and Tandem Liver/Muscle-Specific Promoters Driving Expression of Lanadelumab

Cis plasmids expressing vectorized lanadelumab were packaged in AAV, then rAAV particles evaluated for potency of the transduction by AAV. Each cis plasmid contained lanadelumab (Mab1) antibody light chain and heavy chain which are multicistrons driven by the CAG, ApoE.hAAT (SEQ ID NO:78) or LMTP6 (SEQ ID NO:71) promoter. Full-length lanadelumab antibody light chain and antibody heavy chain genes were separated by a furin 2A linker to ensure separate expression of each antibody chain. The entire cassette is flanked by AAV2 ITRs, and the genome is encapsidated in an AAV8 capsid for delivery to C2C12 cells (1E¹⁰ vg per well). For detection of antibody protein, following transduction, the cells are treated with FITC conjugated anti-Fc (IgG) antibody. The AAV8.CAG.Mab1 and AAV8.LMTP6.Mab1 infected cells show high expression in muscle cells, whereas the AAV8.hAAT.Mab1 infection does not result in expression of the antibody in muscle cells. Cells appeared to be equally confluent and viable in all test wells, as seen by DAPI (DNA) staining.

### Example 13: Antibody Expression and Vector Biodistribution in Mouse Treated with AAV8 Lanadelumab Vectors Driven by Various Promoters

Thyroxine binding globulin (TBG) and alpha-1 antitrypsin (hAAT) promoters have been widely used as liver-specific promoters in previous pre-clinical and clinical gene therapy studies. A panel of designed promoter cassettes derived from multiple promoters and enhancers were generated and tested them in vitro by transfecting Huh7 cells, a human liver cell line. Promoter candidates were selected, which include ApoE.hAAT (SEQ ID NO:78), LSPX1 (SEQ ID NO:66), LSPX2 (SEQ ID NO:67), LTP1 (SEQ ID NO:68) and LMTP6 (SEQ ID NO:71). AAV8 vectors encoding vectorized lanadelumab regulated by these promoter candidates were then generated. CAG (SEQ ID NO:89) and TBG (SEQ ID NO:93) promoters served as controls for ubiquitous and liver-specific promoters, respectfully. Strength of these promoters and vector biodistribution were tested in vivo by measuring lanadelumab protein expression compared to vector genome copy in each wild type mouse.

Vectors were administered intravenously to C57B1/6 mice at equivalent doses (2.5×10¹² vg/kg). Mouse serum was collected biweekly, and lanadelumab protein expression levels were determined by ELISA. Liver samples were harvested at 49 days post vector administration. The presence of viral genomes in each sample was quantified using Lanadelumab probe and primer by Droplet Digital PCR (ddPCR) (the NAICA™ system from Stilla). The genome copy number of glucagon was also measured simultaneously in each sample, the viral genomes were then normalized

| | Male 8-10 week old | | | Bleeds - Day sampled (X) | | | | | | | | | | |
|---|---|---|---|---|---|---|---|---|---|---|---|---|---|---|
| Rat | Wistar Rats (180-200 g) | | | | Day | Day | Day | Day | Day | Day | Day | Day | Day | Day |
| group | Treatment | N | ROA | Day −7 | 0 | 7 | 10 | 14 | 17 | 21 | 28 | 35 | 42 | 49 |
| 1 | AAV8.CAG.LANv2.T2A | 3 | IM | X | Adminis- | X | X | X | X | X | X | X | X | sacrifice |
| | Dose: 1e13 vg/kg | | | | tration | | | | | | | | | |
| 2 | AAV8.CAG.LANv2.T2A | 3 | IV | X | Adminis- | X | X | X | X | X | X | X | X | sacrifice |
| | Dose: 1e13 vg/kg | | | | tration | | | | | | | | | |
| 3 | AAV8.CAG.LANv2.T2A | 3 | IV | X | Adminis- | X | X | X | X | X | X | X | X | sacrifice |
| | Dose: 1e14 vg/kg | | | | tration | | | | | | | | | |

and demonstrated as vector genome copy number per cell (assuming 2 glucagon/cell). Statistical analysis was performed using one-way ANOVA in GraphPad Prism 8.

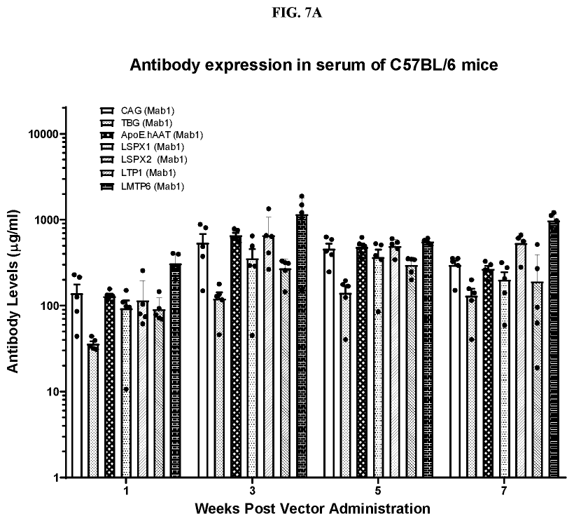

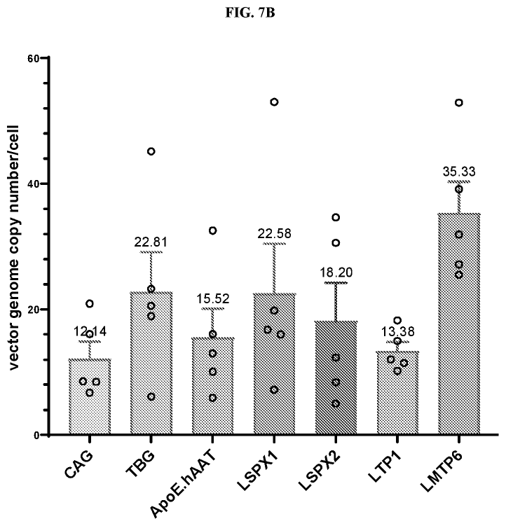

Among the AAV8 vectors with liver-specific promoters, the vectors driven by the ApoE.hAAT (SEQ ID NO:78) and LMTP6 (SEQ ID NO:71) promoters provided the highest amount of protein expression at all time points (FIG. 7A). While for the biodistribution data, there was no significant difference of vector genome copy number per cell in liver samples in animals treated with vectors driven by different promoters (FIG. 7B).

All liver-specific promoters outperform the TBG promoter (SEQ ID NO:93), and the dual-specific LMTP6 promoter (SEQ ID NO:71) consistently shows the highest expression in the serum (μg/ml) (FIG. 7).

### Example 14: Lanadelumab Expression in Rat Serum Following Administration of Vectorized Antibody

A high level of Lanadelumab expression was detected in the serum of mice treated with AAV-Lanadelumab via IV administration. In parts of the study, the lanadelumab expression levels in different rat strains treated with different doses of AAV-Lanadelumab vectors and controls were examined.

Experiment 1 (Wistar rats):

To evaluate the route and the dose of vector administration in rats, a control vector AAV.CAG-LANv2.T2A (CAG.L02; SEQ ID NO:223) was tested in Wistar rat. Eight to ten weeks old male Wistar rats were assigned into three groups (n=3 per group) to receive vector administration via IM or IV injection at a dose of 1×10¹³ vg/kg or 1×10¹⁴ vg/kg. Blood was collected at 7 days before treatment and 7, 10, 14, 17, 21, 28, 35, 42 and 49 days post vector administration and processed into serum.

TABLE 10
Study details for Lanadelumab expression in rat serum, Experiment 1.

| | Male 8-10 week old | | | Bleeds - Day sampled (X) | | | | | | | | | | |
|---|---|---|---|---|---|---|---|---|---|---|---|---|---|---|
| Rat | Wistar Rats (180-200 g) | | | | Day | Day | Day | Day | Day | Day | Day | Day | Day | Day |
| group | Treatment | N | ROA | Day −7 | 0 | 7 | 10 | 14 | 17 | 21 | 28 | 35 | 42 | 49 |
| 1 | AAV8.CAG.LANv2.T2A | 3 | IM | X | Adminis- | X | X | X | X | X | X | X | X | sacrifice |
| | Dose: 1e13 vg/kg | | | | tration | | | | | | | | | |
| 2 | AAV8.CAG.LANv2.T2A | 3 | IV | X | Adminis- | X | X | X | X | X | X | X | X | sacrifice |
| | Dose: 1e13 vg/kg | | | | tration | | | | | | | | | |
| 3 | AAV8.CAG.LANv2.T2A | 3 | IV | X | Adminis- | X | X | X | X | X | X | X | X | sacrifice |
| | Dose: 1e14 vg/kg | | | | tration | | | | | | | | | |

Levels of human IgG antibody in collected rat serum were detected by ELISA. Statistical analysis was done by one-way ANOVA with multiple comparisons at each time point using Prism

TABLE 11
Results of Lanadelumab expression in Wistar rats, Experiment 1

| | AAV8.CAG.Lanv2.T2A | | | AAV8.CAG.Lanv2.T2A | | | AAV8.CAG.Lanv2.T2A | | |
|---|---|---|---|---|---|---|---|---|---|
| Day | 1e13 vg/kg- IM | | | 1e13 vg/kg- IV | | | 1e14 vg/kg- IM | | |
| sampled | Mean | SEM | N | Mean | SEM | N | Mean | SEM | N |
| D 7 | 4.1 | 0.96 | 3 | 8.43 | 0.64 | 3 | 33.6 | 13.86 | 3 |
| D 10 | 7.67 | 1.91 | 3 | 12.3 | 0.81 | 3 | 64.5 | 30.2 | 3 |
| D 14 | 9.37 | 0.73 | 3 | 23.13 | 0.18 | 3 | 123.23 | 69.12 | 3 |
| D 17 | 4.97 | 2.23 | 3 | 92 | 53.52 | 3 | 245.17 | 151.09 | 3 |
| D 21 | 2.04 | 0.9 | 3 | 33.06 | 5.84 | 3 | 252.63 | 149.41 | 3 |
| D 28 | 92.08 | 87.07 | 3 | 65.23 | 41.69 | 3 | 117.97 | 112.47 | 3 |
| D 35 | 14.46 | 12.31 | 3 | 88.1 | 56.68 | 3 | 122.97 | 74.94 | 3 |
| D 42 | 82.29 | 80.46 | 3 | 40.43 | 26.77 | 3 | 108.02 | 99.26 | 3 |
| D 49 | 1.66 | 0.95 | 3 | 81.4 | 39.96 | 3 | 216.30 | 118.18 | 3 |

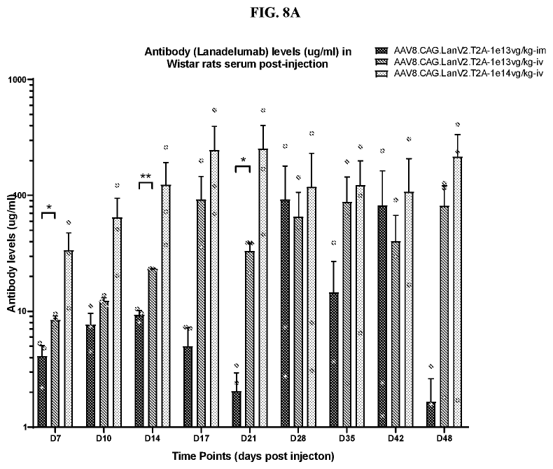

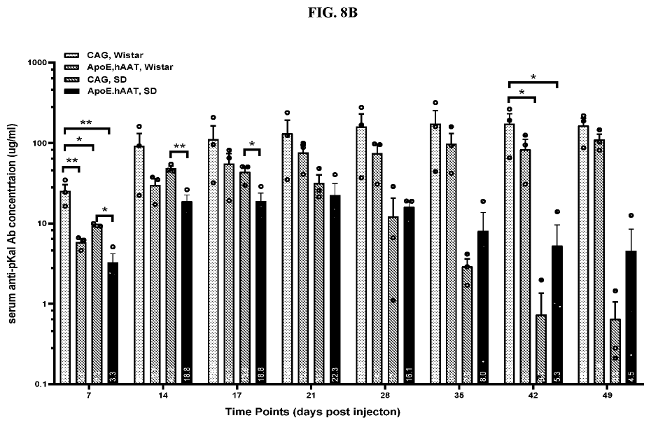

The levels of antibody in rat serum were detectable at 7 days post treatment. It increased over time and reached the peak level at 17 (lower dose) and 21 (higher dose) days post treatment in IV groups and 28 days in IM group. The antibody levels gradually decreased and sustains up to 48 days post treatment in all groups. For animals treated with lower dose (1×10¹³ vg/kg) vector, the antibody expression levels in IV groups are significantly higher than that in IM group at 7, 14 and 21 days post vector administration. For animals received IV administration, the antibody expression levels were dose-dependent at all time points. The highest level of lanadelumab expression was 252.6±149.4 μg/ml, which was detected in animals treated with higher dose (1×10¹⁴ vg/kg) at 21 days post IV administration. See FIG. 8A.

### Experiment 2 (Wistar and Sprague-Dawley Rats):

The aim of this experiment was to investigate the rat strain and the vector dose that will be used for a rat efficacy study. Eight to ten weeks old male Wistar and Sprague-Dawley (SD) rats were assigned into four groups (n=3 per group) to receive treatment of AAV8 vector carrying genome encoding lanadelumab driven by a universal promoter, CAG.L02 (SEQ ID NO:223), or a liver-specific promoter, ApoE.hAAT.L02 (SEQ ID NO:225). Vectors were administered via IV injection at a dose of 5×10¹³ vg/kg. Blood was collected at 7 days before treatment and 7, 10, 14, 17, 21, 28, 35, 42 and 49 days post vector administration and processed into the serum (Table 12). Levels of human IgG antibody in collected rat serum were detected by ELISA. Statistical analysis was done by one-way ANOVA with multiple comparisons at each time point using Prism.

TABLE 12
Study details for lanadelumab expression in rat serum, Experiment 2.

| | Male•8-10•week•old•¶ | | | | | | | | | | | | |
|---|---|---|---|---|---|---|---|---|---|---|---|---|---|
| | Wistar• or• Sprague• | | | | | | | | | | | | |
| | Dawley• (SD)• Rats | | | → •Bleeds•-•Day•sampled•(X)¤ | | | | | | | | | |
| | (180-200 g)¤ | | | | Day• | Day• | | | | | | | |
| Rat• | Treatment¶ | | | | 0¶ | 7¶ | Day• | Day• | Day• | Day• | Day• | Day• | Day• |
| group¤ | Dose:•5e13•vg/kg¤ | N¤ | ROA¤ | Day• −1¤ | ¤ | •¤ | 14¤ | 17¤ | 21•¤ | 28¤ | 35•¤ | 42¤ | 49¤ |
| 1¤ | CAG.L02¶ | 3¤ | IV¤ | X¤ | Adminis- | X¤ | X¤ | X¤ | X¤ | X¤ | X¤ | X¤ | sacrifice¤ |
| | Wistar¤ | | | | tration¤ | | | | | | | | |
| 2¤ | ApoE.hAAT.L02¶ | 3¤ | IV¤ | X¤ | Adminis- | X¤ | X¤ | X¤ | X¤ | X¤ | X¤ | X¤ | sacrifice¤ |
| | Wistar¤ | | | | tration¤ | | | | | | | | |
| 3¤ | CAG.L02¶ | 3¤ | IV¤ | X¤ | Adminis- | X¤ | X¤ | X¤ | X¤ | X¤ | X¤ | X¤ | sacrifice¤ |
| | Sprague•Dawley•(SD)¤ | | | | tration¤ | | | | | | | | |
| 4¤ | ApoE.hAAT.L02¶ | 3¤ | IV¤ | X¤ | Adminis- | X¤ | X¤ | X¤ | X¤ | X¤ | X¤ | X¤ | sacrifice¤ |
| | Sprague•Dawley•(SD)¤ | | | | tration¤ | | | | | | | | |

In this experiment, a control vector (CAG.L02, SEQ ID NO:223) and vector ApoE.hAAT.L02 (SEQ ID NO:225) were tested in Wistar and SD rats, respectively. Lanadelumab expression levels were higher in Wistar rat than SD rat in both vector groups at all time points. At the early time points, animals treated with control vector showed significant higher serum antibody levels than those treated with the liver-specific promoter containing vector. This was observed in Wistar rat at 7 days post treatment, and in SD rat at 7, 14 and 17 days post treatment. In Wistar rats, the concentrations of antibody gradually increased over time in both vectors group. The highest antibody levels were 173.1±78.8 μg/ml and 109.57±18.9 μg/ml at 35 and 49 days respectively in control CAG-Lanadelumab and hAAT-Lanadelumab vector-treated animals. In SD rats, however, the levels of antibody reached peaks at 14 and 21 days in control and lead vector-treated animals, respectively, and decreased gradually afterward in both groups. The highest antibody concentrations were 48.23±3.1 μg/ml and 22.33±8.98 μg/ml in CAG.L02 (SEQ ID NO:223) and ApoE.hAAT.L02 (SEQ ID NO:225) vector groups, respectively. See Table 13 and FIG. 8B.

TABLE 13
Results of Lanadelumab expression in Wistar rats, Experiment 2:

| | | |
|---|---|---|
| | CAG-Wistar | ApoE.hAAT- Wistar |
| Day | 5e13 vg/kg- IV | 5e13 vg/kg- IV |

| sampled | Mean | SEM | N | Mean | SEM | N |
|---|---|---|---|---|---|---|
| D 7 | 25.04 | 5.26 | 3 | 5.80 | 0.61 | 3 |
| D 14 | 91.50 | 39.90 | 3 | 29.73 | 6.36 | 3 |
| D 17 | 111.30 | 51.29 | 3 | 55.10 | 18.60 | 3 |
| D 21 | 132.03 | 58.76 | 3 | 75.80 | 17.94 | 3 |
| D 28 | 159.90 | 69.08 | 3 | 74.27 | 21.84 | 3 |
| D 35 | 173.10 | 78.76 | 3 | 97.67 | 33.89 | 3 |
| D 42 | 173.00 | 57.73 | 3 | 83.10 | 27.67 | 3 |
| D 49 | 163.57 | 39.45 | 3 | 109.57 | 18.87 | 3 |

### Example 15: Characterization of Vectorized Lanadelumab Regulated by Tissue-Specific Promoters Following Intramuscular Administration

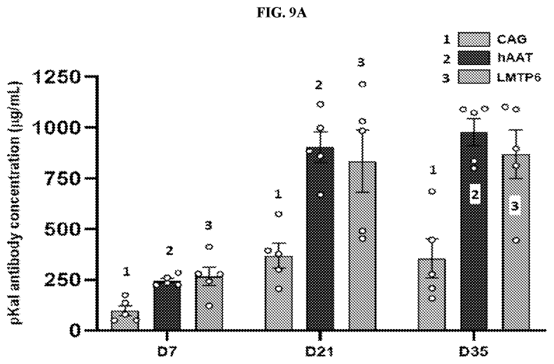

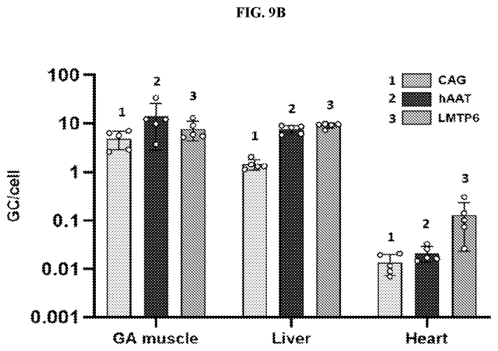

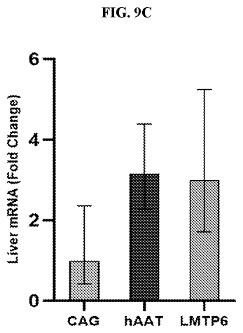

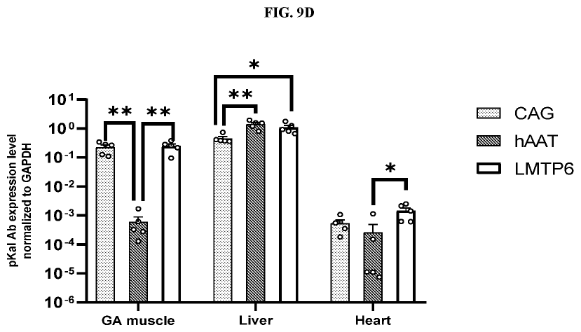

In a previous study, high liver-driven expression of vectorized lanadelumab with AAV8 regulated by the ApoE.hAAT or LMTP6 promoters was identified. The goal of this study was to characterize muscle-driven expression of the LMTP6 promoter following direct injection of lanadelumab vectors into the gastrocnemius (GA) muscle. Animals received bilateral injections of 5×10¹⁰ vg into the GA muscle. Serum was collected biweekly to measure systemic lanadelumab concentration (FIG. 9A). Animals were harvested at 49 days post-injection, and relevant tissues (liver, GA muscle, heart) were analyzed for vector biodistribution and transgene expression.

Vectors regulated by the hAAT and LMTP6 promoters demonstrated significantly increased antibody concentrations in serum compared to CAG at all time points (FIG. 9A). The hAAT and LMTP6 were not significantly different from each other in this experiment. Vector genome copies per cell of vectorized lanadelumab was detected and quantified in GA, liver and heart (FIG. 9B) with a notable difference of higher quantity of genome detected in heart for the dual muscle/liver promoter, LMTP6 vector. Increased liver RNA expression was also detected for all test vectors directly injected into GA muscle at 49 days (relative fold gene expression compared to a reference gene) (FIG. 9C). Gene expression (mRNA μg/mL) data from each of liver, GA muscle, and heart (FIG. 9D) indicates the dual specificity of LMPT6 in liver and muscle tissues following IM administration, whereas the hAAT-driven samples were reduced in muscle compared to LMTP6 and CAG. Significant differences were also seen between the hAAT and LMTP6 groups.

### Example 16: Comparison of Lanadelumab Protein Levels in Mouse Serum Derived from Mice Treated with AAV-Lanadelumab Vectors Produced with Different Production Systems

Different AAV production protocols were developed to identify methods that can increase AAV titer and scalability, as well as assess the quality of vector product. Cis and trans plasmids to generate AAV8.Lanadelumab rAAV vectors (all having the same transgene driven by a CAG promoter) were constructed by well-known methods suitable for HEK293-transfected cell and also baculovirus (BV)/Sf9 insect cell production methods. Three different BV/Sf9 vector systems, BV1, BV2 and BV3, were provided as well as rAAV vector produced by an HEK293 method as a control. Purified rAAV product was injected into wild-type mice for this protein expression study (Table 14).

Young adult C57BL/6 mice (aged 8-10 weeks) were administered with above-mentioned vectors at 2.5E′ 2 vg/kg via tail vein injection (n=5 per group). Serum was collected from each animal at 7, 21, 35, and 49 days post vector administration. Serum collected two days before injection (Day 0) served as baseline control. Levels of antibody (lanadelumab) expression were detected via ELISA. Data analysis was done by one-way ANOVA with multiple comparisons at each time point using Prism.

TABLE 14
Production system expression study design

| | AAV8.Lanadelumab 5e10 | | | | | | | | | |
|---|---|---|---|---|---|---|---|---|---|---|
| | vg (2.5e12 vg/kg) | | | | | | | | | |
| | rAAV | | | Bleeds sampled (X) | | | | | | |
| Mouse | Production | | | | Day 0 | Day 7 | Day 21 | Day 35 | Day 49 | |
| Group | method | N | ROA | Day −2 | (Week 0) | (Week 1) | (Week 3) | (Week 5) | (Week 7) | Day 50 |
| 1 | HEK cells | 5 | IV | X | Injection | X | X | X | X | Serum |
| 2 | BV1 | 5 | IV | X | Injection | X | X | X | X | Serum |
| Werum | BV2 | 5 | IV | X | Injection | X | X | X | X | Serum |
| 3 | | | | | | | | | | |
| 4 | BV3 | 5 | IV | X | Injection | X | X | X | X | Serum |

All production methods tested are viable based on this study, with greater yields from the HEK cell production method at the time points tested. Antibody expression in serum is detectable as early as 7 days post administration in all groups. The average of antibody concentration at Day 7 in the HEK production group is 386 μg/ml, which is significantly higher than other groups (61-102 μg/ml). The levels of antibody expression increase at day 21 by 1-, 6-, 7-, and 4-fold in BV1, BV2 and BV3 groups, respectively. Antibody expression levels sustained at 35 and 49 days post administration. There is no significant difference in between HEK produced vector and BV3 produced vectors at day 21, 35 and 49 time points.

### Example 17: Vectorized Human Anti-pKal Antibody, Lanadelumab, Derived from Mouse Serum Suppressed Human pKal Function

In order to measure pKal function of lanadelumab derived from mouse serum following AAV-lanadelumab administration, a fluorescence-based kinetic enzymatic functional assay was performed. First, activated human plasma kallikrein (Enzyme Research Laboratories) was diluted in sample dilution buffer (SDB; 1×PBS, 3% BSA, 0.1% Tween-20) to top concentration of 100 nM. This pKal was two-fold serially diluted for a total of 12 concentrations in the dilution series (100 nM-0.05 nM). From each dilution, and in duplicate, 254, was placed in one well of a 96-well, opaque flat-bottomed plate along with 254, of SDB. Then, 504, of the fluorogenic substrate Pro-Phe-Arg-7-Amino-4-Methylcoumarin (PFR-AMC) (Bachem) prepared at 100 μM in assay buffer (50 mM Tris, 250 mM NaCl, pH 7.5) was added to each well. The samples were immediately run in kinetic mode for AMC fluorescence at excitation/emission wavelengths of 380/460 nm, respectively, for 3 hours using a SpectraMax 3 fluorescent plate reader.

The signal-to-noise ratio for each pKal concentration RFU (last RFU fluorescent value chosen) was calculated by dividing its RFU by background PFR-AMC substrate fluorescence. The two lowest pKal concentrations with a signal-to-noise ratio ≥2 (6.25 nM and 12.5 nM) were then chosen to evaluate the suppressive effect and range of lanadelumab antibody of pKal function in a lanadelumab dose response. Lanadelumab (GenScript) or human IgG control antibody was diluted in SDB to top concentration of 200 nM and two-fold serially diluted to 0.39 nM. Next, 254, pKal (each of two chosen concentrations) was incubated with 25 μL lanadelumab or human IgG at 30° C. for 1 hour. Antibody-pKal mixture was then given PFR-AMC and immediately run in kinetic mode for AMC fluorescence at excitation/emission wavelengths of 380/460 nm, respectively, for 3 hours using a SpectraMax fluorescent plate reader.

In vitro pKal functional assay. When used, mouse serum was diluted in sample dilution buffer and incubated 1:1 with 6.25 nM (1.56 nM in-well) pKal for 30° C./1 hour. For total IgG purification from mouse serum, antibody was purified using the Protein A Spin Antibody Purification Kit (BioVision) according to manufacturer's protocol. Total antibody concentration was measured using a Nanodrop spectrophotometer, with OD absorbance=280 nM. AMC standard curve was generated by a two-fold downward dilution series of AMC (500 nM, eleven dilutions and blank subtracted) diluted in assay buffer. AMC was read as end point fluorescence at excitation/emission wavelengths of 380/460 nm, respectively. Specific plasma kallikrein activity was calculated as: (adjusted experimental sample Vmax, RFU/sec)×(Conversion factor, AMC standard curve μM/RFU)/(pKal concentration, nM). Percent reduction in pKal activity was derived from calculating day 49 by day −7 pKal activity.

To determine whether AAV-derived lanadelumab can suppress plasma kallikrein function, we developed the in vitro AMC substrate-based functional assay to address this in a proof-of-concept study. In this assay, antibody-containing medium is incubated with activated human pKal, as described above. Antibody-bound pKal is then given the synthetic peptide substrate Pro-Phe-Arg conjugated to AMC (PFR-AMC) and amount of released AMC is measured over time at excitation/emission wavelengths of 380/460 nm, respectively, for 3 hours. The assay showed noticeable lanadelumab-mediated suppression of pKal activity down to 0.1 nM (in-well concentration) at a defined enzyme concentration. We first sought to determine whether serum from mice administered lanadelumab-encoded AAV could suppress pKal activity. Serum from mice 49 days post-administration was diluted 1:25 (in range predicted to be suppressive), incubated with pKal in vitro, and pKal activity was assayed. Serum from mice post-vector administration, as opposed to 7 days pre-administration, suppressed pKal activity, as reflected in a significant reduction of enzyme activity and a ˜50% percent reduction in pKal activity between the two time points.

Further experiments show that suppression was due to the lanadelumab within the serum. Reasoning that the human IgG, namely lanadelumab, would only be found in the day 49 post-administration IgG fraction, but not the day −7 pre-administration samples, purified and total IgG antibody was used from the aforementioned day −7 and day 49 mouse serum samples to test pKal suppression. Indeed, only lanadelumab-containing purified IgG from day 49 post-administration serum, but not IgG from the pre-administration time point, suppressed human pKal function (FIG. 14F).

### Example 18: Effects of AAV-Lanadelumab in Carrageenan Animal Models

### Example 18A: Effects of AAV-Lanadelumab in the Carrageenan Paw Edema Model in

Inflammation models induced by carrageenan are frequently used acute inflammation models. Carrageenan (Cg) is a strong chemical agent that functions in stimulating the release of inflammatory and proinflammatory mediators, including bradykinin, histamine, tachykinins, reactive oxygen, and nitrogen species. Typical signs of inflammation include edema, hyperalgesia, and erythema, which develop immediately following the treatment of carrageenan. This example evaluated the effect of AAV-mediated gene delivery of Lanadelumab on carrageenan-induced paw edema in mice.

In total eighty young adult (8-9 weeks old) male C57BL/6 mice were used for this study. Animals were divided into eight groups as listed in Table 15. Paw edema was induced by a single subcutaneous (s.c.) injection of 30 μL of 0.7% or 1% carrageenan solution. Test vectors and positive control Diclofenac were administered at 21 days and 30 minutes prior to carrageenan treatment. Blood was collected before vectors injection and at 7 and 21 days post injection from mice in groups 1, 3, 4, 5, 7 and 8. Paw volume was measured using a digital Plethysmometer prior to carrageenan injection, and at 2, 4, 6, 8, 24 and 48 hours after injection. All animals were sacrificed 48 hours after carrageenan injection. Liver and paw specimens were also collected at the necropsy.

TABLE 15
Carrageenan (Cg) Paw Edema Mouse Study design

| | | Test Items | | Route, Dose | Pre-treatment | |
|---|---|---|---|---|---|---|
| *Gr. | Group/treatment | Dose level | | volume | time | N |
| 1 | Cg (0.7%)/Vehicle | 0 | | I.P., | 30 min before Cg | 10 |
| | of diclofenac | | | 10 mL/kg | | |
| 2 | Cg (0.7%)/Diclofenac | 60 | mg/kg | I.P., | 30 min before Cg | 10 |
| | | | | 10 mL/kg | | |
| 3 | Cg (0.7%)/Vector 1 | 1e13 | vg/kg | I.V., | 3 weeks before Cg | 10 |
| | | | | 3.333 mL/kg | | |
| 4 | Cg (0.7%)/ | 1e13 | vg/kg | I.V., | 3 weeks before Cg | 10 |
| | AAV8.ApoE.hAAT.Lan | | | 3.333 mL/kg | | |
| 5 | Cg (1.0%)/Vehicle | 0 | | I.P., | 30 min before Cg | 10 |
| | of diclofenac | | | 10 mL/kg | | |
| 6 | Cg (1.0%)/Diclofenac | 60 | mg/kg | I.P., | 30 min before Cg | 10 |
| | | | | 10 mL/kg | | |
| 7 | Cg (1.0%)/Vector 1 | 1e13 | vg/kg | I.V., | 3 weeks before Cg | 10 |
| | | | | 3.333 mL/kg | | |
| 8 | Cg (1.0%)/ | 1e13 | vg/kg | I.V., | 3 weeks before Cg | 10 |
| | AAV8.ApoE.hAAT.Lan | | | 3.333 mL/kg | | |

Vector 1: AAV8-GFP

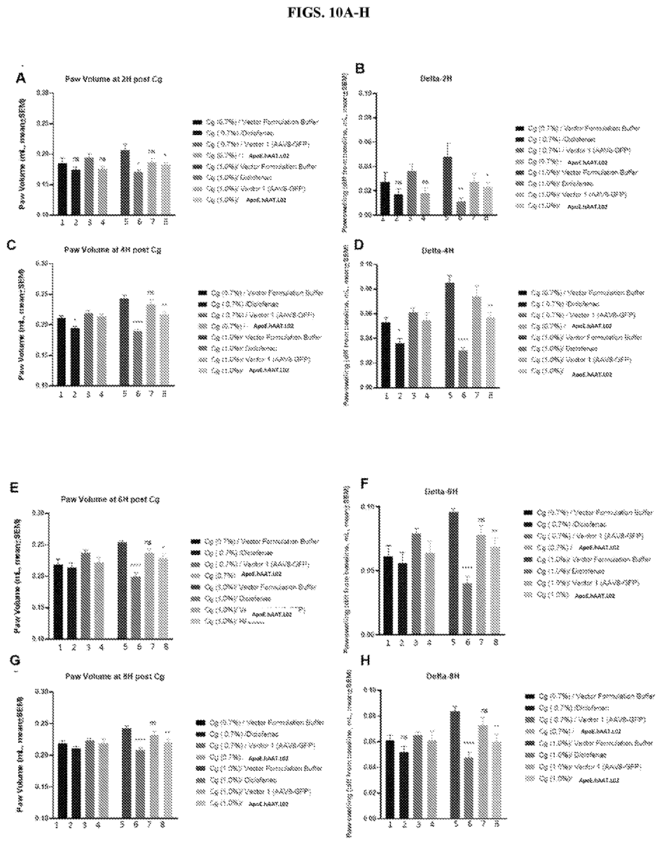

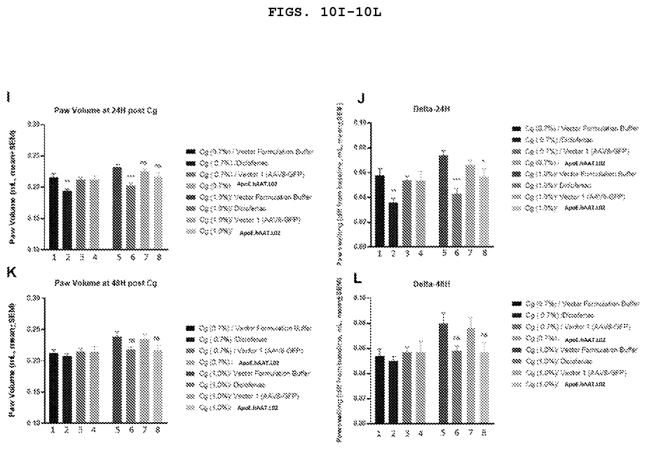

Both 0.7% and 1.0% carrageenan induced swelling in the injected paw; however, swelling was more pronounced with 1.0% carrageenan injection (FIGS. 10, A-L). In the positive control groups (Group 2 and 6), diclofenac treatment significantly decreased the paw volume at 2, 4, 6, 8 and 24 hours post carrageenan injection in 1.0% Cg model (group 2), while a significant decrease on paw volume was observed only at 4 and 24 hours post injection in 0.7% Cg model (group 6).

ApoE.hAAT.L02 (SEQ ID NO:225) treatment significantly reduced the paw volume at 2, 4, 6 and 8 hours post carrageenan injection in 1.0% Cg model when compared with the vehicle control group (group 1, vector formulation buffer). However, no effect of ApoE.hAAT.L02 treatment was observed in 0.7% Cg model at any time points. There is no significant difference in between groups treated with vehicle (groups 1 and 4) or control vector (AAV-GFP, groups 3 and 7) in both 1.0% and 0.7% Cg models. All data was analyzed with One-way ANOVA with Dunnett's post-hoc test for multiple comparisons.

These data indicate that acute inflammation can be successfully induced in mouse paw with a single subcutaneous injection of 1% carrageenan solution. Lanadelumab, a human IgG antibody produced in mouse serum via AAV-mediated gene delivery significantly reduces the severity of inflammation in mouse 1% carrageenan model.

### Example 19: Characterization of Tissue-Restricted Transgene Immunogenicity

The goal of this study is to understand transgene immunogenicity and/or tolerance induction in the context of ubiquitous, tissue-specific, or tandem promoters. Hypothesis: Vectors driven by liver-specific and liver-muscle tandem promoters will demonstrate reduced immunogenicity compared to vectors driven by a ubiquitous promoter. To test this hypothesis, four AAV vectors that drive expression of a highly immunogenic membrane-bound ovalbumin (mOVA) were constructed. These vectors differ in their promoter sequences which includes: a) a ubiquitous CAG promoter (SEQ ID NO:89) b) the liver-specific hAAT promoter with upstream ApoE enhancer (SEQ ID NO:78) c), the muscle-specific CK8 promoter cassette composed of the CK core promoter and three copies of a modified MCK enhancer (SEQ ID NO:90), and d) liver-muscle tandem promoter 6 (LMTP6, SEQ ID NO:71) that contains sequence elements derived from hAAT and CK8. Initial experiments will measure the immune response following intravenous (IV) vector administration within mice. Study endpoints will include characterization of humoral and cell-mediated immune responses against the mOVA transgene product. In addition, tissues will be harvested for vector biodistribution and transgene expression analysis.

### Example 20: Plasma Expression of Vectorized Lanadelumab in Cynomolgus Monkeys

### Methods

Plasma kinetics of lanadelumab expression in non-human primates administered AAV vectors encoding lanadelumab antibodies were assessed. The goal of this study was to assess and select the dose of AAV8.ApoE.hAAT.Lan vector that results in sustained lanadelumab expression of at least 200 μg/ml lanadelumab by three months or more. The cynomolgus monkey were chosen as the test system because of its established usefulness and acceptance as a model for AAV biodistribution studies in a large animal species and for further translation to human. All animals on this study were naïve with respect to prior treatment.

Nine cynomolgus animals were used. Animals judged suitable for experimentation based on clinical sign data and prescreening antibody titers were placed in three study groups, each receiving a different dosage of AAV vector, by body weight using computer-generated random numbers. Each set of three animals were administered a single i.v. dose of the vector AAV8.ApoE.hAAT.Lan vector (described above) at the dose of 1E12 gc/kg (Group 1), 1E13 gc/kg (Group 2), and 1E14 gc/kg (Group 3).

Clinical signs were recorded at least once daily beginning approximately two weeks prior to initiation of dosing and continuing throughout the study period. The animals were observed for signs of clinical effects, illness, and/or death. Additional observations were recorded based upon the condition of the animal at the discretion of the Study Director and/or technicians.

Blood samples were collected from a peripheral vein for bioanalytical analysis prior to dose administration and then at weekly intervals for 10 weeks. The samples were collected in clot tubes and the times were recorded. The tubes were maintained at room temperature until fully clotted, then centrifuged at approximately 2400 rpm at room temperature for 15 minutes. The serum was harvested, placed in labeled vials, frozen in liquid nitrogen, and stored at −60° C. or below.

All animals were sedated with 8 mg/kg of ketamine HCl IM, maintained on an isoflurane/oxygen mixture and provided with an intravenous bolus of heparin sodium, 200 IU/kg. The animals were perfused via the left cardiac ventricle with 0.001% sodium nitrite in saline.

As primary endpoint analysis, plasma samples were assayed for lanadelumab concentration by ELISA and/or western blot, to be reported at least as μg lanadelumab per ml plasma; and lanadelumab activity, for example, kallikrein inhibition, by fluorogenic assay.

The presence of antibodies against lanadelumab (ADAs) in the serum were evaluated by ELISA and lanadelumab binding assays. Biodistribution of the vector and lanadelumab coding transcripts were assessed in necroscopy samples by quantitative PCR and NGS methods. Tissues to be assayed included liver, muscle, and heart. Toxicity assessment was done by full pathology, including assaying liver enzymes, urinalysis, cardiovascular health, and more.

### Results

The optimized expression cassette containing a liver-specific promoter and a codon optimized and CpG depleted transgene with a modified furin-T2A processing signal resulted in dose-dependent serum antibody concentrations when delivered intravenously using an AAV8 vector. Sustained levels of functional anti-kallikrein antibody were achieved in the serum of 7 out of 9 cynomolgus monkeys following IV vector administration at all three doses (1E12 gc/kg, 1E13 gc/kg, and 1E14 gc/kg) (FIG. 1). Functional anti-kallikrein antibody was detected in the serum of all animals regardless of the administered dose. A plateau was reached at 29 days after dose administration with mean maximum levels of 0.144 μg/mL, 0.635 μg/mL, and 35.16 μg/mL being detected in animals 29 days after receiving 1E12 gc/kg, 1E13 gc/kg, and 1E14 gc/kg, respectively.

### EQUIVALENTS

Although the invention is described in detail with reference to specific embodiments thereof, it will be understood that variations which are functionally equivalent are within the scope of this invention. Indeed, various modifications of the invention in addition to those shown and described herein will become apparent to those skilled in the art from the foregoing description and accompanying drawings. Such modifications are intended to fall within the scope of the appended claims. Those skilled in the art will recognize or be able to ascertain using no more than routine experimentation, many equivalents to the specific embodiments of the invention described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference in their entireties.

What is claimed is:

1. A pharmaceutical composition for treating hereditary angioedema, thrombosis, or hypercoagulation in a human subject in need thereof, comprising an adeno-associated virus (AAV) vector having:
(a) a viral capsid that has a tropism for liver and/or muscle cells; and
(b) an artificial genome comprising an expression cassette flanked by AAV inverted terminal repeats (ITRs), wherein the expression cassette comprises a transgene encoding a heavy chain and a light chain of a substantially full-length or full-length anti-factor XII/XIIa mAb, operably linked to one or more regulatory sequences that promote expression of the transgene in human liver and/or muscle cells;
wherein said AAV vector is formulated for systemic administration to said human subject.

2. The pharmaceutical composition of claim 1, wherein the viral capsid is at least 95% identical to the amino acid sequence of AAV3B, AAV5, AAV7 (SEQ ID NO:198), AAV8 (SEQ ID NO:199), AAV9 (SEQ ID NO:200), AAVrh10 (SEQ ID NO:201), AAVrh46 (SEQ ID NO:202), AAVrh73 (SEQ ID NO:203), AAVS3 (SEQ ID NO:205), AAV-LK03 (SEQ ID NO:204), AAVrh8, AAV64R1, or AAVhu37.

3. The pharmaceutical composition of claim 1 or 2 wherein the AAV capsid is AAV8 or AAVS3.

4. The pharmaceutical composition of claims 1 to 3, wherein the regulatory sequence includes a regulatory sequence from Table 1.

5. The pharmaceutical composition of claim 4, wherein the regulator sequence is an ApoE.hAAT (SEQ ID NO:78) regulatory sequence, a LSPX1 promoter (SEQ ID NO:66), a LSPX2 promoter (SEQ ID NO:67), a LTP1 promoter (SEQ ID NO:68), a LTP2 (SEQ ID NO:69) promoter, or a LTP3 (SEQ ID NO:70) promoter.

6. The pharmaceutical composition of any of claims 1 to 5, wherein the transgene comprises a Furin/2A linker between the nucleotide sequences coding for the heavy and light chains of said mAb.

7. The pharmaceutical composition of claim 6, wherein said Furin 2A linker is a Furin/T2A linker having the amino acid sequence RKRR(GSG)EGRGSLLTCGDVEENPGP (SEQ ID NOS:155 or 156).

8. The pharmaceutical composition of any of claims 1 to 7, wherein the transgene encodes a signal sequence at the N-terminus of the heavy chain and the light chain of said antigen-binding fragment that directs secretion and post translational modification in said human liver and/or muscle cells.

9. The pharmaceutical composition of claim 8, wherein said signal sequence is MYRMQLLLLIALSLALVTNS (SEQ ID NO:103) or a signal sequence from Table 2.

10. The pharmaceutical composition of any of claims 1 to 9, wherein transgene has the structure:
signal sequence-Heavy chain-Furin site-2A site-signal sequence-Light chain-PolyA.

11. The pharmaceutical composition of any of claims 1 to 10, wherein the anti-Factor XII antibody is garadacimab, AB042/AB043, AB054, DX-4012, Ab26036, Ab26048, Ab260489, Ab26076, 559C-M0071-F06, 559C-M0179-D04, 559C-M0181-C02, 559C-M0180-G03, 559C-M0184-B04, 620I-X0173-A11, 620I-X0173-C07, 620I-X0173-E07, or 620I-X0173-G11 or an antigen binding fragment thereof

12. The pharmaceutical composition of any of claims 1 to 11, wherein the full-length mAb or the antigen-binding fragment comprises a heavy chain with an amino acid sequence of SEQ ID NO: 1 and optionally an Fc polypeptide with an amino acid sequence of SEQ ID NO:63 and a light chain with an amino acid sequence of SEQ ID NO:2; a heavy chain with an amino acid sequence of SEQ ID NO:3 and a light chain with an amino acid sequence of SEQ ID NO: 4; a heavy chain with an amino acid sequence of SEQ ID NO:5 and optionally an Fc polypeptide with an amino acid sequence of SEQ ID NO: 43 and a light chain with an amino acid sequence of SEQ ID NO:6; a heavy chain with an amino acid sequence of SEQ ID NO:7 and optionally an Fc polypeptide with an amino acid sequence of SEQ ID NO:65 and a light chain with an amino acid sequence of SEQ ID NO:8; a heavy chain with an amino acid sequence of SEQ ID NO:10 and a light chain with an amino acid sequence of SEQ ID NO:11; a heavy chain with an amino acid sequence of SEQ ID NO:12 and a light chain with an amino acid sequence of SEQ ID NO:13; a heavy chain with an amino acid sequence of SEQ ID NO:14 and a light chain with an amino acid sequence of SEQ ID NO:15; a heavy chain with an amino acid sequence of SEQ ID NO: 16 and a light chain with an amino acid sequence of SEQ ID NO:17; a heavy chain with an amino acid sequence of SEQ ID NOS:18, 20, 22, or 24 and a light chain with an amino acid sequence of SEQ ID NO:19; a heavy chain with an amino acid sequence of SEQ ID NO:26 and a light chain with an amino acid sequence of SEQ ID NO:27; a heavy chain with an amino acid sequence of SEQ ID NO:26 and a light chain with an amino acid sequence of SEQ ID NO:27, 28, 29, or 30; or a heavy chain with an amino acid sequence of SEQ ID NO:31 and a light chain with an amino acid sequence of SEQ ID NO:27.

13. A composition comprising an adeno-associated virus (AAV) vector having:
a. a viral AAV capsid, that is optionally at least 95% identical to the amino acid sequence of AAV3B, AAV5, AAV7 (SEQ ID NO:198), AAV8 (SEQ ID NO:199), AAV9 (SEQ ID NO:200), AAVrh10 (SEQ ID NO:201), AAVrh46 (SEQ ID NO:202), AAVrh73 (SEQ ID NO:203), AAVS3 (SEQ ID NO:205), AAV-LK03 (SEQ ID NO:204), AAVrh8, AAV64R1, or AAVhu37; and
b. an artificial genome comprising an expression cassette flanked by AAV inverted terminal repeats (ITRs), wherein the expression cassette comprises a transgene encoding a heavy and a light chain of a substantially full-length or full-length anti-factor XII mAb, operably linked to one or more regulatory sequences that promote expression of the transgene in human liver and/or muscle cells;
c. wherein the transgene encodes a signal sequence at the N-terminus of the heavy chain and the light chain of said mAb that directs secretion and post translational modification of said mAb in liver and/or muscle cells.

14. The composition of claim 13, wherein the anti-factor XII antibody is garadacimab, AB042/AB043, AB054, DX-4012, Ab26036, Ab26048, Ab260489, Ab26076, 559C-M0071-F06, 559C-M0179-D04, 559C-M0181-CO2, 559C-M0180-G03, 559C-M0184-B04, 620I-X0173-A11, 620I-X0173-C07, 620I-X0173-E07, or 620I-X0173-G11 or an antigen binding fragment thereof

15. The composition of claim 13 or 14, wherein the full-length mAb or the antigen-binding fragment comprises a heavy chain with an amino acid sequence of SEQ ID NO:1 and optionally an Fc polypeptide with an amino acid sequence of SEQ ID NO:63 and a light chain with an amino acid sequence of SEQ ID NO:2; a heavy chain with an amino acid sequence of SEQ ID NO:3 and a light chain with an amino acid sequence of SEQ ID NO: 4; a heavy chain with an amino acid sequence of SEQ ID NO:5 and optionally an Fc polypeptide with an amino acid sequence of SEQ ID NO:64 and a light chain with an amino acid sequence of SEQ ID NO: 6; a heavy chain with an amino acid sequence of SEQ ID NO:7 and optionally an Fc polypeptide with an amino acid sequence of SEQ ID NO:65 and a light chain with an amino acid sequence of SEQ ID NO:8; a heavy chain with an amino acid sequence of SEQ ID NO:10 and a light chain with an amino acid sequence of SEQ ID NO:11; a heavy chain with an amino acid sequence of SEQ ID NO:12 and a light chain with an amino acid sequence of SEQ ID NO: 3; a heavy chain with an amino acid sequence of SEQ ID NO:14 and a light chain with an amino acid sequence of SEQ ID NO:15; a heavy chain with an amino acid sequence of SEQ ID NO: 16 and a light chain with an amino acid sequence of SEQ ID NO:17; a heavy chain with an amino acid sequence of SEQ ID NOS:18, 20, 21, or 22 and a light chain with an amino acid sequence of SEQ ID NO:19; a heavy chain with an amino acid sequence of SEQ ID NO:26 and a light chain with an amino acid sequence of SEQ ID NO:27; a heavy chain with an amino acid sequence of SEQ ID NO:26 and a light chain with an amino acid sequence of SEQ ID NO:27, 28, 29, or 30; or a heavy chain with an amino acid sequence of SEQ ID NO:31 and a light chain with an amino acid sequence of SEQ ID NO:27.

16. The composition of any of claims 13 to 15, wherein the transgene comprises a Furin/2A linker between the nucleotide sequences coding for the heavy and light chains of said mAb.

17. The composition of claim 16, wherein the nucleic acid encoding a Furin 2A linker is incorporated into the expression cassette in between the nucleotide sequences encoding the heavy and light chain sequences, resulting in a construct with the structure: Signal sequence-Heavy chain-Furin site-2A site-Signal sequence-Light chain-PolyA.

18. The composition of claims 13 to 17, wherein said Furin 2A linker is a Furin/T2A linker having the amino acid sequence RKRR(GSG)EGRGSLLTCGDVEENPGP (SEQ ID NOS:155 or 156).

19. The composition of any of claims 13 to 18, wherein said signal sequence is MYRMQLLLLIALSLALVTNS (SEQ ID NO:103) or a signal sequence from Tables 2 or 3.

20. A method of treating hereditary angioedema, thrombosis, or hypercoagulation in a human subject in need thereof, comprising intravenously administering to the subject a therapeutically effective amount of a composition comprising a recombinant AAV comprising a transgene encoding garadacimab, AB042/AB043, AB054, DX-4012, Ab26036, Ab26048, Ab260489, Ab26076, 559C-M0071-F06, 559C-M0179-D04, 559C-M0181-C02, 559C-M0180-G03, 559C-M0184-B04, 620I-X0173-A11, 620I-X0173-C07, 620I-X0173-E07, or 620I-X0173-G11, operably linked to one or more regulatory sequences that control expression of the transgene in liver and/or muscle cells, in an amount sufficient to result in expression from the transgene and secretion of garadacimab, AB042/AB043, AB054, DX-4012, Ab26036, Ab26048, Ab260489, Ab26076, 559C-M0071-F06, 559C-M0179-D04, 559C-M0181-C02, 559C-M0180-G03, 559C-M0184-B04, 620I-X0173-A11, 620I-X0173-C07, 620I-X0173-E07, or 620I-X0173-G11 into the bloodstream of the human subject.

21. The method of claim 20, wherein the full-length mAb or the antigen-binding fragment comprises a heavy chain with an amino acid sequence of SEQ ID NO:1 and optionally an Fc polypeptide with an amino acid sequence of SEQ ID NO:63 and a light chain with an amino acid sequence of SEQ ID NO:2; a heavy chain with an amino acid sequence of SEQ ID NO:3 and a light chain with an amino acid sequence of SEQ ID NO:4; a heavy chain with an amino acid sequence of SEQ ID NO:5 and optionally an Fc polypeptide with an amino acid sequence of SEQ ID NO: 43 and a light chain with an amino acid sequence of SEQ ID NO: 6; a heavy chain with an amino acid sequence of SEQ ID NO:7 and optionally an Fc polypeptide with an amino acid sequence of SEQ ID NO:65 and a light chain with an amino acid sequence of SEQ ID NO:8; a heavy chain with an amino acid sequence of SEQ ID NO:10 and a light chain with an amino acid sequence of SEQ ID NO:11; a heavy chain with an amino acid sequence of SEQ ID NO:12 and a light chain with an amino acid sequence of SEQ ID NO:13; a heavy chain with an amino acid sequence of SEQ ID NO:14 and a light chain with an amino acid sequence of SEQ ID NO:15; a heavy chain with an amino acid sequence of SEQ ID NO:16 and a light chain with an amino acid sequence of SEQ ID NO:17; a heavy chain with an amino acid sequence of SEQ ID NOS: 18, 20, 21, or 22 and a light chain with an amino acid sequence of SEQ ID NO:19; a heavy chain with an amino acid sequence of SEQ ID NO:26 and a light chain with an amino acid sequence of SEQ ID NO:27; a heavy chain with an amino acid sequence of SEQ ID NO:26 and a light chain with an amino acid sequence of SEQ ID NO:27, 28, 29, or 30; or a heavy chain with an amino acid sequence of SEQ ID NO:31 and a light chain with an amino acid sequence of SEQ ID NO:27.

22. The method of claim 20 or 21 wherein the recombinant AAV has a viral capsid which is at least 95% identical to the amino acid sequence of AAV3B, AAV5, AAV7 (SEQ ID NO:198), AAV8 (SEQ ID NO:199), AAV9 (SEQ ID NO:200), AAVrh10 (SEQ ID NO: 201), AAVrh46 (SEQ ID NO:202), AAVrh73 (SEQ ID NO:203), AAVS3 (SEQ ID NO:203), AAV-LK03 (SEQ ID NO:204), AAVrh8, AAV64R1, or AAVhu37.

23. The method of any of claims 20 to 22, wherein the AAV capsid is AAV8 or AAVS3.

24. The method of any of claims 20 to 23, wherein the regulatory sequence includes a regulatory sequence from Table 1.

25. The method of claim 24, wherein the regulator sequence is an ApoE.hAAT (SEQ ID NO:78) regulatory sequence, a LSPX1 promoter (SEQ ID NO:66), a LSPX2 promoter (SEQ ID NO:67), a LTP1 promoter (SEQ ID NO:68), a LTP2 (SEQ ID NO:69) promoter, or a LTP3 (SEQ ID NO:70) promoter.

26. The method of any of claims 20 to 25, wherein the transgene comprises a Furin/2A linker between the nucleotide sequences coding for the heavy and light chains of said mAb.

27. The method of claim 26, wherein said Furin 2A linker is a Furin/T2A linker having the amino acid sequence RKRR(GSG)EGRGSLLTCGDVEENPGP (SEQ ID NOS:155 or 156).

28. The method of any of claims 20 to 27, wherein the transgene encodes a signal sequence at the N-terminus of the heavy chain and the light chain of said antigen-binding fragment that directs secretion and post translational modification in said human liver and/or muscle cells.

29. The method of claim 28, wherein said signal sequence is MYRMQLLLLIALSLALVTNS (SEQ ID NO:103) or a signal sequence from Tables 2 or 3.

30. The method of any of claims 20 to 29, wherein transgene has the structure: Signal sequence-Heavy chain-Furin site-2A site-Signal sequence-Light chain-PolyA.

31. The method of any of claims 20 to 30, wherein the mAb is a hyperglycosylated mutant or wherein the Fc polypeptide of the mAb is glycosylated or aglycosylated.

32. The method of claims 20 to 31 wherein the mAb contains an alpha 2,6-sialylated glycan.

33. The method of any of claims 20 to 32 wherein the mAb is glycosylated but does not contain detectable NeuGc and/or α-Gal.

34. The method of any of claims 20 to 32 wherein the mAb contains a tyrosine sulfation.

35. The method of any of claims 20 to 34 in which production of said HuPTM form of said mAb or antigen-binding fragment thereof is confirmed by transducing human liver and/or muscle cells in culture with said recombinant nucleotide expression vector and expressing said mAb or antigen-binding fragment thereof.

36. The method of any of claims 20 to 35, wherein the therapeutically effective amount is determined to be sufficient to reduce HAE attack frequency, reduce progression of angioedema, reduction in coagulation frequency, reduction in thrombosis formation.

37. A method of producing recombinant AAVs comprising:
(a) culturing a host cell containing:
(i) an artificial genome comprising a cis expression cassette flanked by AAV ITRs, wherein the cis expression cassette comprises comprising a transgene encoding a substantially full-length or full-length anti-factor XII mAb, operably linked to one or more regulatory sequences that promote expression of the transgene in human liver and/or muscle cells;
(ii) a trans expression cassette lacking AAV ITRs, wherein the trans expression cassette encodes an AAV rep and an AAV capsid protein operably linked to expression control elements that drive expression of the AAV rep and the AAV capsid protein in the host cell in culture and supply the AAV rep and the AAV capsid protein in trans, wherein the capsid has liver and/or muscle tropism;
(iii) sufficient adenovirus helper functions to permit replication and packaging of the artificial genome by the AAV capsid protein; and
(b) recovering recombinant AAV encapsidating the artificial genome from the cell culture.

38. The method of claim 37, wherein the transgene encodes a substantially full-length or full-length mAb or antigen binding fragment that comprises the heavy and light chain variable domains of garadacimab, AB042/AB043, AB054, DX-4012, Ab26036, Ab26048, Ab260489, Ab26076, 559C-M0071-F06, 559C-M0179-D04, 559C-M0181-C02, 559C-M0180-G03, 559C-M0184-B04, 620I-X0173-A11, 620I-X0173-C07, 620I-X0173-E07, or 620I-X0173-G11.

39. A host cell containing:
a. an artificial genome comprising a cis expression cassette flanked by AAV ITRs, wherein the cis expression cassette comprises comprising a transgene encoding a substantially full-length or full-length anti-Factor XII mAb, operably linked to one or more regulatory sequences that promote expression of the transgene in human liver and/or muscle cells;
b. a trans expression cassette lacking AAV ITRs, wherein the trans expression cassette encodes an AAV rep and an AAV capsid protein operably linked to expression control elements that drive expression of the AAV rep and the AAV capsid protein in the host cell in culture and supply the AAV rep and the AAV capsid protein in trans, wherein the capsid has liver and/or muscle tropism;
c. sufficient adenovirus helper functions to permit replication and packaging of the artificial genome by the AAV capsid protein.

40. The host cell of claim 39, wherein the transgene encodes a substantially full-length or full-length mAb or antigen binding fragment that comprises the heavy and light chain variable domains of garadacimab, AB042/AB043, AB054, DX-4012, Ab26036, Ab26048, Ab260489, Ab26076, 559C-M0071-F06, 559C-M0179-D04, 559C-M0181-C02, 559C-M0180-G03, 559C-M0184-B04, 620I-X0173-A11, 620I-X0173-C07, 620I-X0173-E07, or 620I-X0173-G11.

41. The host cell of claims 39 to 40, wherein the AAV capsid protein is an AAV3B, AAV5, AAV7 (SEQ ID NO:198), AAV8 (SEQ ID NO:199), AAV9 (SEQ ID NO:200), AAVrh10 (SEQ ID NO:201), AAVrh46 (SEQ ID NO:202), AAVrh73 (SEQ ID NO:203), AAVS3 (SEQ ID NO:205), AAV-LK03 (SEQ ID NO:204), AAVrh8, AAV64R1, or AAVhu37 capsid protein.
